(19) United States
(12) Patent Application Publication
(10) Pub. No.: US 2024/0101675 A1
(43) Pub. Date: Mar. 28, 2024

(54) DUAL BINDING ANTIBODIES, METHODS OF PRODUCING DUAL BINDING ANTIBODIES, AND USES THEREOF

(71) Applicant: Biolojic Design Ltd., Rehovot (IL)

(72) Inventors: ALIK DEMISHTEIN, Rosh Haayin (IL); Dotan OMER, Ra'anana (IL); Schmuel BERNSTEIN, Lod (IL); ltay LEVIN, Herzeliya (IL); Yehezkel SASSON, Tzur Yigal (IL); Marek STRAJBL, Jerusalem (IL); Sharon FISCHMAN, Modiin (IL); Guy NIMROD, Tel Aviv (IL); Michael ZEHNIN, Jerusalem (IL); Reut Barak FUCHS, Rehovot (IL); Olga BLUVSHTEIN YERMOLAEV, Rishon-LeZion (IL); Yair FASTMAN, Jerusalem (IL); Oshrat Shir TWITO, Rishon Le Tsiyon (IL); Yanay OFRAN, Tel Aviv (IL)

(73) Assignee: Biolojic Design Ltd., Rehovot (IL)

(21) Appl. No.: 18/262,419

(22) PCT Filed: Jan. 20, 2022
(86) PCT No.: PCT/IL2022/050087

§ 371 Date: Jul. 21, 2023

Related U.S. Application Data
(60) Provisional application No. 63/139,815, filed on Jan. 21, 2021.
(60) Provisional application No. 63/195,021, filed on May 30, 2021.
(60) Provisional application No. 63/295,905, filed on Jan. 2, 2022.

(51) Int. Cl. C07K 16/28

(52) U.S. Cl. CPC C07K 16/2818; C07K 16/2875; C07K 16/2878; C07K 2317/31; C07K 2317/565; C07K 2317/622; C07K 2317/92

(57) ABSTRACT

Described herein are method of generating and uses of polypeptides with dual binding specificity, wherein the polypeptides contain a first binding-site for a first antigen and a second binding-site for a second antigen. The second binding-site comprises amino acid variants of native amino acid sequences of polypeptides that bind to the first antigen, wherein the variants do not abrogate binding to said first antigen. In one embodiment, polypeptides with dual binding specificity to PD1 and OX40 are disclosed herein. In another embodiment, polypeptides with dual binding specificity to PD1 and GITR are disclosed herein.

### SEQUENCE LISTING STATEMENT

The instant application contains a Sequence Listing, which has been submitted electronically in ASCII format and is hereby incorporated by reference in its entirety. Said ASCII copy, created on Jan. 18, 2022, is named P-602267-PC_SL.txt and is 293,460 bytes in size.

### FIELD OF THE INVENTION

The disclosure relates in general to the field of bifunctional antibodies. In one embodiment, the present disclosure describes the making and uses of polypeptides with dual binding specificity.

### BACKGROUND OF THE INVENTION

Immunoglobulins, or antibodies, are monospecific, bivalent antigen-binding molecules (FIG. 1A). Thus, these molecules typically just target a single type of cell or antigen. The therapeutic potential of monoclonal antibodies (mAbs) is enormous but may be limited due to the fact that they are only able to bind one distinct target, while complex diseases in general originate from multiple factors and mediators.

In the last 30 years monoclonal antibodies have been established as one of the main sources of therapeutics. Antibodies in scFV, IgG1, IgG4 and Fab formats have been used for cancer therapy, immunology disorders, infectious diseases and other indications. Like natural antibodies, most of these therapeutic antibodies have specificity to a single antigen, targeting cytokines, membrane receptors, and other molecules of biological importance. The specificity and high affinity of these antibodies can be used to generate several biological effects such as, agonism or antagonism of receptors, blockade of cytokines binding to their cognate receptors, and induction of antibody dependent cellular cytotoxicity ADCC and complement dependent cytotoxicity (CDC).

Results from recent research have suggested that if antibodies could target more than one cell at a time, the efficacy and specificity of the antibody-based therapy would increase. Such bifunctional antibodies, for example, antibodies that can target both tumor cells and cells that destroy the tumor cells, would bring all the right immune system players together at the right place and time to get the job done.

Bispecific antibodies consist of two physically connected antigen binding moieties (i.e., paratopes), which can simultaneously interact with different epitopes on the same or on different antigens. (FIG. 1B) These bispecific antibodies may be produced by biological or chemical methods. Biological production involves fusion of two monoclonal antibody-producing hybridomas. The resulting hybrid hybridomas secrete a mixture of parental monoclonal antibodies and bifunctional antibody. Alternatively, recombinant antibodies may be produced by introducing DNA into a cell, wherein the DNA encodes for two heavy chains and two light chains. Examples of cell types include for example but not limited to a CHO or HEK cell lines. In chemical production, the parental monoclonal antibodies can be broken up and reconstituted to produce the bifunctional antibodies.

Antibodies with two or more specificities have been developed. These antibodies bind two different epitopes of one antigen or two different epitopes on different antigens, and have been generally termed bispecific antibodies. Since bispecific antibodies can bind two different antigens at the same time, they represent promising therapeutic approaches that are not possible using traditional monoclonal antibodies. Bispecific antibodies can bring two targeted antigens into close physical proximity, such as two membrane proteins expressed on different cells, an action being utilized by T cell engager antibodies which direct T cell cytotoxic activity towards cancer cells. An example of this approach is Blinatumomab, a bispecific antibody that activates T cells by agonizing the CD3 T cell co-receptor while bringing the T cells into close proximity of the tumor target by simultaneously binding the tumor cell marker CD19. Another approach that can only be accomplished by bi-specific antibodies is the binding of two different receptors on the same cell which can enhance specificity to the tumor cell and facilitate a more precise antibody-dependent cellular cytotoxicity (ADCC) action. JNJ-372 is an example of such a bispecific that binds both EGFR and cMET on NSCLC cancer cells. Bispecific antibodies have also been used to mimic structural co-factors as in the case of Emicizumab which binds both factor X and factor IX mimicking the function of factor VIII activity.

While bi-specific antibodies (“bispecifics”) have great therapeutic potential, they also face several challenges. Expression of bispecifics as two fused scFV and similar formats is relatively easy, but this format lacks the Fc region and suffers from short half-life in the serum, lack of immune cells Fc mediated engagement, relatively low thermal stability and potential immunogenicity. Bispecifics with classical IgG1 format where the antibody is comprised of two halves of an antibody assembled together (i.e. A type heavy chain and A type light chain connected to an A′ type heavy chain and A′ light chain), do not suffer from these limitations, since in vivo, the various receptors and immune compounds like Fc-gamma receptors, Fc-Rn and the complement system, recognize these bispecifics like natural IgG1. However, production of such bispecifics is complex. Early attempts to produce two antibodies in one cell resulted in only 12.5% bispecific antibodies of the right combination (Aran F. Labrijn, Maarten L. Janmaat et al. Bispecific antibodies: a mechanistic review of the pipeline. Nature Reviews Drug Discovery, 18, 8, 8 (2019)). Since all products have similar biochemical properties, isolation and characterization of the desired product is very complicated.

To overcome these limitations, several methods like knobs into holes (Ridgway J B et al., Knobs-into-holes' engineering of antibody C(H)3 domains for heavy chain heterodimerization. Protein Engineering, 9, 7, 7 (1996)); common light chain (Babb, R et al., US20130045492A1, 2013); SEED (Davis J H et al., SEED bodies: fusion proteins based on strand-exchange engineered domain (SEED) CH3 heterodimers in an Fc analogue platform for asymmetric binders or immunofusions and bispecific antibodies Protein Engineering, Design and Selection, 23, 4, 4 (2010)); and cross Mab (Kienast Y et al., Ang-2-VEGF-A crossmab, a novel bispecific human IgG1 antibody Blocking VEGF-A and Ang-2 functions simultaneously, mediates potent antitumor, antiangiogenic, and antimetastatic efficacy. Clinical Cancer Research, 19, 24, 12 (2013)) were developed. Another widely used method to produce bi-specific antibodies is controlled reduction and oxidation of two antibodies in vitro. Utilizing these methods can reduce the limitations of producing bispecific antibodies to some extent, but the process of developing and producing bispecifics is still much more costly than regular IgGs, requires the development and utilization of extensive antibody analytical inspections for the detection of undesired products, and in some cases, due to mutations in the antibody framework, could be inherently more immunogenic.

Therefore, although bispecific antibodies are promising molecules that might overcome some of the therapeutic limitations experienced with conventional mAbs, the generation of these antibodies is challenging and requires extensive protein-engineering and development of manufacturing process depending on the chosen antibody format. Thus, there remains a need for improved methods of making and using antibodies or polypeptides that have dual binding specificity.

A natural IgG that binds two independent epitopes through its native CDRs could potentially overcome the limitations mentioned above. Such an antibody may bind the two epitopes via two distinct paratopes that may or may not have mutual overlapping residues. As a standard IgG format it would have the ease of production of monoclonal or recombinant IgG, using standard production methods, but it may still be able to bind to different epitopes at the same time, thus having all the potential biological effects of bispecific antibodies mentioned above, or may not bind both antigens at the same time, thus having all the potential biological effects of administering two unrelated therapeutic monospecific antibodies.

### SUMMARY OF THE INVENTION

In one aspect, the present disclosure provides a method of generating polypeptides with dual binding specificity, comprising the steps of:
(a) identifying and providing a first plurality of amino acid sequences from antibodies that bind to a first antigen, said amino acid sequences comprising an identified antigen-binding site binding to said first antigen, said first antigen-binding site comprising variable heavy chain (VH) and variable light chain (VL) domains, each VH and VL domain comprising complementarity determining regions (CDRs) and framework regions (FR), wherein greater than 75% of said CDR positions of the VH or VL domains are non-paratope CDR residues;
(b) identifying continuous surface patches comprising amino acid residues that do not form specific interactions with said first antigen, wherein said amino acid residues provide favorable sites for variant amino acids and where each patch comprises at least one subgroup of amino acid residues forming a continuous surface;
(c) selecting at least one subgroup of amino acid residues comprised within at least one of said patches for introducing one or more amino acid variants; (d) introducing amino acid variants to one or more residues within said one or more said selected subgroups, thereby generating a second plurality of amino acid sequences, each of which comprises the antigen-binding site to said first antigen and said amino acid variants, wherein each amino acid sequence of said second plurality of amino acid sequences comprises between about 2-8 variant amino acid;
(e) generating a high-throughput screening (HTS) library comprising said second plurality of amino acid sequences;
(f) screening said HTS library for binding to said first antigen and binding to a second antigen; and
(g) selecting from said HTS library, candidate polypeptides that preserved binding to said first antigen and confer binding to said second antigen, thereby generating polypeptides with dual binding specificity.

In a related aspect, the patches identified within said first antigen-binding site comprises amino acid residues on a heavy chain variable (VH) region, or a light chain variable (VL) region, or both. In a further related aspect, the patches comprising amino acid residues that do not form specific interactions with said first antigen, comprise one or more amino acid residues in at least one CDR or one or more amino acid residues in at least one framework region (FR) or both. In yet a further related aspect, the one or more amino acid variants is in at least one CDR region. In still a further related aspect, the one or more amino acid variants is within at least one framework region. In another related aspect, the amino acid variants comprise at least two variants, at least one within a CDR region and at least one within a framework region. In still a further related aspect, the patches comprise a set of solvent accessible amino acid residues that are in close proximity. In another further related aspect, the set of solvent accessible amino acid residues that are in close proximity has a length of about 2 to 20 amino acid residues. In yet another further related aspect, the selection of said subgroup of amino acid residues for introducing amino acid variants comprises computational methods or mutational analysis, or a combination thereof.

In another related aspect, identification of the first antigen-binding site comprises one or more of amino acid sequence analysis, structural analysis, mutational analysis, hydrogen-deuterium exchange analysis, computational analysis, or any combination thereof.

In a related aspect, the candidate polypeptides at step (g) comprise polypeptides with dual binding specificity and having at least 800 uM binding affinity for each antigen.

In another related aspect, the method disclosed herein further comprises at least one additional screening step and selecting step following step (g) of said selected candidate polypeptides.

In another related aspect, the method disclosed herein further comprises a maturation affinity step of said candidate polypeptides following step (g), followed by at least one additional screening step and selecting step.

In another related aspect, the binding specificity, binding affinity, or binding avidity of the candidate polypeptides to said first antigen is not reduced by more than about one to three-orders of magnitude after said introduction of amino acid variants. In a further related aspect, the binding specificity, binding affinity, or binding avidity of said candidate polypeptides to said first antigen is not reduced after said introduction of amino acid variants.

In another related aspect, the method further comprises a step expressing candidate polypeptides in the form of an IgG, a single-chain fragment variable (scFv), an Fab, an F(ab′)2, a minibody, a diabody, a triabody, a nanobody, or a single domain antibody. In a further related aspect, the IgG is of the subclass of IgG1, IgG2, IgG3, or IgG4.

In another related aspect, the candidate polypeptides comprising dual binding specificity cannot bind both said first antigen and said second antigen at the same time.

In another related aspect, the candidate polypeptides comprising dual binding specificity that may bind the first antigen and the second antigen at the same time

In another related aspect of the method disclosed herein, the first antigen is selected from the group consisting of PD1, tumor necrosis factor alpha, P-amyloid peptide, CD11a, immunoglobulin E, epidermal growth factor receptor 2, vascular endothelial growth factor A, CD20, nerve growth factor, IL-13, programmed death ligand 1 (PD-L1), and epidermal growth factor receptor. In a further related aspect of the method, the second antigen is selected from the group consisting of OX40, a glucocorticoid-Induced TNFR-Related (GITR) antigen, CTLA4, PDL-1, PD-1, CD25, tumor necrosis factor receptor 2 (TNFR2), VISTA (B7-H5), T cell immunoglobulin and mucin domain-containing protein 3 (TIM3), vascular endothelial growth factor (VEGF), Lymphocyte-activation gene 3 (LAG3), 4-1BB (CD137), DR3 (TNFRSF25), IL-2, and CD3. In still a further related aspect, the first plurality of amino acid sequences comprises one or more sequences set forth in SEQ ID NOs: 3-28.

### BRIEF DESCRIPTION OF THE DRAWINGS

The patent or patent application file contains at least one drawing executed in color. Copies of this patent or patent application publication with color drawing(s) will be provided by the Office upon request and payment of the necessary fee.

The present disclosure of antibodies or polypeptides with dual binding specificity, both as to their generation and method of use, together with objects, features, and advantages thereof, may best be understood by reference to the following detailed description when read with the accompanying drawings in which:

FIGS. 1A-1C present a schematic representation of a WT antibody that can bind one target (FIG. 1A), a schematic representation of a classic bi-specific antibody that can bind two targets (FIG. 1B), and a schematic representation of a dual binding antibody, as disclosed herein, that may be designed to bind different targets under different conditions or to bind different targets dependent on location, or to bind two targets at the same time (FIG. 1C).

FIG. 2 presents a cartoon of one embodiment of a dual antibody functionality, wherein the dual binding antibody is able to provide target dependent release of circulating antigen A (triangles) to tumor tissue expressing antigen B (squares), wherein release of antigen A occurs in the presence of antigen B. This may reduce off tumor effects and toxicity and allow for higher dosing.

FIG. 3 presents a flowchart of one embodiment of a method of generating polypeptides with dual binding specificity.

FIGS. 4A and 4B present FACS analysis of the Dualmab library (described in Example 1) after four rounds of selection, X axis (FITC) indicated level of expression, Y axis (APC) indicates binding to the ligand. FIG. 4A shows the binding profile of the library to 500 nM hGITR. FIG. 4B shows the binding profile of the library to 500 nM hPD-1.

FIG. 5 presents median fluorescence intensity (Em. 660 nM) measurement of yeast clones binding 111 nM PD-1 and 90 nM GITR. The clone numbering along the X-axis represents the clones names as listed in Tables 4 and 6 without the prefix “3222ACGIClone”.

FIG. 6 presents amino sequence alignments of the VH chain between Nivolumab (SEQ ID NO: 15) and the selected PD1/hGITR clones.

FIG. 7 presents amino sequence alignments of the VL chain between Nivolumab (SEQ ID NO: 16) and the selected PD1/hGITR clones.

FIG. 8 presents amino sequence alignments of the VH chain between Nivolumab (SEQ ID NO: 15) and the selected PD1/OX40 clones.

FIG. 9 presents amino sequence alignments of the VL chain between Nivolumab (SEQ ID NO: 16) and the selected PD1/OX40 clones.

FIGS. 10A-10C present size exclusion chromatography profile of the anti GITR-anti PD-1 Dualmabs in hIgG1 format compared to Nivolumab. FIG. 10A shows IgG BDG32.004, FIG. 10B shows IgG BDG32.005, and FIG. 10C shows control starting antibody Nivolumab in IgG1 format.

FIG. 11 presents ELISA binding data for two of the IgG1 dual specific antibodies produced, BDG32.004 and BDG32.005. The results show binding of BDG32.004 and BDG 32.005 towards 100 nM hPD1, 500 nM hIL-2 and 500 nM mTNFR2. Anti hIL-2 was tested against 500 nM hIL-2. Mean 450 nm absorbance and standard deviation of three replicates for PD-1 and two replicates for the other antigens are presented.

FIGS. 12A and 12B present direct ELISA affinity to hGITR and hPD-1. FIG. 12A shows binding of BDG 32.004 and BDG 32.005 to hGITR. FIG. 12B shows binding of Nivolumab, BDG 32.004, and BDG 32.005 to hPD-1. Affinity was calculated with Graphpad nonlinear fit regression with a specific binding regression model.

FIG. 13 presents the results of inhibition of PD-1 signaling of cellular reporter PD1/PDL1 blocking assay. BDG32.005 was tested in comparison with Nivolumab in IgG1 format (positive control) and IgG Isotype (negative control). IC₅₀ was calculated by Graphpad nonlinear fit regression with specific binding regression model.

FIGS. 14A and 14B present FACS analysis of the Dualmab library after four rounds of selection for PD-1 and OX40 binding, X axis (FITC) indicated level of expression, Y axis (APC) indicates binding to ligand. FIG. 14A shows the binding profile of the library to 500 nM hPD-1. FIG. 14B shows the binding profile of the library to 500 nM hOX40.

FIG. 15 presents median fluorescence intensity (Em. 66 nM) of yeast clones binding 30 nM PD-1 and 90 nM OX40. The clones numbering in the X-axis represent the clones name listed in Tables 4 and 6 without the prefix “3212ACOXC”.

FIGS. 16A and 16B present direct ELISA affinity to hOX40 and hPD-1. FIG. 16A shows binding of BDG 32.007 and BDG 32.008 to hOX40. FIG. 16B shows binding of Nivolumab, BDG 32.007, and BDG 32.008 to hPD-1. Affinity was calculated with Graphpad nonlinear fit regression with a specific binding regression model.

FIG. 17 presents inhibition of PD-1 signaling of cellular reporter PD1/PDL1 blocking assay. IC₅₀ was calculated by Graphpad nonlinear fit regression with specific binding regression model.

FIGS. 18A-18B present the template antibody heavy chain (SEQ ID NO: 110) (FIG. 18A) and light chain (SEQ ID NO: 111) amino acid sequences (FIG. 18B), respectively, indicating the framework (FR) and complementarity-determining regions (CDR) regions. For the Heavy (H) chain, the different regions are labeled FR1, CDR1, FR2, CDR2, FR3, CDR3, and FR4, and in some embodiments are referred to as HFR1, HCDR1, HFR2, HCDR2, HFR3, HCDR3, and HFR4. For the Light (L) chain, the different regions are labeled FR1, CDR1, FR2, CDR2, FR3, CDR3, and FR4, and in some embodiments are referred to as LFR1, LCDR1, LFR2, LCDR2, LFR3, LCDR3, and LFR4. Below the template amino acid sequences, the variant amino acids of the engineered dual binding clones are displayed and aligned within the CDR and FR regions.

FIGS. 19A and 19B present bar graphs showing binding of re-epitoped antibodies displayed on yeast to recombinant human IL-13 (rh-IL-13) (FIG. 19A) or recombinant human TSLP (rhTSLP) (FIG. 19B). FIG. 19A shows binding of isolated yeast-surface display anti-IL13 clones to 10 nM rh-IL-13. FIG. 19B shows binding of isolated yeast-surface display anti-TSLP clones to 10 nM rhTSLP. Data was normalized to the yeast surface expression levels of each clone, and to an anti-hIL-13 and anti-hTSLP mean fluorescence intensity (MFI) binding signal of positive control yeast clones.

FIGS. 20A-20F presents size exclusion chromatography (SEC) scans of a human standard IgG1 (FIG. 20A), BDG33.003 (FIG. 20B), BDG33.004 (FIG. 20C), BDG 33.005 (FIG. 20D), BDG33.023 (FIG. 20E), and BDG33.025 (FIG. 20F). The purified IgGs were run on a GE Superdex® 200 10/300 increase (column volume (CV)=25 ml) in PBS buffer at 0.5 ml/min. In the antibody scans shown in FIGS. 20B-20D, the leading peak corresponds to (0.36 CV) that typical of a large aggregate, and a second peak with retention of approximately 13.2 ml (0.528 CV) that is typical of an ordinary human IgG. Area Under the Curve (AUC) peak ratio is approximately 23% misfolded/77% folded IgG fraction, respectively. For the antibody scans shown in FIGS. 20E-20F, the leading peak corresponds to (0.36 CV) that typical of a large diameter aggregate, and a second peak with retention of approximately 13.8 ml (0.55 CV) that is typical of an ordinary human IgG. Area Under the Curve (AUC) peak ratio is 97.3% folded/2.8% misfolded and 98.5% folded/1.5% misfolded for BDG33.023 (FIG. 20E) and BDG33.025 (FIG. 20F) respectively.

FIGS. 21A and 21B present Differential Scanning Fluorimetry (DSF) analysis of the melting point of indicated IgGs BDG33.023 (FIG. 21A) and BDG33.025 (FIG. 21B). Light gray dashed line in the upper graph represents the T-onset and bold gray dashed lines represents the Tm1 and Tm2. The lower graph is the 1st derivative of the measurement. FIG. 21A: DSF of BDG33.023 T-onset of 64.2° C. and first transition point at 67.7° C. FIG. 21B: DSF of BDG33.025 T-onset of 56.4° C., first transition point at 60.9° C. and second transition point at 67.4° C.

FIGS. 22A-22F presents Surface Plasmon Resonance (SPR) analysis of antibodies binding to human IL-13, Cyno IL-13, and human TSLP. Representative SPR sonograms of BDG33.003 and BDG33.004 binding to IL-13 are presented in FIGS. 22A-22D. Recombinant human IL-13 (rh-IL-13) was tested at 800 nM with a 2-fold dilution (FIGS. 22A-22B). Recombinant cyno IL-13 (rc-IL-13) was tested at 200 nM with a 2-fold dilution (FIGS. 22C-22D). Representative SPR sensorgrams of BDG33.003 and BDG33.004 binding to human TSLP (h-TSLP) are presented in FIGS. 22E and 22F. hTSLP served as analyte at concentrations of 3.2 nM to 0.2 nM with two-fold dilutions (FIGS. 22E-22F). Representative SPR sensorgrams of BDG33.023 and BDG33.025 binding to human IL-13 (h-IL-13) are presented in FIGS. 22G and 22H. hIL-13 served as analyte at concentrations of 20 nM to 0.6 nM with tow fold dilutions (FIGS. 22G-222H).

FIGS. 23A-23E present ELISA EC50 binding of BDG33.023 and BDG33.025 to human TSLP, cytomegaly monkey (cyno) TSLP or cytomegaly monkey (cyno) IL-13. Binding of BDG33.023 (filled circles) and BDG33.025 (filled squares) to human TSLP (FIG. 23A-human TSLP). Binding of BDG33.023 to cyno-TSLP (FIGS. 23B-33.023 cyno TSLP). Binding of BDG33.025 to cyno-TSLP (FIGS. 23C-33.025 cyno TSLP). Binding of BDG33.023 to cyno-IL-13 (FIGS. 23D-33.023 cyno IL-13). Binding of BDG33.025 to cyno-IL-13 (FIGS. 23E-33.025 cyno IL-13).

FIGS. 24A-24D present competitive binding assay of antibodies to hTSLP or hIL-13. FIG. 24A: Indicated antibodies (anti-TSLP-control; anti-IL-13 control; BDG33.023; BDG33.025) were pre-incubated with increasing levels of hIL-13 and added to a plate that was pre-coated with hIL-13. BDG33.023 and BDG33.025 binding to plate-bound hIL-13 was inhibited as soluble hIL-13 concentration increased. FIG. 24B: Indicated antibodies (anti-TSLP-control; anti-IL-13 control; BDG33.023; BDG33.025) were pre-incubated with increasing levels of hTSLP and added to a plate that was pre-coated with hTSLP. BDG33.023 and BDG33.025 binding to plate-bound hTSLP was inhibited as soluble hTSLP concentration increased. FIG. 24C: Antibodies (anti-TSLP-control; anti-IL-13 control; BDG33.023; BDG33.025) were pre-incubated with increasing levels of hTSLP and added to a plate pre-coated with hIL-13, BDG33.023 binding to IL-13 was inhibited as soluble hTSLP concentration increased. FIG. 24D: Indicated antibodies (anti-TSLP-control; anti-IL-13 control; BDG33.023; BDG33.025) were pre-incubated with increasing levels of hIL-13 and added to a plate pre-coated with hTSLP. BDG33.023 binding to plate-bound hTSLP was inhibited as soluble hIL-13 concentration increased. Anti-TSLP and anti-IL-13 control antibodies only showed binding with their respective ligands, and only competed with their respective ligands.

FIG. 25 presents the results of an ELISA specificity test that compared non-specific binding to specific binding of BDG330.23 and BDG33.025. The ELISA plate was coated with hIL-13, hTSLP, and the non-related cytokines IL-2, IL-17, and IL-4. BSA binding signal corresponds to assay background level.

FIG. 26 presents the results of an IC50 inhibition assay that measured IgG specific blocking of hTSLP from binding to an ELISA plate coated with TSLP receptor (TSLP-R). The X-axis represents concentration of competitor. Competitors: TSLP-R (black circles) with a resultant IC50=3 nM; and BGD33.023 (black triangles) with a resultant IC50=0.41 nM.

FIG. 27 presents a schematic representation of the HEK-Blue IL-13 system downstream signaling.

FIGS. 28A-28D present hIL-13 pSTAT6 signaling inhibition data. The results are based on stimulation of HEK-Blue cell's IL-13 activation pathway by recombinant rh-IL-13 and inhibition of this stimulation by indicated IgGs. HEK-Blue IL-13 cells (50,000 cells/well) were incubated with rh-IL-13 at a range of concentrations (0 nM-8 nM). IL-13 downstream signaling was quantified with QUANTI-Blue 24 h post incubation (FIG. 28A). hIL-13 downstream inhibition on HEK-BLUE IL-13 cells by engineered dual binding antibodies was analyzed as follows. rh-IL-13 (0.4 nM) was incubated with indicated antibodies at an antibody concentration range of 0 nM-750 nM. Antibodies assayed were BDG33.002 (positive control), BDG33.003 (Clone C2), and BDG33.006 (negative control), respectively (FIG. 28B). Clones BDG33.023 and BDG33.025 were assayed at an antibody concentration range of 0 nM-100 nM (FIGS. 28C and 28D show 33.023 IL-13 pSTAT6 inhibition, and 33.025 IL-13 pSTAT6 inhibition, respectively. After the incubation the hIL-13/IgG mixture was added to the cells, secreted embryonic alkaline phosphatase (SEAP) activity was quantified with QUANTI-Blue 24 h post incubation. Data shown is the mean of triplicate experiments, and error bars represent standard deviation.

FIGS. 29A-29C present TSLP signaling pathway inhibition data. TSLP dependent pSTAT5 signaling activation pathway, and inhibition of the this activation by BDG 33.023 in human leukemia MUTZ5 cells. FIG. 29A shows flow-cytometry analysis of MUTZ 5 CD127 (IL-7a) receptor and TSLP-R receptor expression, as follows. Unstained cells (panel a), cells stained for CD127+ wherein approximately 36% of the total cell population was labeled (panel b), cells stained for TSLP-R+, wherein approximately 96% of the total cell population was labeled (panel c), and cells stained for both TSLP-R+ and CD127+ wherein approximately 41% of total cell population was labeled (panel d) (FIG. 29A). FIG. 29B shows MUTZ5 pSTAT5 activation. EC50 of hTSLP phosphor-STAT5 (pSTAT5) activation in MUTZ5 cells. Percent (%) positive cells represents pSTAT5 positive cells as a percentage of the parent population. FIG. 29C shows inhibition of MUTZ5 pSTAT5 activation. IC50 of BDG33.023 inhibition of TSLP dependent pSTAT5 activation in MUTZ5 cells. TSLP was pre-incubated for 30 min with 0.48 pM to 500 pM of BDG33.023 and added to MUTZ5 cells. Positive cells are representing pSTAT5 positive population as a percentage of the parent population (FIG. 29C).

### DETAILED DESCRIPTION OF THE INVENTION

In the following detailed description, numerous specific details are set forth in order to provide a thorough understanding of the dual binding specific antibodies disclosed herein, and the methods of generating these dual binding specific antibodies. However, it will be understood by those skilled in the art that preparation and uses of antibodies with dual binding specificity may in certain cases be practiced without these specific details. In other instances, well-known methods, procedures, and components have not been described in detail so as not to obscure the disclosure presented herein.

Disclosed herein are methods of generating polypeptides with dual binding specificity from antibodies or fragments thereof that comprise an antigen binding region known to bind a first antigen of interest and not bind a second antigen of interest, and the dual binding peptides generated. Each antigen binding region typically recognizes and binds to a single epitope on its target antigen.

Antigen binding sequences are conventionally located within the heavy chain and light chain variable regions of an antibody. These heavy and light chain variable regions may, in certain instances, be manipulated to create new binding sites, for example to create antibodies or fragments thereof, that bind to a different antigen or to a different epitope of the same antigen. In some embodiments, as described herein, manipulating the sequences of a heavy chain variable region or the sequences of a light chain variable region, or both, would create a new binding site for a second antigen while maintaining the original antibody's specific binding to a first antigen.

As used herein, the term “antibody” may be used interchangeably with the term “immunoglobulin”, having all the same qualities and meanings. An antibody binding domain or an antigen binding site can be a fragment of an antibody or a genetically engineered product of one or more fragments of the antibody, which fragment is involved in specifically binding with the antigen. By “specifically binding” is meant that the binding is selective for the antigen of interest and can be discriminated from unwanted or nonspecific interactions.

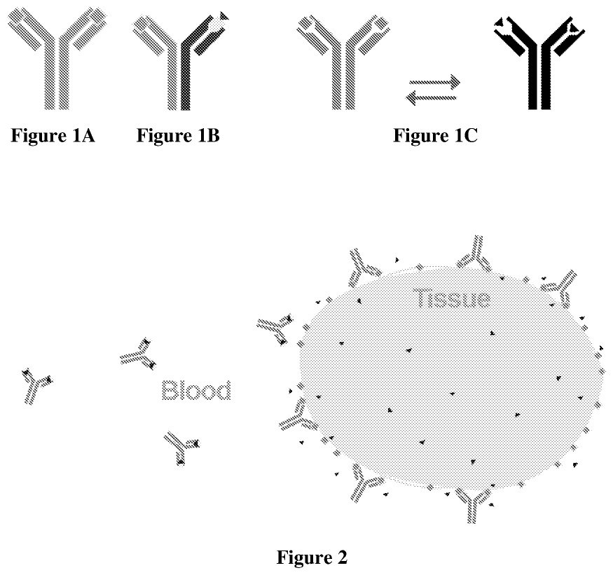

A skilled artisan would appreciate that a dual binding antibody encompasses in its broadest sense an antibody that specifically binds a native antigenic determinant of a first antigen and a second antigen that it previously did not bind to. (FIG. 1C) The skilled artisan would further appreciate that specificity for binding to a first antigen or a second antigen reflects that the binding is selective for each of these antigens and can be discriminated from unwanted or nonspecific interactions. In certain embodiments, the dual binding antibody comprises an antibody fragment or fragments.

As will be described, dual binding antibodies disclosed herein, may be computationally designed fully human IgG antibodies that maintain a natural symmetrical format but can precisely bind to specific epitopes two antigenic targets. In some embodiments, a dual binding antibody binds the first antigen and a second antigen at the same time. In other embodiments, a dual binding antibody cannot bind a first antigen and a second antigen at the same time (FIG. 1C), In some embodiments, a dual binding antibody cannot bind a first antigen and a second antigen at the same time either because of a shared paratope, or because paratopes are close and the antigens cross block each other because of structural interference. In some embodiments, binding of a dual binding antibody to either a first antigen or a second antigen or both is governed by antigen availability and the binding constant of the antibody towards the antigens. In some embodiments, differential binding to a first antigen or a second antigen relates to differences in affinity of the dual binding antibody for each of the target antigens, as well as local physiological antigen availability.

In certain embodiments, a dual binding antibody may bind differentially to their targets under different physiological conditions, for example FIG. 2 illustrates one embodiment of a dual binding antibody described herein designed to bind antigen A and antigen B, wherein the antibody displays targeted dependent release of an antigen A (triangles) in the presence of antigen B (squares). Targeted dependent release of one antigen in the presence of a second antigen may be used to reduce “off” tumor effects and or toxicity, and may allow for higher dosing In some embodiments, a dual binding antibody may bind different targets dependent on the localization of the antibody.

An antigenic determinant on each of these antigens comprises a first epitope or a second epitope, respectively. The term “epitope” includes any determinant, in certain embodiments, a polypeptide determinant, capable of specific binding to an anti-first antigen or anti-second antigen binding domain. An epitope is a region of an antigen that is bound by an antibody or an antigen-binding fragment thereof. In some embodiments, the antigen-binding fragment of an antibody comprises a heavy chain variable region, a light chain variable region, or a combination thereof as described herein.

In certain embodiments, epitope determinants include chemically active surface groupings of molecules such as amino acids, sugar side chains, phosphoryl or sulfonyl, and may in certain embodiments having specific three-dimensional structural characteristics, and/or specific charge characteristics. In certain embodiments, the dual binding antibody is said to specifically bind a first antigen or a second antigen epitope when it preferentially recognizes the first or second antigen in a complex mixture of proteins and/or macromolecules.

In some embodiments, the dual binding antibody is said to specifically bind an epitope when the equilibrium dissociation constant is ≤10⁻⁵, 10⁻⁶, or 10⁻⁷ M. In some embodiments, the equilibrium dissociation constant may be ≤10⁻⁸ M or 10⁻⁹ M. In some further embodiments, the equilibrium dissociation constant may be ≤10⁻¹⁰ M, 10⁻¹¹ M, or 10⁻¹²M. In some embodiments, the equilibrium dissociation constant may be in the range of ≤10⁻⁵ M to 10⁻¹²M.

As used herein, the term “antibody” encompasses an antibody fragment or fragments that retain binding specificity including, but not limited to, IgG, variable heavy chain (VH) fragments, variable light chain (VL) fragments, Fab fragments, F(ab′)₂ fragments, scFv fragments, Fv fragments, a nanobody, minibodies, diabodies, triabodies, tetrabodies (see, e.g., Hudson and Souriau, Nature Med. 9: 129-134 (2003) (hereby incorporated by reference in their entirety), and single domain antibodies. Also encompassed are humanized, primatized, and chimeric antibodies.

In certain embodiment, the dual specific antibody comprises the form of an IgG, a single-chain fragment variable (scFv), a Fab, a F(ab′)2, a minibody, a diabody, a triabody, a nanobody, or a single domain antibody. In some embodiments, an IgG comprises a subclass selected from IgG1, IgG2, IgG3, or IgG4.

In some embodiments, a first plurality of amino acid sequence from a plurality of antibodies comprises sequences from therapeutic antibodies, for example but not limited to the antibodies presented in Table 1 below. In some embodiments, a first plurality of amino acid sequence from a plurality of antibodies comprises sequences from any collection of antibody structures. Examples of these antibody structures include, but are not limited to, the antibody structures disclosed in the PDB database (https://www.rcsb.org/) or the SabDab database (http://opig.stats.ox.ac.uk/webapps/newsabdab/sabdab/).

As used herein, the term “heavy chain variable region” may be used interchangeably with the term “VH domain” or the term “VH”, having all the same meanings and qualities. As used herein, the term “light chain variable region” may be used interchangeably with the term “VL domain” or the term “VL”, having all the same meanings and qualities. A skilled artisan would recognize that a “heavy chain variable region” or “VH” with regard to an antibody encompasses the fragment of the heavy chain that contains three complementarity determining regions (CDRs) interposed between flanking stretches known as framework regions. The framework regions are more highly conserved than the CDRs, and form a scaffold to support the CDRs. Similarly, a skilled artisan would also recognize that a “light chain variable region” or “VL” with regard to an antibody encompasses the fragment of the light chain that contains three CDRs interposed between framework regions.

In certain embodiments, the first antigen binding sites, as described herein, include a heavy chain and a light chain CDR set, respectively, interposed between a heavy chain and a light chain framework region (FR) set which provide support to the CDRs and define the spatial relationship of the CDRs relative to each other.

As used herein, the term “CDR set” refers to the three hypervariable regions of a heavy or light chain variable region. Proceeding from the N-terminus of a heavy or light chain polypeptide, these regions are denoted as “CDR1,” “CDR2,” and “CDR3” respectively. In one embodiment, a first antigen-binding site includes six CDRs, comprising the CDR set from each of a heavy and a light chain variable region. Crystallographic analysis of a number of antigen-antibody complexes has demonstrated that the amino acid residues of CDRs form extensive contact with a bound antigen, wherein the most extensive antigen contact is with the heavy chain CDR3. Thus, the CDR regions are primarily responsible for the specificity of a first antigen-binding site. CDR regions may form structural surface in three-dimension (3D), wherein the structure formed for specifically binding an antigen comprises amino acid residues of more than one CDR region.

As used herein, the term “FR set” refers to the four flanking amino acid sequences which frame the CDRs of a CDR set of a heavy or light chain variable region. Some FR residues may contact bound antigen; however, FRs are primarily responsible for folding the variable region into the antigen-binding site, in this case the first antigen binding site, and following addition of variant amino acid residues, the second antigen binding site. In some embodiments, the FR residues responsible for folding the variable regions comprise the FR residues directly adjacent to the CDRs. Within FRs, certain amino residues and certain structural features are very highly conserved. In this regard, all variable region sequences contain an internal disulfide loop of around 90 amino acid residues. When the variable regions fold into a binding-site, the CDRs are displayed as projecting loop motifs which form an antigen-binding surface. It is generally recognized that there are conserved structural regions of FRs, which influence the folded shape of the CDR loops into certain “canonical” structures regardless of the precise CDR amino acid sequence. Further, certain FR residues are known to participate in non-covalent interdomain contacts which stabilize the interaction of the antibody heavy and light chains.

Wu and Kabat (Tai Te Wu, Elvin A. Kabat. An analysis of the sequences of the variable regions of bence jones proteins and myeloma light chains and their implications for antibody complementarity. Journal of Experimental Medicine, 132, 2, 8 (1970); Kabat E A, Wu TT, Bilofsky H, Reid-Miller M, Perry H. Sequence of proteins of immunological interest. Bethesda: National Institute of Health; 1983. 323 (1983)) pioneered the alignment of antibody peptide sequences, and their contributions in this regard were several-fold: First, through study of sequence similarities between variable domains, they identified correspondent residues that to a greater or lesser extent were homologous across all antibodies in all vertebrate species, inasmuch as they adopted similar three-dimensional structure, played similar functional roles, interacted similarly with neighboring residues, and existed in similar chemical environments. Second, they devised a peptide sequence numbering system in which homologous immunoglobulin residues were assigned the same position number. One skilled in the art can unambiguously assign what is now commonly called Kabat numbering, to any variable domain sequence, without reliance on any experimental data beyond the sequence itself. Third, for each Kabat-numbered sequence position, Kabat and Wu calculated variability, by which is meant the finding of few or many possible amino acids when variable domain sequences are aligned. They identified three contiguous regions of high variability embedded within four less variable contiguous regions. Kabat and Wu formally demarcated residues constituting these variable tracts, and designated these “complementarity determining regions” (CDRs), referring to chemical complementarity between antibody and antigen. A role in three-dimensional folding of the variable domain, but not in antigen recognition, was ascribed to the remaining less-variable regions, which are now termed “framework regions”. Fourth, Kabat and Wu established a public database of antibody peptide and nucleic acid sequences, which continues to be maintained and is well known to those skilled in the art.

Chothia and coworkers (Cyrus Chothia, Arthur M. Lesk. Canonical structures for the hypervariable regions of immunoglobulins. Journal of Molecular Biology, 196, 4, 8 (1987)) found that certain sub portions within Kabat CDRs adopt nearly identical peptide backbone conformations, despite having great diversity at the level of amino acid sequence. These sub portions were designated as L1, L2 and L3 or H1, H2 and H3 where the “L” and the “H” designates the light chain and the heavy chains regions, respectively. These regions may be referred to as Chothia CDRs, which have boundaries that overlap with Kabat CDRs.

More recent studies have shown that virtually all antibody binding residues fall within regions of structural consensus. (Kunik, V. et αι, PloS Computational Biology 8(2):e1002388 (February 2012)) (hereby incorporated by reference in its entirety). In some embodiments, these regions are referred to as antibody binding regions. It was shown that these regions can be identified from the antibody sequence as well. “Paratome”, an implementation of a structural approach for the identification of structural consensus in antibodies, was used for this purpose. (Ofran, Y. et al., J. Immunol. 757:6230-6235 (2008)) (hereby incorporated by reference in its entirety). While residues identified by Paratome cover virtually all the antibody binding sites, the CDRs (as identified by the commonly used CDR identification tools) miss significant portions of them. Antibody binding residues, which were identified by Paratome but were not identified by any of the common CDR identification methods are referred to as Paratome-unique residues. Similarly, antibody binding residues that are identified by any of the common CDR identification methods but are not identified by Paratome are referred to as CDR-unique residues. Paratome-unique residues make crucial energetic contribution to antibody-antigen interactions, while CDRs-unique residues have a rather minor contribution. These results allow for better identification of antigen binding sites.

IMGT® is the international ImMunoGeneTics information System®, (See, Nucleic Acids Res. 2015 January; 43 (Database issue):D413-22. doi: 10.1093/nar/gku1056. Epub 2014 Nov. 5 Free article. PMID: 25378316 LIGM:441 and Dev Comp Immunol. 2003 January; 27(1):55-77). IMGT is a unique numbering system for immunoglobulin and T cell receptor variable domains and Ig superfamily V-like domains, (Lefranc MP1, Pommid C, Ruiz M, Giudicelli V, Foulquier E, Truong L, Thouvenin-Contet V, Lefranc G. Dev Comp Immunol 27: 55-77. (2003)). IMGT® presents a uniform numbering system for these IG and TcR variable domain sequences, based on aligning 5 or more IG and TcR variable region sequences, taking into account and combining the Kabat definition of FRs and CDRs, structural data, and Chothia's characterization of the hypervariable loops. IMGT is considered a universal numbering scheme for antibodies well known in the art.

In some embodiments, identification of potential variant amino acid positions in the VH and VL domains, uses an IMGT system of analysis. In some embodiments, identification of potential variant amino acid positions in the VH and VL domains, uses a Paratome system of analysis. In some embodiments, identification of potential variant amino acid positions in the VH and VL domains, uses a Kabat system of analysis. In some embodiments, identification of potential variant amino acid positions in the VH and VL domains, uses a Clothia system of analysis.

In some embodiments, potential variant amino acid positions are located within continuous stretches of surface (patches) as formed by the 3D structure of amino acid residues of a VH or VL domain. In some embodiments, a continuous surface patch comprises amino acid residues that do not form specific interactions with the native antigen. In some embodiments, a continuous surface patch comprises amino acid residues all of which do not form specific interactions with the native antigen.

Specific interactions occur when amino acid residues are considered “close” or “connected”, wherein residues are considered “close” if there the minimal distance between their heavy atoms is less than 5 Angstroms. Distances are always measured from the heavy atoms (all atoms excluding the hydrogens) of the antigen to the heavy atoms of the antibody. The terms “close”, “close proximity”, and “connected” and the like may in some embodiments, be used interchangeably herein, having all the same qualities and meanings. One skilled in the art would appreciate that lack of a specific interaction occurs when the amino acid residue or residues heavy atoms are structurally separated from the antigen heavy atoms by more than 5 Angstroms.

In some embodiments, continuous surface residue patches are considered to not form a specific interaction when amino acid residues within the patch are structurally separated by more than 5 Angstroms from the native antigen. Based on this lack of specific interaction it could be predicted that amino acid residues within such a patch may be changed, i.e., substituted with a variant amino acid, without abrogating binding to the native antigen.

In describing variant amino acid positions present in the VH and VL domains, in some embodiments the IMGT numbering is used. In describing variant amino acid positions present in the VH and VL domains, in some embodiments the Paratome numbering is used. In describing variant amino acid positions present in the VH and VL domains, in some embodiments the Kabat numbering is used. In describing variant amino acid positions present in the VH and VL domains, in some embodiments the Clothia numbering is used.

Methods of Generating Polypeptides with Dual Specificity

In some embodiments, the present disclosure provides a method of generating polypeptides with dual binding specificity, comprising the steps of:
(a) identifying and providing a first plurality of amino acid sequences from antibodies that bind to a first antigen, said amino acid sequences comprising an identified antigen-binding site binding to said first antigen, said first antigen-binding site comprising variable heavy chain (VH) and variable light chain (VL) domains, each VH and VL domain comprising complementarity determining regions (CDRs) and framework regions (FR), wherein greater than 75% of said CDR positions of the VH or VL domains are non-paratope CDR residues;
(b) identifying continuous surface patches comprising amino acid residues that do not form specific interactions with said first antigen, wherein said amino acid residues provide favorable sites for variant amino acids and wherein each patch comprises at least one subgroup of amino acid residues forming a continuous surface;
(c) selecting at least one subgroup of amino acid residues comprised within at least one of said patches for introducing one or more amino acid variants;
(d) introducing amino acid variants to one or more residues within said one or more said selected subgroups, thereby generating a second plurality of amino acid sequences, each of which comprises the antigen-binding site to said first antigen and said amino acid variants, wherein each amino acid sequence of said second plurality of amino acid sequences comprises between about 2-8 variant amino acids;
(e) generating a high-throughput screening (HTS) library comprising a second plurality of amino acid sequences;
(f) screening said HTS library for binding to said first antigen and binding to said second antigen; and
(g) selecting from said HTS library, candidates polypeptides that preserved binding to said first antigen and confer binding to said second antigen, thereby generating polypeptides with dual binding specificity.

In certain embodiments, said first plurality of amino acid sequences from antibodies, specifically bind to a first antigen and do not specifically bind to a second antigen of interest. In some embodiments, the first antigen comprises the antibody's native antigen.

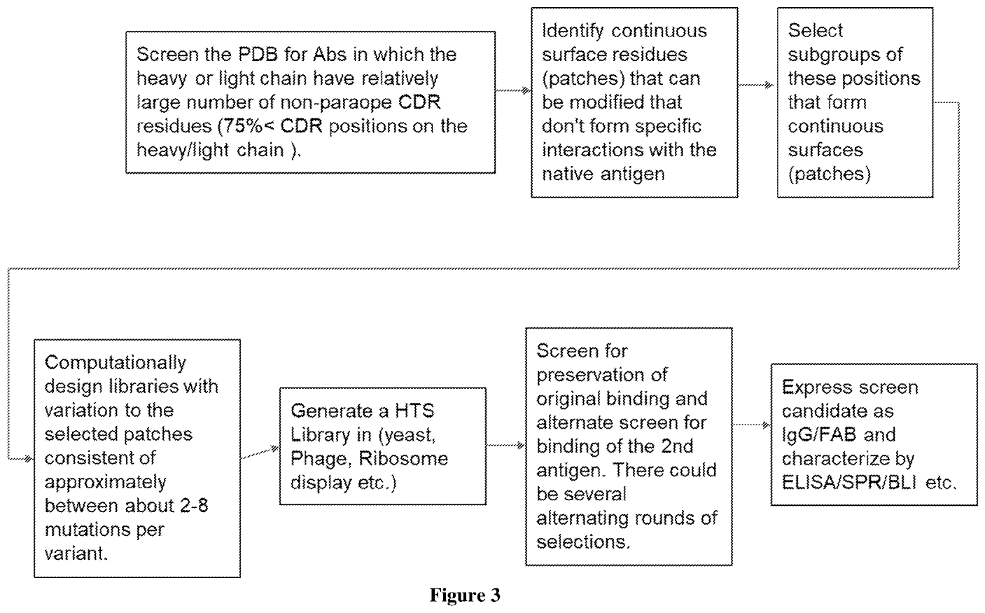

FIG. 3 presents one embodiment of a method of generating polypeptides with dual specificity.

In some embodiments, a step of identifying and providing a first plurality of amino acid sequences comprises screening an antibody library, for example but not limited to the PDB database or the SabDab database, and selecting those antibodies wherein the antigen binding site that binds said first antigen has a relatively large number of non-paratope CDR residues (FIG. 3). In some embodiments, the antigen binding site comprises a VH or VL or both. In some embodiments, identifying and providing a first plurality of amino acid sequences comprises screening an antibody library, and selecting those antibodies wherein the antigen binding site comprising a VH or VL or both comprises greater than 75% non-paratope CDR amino acid residues.

A skilled artisan would appreciate that an antibody library may comprise antibodies or antigen-binding fragments thereof.

In some embodiments, a paratope comprises the amino acids of an antigen binding site that specifically interacts with the native antigen. In some embodiments, a paratope comprises amino acid residues present with the CDRs and or FR residues of an antigen binding site. In some embodiments, a paratope is a 3D structure formed by amino acid residues within the antigen binding site. In some embodiments, a paratope is formed by the 3-D conformation adopted by the interaction of discontiguous amino acid residues. In some embodiments, specific interaction of an amino acid within a paratope comprises structurally being within 5 Angstroms of the antigen. Many antibodies do not utilize all of the amino acid residues of all 6 CDRs within the antigen binding site, to form the paratope. Thus, there are non-paratope CDR amino acid residues. In some embodiments, a non-paratope amino acid residue does not specifically interact with the native antigen. In some embodiments, a non-paratope amino acid residue is structurally separated by more than 5 Angstroms from the native antigen. In some embodiments, a non-paratope amino acid residue may be changed without abrogating binding to the native antigen.

In some embodiments, a first antigen is selected from the group consisting of PD1, tumor necrosis factor alpha, β-amyloid peptide, CD11a, immunoglobulin E, epidermal growth factor receptor 2, vascular endothelial growth factor A, CD20, nerve growth factor, IL-13, programmed death ligand 1 (PD-L1), and epidermal growth factor receptor. In some embodiments, the first plurality of amino acid sequences provided in a method of generating polypeptides with dual binding specificity, comprises at least one of the amino acid sequences set forth in any one of SEQ ID NO: 3 through SEQ ID NO: 28.

TABLE 1

| | | SEQ | | SEQ |
|---|---|---|---|---|
| | VH | ID | VL | ID |
| Name | sequence | NO: | sequence | NO: |
| Bapineuzumab | EVQLLESGGG | 3 | DVVMTQSPLS | 4 |
| | LVQPGGSLRL | | LPVTPGEPAS | |
| | SCAASGFTFS | | ISCKSSQSLL | |
| | NYGMSWVRQA | | DSDGKTYLNW | |
| | PGKGLEWVAS | | LLQKPGQSPQ | |
| | IRSGGGRTYY | | RLIYLVSKLD | |
| | SDNVKGRFTI | | SGVPDRFSGS | |
| | SRDNSKNTLY | | GSGTDFTLKI | |
| | LQMNSLRAED | | SRVEAEDVGV | |
| | TAVYYCVRYD | | YYCWQGTHFP | |
| | HYSGSSDYWG | | RTFGQGTKVE | |
| | QGTLVTVSS | | IK | |
| Durvalumab | EVQLVESGGG | 5 | EIVLTQSPGT | 6 |
| | LVQPGGSLRL | | LSLSPGERAT | |
| | SCAASGFTFS | | LSCRASQRVS | |
| | RYWMSWVRQA | | SSYLAWYQQK | |
| | PGKGLEWVAN | | PGQAPRLLIY | |
| | IKQDGSEKYY | | DASSRATGIP | |
| | VDSVKGRFTI | | DRESGSGSGT | |
| | SRDNAKNSLY | | DFTLTISRLE | |
| | LQMNSLRAED | | PEDFAVYYCQ | |
| | TAVYYCAREG | | QYGSLPWTFG | |
| | GWFGELAFDY | | QGTKVEIK | |
| | WGQGTLVTVS | | | |
| | S | | | |
| Efalizumab | EVQLVESGGG | 7 | DIQMTQSPSS | 8 |
| | LVQPGGSLRL | | LSASVGDRVT | |
| | SCAASGYSFT | | ITCRASKTIS | |
| | GHWMNWVRQA | | KYLAWYQQKP | |
| | PGKGLEWVGM | | GKAPKLLIYS | |
| | IHPSDSETRY | | GSTLQSGVPS | |
| | NQKFKDRFTI | | RFSGSGSGTD | |
| | SVDKSKNTLY | | FTLTISSLQP | |
| | LQMNSLRAED | | EDFATYYCQO | |
| | TAVYYCARGI | | HNEYPLTFGQ | |
| | YFYGTTYFDY | | GTKVEIK | |
| | WGQGTLVTVS | | | |
| | S | | | |
| Golimumab | QVQLVESGGG | 9 | EIVLTQSPAT | 10 |
| | VVQPGRSLRL | | LSLSPGERAT | |
| | SCAASGFIFS | | LSCRASQSVY | |
| | SYAMHWVRQA | | SYLAWYQQKP | |
| | PGNGLEWVAF | | GQAPRLLIYD | |
| | MSYDGSNKKY | | ASNRATGIPA | |
| | ADSVKGRFTI | | RFSGSGSGTD | |
| | SRDNSKNTLY | | FTLTISSLEP | |
| | LQMNSLRAED | | EDFAVYYCQQ | |
| | TAVYYCARDR | | RSNWPPFTFG | |
| | GIAAGGNYYY | | PGTKVDIK | |
| | YGMDVWGQGT | | | |
| | TVTVSS | | | |
| Lebrikizumab | QVTLRESGPA | 11 | DIVMTQSPDS | 12 |
| | LVKPTQTLTL | | LSVSLGERAT | |
| | TCTVSGFSLS | | INCRASKSVD | |
| | AYSVNWIRQP | | SYGNSFMHWY | |
| | PGKALEWLAM | | QQKPGQPPKL | |
| | IWGDGKIVYN | | LIYLASNLES | |
| | SALKSRLTIS | | GVPDRFSGSG | |
| | KDTSKNQVVL | | SGTDFTLTIS | |
| | TMTNMDPVDT | | SLQAEDVAVY | |
| | ATYYCAGDGY | | YCQQNNEDPR | |
| | YPYAMDNWGQ | | TFGGGTKVEI | |
| | GSLVTVSS | | K | |
| Matuzumab | QVQLVQSGAE | 13 | DIQMTQSPSS | 14 |
| | VKKPGASVKV | | LSASVGDRVT | |
| | SCKASGYTFT | | ITCSASSSVT | |
| | SHWMHWVRQA | | YMYWYQQKPG | |
| | PGQGLEWIGE | | KAPKLLIYDT | |
| | FNPSNGRTNY | | SNLASGVPSR | |
| | NEKFKSKATM | | FSGSGSGTDY | |
| | TVDTSTNTAY | | TFTISSLQPE | |
| | MELSSLRSED | | DIATYYCQQW | |
| | TAVYYCASRD | | SSHIFTFGQG | |
| | YDYDGRYFDY | | TKVEIK | |
| | WGQGTLVTVS | | | |
| | S | | | |
| Nivolumab | QVQLVESGGG | 15 | EIVLTQSPAT | 16 |
| | VVQPGRSLRL | | LSLSPGERAT | |
| | DCKASGITFS | | LSCRASQSVS | |
| | NSGMHWVRQA | | SYLAWYQQKP | |
| | PGKGLEWVAV | | GQAPRLLIYD | |
| | IWYDGSKRYY | | ASNRATGIPA | |
| | ADSVKGRFTI | | RFSGSGSGTD | |
| | SRDNSKNTLF | | FTLTISSLEP | |
| | LQMNSLRAED | | EDFAVYYCQQ | |
| | TAVYYCATND | | SSNWPRTFGQ | |
| | DYWGQGTLVT | | GTKVEIK | |
| | VSS | | | |
| Omalizumab | EVQLVESGGG | 17 | DIQLTQSPSS | 18 |
| | LVQPGGSLRL | | LSASVGDRVT | |
| | SCAVSGYSIT | | ITCRASQSVD | |
| | SGYSWNWIRQ | | YDGDSYMNWY | |
| | APGKGLEWVA | | QQKPGKAPKL | |
| | SITYDGSTNY | | LIYAASYLES | |
| | NPSLKGRITI | | GVPSRFSGSG | |
| | SRDDSKNTFY | | SGTDFTLTIS | |
| | LQMNSLRAED | | SLQPEDFATY | |
| | TAVYYCARGS | | YCQQSHEDPY | |
| | HYFGHWHFAV | | TFGQGTKVEI | |
| | WGQGTLVTVS | | K | |
| | S | | | |
| Pertuzumab | EVQLVESGGG | 19 | DIQMTQSPSS | 20 |
| | LVQPGGSLRL | | LSASVGDRVT | |
| | SCAASGFTFT | | ITCKASQDVS | |
| | DYTMDWVRQA | | IGVAWYQQKP | |
| | PGKGLEWVAD | | GKAPKLLIYS | |
| | VNPNSGGSIY | | ASYRYTGVPS | |
| | NQRFKGRFTL | | RFSGSGSGTD | |
| | SVDRSKNTLY | | FTLTISSLQP | |
| | LQMNSLRAED | | EDFATYYCQQ | |
| | TAVYYCARNL | | YYIYPYTFGQ | |
| | GPSFYFDYWG | | GTKVEIK | |
| | QGTLVTVSS | | | |
| Ranibizumab | EVQLVESGGG | 21 | DIQLTQSPSS | 22 |
| | LVQPGGSLRL | | LSASVGDRVT | |
| | SCAASGYDFT | | ITCSASQDIS | |
| | HYGMNWVRQA | | NYLNWYQQKP | |
| | PGKGLEWVGW | | GKAPKVLIYF | |
| | INTYTGEPTY | | TSSLHSGVPS | |
| | AADFKRRFTF | | RFSGSGSGTD | |
| | SLDTSKSTAY | | FTLTISSLQP | |
| | LQMNSLRAED | | EDFATYYCQQ | |
| | TAVYYCAKYP | | YSTVPWTFGQ | |
| | YYYGTSHWYF | | GTKVEIK | |
| | DVWGQGTLVT | | | |
| | VSS | | | |
| Rituximab | QVQLQQPGAE | 23 | QIVLSQSPAI | 24 |
| | LVKPGASVKM | | LSASPGEKVT | |
| | SCKASGYTFT | | MTCRASSSVS | |
| | SYNMHWVKQT | | YIHWFQQKPG | |
| | PGRGLEWIGA | | SSPKPWIYAT | |
| | IYPGNGDTSY | | SNLASGVPVR | |
| | NQKFKGKATL | | FSGSGSGTSY | |
| | TADKSSSTAY | | SLTISRVEAE | |
| | MQLSSLTSED | | DAATYYCQQW | |
| | SAVYYCARST | | TSNPPTFGGG | |
| | YYGGDWYFNV | | TKLEIK | |
| | WGAGTTVTVS | | | |
| | A | | | |
| Tanezumab | QVQLQESGPG | 25 | DIQMTQSPSS | 26 |
| | LVKPSETLSL | | LSASVGDRVT | |
| | TCTVSGFSLI | | ITCRASQSIS | |
| | GYDLNWIRQP | | NNLNWYQQKP | |
| | PGKGLEWIGI | | GKAPKLLIYY | |
| | IWGDGTTDYN | | TSRFHSGVPS | |
| | SAVKSRVTIS | | RFSGSGSGTD | |
| | KDTSKNQFSL | | FTFTISSLQP | |
| | KLSSVTAADT | | EDIATYYCQQ | |
| | AVYYCARGGY | | EHTLPYTFGQ | |
| | WYATSYYFDY | | GTKLEIK | |
| | WGQGTLVTVS | | | |
| | S | | | |
| Tralokinumab | QVQLVQSGAE | 27 | SYVLTQPPSV | 28 |
| | VKKPGASVKV | | SVAPGKTARI | |
| | SCKASGYTFT | | TCGGNIIGSK | |
| | NYGLSWVRQA | | LVHWYQQKPG | |
| | PGQGLEWMGW | | QAPVLVIYDD | |
| | ISANNGDTNY | | GDRPSGIPER | |
| | GQEFQGRVTM | | FSGSNSGNTA | |
| | TTDTSTSTAY | | TLTISRVEAG | |
| | MELRSLRSDD | | DEADYYCQVW | |
| | TAVYYCARDS | | DTGSDPVVFG | |
| | SSSWARWFFD | | GGTKLTVL | |
| | LWGRGTLVTV | | | |
| | SS | | | |

In some embodiments, the antigen-binding site identified in the above method comprises amino acid residues on an antibody heavy chain variable region (VH) and an antibody light chain variable region (VL).

In some embodiments, the above-mentioned identification of antigen-binding site could involve one or more of generally known techniques in the art, including but not limited to, amino acid sequence analysis, structural analysis, mutational analysis, hydrogen-deuterium exchange analysis, computational analysis, or any combination thereof. (BIOVIA, Dassault Systèmes, Discovery Studio Visualizer, v19.1, San Diego: Dassault Systèmes, 2018; BioLuminate®. Zhu, K.; Day, T.; Warshaviak, D.; Murrett, C.; Friesner, R.; Pearlman, D., “Antibody structure determination using a combination of homology modeling, energy-based refinement, and loop prediction,” Proteins, 2014, 82(8), 1646-1655 Salam, N. K.; Adzhigirey, M.; Sherman, W.; Pearlman, D. A., “Structure-based approach to the prediction of disulfide bonds in proteins,” Protein Eng Des Sel, 2014, 27(10), 365-74; Beard, H.; Cholleti, A.; Pearlman, D.; Sherman, W.; Loving, K. A., “Applying Physics-Based Scoring to Calculate Free Energies of Binding for Single Amino Acid Mutations in Protein-Protein Complexes,” PLoS ONE, 2013, 8(12), e82849; Schrödinger Release 2018-1: BioLuminate, Schrödinger, LLC, New York, NY, 2018.)

In some embodiments, the step of identifying continuous surface amino acid patches that do not form specific interactions with the first antigen comprises identifying continuous surface residues within an antigen binding site, wherein the residues don't form specific interactions with the native antigen residues (FIG. 3). In some embodiments, those residues not forming specific interactions with the native antigen provide favorable sites for amino acid modification, wherein modification comprises substituting a different amino acid residue at that site. In some embodiments, residues not forming specific interactions with the native antigen have the potential to be changed without abrogating binding to the first antigen. In some embodiments, the patches identified within said first antigen binding site comprise amino acids on a VH or on a VL or on both a VH and VL.

In some embodiments, the step of identifying an antigen binding site comprising continuous surface amino acid patches comprises one or more of amino acid sequence analysis, structural analysis, mutational analysis, hydrogen-deuterium exchange analysis, computational analysis, or any combination thereof. In some embodiments, continuous surface patches comprises solvent accessible residues that are in close proximity. In some embodiments, the set of solvent accessible amino acid residues that are in close proximity has a length of about 2-20 amino acid residues. Close proximity encompasses a minimal distance of less than 5 Angstroms between residues.

In some embodiments, amino acid residues that could be changed without abrogating binding to the first antigen comprise residues that do not contribute to interactions with the first antigen. These residues therefore have the potential to be mutated for generating polypeptides comprising dual binding specificity.

In some embodiments, a step of selecting a subgroup of amino acid positions comprised within the patches for introducing one or more amino acid variants, comprises selecting residues that are non-interacting with the antigen and form a continuous surface patch comprising amino acid residues. This subgroup of residues need not be sequentially contiguous, as the continuous surface is formed by the 3D structural folding of the antigen binding region. (FIG. 3). In some embodiments, there may be multiple subgroups within a patch. Selection may comprise structural analysis or computational design analysis using tools known in the art. (BIOVIA, Dassault Systèmes, Discovery Studio Visualizer, v19.1, San Diego: Dassault Systèmes, 2018; BioLuminate®. Zhu, K. et al. 2014 ibid; Salam, N. K et al., 2014 ibid; Beard, Het al., 2013, ibid; Schrödinger Release 2018-1: BioLuminate, Schrödinger, LLC, New York, NY, 2018.)

In some embodiments, the subgroup of amino acid residues comprises solvent accessible residues that are in close proximity. In some embodiments, between about 2-8 positions for any given sequence of the first plurality of amino acid sequences are selected. In some embodiments, selection of said subgroup(s) of amino acid residues comprises computational methods or mutational analysis, or a combination thereof.

In some embodiments, the above-mentioned amino acid residues that could be changed without abrogating binding to the first antigen may include one or more amino acid residues in a CDR. In other embodiments, such amino acid residues that could be changed without abrogating binding to the first antigen may include one or more amino acid residues in a framework region (FR). In some embodiments, the amino acid residues that could be changed without abrogating binding to the first antigen comprise one or more amino acid residues in a CDR or a FR or both. In some embodiments, these amino acid residues are located inside or outside of the paratope, and they can be changed without abrogating binding to the first antigen. Changes to these amino acid residues may include, but are not limited to, substitution of different kind of amino acids.

In some embodiments, following selection of a subgroup of amino acids, amino acid variants are introduced into one or more of the residues of the selected subgroup. Introducing a range of amino acid variants into the one or more of the residues of the selected subgroup generates a library of amino acid sequences that may be screen for binding to a second antigen. This library forms a second plurality of amino acid sequences, wherein each of the amino acid sequences comprises the antigen binding site to said first antigen and the amino acid variants, wherein each sequence of the second plurality of amino acid sequences comprises up to 8 sites of amino acid variants. The terms “second plurality of amino acid sequences” and “variants” may in some embodiments be used interchangeable, wherein the skilled artisan would appreciate that these variants comprise the antigen binding site to said first antigen and the subgroups comprising up to 8 sites of amino acid variants.

In certain embodiments, one continuous surface patch is identified. In certain embodiments, several continuous surface patches are identified. In some embodiments, more than one continuous surface patch is identified. In some embodiments, each patch comprises multiple subgroups. In some embodiments, each patch comprises more than one subgroup. Patches and subgroups can be either disjoint or share some positions.

In some embodiments, the step of introducing amino acid variants within one or more of said selected subgroup amino acids comprises computationally designing libraries with variant amino acids at the selected residues within the patches. In some embodiments, the step of introducing amino acid variants within one or more of said selected subgroup amino acids comprises computationally designing libraries with variant amino acids at the selected residues within the patches, wherein said patches consist of between about 2-8 mutations (variant amino acids) per variant amino acid sequence.

As it is generally understood, an amino acid variant comprises a substitution of one amino acid residue for another. For example, an amino acid variant comprises a substitution of a hydrophobic residue with a non-hydrophobic residue. In some embodiments, an amino acid variant comprises a substitution of a charged residue with a non-charged residue. In some embodiments, an amino acid variant comprises a neutral substitution, wherein the amino acid being substituted has similar qualities. In some embodiments, an amino acid variant comprises a substitution of an aromatic residue with a non-aromatic residue. In some embodiments, natural aromatic amino acids such as Trp, Tyr and Phe are substituted with synthetic non-natural acid such as Phenylglycine, TIC, naphthylelanine (Nol), ring-methylated derivatives of Phe, halogenated derivatives of Phe or o-methyl-Tyr. In some embodiments, a variant substitution comprises substituting a modified amino acid or a non-amino acid monomer (e.g. fatty acid, complex carbohydrates etc). A skilled artisan would appreciate that while the choice of amino acid residues at each variant position may in certain embodiments affect the 3D structure of the VH, VL, and/or combination thereof, the choice of amino acid residues at each variant position is considered independently.

In some embodiments, the number of amino acid residues that could be changed within an amino acid sequence from said first plurality of sequences, without abrogating binding to the first antigen, can range from about 2 to about 45. In some embodiments, the number of amino acid residues that could be changed within is an amino acid sequence from said first plurality of sequences, without abrogating binding to the first antigen, comprises between about 2-8 amino acids. In some embodiments, the number of amino acid residues that could be changed within is an amino acid sequence from said first plurality of sequences, without abrogating binding to the first antigen, comprises up to about 8 amino acids.

Throughout this application, various embodiments may be presented in a range format. It should be understood that the description in range format is merely for convenience and brevity and should not be construed as an inflexible limitation on the scope of the invention. Accordingly, the description of a range should be considered to have specifically disclosed all the possible sub ranges as well as individual numerical values within that range. For example, description of a range such as from 1 to 6 should be considered to have specifically disclosed sub ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional or integral numerals there between.

Thus, the number of the above amino acid residues that could be changed without abrogating binding to the first antigen with a range from about 2 to about 45 should be considered to have specifically disclosed sub ranges such as from 2 to 4, from 3 to 5, from 4 to 6, from 5 to 7 etc., as well as individual numbers within that range, for example, 2, 3, 4, 5, 6 etc up to about 45.

In one embodiment, the one or more of the above amino acid variants are introduced in a CDR region. In another embodiment, the one or more amino acid variants are introduced within a framework (FR) region. In yet another embodiment, the amino acid variants include at least two variants, at least one within a CDR region and at least one within a framework (FR) region.

Site-specific mutagenesis allows the production of mutants through the use of specific oligonucleotide sequences which encode the DNA sequence of the desired mutation, as well as a sufficient number of adjacent nucleotides, to provide a primer sequence of sufficient size and sequence complexity to form a stable duplex on both sides of the deletion junction being traversed. Mutations may be employed in a selected polynucleotide sequence to improve, alter, decrease, modify, or otherwise change the properties of the polynucleotide itself, and/or alter the properties, activity, composition, stability, or primary sequence of the encoded polypeptide.

In certain embodiments, mutagenesis of the polynucleotide sequences that encode component parts of a first antigen binding site (VH domains, VL domain, or a combination thereof, as disclosed herein) is contemplated in order to add an additional antigen binding site for a second antigen within the encoded template VH or VL or both, such that the resulting antibody comprises dual specific binding, wherein binding to the first antigen is maintained and binding to a second antigen also occurs.

The techniques of site-specific mutagenesis are well-known in the art and are widely used to create variants of both polypeptides and polynucleotides. For example, site-specific mutagenesis is often used to alter a specific portion of a DNA molecule. In such embodiments, a primer comprising typically about 14 to about 25 nucleotides or so in length is employed, with about 5 to about 10 residues on both sides of the junction of the sequence being altered.

As will be appreciated by those of skill in the art, site-specific mutagenesis techniques have often employed a phage vector that exists in both a single stranded and double stranded form. Typical vectors useful in site-directed mutagenesis include vectors such as the M13 phage. These phages are readily commercially available, and their use is generally well-known to those skilled in the art. Double-stranded plasmids are also routinely employed in site directed mutagenesis that eliminates the step of transferring the gene of interest from a plasmid to a phage.

In general, site-directed mutagenesis in accordance herewith is performed by first obtaining a single-stranded vector or melting apart of two strands of a double-stranded vector that includes within its sequence a DNA sequence that encodes the desired peptide. An oligonucleotide primer bearing the desired mutated sequence is prepared, generally synthetically. This primer is then annealed with the single-stranded vector and subjected to DNA polymerizing enzymes such as E. coli polymerase I Klenow fragment, in order to complete the synthesis of the mutation-bearing strand. Thus, a heteroduplex is formed wherein one strand encodes the original non-mutated sequence and the second strand bears the desired mutation. This heteroduplex vector is then used to transform appropriate cells, such as E. coli cells, and clones are selected which include recombinant vectors bearing the mutated sequence arrangement.

The preparation of sequence variants of the selected peptide-encoding DNA segments using site-directed mutagenesis provides a means of producing potentially useful species and is not meant to be limiting as there are other ways in which sequence variants of peptides and the DNA sequences encoding them may be obtained. In some embodiments, methods of preparing libraries include those known in the art, for example but not limited to methods described in U.S. Pat. No. 9,889,423, which are included herein in their entirety. In some embodiments, a method for designing the sequence variants within a library comprises designing the variant sequences on a computer and then have the sequence synthesized, a method that involves both chemical and biochemical processes.

As used herein, the term “oligonucleotide directed mutagenesis procedure” encompasses template-dependent processes and vector-mediated propagation which result in an increase in the concentration of a specific nucleic acid molecule relative to its initial concentration, or in an increase in the concentration of a detectable signal, such as amplification. As used herein, the term “oligonucleotide directed mutagenesis procedure” encompasses a process that involves the template-dependent extension of a primer molecule. The term “template dependent process” encompasses nucleic acid synthesis of an RNA or a DNA molecule wherein the sequence of the newly synthesized strand of nucleic acid is dictated by the well-known rules of complementary base pairing. Typically, vector mediated methodologies involve the introduction of the nucleic acid fragment into a DNA or RNA vector, the clonal amplification of the vector, and the recovery of the amplified nucleic acid fragment. Examples of such methodologies are provided by U.S. Pat. No. 4,237,224, specifically incorporated herein by reference in its entirety.

In another approach for the production of polypeptide VH and VL variants, recursive sequence recombination, as described in U.S. Pat. No. 5,837,458, may be employed. In this approach, iterative cycles of recombination and screening or selection are performed to “evolve” individual polynucleotide variants having, for example, increased binding affinity. Certain embodiments also provide constructs in the form of plasmids, vectors, transcription or expression cassettes which comprise at least one polynucleotide as described herein.

In certain embodiments, the polynucleotides described herein, e.g., VH, VL, or VH and VL variant polynucleotides, fragments and hybridizing sequences, encoding the amino acid VH, VL, or VH and VL variants, are comprised in first antigen binding antibody. In some embodiments, the first antigen is selected from the group consisting of PD1, tumor necrosis factor alpha, β-amyloid peptide, CD11a, immunoglobulin E, human epidermal growth factor receptor 2, vascular endothelial growth factor A, CD20, nerve growth factor, IL-13, programmed death ligand 1 (PD-L1), and epidermal growth factor receptor.

The polynucleotides described herein, or fragments thereof, regardless of the length of the coding sequence itself, may be combined with other DNA sequences, such as promoters, polyadenylation signals, additional restriction enzyme sites, multiple cloning sites, other coding segments, and the like, such that their overall length may vary considerably. It is therefore contemplated that a nucleic acid fragment of almost any length may be employed, with the total length preferably being limited by the ease of preparation and use in the intended recombinant DNA protocol. For example, illustrative polynucleotide segments with total lengths of about 10,000, about 5000, about 3000, about 2,000, about 1,000, about 500, about 200, about 100, about 50 base pairs in length, and the like, (including all intermediate lengths) are contemplated to be useful.

In some embodiments, an amino acid variant comprises a spontaneous mutation within the first antigen binding site. In some embodiments, an amino acid variant comprises a spontaneous mutation within a CDR of the first antigen binding site. In some embodiments, an amino acid variant comprises a spontaneous mutation within a FR region of the first antigen binding site.

In one embodiment, the above amino acid residues that could be changed without abrogating binding to the first antigen comprise a set of solvent accessible amino acid residues that are in close proximity. In one embodiment, this set of solvent accessible amino acid residues comprises a continuous surface patch. In one embodiment, this set of solvent accessible amino acid residues would have a length of about 2 to 20 amino acid residues, i.e. including sub ranges such as from 2 to 4, from 3 to 5, from 4 to 6, from 5 to 7 etc., as well as individual numbers within that range, for example, 2, 3, 4, 5, 6 etc up to about 20.

In one embodiment, the selection of amino acid sequences for introducing amino acid variants in the above method involve one or more of generally known techniques in the art, including but not limited to, computational methods or mutational analysis, or a combination thereof.

In some embodiments of a method of generating polypeptides with dual binding specificity, following generation of a second plurality of amino acid sequences, the second plurality of sequences is used to generate a high-throughput screening (HTS) library residue (FIG. 3). In some embodiments, a HTS may be generated in yeast, phage, or for ribosome display. A skilled artisan would appreciate that the HTS library generated using the steps provided herein may be advantageous, as it incorporates just relevant residues at certain predetermined positions, wherein these sites have a higher likelihood of not abrogated the binding to the first antigen and have a higher likelihood to form binding/support for a proper CDR conformation. In some embodiments, the HTS library generated comprises a smaller set of amino acid sequences than other HTS libraries generated for antibody screening. In some embodiments, the HTS library generated comprises more variants folded properly and thus will have a higher chance of finding a binder. In some embodiments, the HTS library generated comprises a smaller set of amino acid sequences than other HTS libraries generated for antibody screening, and comprises more variants folded properly and thus will have a higher chance of finding a binder.

In some embodiments, the HTS library is then screened for preservation of binding to the first antigen and for binding to a second antigen. In some embodiments, screens are sequential though the antigen does not need to be alternated with every round of screening. Thus, screening comprises multiple rounds of screening. Following each round of screening candidate polypeptides preserving binding to said first antigen and or binding to said second antigen may be selected.

In some embodiments, screening comprises between about 1-10 screens. In some embodiments, screening comprises between about 2-10 screens. In some embodiments, screening comprises between about 1-7 screens. In some embodiments, screening comprises between about 1-5 screens. In some embodiments, screening comprises about 1, 2, 3, 4, 5, 6, 7, 8, or 10 screens.

In some embodiments, screens alternate between the first antigen and the second antigen. In some embodiments, screens comprise multiple screens for one antigen prior to screening for the second antigen. In some embodiments, multiple screens may comprise between 2-10 screens. In some embodiments, screens comprise multiple screens for a first antigen, then at least one screen for a second antigen, then at least one screen for said first antigen. In some embodiments, screens comprise multiple screens for a first antigen, then multiple screens for a second antigen, then at least one screen for said first antigen. In some embodiments, this process of screening continues until a final group of candidate polypeptides is selected.

In some embodiments, following screening said HTS library, candidate polypeptides are selected that preserve the binding to said first antigen and confer binding to said second antigen. In some embodiments, the candidate polypeptides selected with each screen comprise amino acid sequences have tighter binding, for example showing higher affinity.

In some embodiments, the candidate polypeptides at step (g) comprise polypeptides with dual binding specificity and having at least 400-800 μM binding affinity for each antigen. In some embodiments, the candidate polypeptides at step (g) comprise polypeptides with dual binding specificity and having at least 400 μM binding affinity for each antigen. In some embodiments, the candidate polypeptides at step (g) comprise polypeptides with dual binding specificity and having at least 600 μM binding affinity for each antigen. In some embodiments, the candidate polypeptides at step (g) comprise polypeptides with dual binding specificity and having at least 800 μM binding affinity for each antigen.

In some embodiments, a method of generating polypeptides with dual binding specificity further comprises a step of maturation affinity of said candidate polypeptides, wherein said affinity maturation step is followed by another a screening step. Methods of affinity maturation are well known in the art, for example but not limited to Tabasinezhad M, Talebkhan Y, Wenzel W, Rahimi H, Omidinia E, Mahboudi F. Trends in therapeutic antibody affinity maturation: From in-vitro towards next-generation sequencing approaches. Immunol Let. 2019 August; 212:106-113. doi: 10.1016/j.imlet.2019.06.009. Epub 2019 Jun. 24. PMID: 31247224.

In some embodiments, after introducing amino acid variants into the above first plurality of amino acid sequences, the binding specificity, binding affinity, or binding avidity to the first antigen is not reduced by more than about one to three-orders of magnitude. In other embodiments, after introducing amino acid variants into the above first plurality of amino acid sequences, the binding specificity, binding affinity, or binding avidity to the first antigen is not reduced. Methods for characterizing binding specificity, binding affinity, and or binding avidity are well known in the art, including but not limited to yeast surface displaying, measuring EC50, ELISA, Surface plasmon resonance (SPR), Bio-layer Interferometry (BLI), etc.

In certain embodiments, a method of generating polypeptides comprising dual binding specificity further comprises a step expressing candidate polypeptides in the form of an IgG, a single-chain fragment variable (scFv), an Fab, an F(ab′)2, a minibody, a diabody, a triabody, a nanobody, or a single domain antibody. In certain embodiments, a method of generating polypeptides comprising dual binding specificity further comprises a step expressing candidate polypeptides in the form of an IgG1, IgG2, IgG3, or IgG4.

In some embodiments, the polypeptides with dual binding specificity generated by the above method can be in the form of an IgG, a single-chain fragment variable (scFv), an Fab, an F(ab′)2, a minibody, a diabody, a triabody, a nanobody, or a single domain antibody. The IgG can be of the subclass of IgG1, IgG2, IgG3, or IgG4.

A person of ordinary skill in the art would appreciate that a scFv is a fusion polypeptide comprising the variable heavy chain (VH) and variable light chain (VL) regions of an immunoglobulin, connected by a short linker peptide, the linker may have, for example, 10 to about 25 amino acids. The skilled artisan would also appreciate that the term “Fab” with regard to an antibody generally encompasses that portion of the antibody consisting of a single light chain (both variable and constant regions) bound to the variable region and first constant region of a single heavy chain by a disulfide bond, whereas F(ab′)₂ comprise two antigen-binding F(ab) portions linked together by disulfide bonds, and therefore are divalent.

As it is generally known in the art, minibody is a class of bispecific fragments, scFv-derived bispecific molecules. It is a bivalent fusion molecule with two scFvs fused to CH3. The scFv targeting antigen A is fused to the N-terminus of one of the CH3 domains and the scFv targeting antigen B to the other CH3. In one embodiment, the knob-into-holes technology can be used to force the CH3 domains heterodimerization. A person of ordinary skill in the art would also readily appreciate the terms “diabody”, “triabody”, “nanobody”, or “single domain antibody” as it is generally understood in the art.

In certain embodiments, the method disclosed herein would generate polypeptides comprising VH and VL domains. These polypeptides could be dimerized under suitable conditions. For example, the VH and VL domains may be combined in a suitable buffer and dimerized through appropriate interactions such as hydrophobic interactions. In another embodiment, the VH and VL domains may be combined in a suitable buffer containing an enzyme and/or a cofactor which can promote dimerization of the VH and VL domains. In another embodiment, the VH and VL domains may be combined in a suitable vehicle that allows them to react with each other in the presence of a suitable reagent and/or catalyst.

In certain embodiments, the VH and VL domains may be contained within longer polypeptide sequences, that may include for example but not limited to, constant regions, hinge regions, linker regions, Fc regions, or disulfide binding regions, or any combination thereof. A constant domain is an immunoglobulin fold unit of the constant part of an immunoglobulin molecule, also referred to as a domain of the constant region (e.g. CH1, CH2, CH3, CH4, Ck, Cl). In some embodiments, the longer polypeptides may comprise multiple copies of one or both of the VH and VL domains generated according to the method disclosed herein; for example, when the polypeptides generated herein are used to forms a diabody or a triabody.

In some embodiments, the polypeptides with dual binding specificity generated by the above method can bind to a first antigen and a second antigen at the same time. In other embodiments, such polypeptides with dual binding specificity can bind to either a first antigen or a second antigen.

In one embodiment, the first antigen can be PD1, tumor necrosis factor alpha, p-amyloid peptide, CD11a, immunoglobulin E, epidermal growth factor receptor 2, vascular endothelial growth factor A, CD20, nerve growth factor, IL-13, programmed death ligand 1 (PD-L1), or epidermal growth factor receptor. In another embodiment, the second antigen can be OX40, a glucocorticoid-Induced TNFR-Related (GITR) antigen, CTLA4, PDL-1, PD-1, CD25, tumor necrosis factor receptor 2 (TNFR2), VISTA (B7-H5), T cell immunoglobulin and mucin domain-containing protein 3 (TIM3), vascular endothelial growth factor (VEGF), Lymphocyte-activation gene 3 (LAG3), 4-1BB (CD137), DR3 (TNFRSF25), IL-2, or CD3.

In another embodiment, the present disclosure also provides a method of generating polypeptides with dual binding specificity to a PD1 (Programmed cell death protein 1) antigen and a second antigen. The method includes the steps of:
(a) providing a first plurality of amino acid sequences from antibodies that bind to PD1;
(b) identifying from each of the above amino acid sequences a first antigen-binding site for binding to PD1, wherein this first antigen-binding site comprising complementarity determining regions (CDRs) and framework regions and further identifying continuous surface amino acid residue patches within the antigen binding site that do not form specific interactions with PD1, wherein amino acid residues comprised within said patches provide favorable sites for substituting an amino acid;
(c) selecting a subgroup of amino acid sequences for introducing one or more amino acid variants, wherein the selection is on sequences comprising the above-identified amino acid residues that provide favorable sites for change without abrogating binding to PD1, and introducing amino acid variants to said one or more such amino acid residues that provide favorable sites for change, thereby generating a second plurality of amino acid sequences are generated, each of which comprises an antigen-binding site to PD1 and the above amino acid variants;
(d) generating a HTS library comprising said second plurality of amino acid sequences;
(e) screening said HTS library for binding to PD1 and binding to a second antigen; and
(f) selecting from the above HTS library comprising said second plurality of amino acid sequences, candidate polypeptides that confer binding to PD1 and to a second antigen, thereby generating polypeptides with dual binding specificity.

In some embodiments, additional steps and elements may be included in the method of generating a PD1 binding polypeptide with dual binding specificity, as has been described in detail above.

In one embodiment, the above-mentioned first plurality of amino acid sequences could include one or both of SEQ ID NO: 15 and SEQ ID NO: 16.

In one embodiment, the antigen-binding site for PD1 identified in the above method comprises amino acid residues in a heavy chain variable region and light chain variable region.

In one embodiment, the above-mentioned identification of antigen-binding site could involve one or more of generally known techniques in the art, including but not limited to, amino acid sequence analysis, structural analysis, mutational analysis, hydrogen-deuterium exchange analysis, computational analysis, or any combination thereof.

In one embodiment, the above-mentioned amino acid residues that could be changed without abrogating binding to PD1 may include one or more amino acid residues in a CDR. In another embodiment, such amino acid residues that could be changed without abrogating binding to PD1 may include one or more amino acid residues in a framework region.

In one embodiment, the number of amino acid residues that could be changed without abrogating binding to PD1 identified in the above method can range from about 2 to about 28. As discussed above, the range of about 2 to about 28 should be considered to have specifically disclosed sub ranges such as from 2 to 4, from 3 to 5, from 4 to 6, from 5 to 7 etc., as well as individual numbers within that range, for example, 2, 3, 4, 5, 6 etc up to about 28.

In one embodiment, the one or more of the above-mentioned amino acid variants are introduced in a CDR region. In another embodiment, the one or more amino acid variants are introduced within a framework region. In yet another embodiment, the amino acid variants include at least two variants, at least one within a CDR region and at least one within a framework region.

In one embodiment, the amino acid residues that could be changed without abrogating binding to said PD1 as identified in the above method comprise a set of solvent accessible amino acid residues that are in close proximity. In one embodiment, this set of solvent accessible amino acid residues comprises a continuous surface patch. In one embodiment, this set of solvent accessible amino acid residues would have a length of about 2 to 20 amino acid residues, i.e. including sub ranges such as from 2 to 4, from 3 to 5, from 4 to 6, from 5 to 7 etc., as well as individual numbers within that range, for example, 2, 3, 4, 5, 6 etc up to about 20.

In one embodiment, the selection of amino acid sequences for introducing amino acid variants in the above method involves one or more of generally known techniques in the art, including but not limited to, computational methods or mutational analysis, or a combination thereof.

In one embodiment, after introducing amino acid variants into the above first plurality of amino acid sequences that bind to PD1, the binding specificity, binding affinity, or binding avidity to PD1 is not reduced by more than about one to three-orders of magnitude. In another embodiment, after introducing amino acid variants into the above first plurality of amino acid sequences, the binding specificity, binding affinity, or binding avidity to PD1 is not reduced.

In one embodiment, the polypeptides (with binding specificity to PD1 and another antigen) generated by the above method can be in the form of an IgG, a single-chain fragment variable (scFv), an Fab, an F(ab′)2, a minibody, a diabody, a triabody, a nanobody, or a single domain antibody. The IgG can be of the subclass of IgG1, IgG2, IgG3, or IgG4.

In one embodiment, the polypeptides with dual binding specificity generated by the above method can bind to PD1 and a second antigen at the same time. In another embodiment, such polypeptides with dual binding specificity can bind to either PD1 or a second antigen. In one embodiment, the second antigen can be OX40, a glucocorticoid-Induced TNFR-Related (GITR) antigen, CTLA4, PDL-1, PD-1, CD25, tumor necrosis factor receptor 2 (TNFR2), VISTA (B7-H5), T cell immunoglobulin and mucin domain-containing protein 3 (TIM3), vascular endothelial growth factor (VEGF), Lymphocyte-activation gene 3 (LAG3), 4-1BB (CD137), DR3 (TNFRSF25), IL-2, or CD3

Polypeptides with Dual Specificity

In another embodiment, the present disclosure also provides isolated polypeptides with dual binding specificity. These polypeptides comprise a first binding-site for a first antigen and a second binding-site for a second antigen, wherein the second binding-site comprises amino acid variants of native amino acid sequences of polypeptides that bind to the first antigen, and the amino acid variants do not abrogate binding to the first antigen. In one embodiment, the polypeptides can be in the form of an IgG, a single-chain fragment variable (scFv), an Fab, an F(ab′)2, a minibody, a diabody, a triabody, a nanobody, or a single domain antibody. For example, the IgG can be of the subclass of IgG1, IgG2, IgG3, or IgG4.

In some embodiments, the first antigen can be PD1, tumor necrosis factor alpha, p-amyloid peptide, CD11a, immunoglobulin E, human epidermal growth factor receptor 2, vascular endothelial growth factor A, CD20, nerve growth factor, IL-13, programmed death ligand 1 (PD-L1), or epidermal growth factor receptor. In one embodiment, the second antigen can be OX40, a glucocorticoid-Induced TNFR-Related (GITR) antigen, CTLA4, PDL-1, PD-1, CD25, tumor necrosis factor receptor 2 (TNFR2), VISTA (B7-H5), T cell immunoglobulin and mucin domain-containing protein 3 (TIM3), vascular endothelial growth factor (VEGF), Lymphocyte-activation gene 3 (LAG3), 4-1BB (CD137), DR3 (TNFRSF25), IL-2, or CD3. In some embodiments, the polypeptides with dual binding specificity can bind to a first antigen and a second antigen at the same time. In another embodiment, such polypeptides with dual binding specificity can bind to either a first antigen or a second antigen. In yet another embodiment, these polypeptides can be generated by the method of generating polypeptides with dual binding specificity as discussed above.

In certain embodiments, the present disclosure also provides isolated polypeptides with dual binding specificity, wherein the polypeptides comprise a binding-site for a PD1 (Programmed cell death protein 1) antigen and a binding-site for a second antigen. In one embodiment, these polypeptides comprise one or more amino acid sequences as set forth in any of SEQ ID NOs: 30, 31, 33, 34, 36, 37, 39, 40, 42, 43, 45, 46, 48, 49, 51, 52, 54, 55, 57, 58, 60, 61, 63, 64, 66, 67, 69, 70, 72, 73, 75, 76, 78, 79, 81, 82, 84, 85, 87, 88, 90, 91, 93, 94, 96, 97, 99, 100, 102, 103, 105, 106, 108, and 109. In one embodiment, the polypeptides can be in the form of an IgG, a single-chain fragment variable (scFv), an Fab, an F(ab′)2, a minibody, a diabody, a triabody, a nanobody, or a single domain antibody. For example, the IgG can be of the subclass of IgG1, IgG2, IgG3, or IgG4. In certain embodiments, the second antigen can be OX40, a glucocorticoid-Induced TNFR-Related (GITR) antigen, CTLA4, PDL-1, CD25, tumor necrosis factor receptor 2 (TNFR2), VISTA (B7-H5), T cell immunoglobulin and mucin domain-containing protein 3 (TIM3), vascular endothelial growth factor (VEGF), Lymphocyte-activation gene 3 (LAG3), 4-1BB (CD137), DR3 (TNFRSF25), IL-2, or CD3.

In certain embodiments, when the second antigen is OX40, the above dual-specific polypeptides could include one or more amino acid sequences as set forth in SEQ ID NOs: 33, 34, 36, 37, 39, 40, 54, 55, 57, 58, 60, 61, 63, 64, 66, 67, 69, 70, 72, 73, 75, 76, 78, 79, 81, 82, 84, 85, 87, and 88. In another embodiment, when the second antigen is GITR, the above dual-specific polypeptides could include one or more amino acid sequences as set forth in SEQ ID NOs: 42, 43, 45, 46, 48, 49, 51, 52, 90, 91, 93, 94, 96, 97, 99, 100, 102, 103, 105, 106, 108, and 109. In one embodiment, these polypeptides with dual binding specificity can bind to PD1 and a second antigen at the same time. In certain embodiments, such polypeptides with dual binding specificity can bind to either PD1 or a second antigen.

### Compositions of Use

The polypeptides with dual binding specificity generated and disclosed herein can be administered to a subject (e.g. a mammal) alone, or in combination with a carrier, i.e., a pharmaceutically acceptable carrier. By pharmaceutically acceptable is meant a material that is not biologically or otherwise undesirable, i.e., the material can be administered to a subject without causing any undesirable biological effects or interacting in a deleterious manner with any of the other components of the pharmaceutical composition in which it is contained. As would be well-known to one of ordinary skill in the art, the carrier is selected to minimize any degradation of the polypeptides disclosed herein and to minimize any adverse side effects in the subject. The pharmaceutical compositions may be prepared by methodology well known in the pharmaceutical art.

The above pharmaceutical compositions comprising the polypeptides with dual binding specificity disclosed herein can be administered (e.g., to a mammal, a cell, a tissue, or a tumor) in any suitable manner depending on whether local or systemic treatment is desired. For example, the composition can be administered topically (e.g. ophthalmically, vaginally, rectally, intranasally, transdermally, and the like), orally, by inhalation, or parenterally (including by intravenous drip or subcutaneous, intracavity, intraperitoneal, intradermal, or intramuscular injection). Topical intranasal administration refers to delivery of the compositions into the nose and nasal passages through one or both of the nares. The composition can be delivered by a spraying mechanism or droplet mechanism, or through aerosolization. Delivery can also be directed to any area of the respiratory system (e.g., lungs) via intubation. Alternatively, administration can be intratumoral, e.g. local or intravenous injection.

If the composition is to be administered parenterally, the administration is generally by injection. Injectables can be prepared in conventional forms, either as liquid solutions or suspensions, solid forms suitable for suspension in liquid prior to injection, or as emulsions. Additionally, parental administration can involve preparation of a slow-release or sustained-release system so as to maintain a constant dosage.

The polypeptides with dual binding specificity disclosed herein may be used in therapeutic methods. In one embodiment, the polypeptides of the present disclosure can be used as immunotherapeutic agents, for example in the treatment of cancers. In one embodiment, the polypeptides of the present disclosure can be used alone or in combination with other anti-cancer therapies, such as chemotherapy or radiotherapy. The present polypeptides with dual binding specificity can be administered to a mammal directly, or by administering to the mammal a nucleic acid sequence encoding the polypeptides, such nucleic acid sequence may be carried by a vector.

The exact amount of the present polypeptides or compositions thereof required to elicit the desired effects will vary from mammal to mammal, depending on the species, age, gender, weight, and general condition of the mammal, the particular polypeptides, the route of administration, and whether other drugs are included in the regimen. Thus, it is not possible to specify an exact amount for every composition. However, an appropriate amount can be determined by one of ordinary skill in the art using routine experimentation. Dosages can vary, and the polypeptides can be administered in one or more (e.g., two or more, three or more, four or more, or five or more) doses daily, for one or more days. Guidance in selecting appropriate doses for antibodies can be readily found in the literature.

### Methods of Use

In some embodiments, a method of treating an allergic or respiratory condition, an inflammatory and/or autoimmune condition of the skin or gastrointestinal organs; scleroderma; or tumors or cancers in a subject, or any combination thereof, comprises a step of administering a pharmaceutical composition described above comprising the polypeptides with dual binding specificity disclosed herein.

A person of ordinary skill in the art would appreciate that the term “treating” and grammatical forms thereof, may in some embodiments encompass both therapeutic treatment and prophylactic or preventative measures with respect to a disease or condition, wherein the object is to treat, prevent, reduce, or alleviate, the disease or symptoms thereof, or a combination thereof. Thus, in some embodiments, treating may include directly affecting or curing, suppressing, inhibiting, preventing, reducing the severity of, delaying the onset of, reducing symptoms associated with the disease, disorder or condition, or a combination thereof. In some embodiments, “treating” encompasses enhancing the ability of host immune cells to destroy the pathogens or tumors.

In some embodiments, “preventing” encompasses delaying the onset of symptoms or an allergic or respiratory condition. In some embodiments, “suppressing” or “inhibiting”, encompass reducing the severity of symptoms, reducing the severity of an acute episode, reducing the number of symptoms, reducing the incidence of disease-related symptoms, reducing the latency of symptoms, ameliorating symptoms, reducing secondary symptoms, reducing secondary infections, prolonging patient survival, or a combination thereof.

### EXAMPLES

The Examples presented below, exemplify a method of screening existing antibodies to define the candidates most suitable to engineering of a second paratope while protecting the original paratope, which enables the generation of dual-binding antibodies. Further exemplified, is the utilization of this method to create two Nivolumab-based dual binders anti PD-1-OX40 and an anti PD1-GITR.

### Example 1

### Engineering and Production of Dual Specific Antibodies

Objective: To identify antibodies with the potential for engineering dual specificity.

Methods:

### Library Construction

Twist Library: The library was prepared by Twist (Twist Biosciences, USA) as a sc FV. DNA fragments with the desired mutations, positions and frequency were described below. The synthesized scFV library had a diversity of 1×10¹⁰. The library was PCR amplified using Phusion® High-Fidelity DNA Polymerase (New England Biolabs, USA) using forward and reverse primers to add sequences at the 5′ and 3′ of the scFv library that are homologous to the yeast surface display vector, allowing efficient homologous recombination in yeast cells. Library transformation was carried out as published (Benatuil et al., Protein Eng. Des. Sel. 23, 155-159 (2010)). Briefly, 400 μl of a yeast suspension (EBY100, ATCC, USA) per 0.2 cm cuvette (cell projects) was electroporated (BioRad, USA, GenePulser) with 4 μg linearized vector (pCTcon3) and 12 μg DNA insert (Twist scFv Library) in a 1:3 vector to insert ratio (Chao, G. et al., Nat. Protoc. 1, 755-768 (2006)). The number of transformants of the library was determined to 1×10⁹ by serial dilutions of transformed cells (10 cuvette electroporation reactions in a single experiment with an average of 1×10⁸ transformants per reaction).

Oligonucleotide Library: Library was constructed on the synthetic DNA template based on Nivolumab sequence (PDB i.d. 5GGR) by overlapping extension PCR with degenerate oligonucleotides encoding the diversity 1.1×10¹². PCR-introduced diversity was done using Phusion high fidelity DNA polymerase (New England Biolabs USA, Cat: M0530) according to manufacturer instructions in a 3-step reaction (98° C. for 30 sec, 65° C. for 20 sec, 72° C. for 30 sec, 30 cycles). The PCR products were gel purified by gel purification kit and assembled (100 ng from each) in equimolar ratios in a 3-step PCR reaction, as above, in the absence of primers. The assembled PCR product was reused as the template for PCR amplifying the full scFv library, using forward and reverse primers to add sequences at the 5′ and 3′ of the scFv library that are homologous to the yeast surface display vector, thus allowing efficient homologous recombination in yeast cells. Library transformation into yeast was conducted as described above. The number of transformants of the library was determined to 8.32×10⁷, in a single electroporation reaction, by serial dilutions of transformed cells. Both Twist library and Oligonucleotide library were mixed before the selection.

Affinity Maturation Libraries: The libraries were designed as described below. Clone G2 from the selections on GITR and 1B_seq96_13753_B01_4279975 from selections on OX40 were taken as template for the affinity maturation libraries. Libraries were constructed using the same methods described above for the Oligonucleotide library. The theoretical diversity was 4×10¹³ for the GITR library and 1×10¹³ for the OX40 library. The number of transformants of the library was determined by serial dilutions of transformed cells to be 1.36×10⁹ and 1.52×10⁹ for GITR and OX40 libraries respectively (in a multiple electroporation reaction for each library).

### Yeast Surface Display

Yeast-displayed scFv libraries were grown in a SD-CAA selective medium and induced for expression with 2% w/v galactose at 30° C. overnight according to established protocols (Chao et al., (2006) ibid). The library was screened on BioRad S3e Fluorescence Activated Cell Sorter (FACS) for high affinity binders of recombinant h-GITR-His, or recombinant h-PD1-His, or recombinant h-OX40-His (Reprokine) using mouse anti Myc-FITC (Santa Cruze, USA) and goat anti-His-APC (Miltenyi Biotec, Germany) for fluorescence labeling. Isolated clones from the final sort were plated on tryptophan depleted defined plate, and sequenced subsequent to extraction of plasmid DNA from the yeast clones using a Zymoprep kit (Zymo Research, USA). The chosen clones were incubated for 1 hour at room temperature with recombinant h-GITR-His, or hPD1-His, or hOX40-His, at the concentrations described in the example section. Cells were washed and resuspended in ice-cold PBS 0.1% BSA buffer containing a fluorescent labeled secondary antibody as described above for 20 min and analyzed using a flow cytometer. The values obtained were normalized to expression levels.

### IgG Production

Reformating: Selected scFv clones were reformatted to human IgG1 format. The sequences of the light chain (LC) and heavy chain (HC) variable regions were optimized to mammalian codon usage and ordered as genblocks (GB) from IDT (Integrated DNA Technologies. Coralville, Iowa USA). The GB were cloned using standard cloning techniques into pSF-CMV-HuIgG1_HC (HC plasmid) and pSF-CMV-HuLambda_LC (LC plasmid) (Oxford genetics, Oxford UK).

IgG Expression: Expi-CHO cells (Thermo Fisher Scientific, USA) were transfected with LC and HC plasmids at a ratio of 2:1 and expression was done according to the manufacturer's instructions. Briefly: 25 ml Expi-CHO cells were cultured at 37° C., 120 rpm, CO₂ 8% to a density of 6×10⁶ cell/ml. Then, 25 μg of expression plasmid at a ratio of 1:2 HC:LC were transfected into CHO cells. Post transfections, a booster and feed was added to the culture, and growth conditions were changed to 32° C., 120 rpm, 5% CO₂. The cells were harvested 10 days after transfection. The IgGs were purified from the supernatant using proteinA beads (Tosoh Bioscience GmbH, Germany), followed by size exclusion chromatography (SEC) purification on superdex 200 10/300 increase column, (GE healthcare, USA).

Size Exclusion Chromatography: To analyze and purify the IgGs, samples were loaded on a Superdex 200 10/300 increase column (GE healthcare, USA) at a flow rate of 0.8 ml/min on a GE AKTA Explorer chromatography system (GE healthcare, USA), when applicable 0.5 ml fractions of the peak corresponding to a folded IgG were collected. Monitoring of antibody retention time was done at 280 nm.

### Ligand Binding ELISA

IgG binding affinity was examined by ELISA experiments. ELISA plates (Greiner Bio-One-high binding) were coated with 50 ul (100 ng) of the tested antibody and incubated for 1 hour at room temperature. Then plates were washed three times with 300 μl PBS buffer containing 0.05% Tween 20 (PBS-T), blocked with 300μ PBS-T supplemented with 1% to 3% BSA, and incubated for 1 hour at room temperature (RT). Antibodies-coated plates were then washed three times with 300 μl PBS-T and incubated with serial dilutions of test ligand either hPD-1-His, or hOX40-His, or hGITR-His (Reprokine, Israel) in a final volume of 50 ul for 1-3 hours. Plates were then washed three times with 300 μl PBS-T and incubated with 50 μl anti-HIS-HRP conjugate that was diluted 1:300 in PBS (Santa Cruz Biotechnology, USA). After an additional wash step, the reaction was developed with 50 ul tetrametylbenzidine (TMB) reagent (Southern biotech, USA), and stopped with 50 ul 0.5N H₂SO₄. Detection was done on a Synergy LX BioTek (BioTek, USA) plate reader with an absorbance filter set to 450 nM. The binding affinity was determined by fitting the data to a specific binding non-linear regression model on Prisma 8 GraphPad software.

### Binding Specificity of BGD 32.004 and 32.005.

ELISA plates (Greiner Bio-One—high binding) were coated with 0.5 μg 32.004, 32.005 antibody or 17.003 (an anti-hIL2) in PBS and incubated for 1 hour at room temperature. Subsequently, the plates were washed three times with 300 μl PBS buffer containing 0.05% Tween 20 (PBS-T). Wells were blocked with 300 μl PBS-T supplemented with 1% BSA and incubated for 1 hour at room temperature (RT) and the plate was washed three times with 300 μl PBS-T. Then 50 μl of 100 nM hPD-1-His (Reprokine, Israel), 500 nM hIL-2-His (Reprokine, Israel) and 500 nM mouse TNFR2-His (Reprokine, Israel) were added to the plate and the plate was incubated for 1 hour at RT. After the incubation, the plate was washed three times with 300 μl PBS-T and 50 μl of anti-His HRP conjugated antibody (Santa Cruz Biotechnology, USA) diluted 1:300 in PBS was added for an incubation of 20 min at RT and the plate was washed three times with 300 μl PBS-T. The reaction was developed with 50 ul tetrametylbenzidine (TMB) reagent (Southern biotech, USA). and stopped with 50 ul 0.5N H2SO4. Detection was done on a Synergy LX BioTek (BioTek, USA) plate reader by reading absorbance signal at 450 nM.

### PD1/PDL1 Blocking Reporter Assay

To test clones for their ability to block PD1/PDL1 interaction, the PD-1/PD-L1 Blockade Bioassay (cat:J1255, Promega, USA) was used. The assay was performed according to manufacturer protocol. In brief, PD-L1 aAPC/CHO-K1 cells were seeded in a white sterile 96 well-plate at 37° C., CO₂ 5%, for 18 hours. The next day serial dilutions of reference and test antibodies were prepared. Then, the PD-1 effector cell and the antibodies were added to the 96 well-plates containing the pre-incubated PD-L1 aAPC/CHO-K1 cells. After six hours of incubation at 37° C., CO₂ 5%, Bio-glow Luminescence substrate was added, and the luminescence signal was monitored on a Biotek Synergy LX (Biotek, USA). The results were analyzed by fitting the data to non-linear dose-response four-parameters regression using Prisma 8 GraphPad software.

### Library Design

A computational based screening of antibody structures was performed to identify antibodies with the potential for engineering dual specificity. In this example the 3D structure of the listed antibodies was screened based on their structural properties. In certain embodiments, other screening methods, such as sequence-based computational screens, mutational analyses, or H/D exchange, for example, could be used.

The PDB (Protein Data Bank) database was searched for X-ray structures of therapeutic antibodies in complex with their native antigen. A list of 492 therapeutic antibodies and their sequences that are in Phase I clinical trials or above was compiled and information was obtained about their antigen. Using the sequences of heavy and light chains as a query, a NCBI BLAST search on the PDB database was performed to retrieve existing structures of these antibodies. Specifically, structures of antibodies complexed with antigen (114 different antibodies) were retrieved.

To identify antibodies amenable to introducing dual specificity, the paratopes of each antibody were analyzed and antibodies were screened for cases where the heavy or light chain have relatively large number of non-paratope CDR residues. i.e. over 75% of the CDR positions on either the heavy or light chain or both, do not form specific interactions with the antigen. Contact residues were defined as residues that have at least one heavy atom within 5 Angstroms of the antigen heavy atoms, as described herein.

While therapeutic antibodies were chosen as a starting point in this example, this should not be considered limiting; any collection of antibody structures, such as all antibodies in the PDB or the SabDab database, for example, could be used.

In order to identify amino acid residues within the complementarity-determining regions (CDR) that do not contribute to interactions with the antigen and thereby have the potential to be mutated for dual specificity, a calculation was performed to identify which CDR amino acids are engaged in interactions with the antigen and the total number of CDR amino acids residues of each CDR that maintain contact with the antigen. In one embodiment, antibody-antigen interacting residues were defined as amino acid residues in the antibody-antigen complex with at least one pair of heavy atoms at distance of 5 A or less (for example see BIOVIA, Dassault Systèmes, Discovery Studio Visualizer, v19.1, San Diego: Dassault Systèmes, 2018. BioLuminate: Zhu, K.; Day, T.; Warshaviak, D.; Murrett, C.; Friesner, R.; Pearlman, D., “Antibody structure determination using a combination of homology modeling, energy-based refinement, and loop prediction,” Proteins, 2014, 82(8), 1646-1655. Schrödinger Release 2018-1: BioLuminate, Schrödinger, LLC, New York, NY, 2018.).

As it is generally known, CDRs can be defined based on IMGT numbering: CDR1 27-38, CDR2 56-65 and CDR3 105-117. Moreover, one of ordinary skill in the art would readily determine CDRs and antigen binding sites based on other references such as Kunik et al., Comput. Biol. (2012; ibid), or Kabat definitions or Chothia definitions.

This structural screen allowed for the identification of antibodies having a significant number of CDR residues, preferably more than 75% of the residues, on one chain that do not interact directly with the antigen and are potentially amenable to engineering for new specificity. In some embodiments, between 65%-80% CDR residues on either a VH or VL chain do not interact direction with the antigen and are potentially amenable to engineering for new (e.g., second antigen) specificity. In some embodiments, about 65%, 70%, 75%, or 80% CDR residues on either a VH or VL chain do not interact direction with the antigen and are potentially amenable to engineering for new (e.g., second antigen) specificity. Presumably, mutating these residues would not affect the original antibody specificity.

In some embodiments, the term “about”, refers to a deviance of between 0.0001-5% from the indicated number or range of numbers. In some embodiments, the term “about”, refers to a deviance of between 1-10% from the indicated number or range of numbers. In some embodiments, the term “about”, refers to a deviance of up to 25% from the indicated number or range of numbers.

Table 2 lists antibodies that have CDR usage (interaction with antigen) of 25% or less on one of the chains.

TABLE 2
List of Potential Candidates For Dual Specificity Engineering

| Antibody Name | PDB id |
|---|---|
| bapineuzumab | 4HIX |
| durvalumab | 5XJ4 |
| efalizumab | 3EOB |
| golimumab | 5YOY |
| lebrikizumab | 4I77 |
| matuzumab | 3C09 |
| nivolumab | 5GGR |
| omalizumab | 5HYS |
| pertuzumab | 1S78 |
| ranibizumab | 1CZ8 |
| rituximab | 2OSL |
| tanezumab | 4EDW |
| tralokiumab | 5L6Y |

In one embodiment, the present disclosure describes using Nivolumab (anti-PD1) to exemplify methods described herein, wherein Nivolumab comprises a first antigen binding site to PD1. In one embodiment, amino acid residues in the CDR or in regions proximal to the CDR that do not contact the original antigen (e.g. 5 A or less) may be chosen for variability. Thus, in one embodiment, amino acid variability was introduced to Nivolumab at the following amino acid positions (IMGT numbering) through DNA mutations (see Table 3). The library with theoretical diversity of 1×10¹² and actual size of about 1×10⁸ transformants, included approximately up to 8 amino acid substitution mutations per variant.

TABLE 3
Amino Acid Residue Positions for Introduced Variability

| Heavy chain | Light chain |
|---|---|
| TRP57 | GLN27 |
| LYS64 | SER28 |
| ARG65 | SER36 |
| TYR66 | SER37 |
| | TYR38 |
| | ASP56 |
| | ALA57 |
| | SER65 |
| | THR85 |
| | SER107 |
| | SER108 |
| | ASN109 |
| | TRP114 |

### Screening the Library for PD-1-GITR Binders

Following introduction of variants at the identified residues (see Table 3), the library was screened in a yeast surface display format to identify clones that bind a glucocorticoid-induced TNFR-related protein (GITR) antigen while maintaining the binding to PD-1. The yeast library was grown to a cell number of 3×10⁹, induced on yeast display expression medium (SG media) to express the scFV (Nivolumab scFV with variants residues) at 20° C. and labeled with 0.5 μM of recombinant human PD-1 tagged with histidine tag (hPD-1-His) as described herein. Yeast binders were selected on magnetic beads, 2×10⁶ yeast cells were eluted from the magnetic column indicating enrichment factor of approximately 1000-fold. A second round of selection was done by labeling yeast cells with 0.5 μM (hGITR-His) using anti His-APC and anti Myc_FITC and selecting on a S3e Fluorescence Activated Cell Sorter (FACS) as described herein. Approximately, the top 1% of APC labeled e yeast cells were selected. Third and fourth rounds of selection were conducted in a similar fashion with 500 nM (hGITR-His).

At the end of the fourth round of selection, the yeast cells were plated on tryptophan depleted synthetic medium and individual clones were isolated, sequenced, and tested for specific binding to both 250 nM human recombinant PD-1 tagged with histidine tag (hPD-1-His) and 250 nM hGITR-His. Binders that showed enhanced binding for both targets are listed in Table 4.

### Screening the Library for PD-1-OX40 Binders

Screening for PD1-OX40 binders followed the steps as described above for PD1-GITR bindings.

### Affinity Maturation

Library design: Selected clones from the OX40 and GITR projects were then subject to affinity maturation, where Clone G2 from the selections on GITR and 1B_seq96_13753_B01_4279975 from selections on OX40 were taken as templates. Mutations were introduced to CDR positions that were not expected to negatively affect binding to PD1, in all CDR positions as well as positions that are proximal to the CDRs. Variant was by examining any of the 20 standard amino acids or based on amino acids observed at the specific position based on sequence conservation. The library was generated as described herein. In one embodiment, theoretical diversity of the affinity maturation library was 4×10¹³ for the GITR library and 1×10¹³ for the OX40. The actual YSD library screened was 1.36×10⁹ and 1.52×10⁹ for GITR and OX40 libraries respectively.

To screen for affinity matured PD-1—GITR dual binders, the yeast cells were grown to a cell number of 1×10¹⁰ induced with SG media to express scFV at 20° C. and labeled with 100 nM hGITR-His as described herein. hGITR-His binding yeast cells were selected on magnetic beads, 3×10⁷ yeast cells were eluted from the magnetic column indicating enrichment factor of approximately 300-fold. Since the number of eluted yeast cells in this round was too high for a practical screen in a FACS, a second round of magnetic beads selection was performed on 3×10⁸ cells labeled with 100 nM hGITR-His as described herein. hGITR-His binding yeast cells were panned using magnetic beads, 3×10⁵ yeast cells were eluted from the magnetic column indicating enrichment factor of approximately 1000-fold.

Third and fourth rounds of selection were done by labeling yeast cells with 100 nM (hGITR-His) with anti His-APC and anti Myc-FITC secondary labeling and sorting with a S3e Fluorescence Activated Cell Sorter as described herein. In these rounds the top 1% of the yeast cells were collected. The fifth round of selection was conducted in a similar fashion, this time yeast cells were labeled with 30 nM (hPD-1His), top 0.8% binders of the yeast cells were collected. A final round of selection was done with the yeast cells labeled with 10 nM hGITR-His. After 1 hour incubation with the hGITR-His, the yeast cells were washed and incubated in 1 ml PBS-F buffer for 1 hour and then labeled with anti-His-APC and anti-Myc-FITC and sorted. Subsequently to the fifth round of selection the yeast cells were plated on tryptophan depleted synthetic medium and individual clones were isolated, sequenced and tested for specific binding to both 111 nM human recombinant PD-1 tagged with histidine tag (hPD-1-His) and 90 nM hGITR-His as shown in FIG. 5.

### Results

As a first step, antibodies were identified that had a CDR usage (interaction with antigen) of 25% or less on one of the chains (Variable Heavy or Variable Light chains). Table 1 presents a list of 14 antibodies identified along with the PDB database ID numbers (PDB id).

In one embodiment, Nivolumab was selected as a candidate for engineering dual-specificity. Two libraries were designed. Positions that are predicted to undergo somatic hypermutation in the Ab family that do not interact with the original antigen were chosen for mutation. Alternately, all CDR and proximal residues that do not contact the original antigen may be chosen. As a result, variability was introduced to the following amino acid positions (IMGT numbering) through DNA mutations (shown in Table 3).

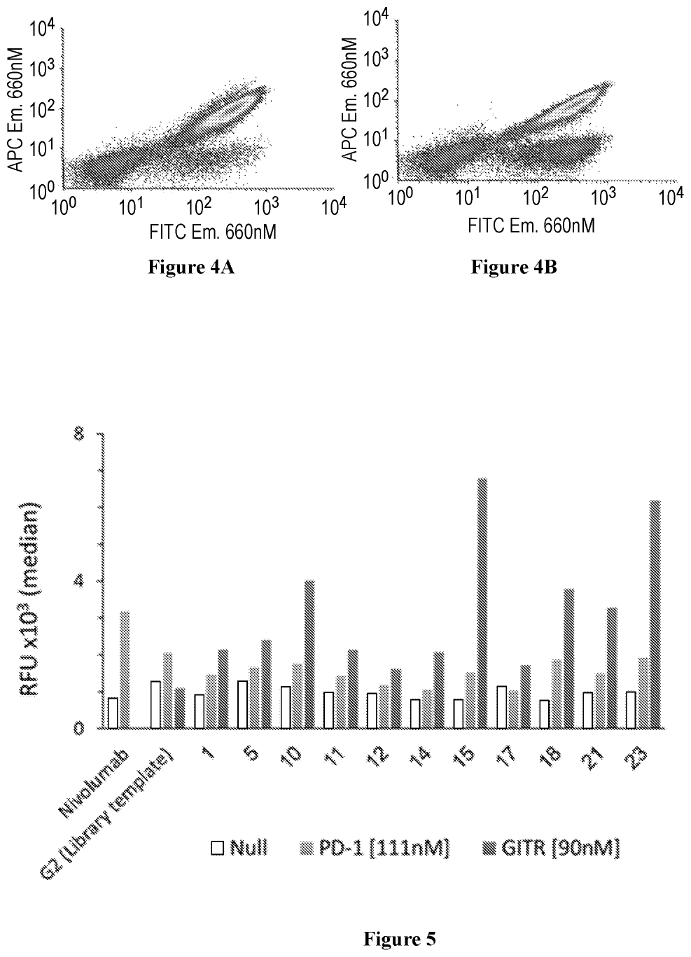

FIGS. 4A and 4B show the analysis of PD-1 binding (FIG. 4B) or GITR binding (FIG. 4A) of the of variant sequences comprised within the Library resulted in more than 50% of the library population binding to PD-1 and/r GITR.

Binders that showed enhanced binding for both targets (PD-1 and OX40, or PD-1 and GITR) are listed in Table 4.

TABLE 4
First Generation scFV DNA Sequences
Encoding Antibody Binding Sequences
Showing Dual Specificity

| | | SEQ |
|---|---|---|
| Clone name | ScFv DNA | ID NO: |
| 1A_seq96_13753 | CAGGTACAATTGGTAGAGTC | 32 |
| A01_4279974 | CGGTGGTGGCGTTGTCCAGC | |
| OX40 Library | CAGGCCGTTCCCTGCGTCTG | |
| | GACTGCAAAGCATCTGGCAT | |
| | CACCTTCTCCAACAGCGGTA | |
| | TGCACTGGGTGCGTCAGGCC | |
| | CCAGGCAAAGGGCTGGAGTG | |
| | GGTCGCCGTGATCGGTTATG | |
| | ATGGGTCTAGAGTCAAGTAT | |
| | GCTGACTCTGTAAAAGGCCG | |
| | TTTCACCATCAGCCGTGACA | |
| | ATAGCAAGAACACCCTGTTC | |
| | TTACAGATGAATTCCCTGCG | |
| | TGCTGAAGATACCGCCGTAT | |
| | ACTACTGCGCTACTAACGAC | |
| | GATTATTGGGGCCAAGGTAC | |
| | CCTGGTCACCGTCTCCTCTG | |
| | GCGGTGGTGGTAGCGGAGGC | |
| | GGAGGATCAGGTGGAGGCGG | |
| | CAGTGAAATTGTGCTGACCC | |
| | AAAGCCCAGCAACCCTGAGC | |
| | CTGTCTCCAGGCGAACGTGC | |
| | CACTCTGTCCTGCCGTGCAA | |
| | GCAGACCTGTCTCTTCTAAT | |
| | CTGGCATGGTACCAGCAGAA | |
| | GCCAGGGCAAGCCCCACGTC | |
| | TGTTAATCTACCGTGCAAGC | |
| | AATCGTGCCACCGGTATTCC | |
| | AGCCCGTTTTTCTGGCAGCG | |
| | GTTCCGGCACCGATTTCACT | |
| | CTGACCATCTCCTCCCTGGA | |
| | ACCAGAAGACTTCGCCGTTT | |
| | ACTACTGTCAACAGTCAAGA | |
| | AAGTGGCCACGTACTTTTGG | |
| | TCAGGGCACCAAAGTTGAGA | |
| | TTAAA | |
| 1C_seq96_13753_ | CAGGTACAATTGGTAGAGTC | 35 |
| C01_4279976 | CGGTGGTGGCGTTGTCCAGC | |
| OX40 Library | CAGGCCGTTCCCTGCGTCTG | |
| | GACTGCAAAGCATCTGGCAT | |
| | CACCTTCTCCAACAGCGGTA | |
| | TGCACTGGGTGCGTCAGGCC | |
| | CCAGGCAAAGTGCTGGAGTG | |
| | GGTCGCCGTGATCTGGTATG | |
| | ATTGGTCTAAACGTTACTAT | |
| | GCTGACTCTGTAAAAGGCCG | |
| | TTTCACCATCAGCCGTGACA | |
| | ATAGCAAGAACACCCTGTTC | |
| | TTACAGATGAATTCCCTGCG | |
| | TGCTGAAGATACCGCCGTAT | |
| | ACTACTGCGCTACTAACGAC | |
| | GATTATTGGGGCCAAGGTAC | |
| | CCTGGTCACCGTCTCCTCTG | |
| | GCGGTGGTGGTAGCGGAGGC | |
| | GGAGGATCAGGTGGAGGCGG | |
| | CAGTGAAATTGTGCTGACCC | |
| | AAAGCCCAGCAACCCTGAGC | |
| | CTGTCTCCAGGCGAACGTGC | |
| | CACTCTGTCCTGCCGTGCAA | |
| | GCCAGATGGTCGGTTCTAAG | |
| | CTGGCATGGTACCAGCAGAA | |
| | GCCAGGGCAAGCCCCACGTC | |
| | TGTTAATCTACAGTGCAAGC | |
| | AATCGTGCCACCGGTATTCC | |
| | AGCCCGTTTTTCTGGCAGCG | |
| | GTTCCGGCAGAGATTTCACT | |
| | CTGACCATCTCCTCCCTGGA | |
| | ACCAGAAGACTTCGCCGTTT | |
| | ACTACTGTCAACAGTCAGTT | |
| | AACTTGCCACGTACTTTTGG | |
| | TCAGGGCACCAAAGTTGAGA | |
| | TTAAA | |
| 1B_seq96_13753 | CAGGTACAATTGGTAGAGTC | 38 |
| B01_4279975 | CGGTGGTGGCGTTGTCCAGC | |
| OX40 Library | CAGGCCGTTCCCTGCGTCTG | |
| | GACTGCAAAGCATCTGGCAT | |
| | CACCTTCTCCAACAGCGGTA | |
| | TGCACTGGGTGCGTCAGGCC | |
| | CCAGGCAAAGGGCTGGAGTG | |
| | GGTCGCCGTGATCTGGTATG | |
| | ATGGGTCTAAACGTTACTAT | |
| | GCTGACTCTGTAAAAGGCCG | |
| | TTTCACCATCAGCCGTGACA | |
| | ATAGCAAGAACACCCTGTTC | |
| | TTACAGATGAATTCCCTGCG | |
| | TGCTGAAGATACCGCCGTAT | |
| | ACTACTGCGCTACTAACGAC | |
| | GATTATTGGGGCCAAGGTAC | |
| | CCTGGTCACCGTCTCCTCTG | |
| | GCGGTGGTGGTAGCGGAGGC | |
| | GGAGGATCAGGTGGAGGCGG | |
| | CAGTGAAATTGTGCTGACCC | |
| | AAAGCCCAGCAACCCTGAGC | |
| | CTGTCTCCAGGCGAACGTGC | |
| | CACTCTGTCCTGCCGTGCAA | |
| | GCCAGTCTGTCATCCACTAC | |
| | CTGGCATGGTACCAGCAGAA | |
| | GCCAGGGCAAGCCCCACGTC | |
| | TGTTAATCTACGACGCAAGC | |
| | AATCGTGCCACCGGTATTCC | |
| | AGCCCGTTTTTCTGGCAGCG | |
| | GTTCCGGCACCGATTTCACT | |
| | CTGACCATCTCCTCCCTGGA | |
| | ACCAGAAGACTTCGCCGTTT | |
| | ACTACTGTCAACAGTATTCA | |
| | AACTGGCCACTTACTTTTGG | |
| | TCAGGGCACCAAAGTTGAGA | |
| | TTAAA | |
| G1_GITR Library | CAGGCACAATTGGTAGAGTC | 41 |
| | CGGGGGTGGCGTTGTCCAGG | |
| | CAGGCCGTTCCCTGCGTCTG | |
| | GACTGCAAAGCATCTGGCAT | |
| | CACCTTCTCCAACAGGGGTA | |
| | TGCACTGGGTGCGTCAGGCC | |
| | CCAGGCAAAGGGCTGGAGTG | |
| | GGTCGCCGTGATCTGGTATG | |
| | ATGGGTCTCTGCGTTACTAT | |
| | GCTGACTCTGTAAAAGGCCG | |
| | TTTCACCATCAGCCGTGACA | |
| | ATAGCAAGAACACCCTGTTC | |
| | TTACAGATGAATTCCCTGCG | |
| | TGCTGAAGATACCGCCGTAT | |
| | ACTACTGCGCTACTAACGAC | |
| | GATTATTGGGGCCAAGGTAC | |
| | CCTGGTCACCGTCTCCTCTG | |
| | GCGGTGGTGGTAGCGGAGGC | |
| | GGAGGATCAGGTGGAGGCGG | |
| | CAGTGAAATTGTGCTGACCC | |
| | AAAGCCCAGCAACCCTGAGC | |
| | CTGTCTCCAGGCGAACGTGC | |
| | CACTCTGTCCTGCCGTGCAA | |
| | GCCAGTCTGTCTCTGACAAC | |
| | CTGGCATGGTACCAGCAGAA | |
| | GCCAGGGCAAGCCCCACGTC | |
| | TGTTAATCTACGACGCAGAC | |
| | AATCGTGCCACCGGTATTCC | |
| | AGCCCGTTTTTCTGGCAGCG | |
| | GTTCCGGCACCGATTTCACT | |
| | CTGACCATCTCCTCCCTGGA | |
| | ACCAGAAGACTTCGCCGTTT | |
| | ACTACTGTCAACAGTCATCA | |
| | AACTTACCACGTACTTTTGG | |
| | TCAGGGCACCAAAGTTGAGA | |
| | TTAAA | |
| G2_GITR Library | CAGGTACAATTGGTAGAGTC | 44 |
| | CGGTGGTGGCGTTGTCCAGC | |
| | CAGGCCGTTCCCTGCGTCTG | |
| | GACTGCAAAGCATCTGGCAT | |
| | CACCTTCTCCAACAGCGGTA | |
| | TGCACTGGGTGCGTCAGGCC | |
| | CCAGGCAAAGGGCTGGAGTG | |
| | GGTCGCCGTGATCTGGTATG | |
| | ATGGGTCTCTGCGTTACTAT | |
| | GCTGACTCTGTAAAAGGCCG | |
| | TTTCACCATCAGCCGTGACA | |
| | ATAGCAAGAACACCCTGTTC | |
| | TTACAGATGAATTCCCTGCG | |
| | TGCTGAAGATACCGCCGTAT | |
| | ACTACTGCGCTACTAACGAC | |
| | GATTATTGGGGCCAAGGTAC | |
| | CCTGGTCACCGTCTCCTCTG | |
| | GCGGTGGTGGTAGCGGAGGC | |
| | GGAGGATCAGGTGGAGGCGG | |
| | CAGTGAAATTGTGCTGACCC | |
| | AAAGCCCAGCAACCCTGAGC | |
| | CTGTCTCCAGGCGAACGTGC | |
| | CACTCTGTCCTGCCGTGCAA | |
| | GCCAGTCTGTCTCTGACAAC | |
| | CTGGCATGGTACCAGCAGAA | |
| | GCCAGGGCAAGCCCCACGTC | |
| | TGTTAATCTACGACGCAGAC | |
| | AATCGTGCCACCGGTATTCC | |
| | AGCCCGTTTTTCTGGCAGCG | |
| | GTTCCGGCACCGATTTCACT | |
| | CTGACCATCTCCTCCCTGGA | |
| | ACCAGAAGACTTCGCCGTTT | |
| | ACTACTGTCAACAGTCATCA | |
| | AACTTACCACGTACTTTTGG | |
| | TCAGGGCACCAAAGTTGAGA | |
| | TTAAA | |
| G5_GITR Library | CaGGTCCAATTGGTAGAGTC | 47 |
| | CGGTGGTGGCGTTGTCCATC | |
| | CAGGCCGTTCCCTGCGTGTG | |
| | GACTGCAAAGCATCTGGCAT | |
| | CACCTTCTCCAACAGCGGTA | |
| | TGCACTGGGTGCGTCAGGCC | |
| | CCAGGCAAAGGGCTGGAGTG | |
| | GGTCGCCGTGATCTGGTATG | |
| | ATGGGTCTCTGCGTTACTAT | |
| | GCTGACTCTGTAAAAGGCCG | |
| | TTTCACCATCAGCCGTGACA | |
| | ATAGCAAGAACACCCTGTTC | |
| | TTACAGATGAATTCCCTGCG | |
| | TGCTGAAGATACCGCCGTAT | |
| | ACTACTGCGCTACTAACGAC | |
| | GATTATTGGGGCCAAGGTAC | |
| | CCTGGTCACCGTCTCCTCTG | |
| | GCGGTGGTGGTAGCGGAGGC | |
| | GGAGGATCAGGTGGAGGCGG | |
| | CAGTGAAATTGTGCTGACCC | |
| | AAAGCCCAGCAACCCTGAGC | |
| | CTGTCTCCAGGCGAACGTGC | |
| | CACTCTGTCCTGCCGTGCAA | |
| | GCCAGTCTGTCTCTGACAAC | |
| | CTGGCATGGTACCAGCAGAA | |
| | GCCAGGGCAAGCCCCACGTC | |
| | TGTTAATCTACGACGCAGAC | |
| | AATCGTGCCACCGGTATTCC | |
| | AGCCCGTTTTTCTGGCAGCG | |
| | GTTCCGGCACCGATTTCACT | |
| | CTGACCATCTCCTCCCTGGC | |
| | ACCAGAAGACTTCGCCGTTT | |
| | ACTACTGTCAACAGTCATCA | |
| | aACTTACCACGTACTTTTGG | |
| | TCAGGGCACCAAAGTTGAGA | |
| | TTAAA | |
| G8_GITR Library | CAGGTACAATTGGTAGAGTC | 50 |
| | CGGTGGTGGCGTTGTCCAGC | |
| | CTGGCCGTTCCCTGCGTCTG | |
| | GACTGCAAAGCATCTGGCAT | |
| | CACCTTCTCCAACAGCGGTA | |
| | TGCACTGGGTGCGTCAGGCC | |
| | CCAGGCAAAGGGCTGGAGTG | |
| | GGTCGCCGTGATCTGGTATG | |
| | ATGGGTCTCTGCGTTACTAT | |
| | GCTGACTCTGTAAAAGGCCG | |
| | TTTCACCATCAGCCGTGACA | |
| | ATAGCAAGAACACCCTGTTC | |
| | TTACAGATGAATTCCCTGCG | |
| | TGCTGAAGATACCGCCGTAT | |
| | ACTACTGCGCTTCTAACGAC | |
| | GATTATTGGGGCCAAGGTAC | |
| | CCTGGTCACCGTCTCCTCTG | |
| | GCGGTGGTGGTAGCGGAGGC | |
| | GGAGGATCAGGTGGAGGCGG | |
| | CAGTGAAATTGTGCTGACCC | |
| | AAAGCCCAGCAACCCTGAGC | |
| | CTGTCTCCAGGCGAACGTGC | |
| | CACTCTGTCCTGCCGTGCAA | |
| | GCCAGTCTGTCTCTGACAAC | |
| | CTGGCATGGTACCAGCAGAA | |
| | GCCAGGGCAAGCCCCACGTC | |
| | TGTTAATCTACGACGCAGAC | |
| | AATCGTGCCACCGGTATTCC | |
| | AGCCCGTTTTTCTGGCAGCG | |
| | GTTCCGGCACCGATTTCACT | |
| | CTGACCATCTCCTCCCTGGA | |
| | ACCAGAAGACTTCGCCGTTT | |
| | ACTACTGTCAACAGTCATCA | |
| | AACTTACCACGTACTTTTGG | |
| | TCAGGGCACCAAAGTTGAGA | |
| | TTAAA | |

The amino acid sequences of the VH and VL chains for the clones listed in Table 3 are provided in SEQ ID NOs: 33, 34, 36, 37, 39, 40, 42, 43, 45, 46, 48, 49, 51, and 52, respectively by pairs.

The initial clones isolated were further screened using rounds of a maturation affinity method, as described above. Subsequent to the fifth round of selection, the yeast were plated on tryptophan depleted synthetic medium and individual clones were isolated, sequenced and tested for specific binding to both 111 nM human recombinant PD-1 tagged with histidine tag (hPD-1-His) and 90 nM hGITR-His as shown in FIG. 5 (second Generation Clones).

Second Generation binders that showed binding enhanced binding for both targets (PD1 and GITR, and PD1 and OX40) are listed in Table 5.

TABLE 5
Second Generation scFV DNA sequences of
Clones with Enhanced Dual Binding

| | | SEQ |
|---|---|---|
| Clone | | ID |
| name | ScFv DNA | NO: |
| 3212ACO | CAGGTACAATTGGTAGAGTCCGGTG | 53 |
| XC51 | GTGGCGTTGTCCAGCCAGGCCGTTC | |
| OX40 | CCTGCGTCTGGACTGCAAAGCATCT | |
| library | GGCATCACCTTCTCCAACAGCGGTA | |
| | TGCACTGGGTGCGTCAGGCCCCAGG | |
| | CAAAGGGCTGGAGTGGGTCGCCGTG | |
| | ATCTGGTATGATGGGTCTAAAAGCT | |
| | ACTATGCTGACTCTGTAAAAGGCCG | |
| | TTTCACCATCAGCCGTGACAATAGC | |
| | AAGAACACCCTGTTCTTACAGATGA | |
| | ATTCCCTGCGTGCTGAAGATACCGC | |
| | CGTATACTACTGCGCTACTAACGAC | |
| | GATTATTGGGGCCAAGGTACCCTGG | |
| | TCACCGTCTCCTCTGGCGGTGGTGG | |
| | TAGCGGAGGCGGAGGATCAGGTGGA | |
| | GGCGGCAGTGAAATTGTGCTGACCC | |
| | AAAGCCCAGCAACCCTGAGCCTGTC | |
| | TCCAGGCGAACGTGCCACTCTGTCC | |
| | TGCCGTGCAAGCCAGTCTGTCCGCC | |
| | ACTACCTGGCATGGTACCAGCAGAA | |
| | GCCAGGGCAAGCCCCACGTCTGTTA | |
| | ATCTACGACGCAATGAATCGTGCCA | |
| | CCGGTATTCCAGCCCGTTTTTCTGG | |
| | CAGCGGTTCCGGCACCGATTTCACT | |
| | CTGACCATCTCCTCCCTGGAACCAG | |
| | AAGACTTCGCCGTTTACTACTGTCA | |
| | ACAGTATGAGGACTGGCCACTTACT | |
| | TTTGGTCAGGGCACCAAAGTTGAGA | |
| | TTAAA | |
| 3212ACO | CAGGTACAATTGGTAGAGTCCGGTG | 56 |
| XC61 | GTGGCGTTGTCCAGCCAGGCCGTTC | |
| OX40 | CCTGCGTCTGGACTGCAAAGCATCT | |
| library | GGCATCGCGTTCTCCAACAGCGGTA | |
| | TGCACTGGGTGCGTCAGGCCCCAGG | |
| | CAAAGGGCTGGAGTGGGTCGCCGTG | |
| | ATCTGGTATGATGGGTCTAAAACGT | |
| | ACTATGCTGACTCTGTAAAAGGCCG | |
| | TTTCACCATCAGCCGTGACAATAGC | |
| | AAGAACACCCTGTTCTTACAGATGA | |
| | ATTCCCTGCGTGCTGAAGATACCGC | |
| | CGTATACTACTGCGCTACTAACGAC | |
| | GATTATTGGGGCCAAGGTACCCTGG | |
| | TCACCGTCTCCTCTGACGGTGGTGG | |
| | TAGCGGAGGCGGAGGATCAGGTGGA | |
| | GGCGGCAGTGAAATTGTGCTGACCC | |
| | AAAGCCCAGCAACCCTGAGCCTGTC | |
| | TCCAGGCGAACGTGCCACTCTGTCC | |
| | TGCCGTGCAAGCCAGTCTGTCATCC | |
| | ACTACCTGGCATGGTACCAGCAGAA | |
| | GCCAGGGCAAGCCCCACGTCTGTTA | |
| | ATCTACGACGCAAGCAATCGTGCCA | |
| | CCGGTATTCCAGCCCGTTTTTCTGG | |
| | CAGCGGTTCCGGCACCGATTTCACT | |
| | CTGACCATCTCCTCCCTGGAACCAG | |
| | AAGACTTCGCCGTTTACTACTGTCA | |
| | ACAGTATACCAACTGGCCACTTACT | |
| | TTTGGTCAGGGCACCAAAGTTGAGA | |
| | TTAAA | |
| 3212ACO | CAGGTACAATTGGTAGAGTCCGGTG | 59 |
| XC81 | GTGGCGTTGTCCAGCCAGGCCGTTC | |
| OX40 | CCTGCGTCTGGACTGCAAAGCATCT | |
| library | GGCATCGACTTCTCCAACAGCGGTA | |
| | TGCACTGGGTGCGTCAGGCCCCAGG | |
| | CAAAGGGCTGGAGTGGGTCGCCGTG | |
| | ATCTGGTATGATGGGTCTACGCGTT | |
| | ACTATGCTGACTCTGTAAAAGGCCG | |
| | TTTCACCATCAGCCGTGACAATAGC | |
| | AAGAACACCCTGTTCTTACAGATGA | |
| | ATTCCCTGCGTGCTGAAGATACCGC | |
| | CGTATACTACTGCGCTACTAACGAC | |
| | GATTATTGGGGCCAAGGTACCCTGG | |
| | TCACCGTCTCCTCTGGCGGTGGTGG | |
| | TAGCGGAGGCGGAGGATCAGGTGGA | |
| | GGCGGCAGTGAAATTGTGCTGACCC | |
| | AAAGCCCAGCAACCCTGAGCCTGTC | |
| | TCCAGGCGAACGTGCCACTCTGTCC | |
| | TGCCGTGCAAGCCAGTCTGTCATCC | |
| | ACTACCTGGCATGGTACCAGCAGAA | |
| | GCCAGGGCAAGCCCCACGTCTGTTA | |
| | ATCTACGACGCAAGCAATCGTGCCA | |
| | CCGGTATTCCAGCCCGTTTTTCTGG | |
| | CAGCGGTTCCGGCACCGATTTCACT | |
| | CTGACCATCTCCTCCCTGGAACCAG | |
| | AAGACTTCGCCGTTTACTACTGTCA | |
| | ACAGTATGACAACTGGCCACTTACT | |
| | TTTGGTCAGGGCACCAAAGTTGAGA | |
| | TTAAA | |
| 3212ACO | CAGGTACAATTGGTAGAGTCCGGTG | 62 |
| XC5A | GTGGCGTTGTCCAGCCAGGCCGTTC | |
| OX40 | CCTGCGTCTGGACTGCAAAGCATCT | |
| library | GGCATCACCTTCTCCAACAGCGGTA | |
| | TGCACTGGGTGCGTCAGGCCCCAGG | |
| | CAAAGGGCTGGAGTGGGTCGCCGTG | |
| | ATCTGGTATGATGGGTCTAAAACGT | |
| | ACTATGCTGACTCTGTAAAAGGCCG | |
| | TTTCACCATCAGCCGTGACAATAGC | |
| | AAGAACACCCTGTTCTTACAGATGA | |
| | ATTCCCTGCGTGCTGAAGATACCGC | |
| | CGTATACTACTGCGCTACTAACGAC | |
| | GATTATTGGGGCCAAGGTACCCTGG | |
| | TCACCGTCTCCTCTGGCGGTGGTGG | |
| | TAGCGGAGGCGGAGGATCAGGTGGA | |
| | GGCGGCAGTGAAATTGTGCTGACCC | |
| | AAAGCCCAGCAACCCTGAGCCTGTC | |
| | TCCAGGCGAACGTGCCACTCTGTCC | |
| | TGCCGTGCAAGCCAGTCTGTCGAGC | |
| | ACTACCTGGCATGGTACCAGCAGAA | |
| | GCCAGGGCAAGCCCCACGTCTGTTA | |
| | ATCTACGACGCAGGGAATCGTGCCA | |
| | CCGGTATTCCAGCCCGTTTTTCTGG | |
| | CAGCGGTTCCGGCACCGATTTCACT | |
| | CTGACCATCTCCTCCCTGGAACCAG | |
| | AAGACTTCGCCGTTTACTACTGTCA | |
| | ACAGTATAGCGACTGGCCACTTACT | |
| | TTTGGTCAGGGCACCAAAGTTGAGA | |
| | TTAAA | |
| 3212ACO | CAGGTACAATTGGTAGAGTCCGGTG | 65 |
| XC7A | GTGGCGTTGTCCAGCCAGGCCGTTC | |
| OX40 | CCTGCGTCTGGACTGCAAAGCATCT | |
| library | GGCATCGACTTCTCCAACAGCGGTA | |
| | TGCACTGGGTGCGTCAGGCCCCAGG | |
| | CAAAGGGCTGGAGTGGGTCGCCGTG | |
| | ATCTGGTATGATGGGTCTTTGCGTT | |
| | ACTATGCTGACTCTGTAAAAGGCCG | |
| | TTTCACCATCAGCCGTGACAATAGC | |
| | AAGAACACCCTGTTCTTACAGATGA | |
| | ATTCCCTGCGTGCTGAAGATACCGC | |
| | CGTATACTACTGCGCTACTAACGAC | |
| | GATTATTGGGGCCAAGGTACCCTGG | |
| | TCACCGTCTCCTCTGGCGGTGGTGG | |
| | TAGCGGAGGCGGAGGATCAGGTGGA | |
| | GGCGGCAGTGAAATTGTGCTGACCC | |
| | AAAGCCCAGCAACCCTGAGCCTGTC | |
| | TCCAGGCGAACGTGCCACTCTGTCC | |
| | TGCCGTGCAAGCCAGTCTGTCGAGC | |
| | ACTACCTGGCATGGTACCAGCAGAA | |
| | GCCAGGGCAAGCCCCACGTCTGTTA | |
| | GTCTACGACGCAAGCAATCGTGCCA | |
| | CCGGTATTCCAGCCCGTTTTTCTGG | |
| | CAGCGGTTCCGGCACCGATTTCACT | |
| | CTGACCATCTCCTCCCTGGAACCAG | |
| | AAGACTTCGCCGTTTACTACTGTCA | |
| | ACAGTATTCAAACTGGCCACTTACT | |
| | TTTGGTCAGGGCACCAAAGTTGAGA | |
| | TTAAA | |
| 3212ACO | CAGGTACAATTGGTAGAGTCCGGTG | 68 |
| XC10 | GTGGCGTTGTCCAGCCAGGCCGTTC | |
| OX40 | CCTGCGTCTGGACTGCAAAGCATCT | |
| library | GGCATCGACTTCTCCAACAGCGGTA | |
| | TGCACTGGGTGCGTCAGGCCCCAGG | |
| | CAAAGGGCTGGAGTGGGTCGCCGTG | |
| | ATCTGGTATGATGGGTCTAAACGTT | |
| | TGTATGCTGACTCTGTAAAAGGCCG | |
| | TTTCACCATCAGCCGTGACAATAGC | |
| | AAGAACACCCTGTTCTTACAGATGA | |
| | ATTCCCTGCGTGCTGAAGATACCGC | |
| | CGTATACTACTGCGCTTCCAACGAC | |
| | GATTATTGGGGCCAAGGTACCCTGG | |
| | TCACCGTCTCCTCTGGCGGTGGTGG | |
| | TAGCGGAGGCGGAGGATCAGGTGGA | |
| | GGCGGCAGTGAAATTGTGCTGACCC | |
| | AAAGCCCAGCAACCCTGAGCCTGTC | |
| | TCCAGGCGAACGTGCCACTCTGTCC | |
| | TGCCGTGCAAGCCAGTCTGTCATCC | |
| | ACTACCTGGCATGGTACCAGCAGAA | |
| | GCCAGGGCAAGCCCCACGTCTGTTA | |
| | ATCTACGACGCAAGCAATCGTGCCA | |
| | CCGGTATTCCAGCCCGTTTTTCTGG | |
| | CAGCGGTTCCGGCACCGATTTCACT | |
| | CTGACCATCTCCTCCCTGGAACCAG | |
| | AAGACTTCGCCGTTTACTACTGTCA | |
| | ACAGTATGACAACTGGCCACTTACT | |
| | TTTGGTCAGGGCACCAAAGTTGAGA | |
| | TTAAA | |
| 3212ACO | CAGGTACAATTGGTAGAGTCCGGTG | 71 |
| XC14 | GTGGCGTTGTCCAGCCAGGCCGTTC | |
| OX40 | CCTGCGTCTGGACTGCAAAGCATCT | |
| library | GGCATCGAGTTCTCCAACAGCGGTA | |
| | TGCACTGGGTGCGTCAGGCCCCAGG | |
| | CAAAGGGCTGGAGTGGGTCGCCGTG | |
| | ATCTGGTATGATGGGTCTAAACGTT | |
| | ACTATGCTGACTCTGTAAAAGGCCG | |
| | TTTCACCATCAGCCGTGACAATAGC | |
| | AAGAACACCCTGTTCTTACAGATGA | |
| | ATTCCCTGCGTGCTGAAGATACCGC | |
| | CGTATACTACTGCGCTACTAACGAC | |
| | GATTATTGGGGCCAAGGTACCCTGG | |
| | TCACCGTCTCCTCTGGCGGTGGTGG | |
| | TAGCGGAGGCGGAGGATCAGGTGGA | |
| | GGCGGCAGTGAAATTGTGCTGACCC | |
| | AAAGCCCAGCAACCCTGAGCCTGTC | |
| | TCCAGGCGAACGTGCCACTCTGTCC | |
| | TGCCGTGCAAGCCAGTCTGTCGAGC | |
| | ACTACCTGGCATGGTACCAGCAGAA | |
| | GCCAGGGCAAGCCCCACGTCTGTTA | |
| | ATCTACGACGCAAGCAATCGTGCCA | |
| | CCGGTATTCCAGCCCGTTTTTCTGG | |
| | CAGCGGTTCCGGCACCGATTTCACT | |
| | CTGACCATCTCCTCCCTGGAACCAG | |
| | AAGACTTCGCCGTTTACTACTGTCA | |
| | ACAGTATTCAAACTGGCCACTTACT | |
| | TTTGGTCAGGGCACCAAAGTTGAGA | |
| | TTAAA | |
| 3212ACO | CAGGTACAATTGGTAGAGTCCGGTG | 74 |
| XC21 | GTGGCGTTGTCCAGCCAGGCCGTTC | |
| OX40 | CCTGCGTCTGGACTGCAAAGCATCT | |
| library | GGCATCGAGTTCTCCAACAGCGGTA | |
| | TGCACTGGGTGCGTCAGGCCCCAGG | |
| | CAAAGGGCTGGAGTGGGTCGCCGTG | |
| | ATCTGGTATGATGGGTCTAAACGTT | |
| | ACTATGCTGACTCTGTAAAAGGCCG | |
| | TTTCACCATCAGCCGTGACAATAGC | |
| | AAGAACACCCTGTTCTTACAGATGA | |
| | ATTCCCTGCGTGCTGAAGATACCGC | |
| | CGTATACTACTGCGCTAGCAACGAC | |
| | GATTATTGGGGCCAAGGTACCCTGG | |
| | TCACCGTCTCCTCTGGCGGTGGTGG | |
| | TAGCGGAGGCGGAGGATCAGGTGGA | |
| | GGCGGCAGTGAAATTGTGCTGACCC | |
| | AAAGCCCAGCAACCCTGAGCCTGTC | |
| | TCCAGGCGAACGTGCCACTCTGTCC | |
| | TGCCGTGCAAGCCAGTCTGTCGACC | |
| | ACTACCTGGCATGGTACCAGCAGAA | |
| | GCCAGGGCAAGCCCCACGTCTGTTA | |
| | ATCTACGACGCAAGCAATTTCGCCA | |
| | CCGGTATTCCAGCCCGTTTTTCTGG | |
| | CAGCGGTTCCGGCACCGATTTCACT | |
| | CTGACCATCTCCTCCCTGGAACCAG | |
| | AAGACTTCGCCGTTTACTACTGTCA | |
| | ACAGTATTCAAACTGGCCACTTACT | |
| | TTTGGTCAGGGCACCAAAGTTGAGA | |
| | TTAAA | |
| 3212ACO | CAGGTACAATTGGTAGAGTCCGGTG | 77 |
| XC23 | GTGGCGTTGTCCAGCCAGGCCGTTC | |
| OX40 | CCTGCGTCTGGACTGCAAAGCATCT | |
| library | GGCATCGACTTCTCCAACAGCGGTA | |
| | TGCACTGGGTGCGTCAGGCCCCAGG | |
| | CAAAGGGCTGGAGTGGCTGGCCGTG | |
| | ATCTGGTATGATGGGTCTAAACGTT | |
| | ACTATGCTGACTCTGTAAAAGGCCG | |
| | TTTCACCATCAGCCGTGACAATAGC | |
| | AAGAACACCCTGTTCTTACAGATGA | |
| | ATTCCCTGCGTGCTGAAGATACCGC | |
| | CGTATACTACTGCGCTTCCAACGAC | |
| | GATTATTGGGGCCAAGGTACCCTGG | |
| | TCACCGTCTCCTCTGGCGGTGGTGG | |
| | TAGCGGAGGCGGAGGATCAGGTGGA | |
| | GGCGGCAGTGAAATTGTGCTGACCC | |
| | AAAGCCCAGCAACCCTGAGCCTGTC | |
| | TCCAGGCGAACGTGCCACTCTGTCC | |
| | TGCCGTGCAAGCCAGTCTGTCATCC | |
| | ACTACCTGGCATGGTACCAGCAGAA | |
| | GCCAGGGCAAGCCCCACGTCTGTTA | |
| | ATCTACGACGCAAGCAATCGTGCCA | |
| | CCGGTATTCCAGCCCGTTTTTCTGG | |
| | CAGCGGTTCCGGCACCGATTTCACT | |
| | CTGACCATCTCCTCCCTGGAACCAG | |
| | AAGACTTCGCCGTTTACTACTGTCA | |
| | ACAGTATGAGAACTGGCCACTTACT | |
| | TTTGGTCAGGGCACCAAAGTTGAGA | |
| | TTAAA | |
| 3212ACO | CAGGTACAATTGGTAGAGTCCGGTG | 80 |
| XC31 | GTGGCGTTGTCCAGCCAGGCCGTTC | |
| OX40 | CCTGCGTCTGGACTGCAAAGCATCT | |
| library | GGCATCACCTTCTCCAACAGCGGTA | |
| | TGCACTGGGTGCGTCAGGCCCCAGG | |
| | CAAAGGGCTGGAGTGGGTCGCCGTG | |
| | ATCTGGTATGATGGGTCTAAAGCGT | |
| | ACTATGCTGACTCTGTAAAAGGCCG | |
| | TTTCACCATCAGCCGTGACAATAGC | |
| | AAGAACACCCTGTTCTTACAGATGA | |
| | ATTCCCTGCGTGCTGAAGATACCGC | |
| | CGTATACTACTGCGCTACTAACGAC | |
| | GATTATTGGGGCCAAGGTACCCTGG | |
| | TCACCGTCTCCTCTGGCGGTGGTGG | |
| | TAGCGGAGGCGGAGGATCAGGTGGA | |
| | GGCGGCAGTGAAATTGTGCTGACCC | |
| | AAAGCCCAGCAACCCTGAGCCTGTC | |
| | TCCAGGCGAACGTGCCACTCTGTCC | |
| | TGCCGTGCAAGCCAGTCTGTCTACC | |
| | ACTACCTGGCATGGTACCAGCAGAA | |
| | GCCAGGGCAAGCCCCACGTCTGTTA | |
| | ATCTACGACGCAGAGAATCGTGCCA | |
| | CCGGTATTCCAGCCCGTTTTTCTGG | |
| | CAGCGGTTCCGGCACCGATTTCACT | |
| | CTGACCATCTCCTCCCTGGAACCAG | |
| | AAGACTTCGCCGTTTACTACTGTCA | |
| | ACAGTATAACGACTGGCCACTTACT | |
| | TTTGGTCAGGGCACCAAAGTTGAGA | |
| | TTAAA | |
| OX- | CAGGTACAATTGGTAGAGTCCGGTG | 83 |
| Clone36 | GTGGCGTTGTCCAGCCAGGCCGTTC | |
| OX40 | CCTGCGTCTGGACTGCAAAGCATCT | |
| library | GGCATCGACTTCTCCAACAGCGGTA | |
| | TGCACTGGGTGCGTCAGGCCCCAGG | |
| | CAAAGGGCTGGAGTGGGTCGCCGTG | |
| | ATCTGGTATGATGGGTCTAAACGTT | |
| | ACTATGCTGACTCTGTAAAAGGCCG | |
| | TTTCACCATCAGCCGTGACAATAGC | |
| | AAGAACACCCTGTTCTTACAGATGA | |
| | ATTCCCTGCGTGCTGAAGATACCGC | |
| | CGTATACTACTGCGCTTCCAACGAC | |
| | GATTATTGGGGCCAAGGTACCCTGG | |
| | TCACCGTCTCCTCTGGCGGTGGTGG | |
| | TAGCGGAGGCGGAGGATCAGGTGGA | |
| | GGCGGCAGTGAAATTGTGCTGACCC | |
| | AAAGCCCAGCAACCCTGAGCCTGTC | |
| | TCCAGGCGAACGTGCCACTCTGTCC | |
| | TGCCGTGCAAGCCAGTCTGTCATCC | |
| | ACTACCTGGCATGGTACCAGCAGAA | |
| | GCCAGGGCAAGCCCCACGTCTGTTA | |
| | ATCTACGACGCAAGCAATCGTGCCA | |
| | CCGGTATTCCAGCCCGTTTTTCTGG | |
| | CAGCGGTTCCGGCACCGATTTCACT | |
| | CTGACCATCTCCTCCCTGGAACCAG | |
| | AAGACTTCGCCGTTTACTACTGTCA | |
| | ACAGTATGACAACTGGCCACTTACT | |
| | TTTGGTCAGGGCACCAAAGTTGAGA | |
| | TTAAA | |
| OX- | CAGGTACAATTGGTAGAGTCCGGTG | 86 |
| Clone71 | GTGGCGTTGTCCAGCCAGGCCGTTC | |
| OX40 | CCTGCGTCTGGACTGCAAAGCATCT | |
| library | GGCATCGACTTCTCCAACAGCGGTA | |
| | TGCACTGGGTGCGTCAGGCCCCAGG | |
| | CAAAGGGCTGGAGTGGGTCGCCGTG | |
| | ATCTGGTATGATGGGTCTAAACGTT | |
| | ACTATGCTGACTCTGTAAAAGGCCG | |
| | TTTCACCATCAGCCGTGACAATAGC | |
| | AAGAACACCCTGTTCTTACAGATGA | |
| | ATTCCCTGCGTGCTGAAGATACCGC | |
| | CGTATACTACTGCGCTTCCAACGAC | |
| | GATTATTGGGGCCAAGGTACCCTGG | |
| | TCACCGTCTCCTCTGGCGGTGGTGG | |
| | TAGCGGAGGCGGAGGATCAGGTGGA | |
| | GGCGGCAGTGAAATTGTGCTGACCC | |
| | AAAGCCCAGCAACCCTGAGCCTGTC | |
| | TCCAGGCGAACGTGCCACTCTGTCC | |
| | TGCCGTGCAAGCCAGTCTGTCGAGC | |
| | ACTACCTGGCATGGTACCAGCAGAA | |
| | GCCAGGGCAAGCCCCACGTCTGTTA | |
| | ATCTACGACGCAAGCAATCCCGCCA | |
| | CCGGTATTCCAGCCCGTTTTTCTGG | |
| | CAGCGGTTCCGGCACCGATTTCACT | |
| | CTGACCATCTCCTCCCTGGAACCAG | |
| | AAGACTTCGCCGTTTACTACTGTCA | |
| | ACAGTATTCAAACTGGCCACTTACT | |
| | TTTGGTCAGGGCACCAAAGTTGAGA | |
| | TTAAA | |
| 3222ACGI | CAGGTACAATTGGTAGAGTCCGGTG | 89 |
| Clone12 | GTGGCGTTGTCCAGCCAGGCCGTTC | |
| GITR | CCTGCGTCTGGACTGCAAAGCATCT | |
| library | GGCATCACCTTCTCCAACAGCGGTA | |
| | TGCACTGGGTGCGTCAGGCCCCAGG | |
| | CAAAGGGCTGGAGTGGGTCGCCGTG | |
| | ATCTGGTATGATGGGTCTCTGCGTT | |
| | ACTATGCTGACTCTGTAAAAGGCCG | |
| | TTTCACCATCAGCCGTGACAATAGC | |
| | AAGAACACCCTGTTCTTACAGATGA | |
| | ATTCCCTGCGTGCTGAAGATACCGC | |
| | CGTATACTACTGCGCTACTAACGAC | |
| | GATTATTGGGGCCAAGGTACCCTGG | |
| | TCACCGTCTCCTCTGGCGGTGGTGG | |
| | TAGCGGAGGCGGAGGATCAGGTGGA | |
| | GGCGGCAGTGAAATTGTGCTGACCC | |
| | AAAGCCCAGCAACCCTGAGCCTGTC | |
| | TCCAGGCGAACGTGCCACTCTGTCC | |
| | TGCCGTGCAAGCCAGTCTGTCTCTG | |
| | ACAACCTGGCGTGGTACCAGCAGAA | |
| | GCCAGGGCAAGCCCCACGTCTGTTA | |
| | ATCTACGACGCAGACACCCGTGCCA | |
| | CCGGTATTCCAGCCCGTTTTTCTGG | |
| | CAGCGGTTCCGGCACCGATTTCACT | |
| | CTGACCATCTCCTCCCTGGAACCAG | |
| | AAGACTTCGCCGTTTACTACTGTTG | |
| | CCAGTCATCAAACTTACCACGTACT | |
| | TTTGGTCAGGGCACCAAAGTTGAGA | |
| | TTAAA | |
| 3222ACGI | CAGGTACAATTGGTAGAGTCCGGTG | 92 |
| Clone2 | GTGGCGTTGTCCAGCCAGGCCGTTC | |
| GITR | CCTGCGTCTGGACTGCAAAGCATCT | |
| library | GGCATCACCTTCTCCAACAGCGGTA | |
| | TGCACTGGGTGCGTCAGGCCCCAGG | |
| | CAAAGGGCTGGAGTGGGTCGCCGTG | |
| | ATCTGGTATGATGGGTCTCTGCGTT | |
| | ACTATGCTGACTCTGTAAAAGGCCG | |
| | TTTCACCATCAGCCGTGACAATAGC | |
| | AAGAACACCCTGTTCTTACAGATGA | |
| | ATTCCCTGCGTGCTGAAGATACCGC | |
| | CGTATACTACTGCGCTACTAACGAC | |
| | GATTATTGGGGCCAAGGTACCCTGG | |
| | TCACCGTCTCCTCTGGCGGTGGTGG | |
| | TAGCGGAGGCGGAGGATCAGGTGGA | |
| | GGCGGCAGTGAAATTGTGCTGACCC | |
| | AAAGCCCAGCAACCCTGAGCCTGTC | |
| | TCCAGGCGAACGTGCCACTCTGTCC | |
| | TGCCGTGCAAGCGAGTCTGTCTCTG | |
| | ACAACCTGCAGTGGTACCAGCAGAA | |
| | GCCAGGGCAAGCCCCACGTCTGTTA | |
| | ATCTACGACGCAGACAATCGTGCCA | |
| | CCGGTATTCCAGCCCGTTTTTCTGG | |
| | CAGCGGTTCCGGCACCGATTTCACT | |
| | CTGACCATCTCCTCCCTGGAACCAG | |
| | AAGACTTCGCCGTTTACTACTGTCA | |
| | ACAGTCATCAAACTTACCACGTACT | |
| | TTTGGTCAGGGCACCAAAGTTGAGA | |
| | TTAAA | |
| 3222ACGI | CAGGTACAATTGGTAGAGTCCGGTG | 95 |
| Clone23 | GTGGCGTTGTCCAGCCAGGCCGTTC | |
| GITR | CCTGCGTCTGGACTGCAAAGCATCT | |
| library | GGCATCACCTTCTCCAACAGCGGTA | |
| | TGCACTGGGTGCGTCAGGCCCCAGG | |
| | CAAAGGGCTGGAGTGGGTCGCCGTG | |
| | ATCTGGTATGATGGGTCTCTGCGTT | |
| | ACTATGCTGACTCTGTAAAAGGCCG | |
| | TTTCACCATCAGCCGTGACAATAGC | |
| | AAGAACACCCTGTTCTTACAGATGA | |
| | ATTCCCTGCGTGCTGAAGATACCGC | |
| | CGTATACTACTGCGCTACTAACGAC | |
| | GATTATTGGGGCCAAGGTACCCTGG | |
| | TCACCGTCTCCTCTGGCGGTGGTGG | |
| | TAGCGGAGGCGGAGGATCAGGTGGA | |
| | GGCGGCAGTGAAATTGTGCTGACCC | |
| | AAAGCCCAGCAACCCTGAGCCTGTC | |
| | TCCAGGCGAACGTGCCACTCTGTCC | |
| | TGCCGTGCAAGCCAGTCTGTCTCTG | |
| | ACAACCTGCAGTGGTACCAGCAGAA | |
| | GCCAGGGCAAGCCCCACGTCTGTTA | |
| | ATCTACGACGCAGACACCCGTGCCA | |
| | CCGGTATTCCAGCCCGTTTTTCTGG | |
| | CAGCGGTTCCGGCACCGATTTCACT | |
| | CTGACCATCTCCTCCCTGGAACCAG | |
| | AAGACTTCGCCGTTTACTACTGTCA | |
| | ACAGTCATCAAACTTACCACGTACT | |
| | TTTGGTCAGGGCACCAAAGTTGAGA | |
| | TTAAA | |
| 3222ACGI | CAGGTACAATTGGTAGAGTCCGGTG | 98 |
| Clone10 | GTGGCGTTGTCCAGCCAGGCCGTTC | |
| GITR | CCTGCGTCTGGACTGCAAAGCATCT | |
| library | GGCTTCACCTTCTCCAACAGCGGTA | |
| | TGCACTGGGTGCGTCAGGCCCCAGG | |
| | CAAAGGGCTGGAGTGGGTCGCCGTG | |
| | ATCTGGTATGATGGGTCTCTGCGTT | |
| | ACTATGCTGACTCTGTAAAAGGCCG | |
| | TTTCACCATCAGCCGTGACAATAGC | |
| | AAGAACACCCTGTTCTTACAGATGA | |
| | ATTCCCTGCGTGCTGAAGATACCGC | |
| | CGTATACTACTGCGCTACTAACGAC | |
| | GATTATTGGGGCCAAGGTACCCTGG | |
| | TCACCGTCTCCTCTGGCGGTGGTGG | |
| | TAGCGGAGGCGGAGGATCAGGTGGA | |
| | GGCGGCAGTGAAATTGTGCTGACCC | |
| | AAAGCCCAGCAACCCTGAGCCTGTC | |
| | TCCAGGCGAACGTGCCACTCTGTCC | |
| | TGCCGTGCAAGCCAGTCTGTCTCTG | |
| | ACAACCTGCAGTGGTACCAGCAGAA | |
| | GCCAGGGCAAGCCCCACGTCTGTTA | |
| | ATCTACGACGCAGACACGCGTGCCA | |
| | CCGGTATTCCAGCCCGTTTTTCTGG | |
| | CAGCGGTTCCGGCACCGATTTCACT | |
| | CTGACCATCTCCTCCCTGGAACCAG | |
| | AAGACTTCGCCGTTTACTACTGTCA | |
| | ACAGTCATCAAACTTACCACGTACT | |
| | TTTGGTCAGGGCACCAAAGTTGAGA | |
| | TTAAA | |
| 3222ACGI | CAGGTACAATTGGTAGAGTCCGGTG | 101 |
| Clone15 | GTGGCGTTGTCCAGCCAGGCCGTTC | |
| GITR | CCTGCGTCTGGACTGCAAAGCATCT | |
| library | GGCATCACCTTCTCCAACAGCGGTA | |
| | TGCACTGGGTGCGTCAGGCCCCAGG | |
| | CAAAGGGCTGGAGTGGGTCGCCGTG | |
| | ATCTGGTATGATGGGTCTCTGCGTT | |
| | ACTATGCTGACTCTGTAAAAGGCCG | |
| | TTTCACCATCAGCCGTGACAATAGC | |
| | AAGAACACCCTGTTCTTACAGATGA | |
| | ATTCCCTGCGTGCTGAAGATACCGC | |
| | CGTATACTACTGCGCTACTAACGAC | |
| | GATTATTGGGGCCAAGGTACCCTGG | |
| | TCACCGTCTCCTCTGGCGGTGGTGG | |
| | TAGCGGAGGCGGAGGATCAGGTGGA | |
| | GGCGGCAGTGAAATTGTGCTGACCC | |
| | AAAGCCCAGCAACCCTGAGCCTGTC | |
| | TCCAGGCGAACGTGCCACTCTGTCC | |
| | TGCCGTGCAAGCCAGTCTGTCTCTG | |
| | ACAACCTGGCATGGTACCAGCAGAA | |
| | GCCAGGGCAAGCCCCACGTCTGTTA | |
| | ATCTACGACGCAGACAATCGTGCCA | |
| | CCGGTATTCCAGCCCGTTTTTCTGG | |
| | CAGCGGTTCCGGCACCGATTTCACT | |
| | CTGACCATCTCCTCCCTGGAACCAG | |
| | AAGACTTCGCCGTTTACTACTGTAT | |
| | GCAGTCATCAAACTTAGCCCGTACT | |
| | TTTGGTCAGGGCACCAAAGTTGAGA | |
| | TTAAA | |
| 3222ACGI | CAGGTACAATTGGTAGAGTCCGGTG | 104 |
| Clone1 | GTGGCGTTGTCCAGCCAGGCCGTTC | |
| GITR | CCTGCGTCTGGACTGCAAAGCATCT | |
| library | GGCATCGCCTTCTCCAACAGCGGTA | |
| | TGCACTGGGTGCGTCAGGCCCCAG | |
| | GCAAAGGGCTGGAGTGGGTCGCCGT | |
| | GATCTGGTATGATGGGTCTCTGCGT | |
| | TACTATGCTGACTCTGTAAAAGGCC | |
| | GTTTCACCATCAGCCGTGACAATAG | |
| | CAAGAACACCCTGTTCTTACAGATG | |
| | AATTCCCTGCGTGCTGAAGATACCG | |
| | CCGTATACTACTGCGCTACTAACGA | |
| | CGATTATTGGGGCCAAGGTACCCTG | |
| | GTCACCGTCTCCTCTGGCGGTGGTG | |
| | GTAGCGGAGGCGGAGGATCAGGTGG | |
| | AGGCGGCAGTGAAATTGTGCTGACC | |
| | CAAAGCCCAGCAACCCTGAGCCTGT | |
| | CTCCAGGAGAACGTGCCACTCTGTC | |
| | CTGCCGTGCAAGCCAGTCTGTCTCT | |
| | GACAACCTGGCATGGTACCAGCAGA | |
| | AGCCAGGGCAAGCCCCACGTCTGTT | |
| | AATCTACGACGCAGACAATCGTGCC | |
| | ACCGGTATTCCAGCCCGTTTTTCTG | |
| | GCAGCGGTTCCGGCACCGATTTCAC | |
| | TCTGACCATCTCCTCCCTGGAACCA | |
| | GAAGACTTCGCCGTTTACTACTGTT | |
| | GCCAGTCATCAAACTTACCACGTAC | |
| | TTTTGGTCAGGGCACCAAAGTTGAG | |
| | ATTAAA | |
| 3222ACGI | CAGGTACAATTGGTAGAGTCCGGTG | 107 |
| Clone5 | GTGGCGTTGTCCAGCCAGGCCGTTC | |
| GITR | CCTGCGTCTGGACTGCAAAGCATCT | |
| library | GGCATCACCTTCTCCAACAGCGGTA | |
| | TGCACTGGGTGCGTCAGGCCCCAGG | |
| | CAAAGGGCTGGAGTGGGTCGCCGTG | |
| | ATCTGGTATGATGGGTCTCTGCGTT | |
| | ACTATGCTGACTCTGTAAAAGGCCG | |
| | TTTCACCATCAGCCGTGACAATAGC | |
| | AAGAACACCCTGTTCTTACAGATGA | |
| | ATTCCCTGCGTGCTGAAGATACCGC | |
| | CGTATACTACTGCGCTACTAACGAC | |
| | GATTATTGGGGCCAAGGTACCCTGG | |
| | TCACCGTCTCCTCTGGCGGTGGTGG | |
| | TAGCGGAGGCGGAGGATCAGGTGGA | |
| | GGCGGCAGTGAAATTGTGCTGACCC | |
| | AAAGCCCAGCAACCCTGAGCCTGTC | |
| | TCCAGGCGAACGTGCCACTCTGTCC | |
| | TGCCGTGCAAGCCTGTCTGTCTCTG | |
| | ACAACCTGGCATGGTACCAGCAGAA | |
| | GCCAGGGCAAGCCCCACGTCTGTTA | |
| | ATCTACGACGCAGACAACCGTGCCA | |
| | CCGGTATTCCAGCCCGTTTTTCTGG | |
| | CAGCGGTTCCGGCACCGATTTCACT | |
| | CTGACCATCTCCTCCCTGGAACCAG | |
| | AAGACTTCGCCGTTTACTACTGTAT | |
| | GCAGTCATCAAACTTACCACGTACT | |
| | TTTGGTCAGGGCACCAAAGTTGAGA | |
| | TTAAA | |

The amino acid sequences of the VH and VL chains for the second-generation clones listed above are provided in SEQ ID NOs:54, 55, 57, 58, 60, 61, 63, 64, 66, 67, 69, 70, 72, 73, 75, 76, 78, 79, 81, 82, 84, 85, 87, 88, 90, 91, 93, 94, 96, 97, 99, 100, 102, 103, 105, 106, 108, and 109.

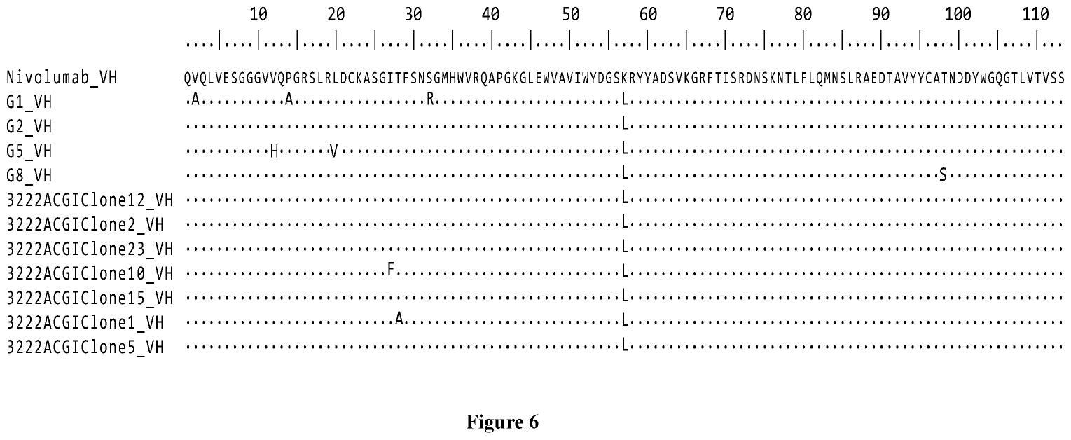

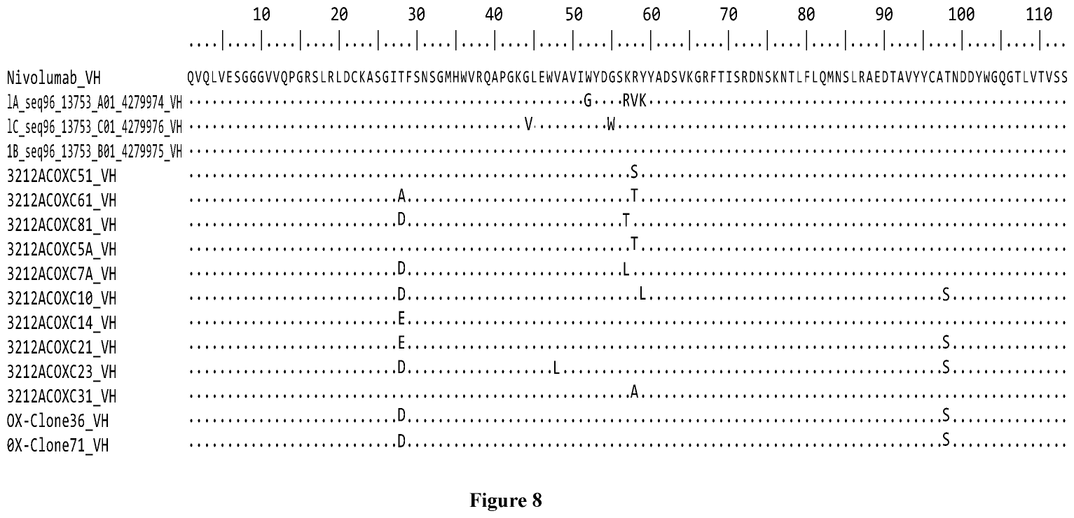

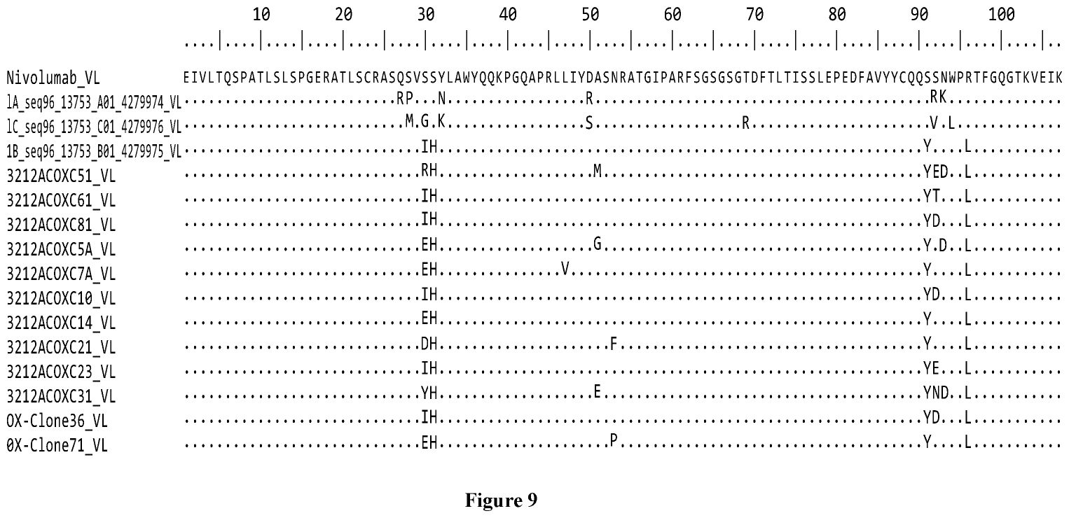

The amino acid sequence alignments within the VH and VL regions between Nivolumab and the selected clones for dual specific binders against PD-1/hGITR and PD-1/OX40 are presented in FIGS. 6, 7, 8, and 9.

Summary: The methods and results provided here demonstrate the ability to effectively select a candidate antibody as a potential dual binder, and through a series of steps produce a dual binding antibody that binds two independent epitopes in the region comprising the original antibody binding site.

### Example 2

### Analysis of Dual Binding PD-1/hGITR Antibodies

Objective: To analyze the physical and binding properties of dual binding PD-1/hGITR clones produced in Example 1.

Methods:

### GITR-PD-1 IgG Production and Characterization:

Clones identified in Example 1 that showed the most promising binding in YSD scFV format were reformatted to human IgG1 format, transiently expressed in expi-CHO cells according to the manufacturer's instructions and purified as described herein. Size exclusion chromatography on a Superdex 10/300 increase column, using PBS as a mobile phase was performed to analyze IgG1 antibodies.

To test whether the binding of BDG32.004 and BDG32.005 are specific, they were tested for binding to 100 nM hPD-1-His, vs 500 nM human IL-2 and 500 nM mouse TNFR2 in an ELISA assay as described herein, with an anti hIL-2 antibody serving as a positive control.

ELISA assays were used to analyze the binding affinity of the IgG1 antibodies. Briefly, BDG32.004 and BDG32.005 were coated directly on the surface of the ELISA plate and blocked, subsequently PD-1 or hGITR-his were added at concentrations ranging from 0 to 200 nM and 0 to 865 nM, respectively. As a reference Nivolumab in IgG1 format was tested against hPD-1 under the same condition.

### Inhibition of PD-1 Signaling

A cellular reporter PD1/PDL1 blocking assay was used to test the ability of IgG1 Abs to inhibit PD-1 to PD-1 interaction in live cells, where PD-1 is located on the cellular membrane in its biologically relevant conformation. Detailed description is provided above. Briefly, the reporter assay monitors the luminescence of engineered Jurket effector cells expressing the PD-1 NFAT-mediated luciferase reporter system. When these cells are mixed with CHO-K1 cells expressing PD-L1 the PD-1/PD-L1 interaction inhibits TCR signaling and NFAT-mediated luciferase activity.

Results:

The amino acid sequences of the VH and VL regions of representative examples of the clones reformatted to human IgG1 format are presented in Table 6.

TABLE 6
VH and VL Amino Acid Sequences of Second-
Generation PD-1-GITR Dual Antibody Clones

| | Antibody | Heavy chain variable | SEQ | Light chain variable | SEQ |
|---|---|---|---|---|---|
| Antibody | YSD | region amino acid | ID | region amino acid | ID |
| I.D. | identifier | sequence | NO: | sequence | NO: |
| BDG32.004 | Gl_c10 | QVQLVESGGGVVQP | 99 | EIVLTQSPATLSLSPGE | 100 |
| | | GRSLRLDCKASGFTFS | | RATLSCRASQSVSDNL | |
| | | NSGMHWVRQAPGKG | | QWYQQKPGQAPRLLI | |
| | | LEWVAVIWYDGSLR | | YDADTRATGIPARFSG | |
| | | YYADSVKGRFTISRD | | SGSGTDFTLTISSLEPE | |
| | | NSKNTLFLQMNSLRA | | DFAVYYCQQSSNLPRT | |
| | | EDTAVYYCATNDDY | | FGQGTKVEIK | |
| | | WGQGTLVTVSS | | | |
| BDG32.005 | Gl_c15 | QVQLVESGGGVVQP | 102 | EIVLTQSPATLSLSPGE | 103 |
| | | GRSLRLDCKASGITFS | | RATLSCRASQSVSDNL | |
| | | NSGMHWVRQAPGKG | | AWYQQKPGQAPRLLI | |
| | | LEWVAVIWYDGSLR | | YDADNRATGIPARFSG | |
| | | YYADSVKGRFTISRD | | SGSGTDFTLTISSLEPE | |
| | | NSKNTLFLQMNSLRA | | DFAVYYCMQSSNLAR | |
| | | EDTAVYYCATNDDY | | TFGQGTKVEIK | |
| | | WGQGTLVTVSS | | | |
| BDG32.006 | Gl_c23 | QVQLVESGGGVVQP | 96 | EIVLTQSPATLSLSPGE | 97 |
| | | GRSLRLDCKASGITFS | | RATLSCRASQSVSDNL | |
| | | NSGMHWVRQAPGKG | | QWYQQKPGQAPRLLI | |
| | | LEWVAVIWYDGSLR | | YDADTRATGIPARFSG | |
| | | YYADSVKGRFTISRD | | SGSGTDFTLTISSLEPE | |
| | | NSKNTLFLQMNSLRA | | DFAVYYCQQSSNLPRT | |
| | | EDTAVYYCATNDDY | | FGQGTKVEIK | |
| | | WGQGTLVTVSS | | | |
| BDG32.018 | Gl_C5 | QVQLVESGGGVVQP | 108 | EIVLTQSPATLSLSPGE | 109 |
| | | GRSLRLDCKASGITFS | | RATLSCRASLSVSDNL | |
| | | NSGMHWVRQAPGKG | | AWYQQKPGQAPRLLI | |
| | | LEWVAVIWYDGSLR | | YDADNRATGIPARFSG | |
| | | YYADSVKGRFTISRD | | SGSGTDFTLTISSLEPE | |
| | | NSKNTLFLQMNSLRA | | DFAVYYCMQSSNLPR | |
| | | EDTAVYYCATNDDY | | TFGQGTKVEIK | |
| | | WGQGTLVTVSS | | | |

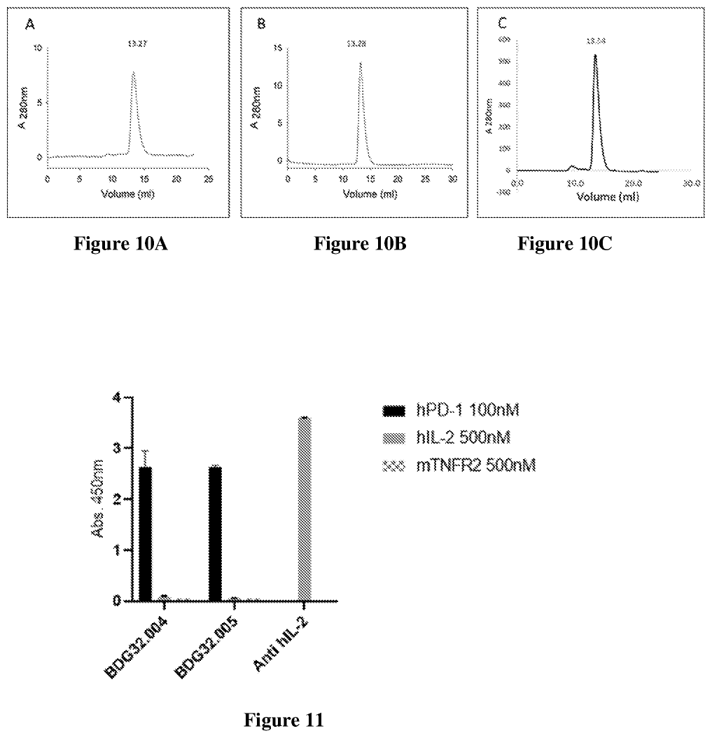

BDG32.004 and BDG32.005 were analyzed on a Superdex 10/600 increase, Size Exclusion Chromatography (SEC) column, using PBS as a mobile phase. These antibodies showed retention times comparable to Nivolumab that was produced in an IgG1 format, and as can be seen in FIGS. 10A-10C, had less than 1% aggregation as evident from peak integration analysis.

To test if BDG32.004 and BDG32.005 show specific PD1 binding, they were tested for binding to 100 nM hPD-1-His, vs 500 nM human IL-2 and 500 nM mouse TNFR2 in an ELISA assay, with an anti hIL-2 antibody serving as a positive control. As can be seen in FIG. 11 both BDG32.004 and BDG32.005 specifically binds PD1 with very minimal nonspecific binding to hIL-2 and mTNFR2.

Affinity to PD-1 and GITR

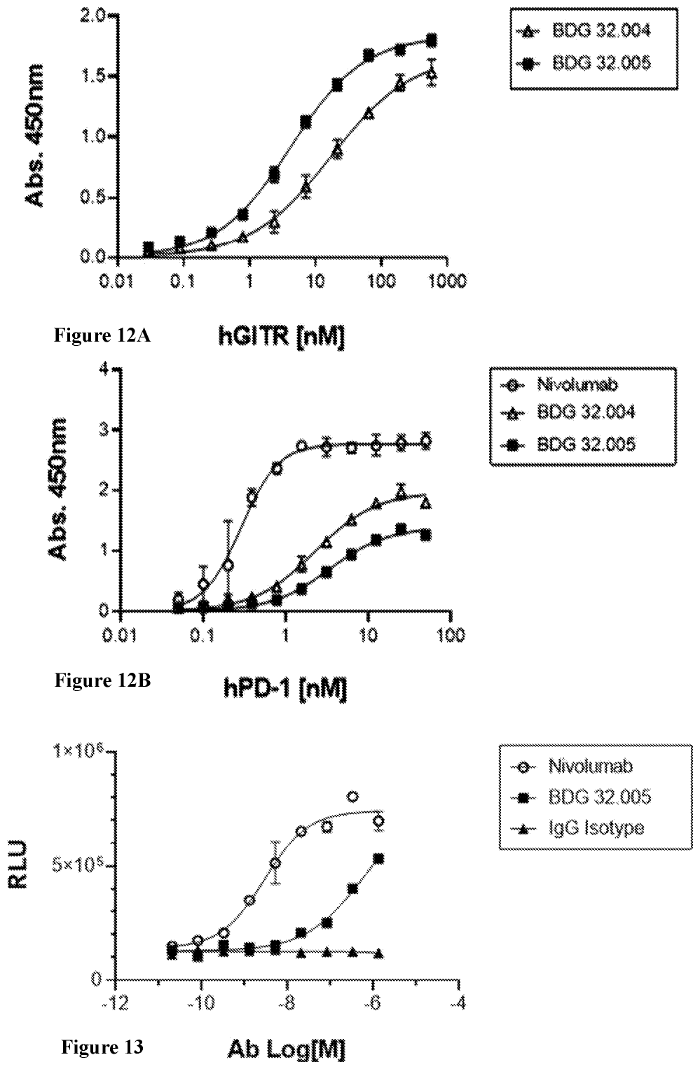

BDG32.004 and BDG32.005 binding to both GITR and PD-1 was tested by direct ELISA. As can be seen in FIGS. 12A and 12B, BDG32.004 and BDG32.005 had an EC₅₀ of approximately 13 nM and 4 nM towards hGITR-His, respectively. Their apparent affinity to hPD1-His was 29 nM and 16 nM, respectively, which is approximately within one order of magnitude of the 1.2 nM measured affinity of Nivolumab in IgG1 format. This indicates that gaining specific and tight binding to GITR did not reduce significantly binding of BDG32.004 and BDG32.005 to hPD1.

### Inhibition of PD-1 Signaling

To test IgG1 BDG32.005 ability to inhibit PD-1—PD-L1 interaction in a live cell setting, where PD-1 is located on the cellular membrane in its biologically relevant conformation, a cellular reporter PD1/PDL1 blocking assay was used. As can be seen in FIG. 13, BDG32.005 and Nivolumab are activating the PD-1 dependent luminescence whereas an IgG1 isotype control does not. The IC₅₀ for Nivolumab is approximately 3 nM and for BDG32.005 is approximately 614 nM. This experiment suggests that BDG32.005 is capable of binding and inhibiting PD-1 in a biologically relevant cellular context.

Summary: The data presented here shows dual specificity of binding with negligible non-specific binding for dual binding PD-1/hGITR antibodies, wherein binding and inhibiting PD-1 in a relevant biological context was also demonstrated.

### Example 3

### Analysis of Dual Binding PD-1/OX40 Antibodies

Objective: To analyze the physical and binding properties of dual binding PD-1/OX40 clones produced in Example 1.

Methods:

### Screening for PD-1 OX40 Binders

The library described in Example 1 was screened in a yeast surface display format to identify clones that bind OX40 while maintaining the binding to PD-1. The yeast library was grown to a cell number of 3×10⁹ induced on SG media to express the scFV at 20 C and labeled with 0.5 μM of recombinant human PD-1 tagged with histidine tag (hPD-1-His). Yeast binders were selected on magnetic beads, roughly 2×10⁶ yeast cells were eluted from the magnetic column indicating enrichment factor of approximately 1000-fold. A second round of selection was done by labeling yeast cells with 0.5 μM (hOX40-His) using anti His-APC and anti MycFITC secondary antibodies, and selecting on a S3e Fluorescence Activated Cell Sorter (FACS). Approximately, the top 1% of the yeast cells were selected. Third and fourth rounds of selection were conducted in a similar fashion with 500 nM hOX40-His.

At the end of the fourth round of selection the yeast were plated on tryptophan depleted synthetic medium and individual clones were isolated, sequenced and tested for specific binding to both 500 nM human recombinant PD-1 tagged with histidine tag (hPD-1-His) and 500 nM hOX40-His.

Affinity maturation library design is as described above. To screen for affinity matured PD-1, OX40 dual binders, the yeast were grown to a cell number of 1×10¹⁰ induced with SG media to express scFV at 20° C. and labeled with 100 nM hOX40-His. hOX40-His binding yeast were selected on magnetic beads, 1×10⁸ yeast cells were eluted from the magnetic column indicating enrichment factor of approximately 100-fold.

Since the number of MACS selected clones was high, a second round of magnetic beads selection was performed on 1×10⁹ cells labeled with 10 nM hOX40-His. About 5×10⁶ yeast cells were eluted from the magnetic column indicating enrichment factor of approximately 200-fold. A third round of selection was done by labeling yeast cells with 100 nM (hOX40-His) with anti His-APC and anti Myc-FITC secondary labeling, and selecting on a S3e Fluorescence Activated Cell Sorter (FACS). The top 2% binders of the yeast cells were selected. Then a fourth round of selection was performed by labeling the yeast with 30 nM hPD-1-His, the top 20% binders were selected. The fifth round of selection was executed by labeling the yeast with 30 nM hPD-1 His and the top 3% yeast binders were elected. A final round of selection was done with the yeast labeled with 100 nM hOX40-His, then after 1 hour incubation with the hOX40-His, the yeast were washed and incubated in 1 ml PBS-F buffer for 1 hour and only then labeled with anti His-APC and anti Myc-FITC and sorted.

Subsequently to the final round of selection, the yeast were plated on tryptophan-depleted synthetic medium and individual clones were isolated, sequenced and tested for specific binding to both 30 nM human recombinant PD-1 tagged with histidine tag (hPD-1-His) and 90 nM hOX-His (see FIG. 15).

### OX40-PD-1 IgG Production and Characterization:

Clones that showed the most promising binding in yeast surface display (YSD) scFV format were reformatted to human IgG1 format, transiently expressed in expi-CHO cells according to the manufacturer's instructions and purified.

### Affinity to PD-1 and OX40

Affinity of BDG32.007 and BDG32.008 to both OX40 and PD-1 was tested by direct ELISA. Briefly, BDG32.007 and BDG32.008 were coated directly on the surface of the ELISA plate and blocked, subsequently PD-1 or hOX40-his were added at concentration ranging from 0 to 50 nM and 0 to 500 nM, respectively. As a reference Nivolumab in IgG1 format was tested against hPD-1 under the same condition.

### Inhibition of PD-1 Signaling in Cells

A cellular reporter PD1/PDL1 blocking assay was performed as described above.

### Results

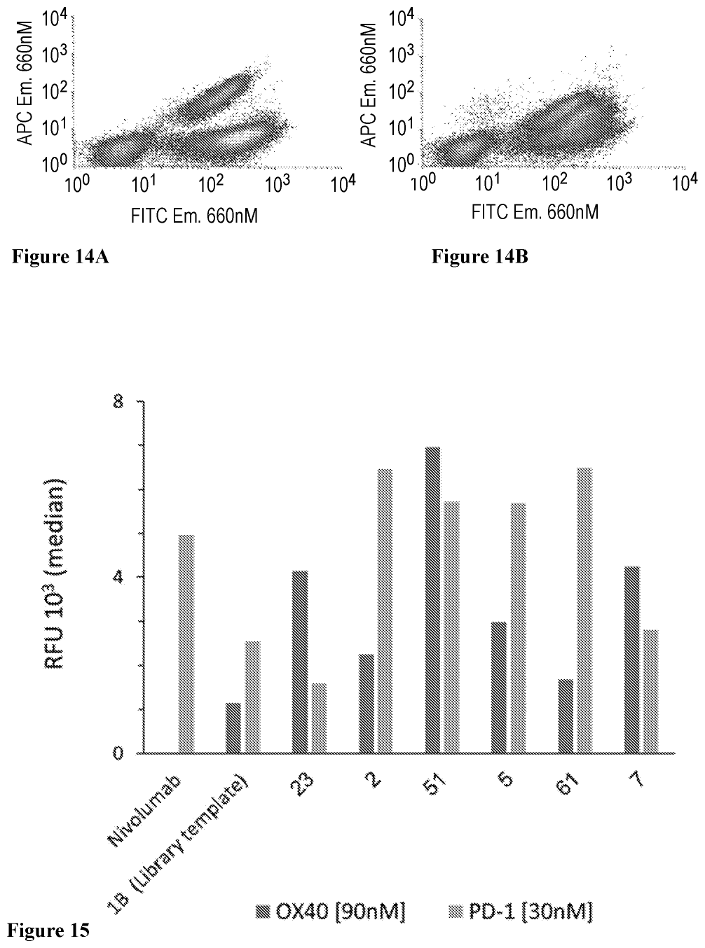

As can be seen in FIGS. 14A and 14B, FACS analysis of the library against either PD-1 or OX40 resulted in more than 40% of the library population binding to OX40 and more than 20% of the population binding to PD-1. Binders that showed enhanced binding for both PD-1 and OX40 targets are listed herein. The amino acid sequences of the VH and VL regions are set forth in any one of SEQ ID NOs: 54, 55, 57, 58, 60, 61, 63, 64, 66, 67, 69, 70, 72, 73, 75, 76, 78, 79, 81, 82, 84, 85, 87, and 88.

The amino acid sequences of the VH and VL regions of representative examples of the clones reformatted to human IgG1 format are presented in Table 7.

TABLE 7
VH and VL Amino Acid Sequences of Second-
Generation PD-1-OX40 Dual Antibody Clones

| IgG BDG | | | SEQ ID | | SEQ ID |
|---|---|---|---|---|---|
| name | Clone name | VH | NO: | VL | NO: |
| BDG32.014 | 3212ACOX | QVQLVESGGGVVQPG | 54 | EIVLTQSPATLSLSP | 55 |
| | C51 OX40 | RSLRLDCKASGITFS | | GERATLSCRASQSVR | |
| | library | NSGMHWVRQAPGKGL | | HYLAWYQQKPGQAPR | |
| | | EWVAVIWYDGSKSYY | | LLIYDAMNRATGIPA | |
| | | ADSVKGRFTISRDNS | | RFSGSGSGTDFTLTI | |
| | | KNTLFLQMNSLRAED | | SSLEPEDFAVYYCQQ | |
| | | TAVYYCATNDDYWGQ | | YEDWPLTFGQGTKVE | |
| | | GTLVTVSS | | IK | |
| BDG32.015 | 3212ACOX | QVQLVESGGGVVQPG | 57 | EIVLTQSPATLSLSP | 58 |
| | C61 OX40 | RSLRLDCKASGIAFS | | GERATLSCRASQSVI | |
| | library | NSGMHWVRQAPGKGL | | HYLAWYQQKPGQAPR | |
| | | EWVAVIWYDGSKTYY | | LLIYDASNRATGIPA | |
| | | ADSVKGRFTISRDNS | | RFSGSGSGTDFTLTI | |
| | | KNTLFLQMNSLRAED | | SSLEPEDFAVYYCQQ | |
| | | TAVYYCATNDDYWGQ | | YTNWPLTFGQGTKVE | |
| | | GTLVTVSS | | IK | |
| BDG32.016 | 3212ACOX | RDNSKNTLFLQMNS | 60 | EIVLTQSPATLSLSP | 61 |
| | C81 OX40 | LRAEQVQLVESGGGV | | GERATLSCRASQSVI | |
| | library | VQPGRSLRLDCKASG | | HYLAWYQQKPGQAPR | |
| | | IDFSNSGMHWVRQAP | | LLIYDASNRATGIPA | |
| | | GKGLEWVAVIWYDGS | | RFSGSGSGTDFTLTI | |
| | | TRYYADSVKGRFTIS | | SSLEPEDFAVYYCQQ | |
| | | DTAVYYCATNDDYWG | | YDNWPLTFGQGTKVE | |
| | | QGTLVTVSS | | IK | |
| BDG32.007 | 3212ACOX | QVQLVESGGGVVQPG | 63 | EIVLTQSPATLSLSP | 64 |
| | C5A OX40 | RSLRLDCKASGITFS | | GERATLSCRASQSVE | |
| | library | NSGMHWVRQAPGKGL | | HYLAWYQQKPGQAPR | |
| | | EWVAVIWYDGSKTYY | | LLIYDAGNRATGIPA | |
| | | ADSVKGRFTISRDNS | | RFSGSGSGTDFTLTI | |
| | | KNTLFLQMNSLRAED | | SSLEPEDFAVYYCQQ | |
| | | TAVYYCATNDDYWGQ | | YSDWPLTFGQGTKVE | |
| | | GTLVTVSS | | IK | |
| BDG32.008 | 3212ACOX | QVQLVESGGGVVQPG | 66 | EIVLTQSPATLSLSP | 67 |
| | C7A OX40 | RSLRLDCKASGIDFS | | GERATLSCRASQSVE | |
| | library | NSGMHWVRQAPGKGL | | HYLAWYQQKPGQAPR | |
| | | EWVAVIWYDGSLRYY | | LLVYDASNRATGIPA | |
| | | ADSVKGRFTISRDNS | | RFSGSGSGTDFTLTI | |
| | | KNTLFLQMNSLRAED | | SSLEPEDFAVYYCQQ | |
| | | TAVYYCATNDDYWGQ | | YSNWPLTFGQGTKVE | |
| | | GTLVTVSS | | IK | |
| BDG32.009 | 3212ACOX | QVQLVESGGGVVQPG | 69 | EIVLTQSPATLSLSP | 70 |
| | C10 OX40 | RSLRLDCKASGIDFS | | GERATLSCRASQSVI | |
| | library | NSGMHWVRQAPGKGL | | HYLAWYQQKPGQAPR | |
| | | EWVAVIWYDGSKRLY | | LLIYDASNRATGIPA | |
| | | ADSVKGRFTISRDNS | | RFSGSGSGTDFTLTI | |
| | | KNTLFLQMNSLRAED | | SSLEPEDFAVYYCQQ | |
| | | TAVYYCASNDDYWGQ | | YDNWPLTFGQGTKVE | |
| | | GTLVTVSS | | IK | |
| BDG32.010 | 3212ACOX | QVQLVESGGGVVQPG | 72 | EIVLTQSPATLSLSP | 73 |
| | C14 OX40 | RSLRLDCKASGIEFS | | GERATLSCRASQSVE | |
| | library | NSGMHWVRQAPGKGL | | HYLAWYQQKPGQAPR | |
| | | EWVAVIWYDGSKRYY | | LLIYDASNRATGIPA | |
| | | ADSVKGRFTISRDNS | | RFSGSGSGTDFTLTI | |
| | | KNTLFLQMNSLRAED | | SSLEPEDFAVYYCQQ | |
| | | TAVYYCATNDDYWGQ | | YSNWPLTFGQGTKVE | |
| | | GTLVTVSS | | IK | |
| BDG32.011 | 3212ACOX | RDNSKNTLFLQMNS | 75 | EIVLTQSPATLSLSP | 76 |
| | C21 OX40 | LRAEQVQLVESGGGV | | GERATLSCRASQSVD | |
| | library | VQPGRSLRLDCKASG | | HYLAWYQQKPGQAPR | |
| | | IEFSNSGMHWVRQAP | | LLIYDASNFATGIPA | |
| | | GKGLEWVAVIWYDGS | | RFSGSGSGTDFTLTI | |
| | | KRYYADSVKGRFTIS | | SSLEPEDFAVYYCQQ | |
| | | DTAVYYCASNDDYWG | | YSNWPLTFGQGTKVE | |
| | | QGTLVTVSS | | IK | |
| BDG32.012 | 3212ACOX | QVQLVESGGGVVQPG | 78 | EIVLTQSPATLSLSP | 79 |
| | C23 OX40 | RSLRLDCKASGIDFS | | GERATLSCRASQSVI | |
| | library | NSGMHWVRQAPGKGL | | HYLAWYQQKPGQAPR | |
| | | EWLAVIWYDGSKRYY | | LLIYDASNRATGIPA | |
| | | ADSVKGRFTISRDNS | | RFSGSGSGTDFTLTI | |
| | | KNTLFLQMNSLRAED | | SSLEPEDFAVYYCQQ | |
| | | TAVYYCASNDDYWGQ | | YENWPLTFGQGTKVE | |
| | | GTLVTVSS | | IK | |
| BDG32.013 | 3212ACOX | QVQLVESGGGVVQPG | 81 | EIVLTQSPATLSLSP | 82 |
| | C31 OX40 | RSLRLDCKASGITFS | | GERATLSCRASQSVY | |
| | library | NSGMHWVRQAPGKGL | | HYLAWYQQKPGQAPR | |
| | | EWVAVIWYDGSKAYY | | LLIYDAENRATGIPA | |
| | | ADSVKGRFTISRDNS | | RFSGSGSGTDFTLTI | |
| | | KNTLFLQMNSLRAED | | SSLEPEDFAVYYCQQ | |
| | | TAVYYCATNDDYWGQ | | YNDWPLTFGQGTKVE | |
| | | GTLVTVSS | | IK | |
| BDG32.023 | OX- | QVQLVESGGGVVQPG | 84 | EIVLTQSPATLSLSP | 85 |
| | Clone36 | RSLRLDCKASGIDFS | | GERATLSCRASQSVI | |
| | OX40 | NSGMHWVRQAPGKGL | | HYLAWYQQKPGQAPR | |
| | library | EWVAVIWYDGSKRYY | | LLIYDASNRATGIPA | |
| | | ADSVKGRFTISRDNS | | RFSGSGSGTDFTLTI | |
| | | KNTLFLQMNSLRAED | | SSLEPEDFAVYYCQQ | |
| | | TAVYYCASNDDYWGQ | | YDNWPLTFGQGTKVE | |
| | | GTLVTVSS | | IK | |
| BDG32.024 | OX- | QVQLVESGGGVVQPG | 87 | EIVLTQSPATLSLSP | 88 |
| | Clone71 | RSLRLDCKASGIDFS | | GERATLSCRASQSVE | |
| | OX40 | NSGMHWVRQAPGKGL | | HYLAWYQQKPGQAPR | |
| | library | EWVAVIWYDGSKRYY | | LLIYDASNPATGIPA | |
| | | ADSVKGRFTISRDNS | | RFSGSGSGTDFTLTI | |
| | | KNTLFLQMNSLRAED | | SSLEPEDFAVYYCQQ | |
| | | TAVYYCASNDDYWGQ | | YSNWPLTFGQGTKVE | |
| | | GTLVTVSS | | IK | |

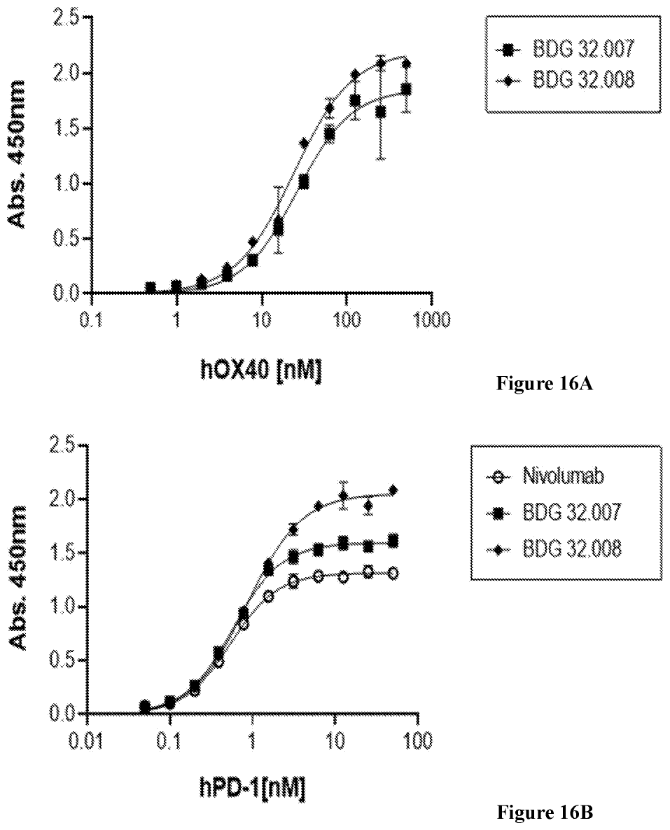

As can be seen in FIGS. 16A and 16B, BDG32.007 and BDG32.008 had an EC₅₀ of approximately 26 nM and 24 nM towards hOX40-His, respectively. The apparent affinity to hPD1-His was 0.9 nM and 0.6 nM, respectively, almost identical to the PD-1 affinity of Nivolumab in IgG1 format that was 0.5 nM in this assay. This indicates that gaining specific and tight binding to OX40) did not reduce binding of BDG32.007 and BDG32.008 to hPD1.

### Inhibition of PD-1 Signaling

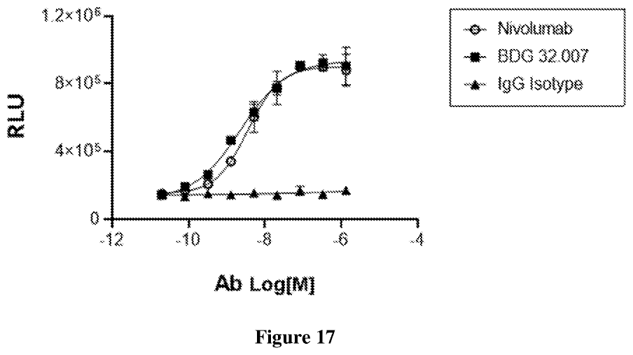

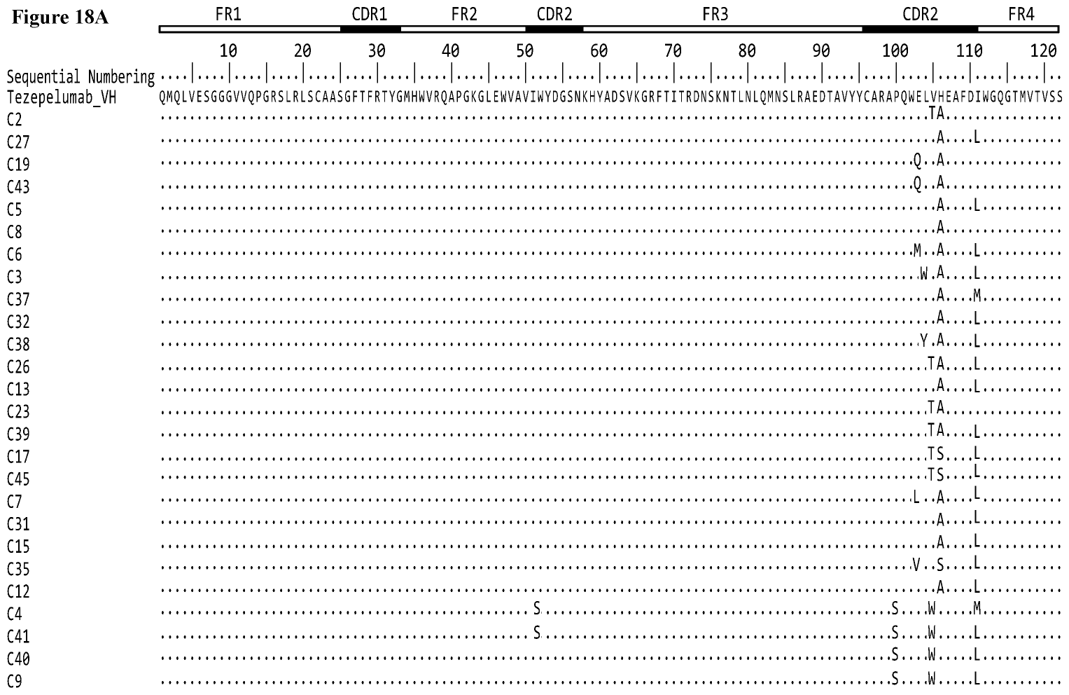

To test IgG1 BDG32.007 ability to inhibit PD-1—PD-L1 interaction in a live cell setting, where PD-1 is located on the cellular membrane in its biologically relevant conformation, a cellular reporter PD1/PDL1 blocking assay was used. As can be seen in FIG. 17, BDG 32.007 and Nivolumab inhibited the PD-1 dependent luminescence whereas an IgG1 isotype control did not. The IC₅₀ for Nivolumab was approximately 3.6 nM and for BDG 32.007 was approximately 2.5 nM. This experiment suggests that BDG 32.007 is capable of binding and inhibiting PD-1 in a biologically relevant cellular context on a comparable level to Nivolumab.

Summary: The data presented here shows dual specificity of binding with negligible non-specific binding for dual binding PD-1/OX40) antibodies, wherein binding and inhibiting PD-1 in a relevant biological context was also demonstrated.

### Example 4

### Dual Binding IL-13/TSLP Antibodies—Experimental Procedures

Objective: To generate unique, dual binding antibodies.

Methods: Libraries were designed using the sequence of the variable domains of a template antibody (SEQ ID NO: 110—template variable heavy chain sequence; SEQ ID NO: 111—template variable light chain sequence)) as a starting point.

Template Variable Heavy Chain:

(SEQ ID NO: 110)
QMQLVESGGGVVQPGRSLRLSCAASGFTFRTYGMHWVRQA
PGKGLEWVAVIWYDGSNKHYADSVKGRFTITRDNSKNTLN
LQMNSLRAEDTAVYYCARAPQWELVHEAFDIWGQGTMVTV
SS.
Template Variable Light Chain:
(SEQ ID NO: 111)
SYVLTQPPSVSVAPGQTARITCGGNNLGSKSVHWYQQKPG
QAPVLVVYDDSDRPSWIPERFSGSNSGNTATLTISRGEAG
DEADYYCQVWDSSSDHVVFGGGTKLTVL.

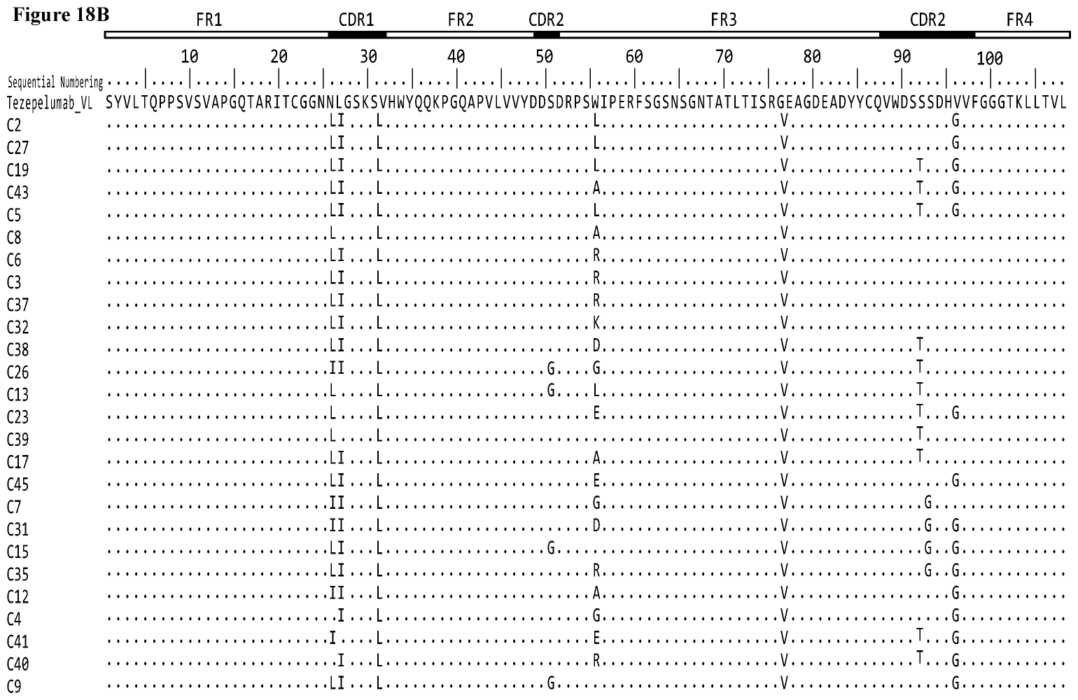

Each library contained 21 positions that were chosen for variation with respect to the template original sequences. These positions are located in both CDRs (H2, H3, L1, L2, and L3) and framework (FIGS. 18A-18B). The following positions were chosen for variation in the libraries (IMGT® numbering scheme [the international ImMunoGeneTics information System® http://www.imgt.org):

Variable H chain (SEQ ID NO: 110): 57(H2), 107(H3), 108(H3), 109(H3), 110(H3), 111(H3), 111A(H3), 112A(H3), 112(H3), 113(H3), 114(H3), 117(H3).

Variable L chain (SEQ ID NO: 111): 27(L1), 28(L1), 38 (FR2), 65(L2), 70(FR3), 94(FR3), 109(L3), 110(L3), 115(L3).

The resulting IL13/TSLP binding antibodies comprising variant heavy chain/variant light chain pairs, included a clone (C2) that contained 8 mutations relative to the template starting sequences (See, FIGS. 18A and 18B).

Library Construction Methods:

Libraries were constructed on the 5J13 template (PDB5J13) by overlapping extension PCR with degenerate oligonucleotides encoding the diversity 2*10\hat{ }14. PCR to introduce diversity was done using Phusion high fidelity DNA polymerase (New England Biolabs USA, Cat: M0530) according to manufacturer instructions in a 3-step reaction (98° C. for 30 sec, 65° C. for 20 sec, 72° C. for 30 sec, 30 cycles). The PCR products were gel purified by gel purification kit and assembled (100 ng from each) in equimolar ratios in a 3-step PCR reaction, as above, in the absence of primers. The assembled PCR product was reused as the template for PCR amplifying the full scFv library, as above, using forward and reverse primers adding vector sequences 5′ and 3′ to the scFv library to efficiently perform homologous recombination in yeast cells.

Library transformation was carried out as published (Benatuil et al., (2010) An improved yeast transformation method for the generation of very large human antibody libraries. Protein Eng. Des. Sel. 23, 155-159. 400 μl of a yeast suspension (EBY100, ATCC, USA) per 0.2 cm cuvette (cell projects) was electroporated (BioRad, USA, GenePulser) with 4 μg linearized vector (pCTcon3) and 12 μg DNA insert (scFv Library) in a 1:3 vector to insert ratio (Chao, G. et al. Isolating and engineering human antibodies using yeast surface display. Nat. Protoc. 1, 755-768 (2006)). The number of transformants of each library was determined to ˜1×10⁸ by serial dilutions of transformed cells (Benatuil et al. (2010) ibid)

Methods of Screening and Selection Using Yeast Surface Display:

Yeast-displayed scFv libraries were grown in a SDCAA selective medium and induced for expression with 2% w/v galactose at 30° C. overnight according to established protocols (Chao et al., (2006) ibid) The library was screened on BioRad S3e Fluorescence Activated Cell Sorter for high affinity binders of rh-IL-13-Fc (Reprokine, Israel) using mouse anti Myc-FITC (Santa Cruze, USA) and goat anti human Fc-APC (Jackson Immuno research, USA). Isolated clones from the final sort were sequenced by extraction of plasmid DNA from the yeast clones using a Zymoprep kit (Zymo Research, USA) and the DNA was sequenced. The chosen clones were incubated with either 10 nM recombinant human IL-13 (rh-IL-13)-Fc or 10 nM recombinant human TSLP (hTSLP)-Fc for 1 hour at room temperature. Cells were washed and resuspended in ice-cold PBS 0.1% BSA buffer containing a fluorescent labeled secondary antibody as described above for 20 min and analyzed using a flow cytometer. The values obtained were normalized to expression levels and to a positive control (an anti-IL-13 or anti TSLP binding antibody).

Methods of IgG Production—Production of the IgGs Including the Light Chain (LC) and Heavy Chain (HC) Variable Regions:

Sequences of the selected clones were synthesized as GeneBlock (GB) with 5′ 25 bp region homologous to the cloning regions of pSF-CMV-HuIgG1_HC and pSF-CMV-HuLambda_LC (Oxford genetics, Oxford UK), the GB codon usage was optimized for mammalian expression (integrated DNA Technologies. Coralville, Iowa USA). The pSF-CMV-HuIgG1_HC and pSF-CMV-HuLambda_LC were digested with using BseRI and NcoI, and the LC and HC variable region DNA fragments were cloned into the expression corresponding vectors using NEBuilder (NEB Ipswich, Massachusetts, USA). The expression vectors were transfected and expressed in ExpiCHO Expression System (ThermoFisher Scientific, USA) according to the manufacturer's instructions. Briefly: 25 ml CHO cells were grown at 37° C. to a density of 6*10\hat{ }6 cell/ml, 25 μg expression vector 1:2 HC/LC ratio were transfected into CHO cells, 20 hours post transfection the cells growth conditions were changed to 32° C. with 120 rpm shaking for 10 days. Subsequently the cells were centrifuged and IgGs were purified from the supernatant using proteinA beads, followed by size exclusion chromatography on a Superdex® 200 10/300 increase column (GE) with PBS serving as mobile phase.

Methods of Determination of IgG EC50 Binding to Human and Cynomolgus Monkey TSLP

Plates (Greiner Bio-One Cat:655081) were coated with 45.5 ng/well human or cynomolgus monkey (cyno) TSLP antigen, then washed and blocked with 3% skim milk in PBS with 0.05% tween. Post blocking the tested IgG was added to the wells in a concentration range of 1 nM-1000 nM and incubated for 1 hour at room temperature (RT). The plates were washed and goat anti-human Fc-HRP conjugated secondary antibody (Jackson cat:109-035-008) diluted 1:20000 in PBS, was added. The reaction was developed and stopped using TMB (Southern-Biotech cat:0410-01) and stop solution (Southern-Biotech cat:0412-01) respectively and read at 450 nm.

Methods of Determination of IC50 Competition Between IL-13 and TSLP

Plates were coated with 1 ng/ul hTSLP washed with TBS 0.05% tween (TBS-T) and blocked with TBS-T 2% BSA. 20 nM of tested IgG was incubated with rhIL-13 at a concertation range of 0.78 nM to 200 nM for 1 hour, then the mixture was loaded on the plates for 10 minutes and the wells were washed and a bound IgG was detected using anti human Fc-HRP conjugate as described for the ELISA EC50 experiment above. A reciprocal competition experiment was conducted using the same conditions except this time IL-13 was coated on the wells, and TSLP served as free competing ligand at the same concentration range.

Methods of Determination of IgG IC50 Inhibition Constant of Blocking TSLP from Binding to TSLP-R:

150 ng/well of TSLP-R-Fc tag (ACRO biosystems TSR-H525a) was diluted in 0.015M NaHCO₃, pH=9.5, and was then used to coat the wells of a 96 well plate (Greiner Bio-One Cat:655081). Wells were then washed three times with TBS 0.05% tween (TBS-T) and blocked with TBS-T containing 2% BSA (w/v). Competitor IgG at a concentration range of 0.11 nM to 300 nM was mixed with 3 nM hTSLP-His (ACRO biosystems cat: TSP-H52Hb) for one hour, then the mixture was loaded into the wells of the 96 well plate, incubated for 10 minutes, followed by washing the plate three times with TBS-T. Subsequently 1:200 anti-His-HRP conjugated secondary antibody was added (Santa Cruz Biothechnology cat SC-8036). The reaction was developed and stopped using TMB (Southern-Biotech cat:0410-01) and stop solution (Southern-Biotech cat:0412-01) respectively, and read at 450 nm.

Methods of Specificity Determination by ELISA

96 well plates (Greiner Bio-One Cat:655081) were coated with a total of 250 ng ligand, blocked with PBS-T containing 0.5% (w/v) BSA, and incubated with 100 nM IgG. Plates were developed using the same reagents and conditions as in the TSLP EC50 experiment described herein.

Methods of Calibration of MUTZ5 TSLP Reporter Cell Line:

The in-vitro activity of anti-TSLP blockade of TSLP binding to its cognate receptor is based on detection of pSTAT5 activation by human TSLP in MUTZ5 human leukemia cell line (Francis et al., (2016) Hematopoiesis, 101(4):417-426). In order to determine the EC50 value of hTSLP STAT5 activation of MUTZ5 cell line, cells were inoculated in a total volume of 150 μl, 250×10⁵ cells/well and incubated for 1 hr at 37° C. 5% CO₂ in a 96 well plate. Then TSLP at concentration range of 0.1 pg/mml to 1000 pg/ml was added for 30 minutes. Subsequently cells were washed, blocked with Fc blocker (BD bioscience FC Blocker-MIX cat #BD564220), and fixated with cytofix fixation buffer (BD bioscience Cat #554655). The cells were permeabilized with 90% methanol, washed and labeled with anti-pSTAT5-PE (BD bioscience cat #562077). Treated MUTZ5 cells were analyzed for pSTST5 activation on a CytoFLEX S flow cytometer (Beckman). Cells gated for singlets and pSTAT5 were marked as pSTAT5 positive.

Methods of Determination of IgG IC50 Inhibition of MUTZ5 pSTAT5 Activation by hTSLP

To test functional blocking of pSTAT5 activation, IgG at a concentration range of 0.48 pM to 500 pM, was mixed with 14 pM hTSLP (ACROBiosystems, cat #TSP-H52Hb) and incubated for 30 minutes, then added to the cells for another 60 minutes. Subsequently the cells were washed, fixated, labeled, and analyzed as described for the calibration of MUTZ5 cells.

Methods of Surface Plasmon Resonance (SPR) Analysis

Measurements of IgG binding to human IL-13: The SPR analysis was done on Biacore 200 (GE) on CM5 chips cat:br10005-30 (GE), the chip was crosslinked with primary capture Ab (Cat: br-1008-39 GE) to a target of 8000RU, after cross linking of the primary Ab, the tested antibodies were immobilized on the primary Ab to a target of 500RU. The hIL-13 (Peprotech) analyte was streamed in HEB-EP buffer at concentrations ranging from 800 nM to 1.6 nM in a series of two-fold dilutions, one concentration for each cycle. Subsequent to a cycle, the analyte and tested antibody were stripped from the chip and new tested Ab was loaded on the chip as described above. KD was determined at a steady state condition.

Measurements of Binding to Cynomolgus Monkey IL-13 (cIL-13, Sino Biological, USA) and Human TSLP

The SPR analysis was done on ProteOn™ XPR36 (BioRad) on a GLC chips cat:176-5011 (BioRad). The chip was crosslinked with primary capture Ab (Cat: br-1008-39 GE) to a target of 5500RU. After cross-linking of the primary Ab tested, antibodies 33.003 and 33.004 were immobilized on the primary Ab to a target of 2000RU. The cyno IL-13 analyte was streamed in HEB-EP buffer at concentrations ranging from 200 nM to 12.5 nM in a series of two-fold dilutions. KD was determined at a steady state condition. For measurements of binding kinetics to hTSLP, the same conditions were used but with TSLP serving as analyte at concentrations ranging from 3.2 nM to 0.2 nM in a series of two-fold dilutions.

Method of Dynamic Scanning Fluorescence (DSF) Dynamic Scanning Fluorescence was measured as reported by (Niedziela-Majka et al., 2015) with minor modifications. Briefly: 0.3 mg/ml tested antibody in sodium acetate pH 5.5 buffer was mixed 1:1 with 20xsypro orange (Thermo Fisher, USA cat #S6650) in the same buffer. Changes in fluorescence were monitored on a Bio-Rad cfx96 light cycler with setting of 0.5° C./min from 25° C.-100′C. Tm was determined as the temperature corresponding to the maximum value of the first derivative of the DSF melting curve. Where mentioned, antibodies were diluted to 0.5 mg/ml in PBS and analyzed using NanoDSF Prometheus NT.48 (Nanotemper, Germany) in a temperature elevation rate of 1° C./min.

Methods of Cell Based Assays

HEK-Blue IL-4/IL-13 Cells (Invivogen, France Catalog #hkb-il413) were used to determined IL-13 inhibition. HEK-Blue cells were cultured in growth medium comprising of DMEM, 4.5 g/l glucose, 10% (v/v) fetal bovine serum (FBS), 50 U/ml penicillin, 50 mg/ml streptomycin, 100 mg/ml Normocin, 2 mM L-glutamine, 10 μg/ml of blasticidin and 100 μg/ml of Zeocin. HEK-Blue IL-4/IL-13 cells are specifically designed to monitor the activation of the STAT6 pathway induced by IL-4 and IL-13. These cells were generated by stably introducing the human STAT6 gene into HEK293 cells to obtain a fully active STAT6 signaling pathway. The other genes of the pathway are naturally expressed in sufficient amounts. HEK-BlueIL-4/dual cells stably express the reporter gene, secreted embryonic alkaline phosphatase (SEAP), under the control of the IFNβ minimal promoter fused to four STAT6 binding sites. Activation of the STAT6 pathway in HEK-Blue IL-4/IL-13 cells induces the expression of the reporter gene. SEAP, which is secreted in the supernatant is easily detectable when using QUANTI-Blue, a medium that turns purple/blue in the presence of SEAP.

Methods of Calibration of HEK-Blue IL-4/IL-13 System

In order to determine the EC₅₀ value for rh-IL-13 on HEK-Blue IL-4/IL-13 cells, 50000 cells (5*10\hat{ }4/ELISA well) were incubated with rh-IL-13 antibody (Peprotech, Israel) at concentration of 0 nM to 8.13 nM for 24 hrs at 37° C., 5% CO₂ in a 96 well plate. At the end of the incubation, 20 ul of the cell's supernatant was incubated with 180 μl of QUANTI-Blue reagent (Invivogen, France) for an additional 2 hrs, and the reaction was analyzed by measuring the absorbance at 620-655 nm using a plate reader spectrophotometer (Synergy Neo2, BioTek Instruments, Inc. USA). Data shown is the mean of triplicate experiments, and error bars represent standard deviation.

IC₅₀ of Antibody Inhibition of IL-13 Downstream Signaling:

0.4 nM of rh-IL-13 was incubated with antibodies at a range of concentrations for 1 hr at room temperature. After the incubation, the mixture of rh-IL-13-antibody was added to a total volume of 200 μl, 50,000 cells/well and incubated for 24 hrs at 37 C 5% CO₂ in a 96 well plate. At the end of the incubation, 20 μl of the cell's supernatant was incubated with 180 μl of QUANTI-Blue reagent for additional 2 hrs, and the reaction was analyzed by measuring the absorbance at 620-655 nm using a plate reader spectrophotometer. Data shown is the mean of triplicate experiments, and error bars represent standard deviation.

### Example 5

### Screening and Selection of TSLP/IL-13 Dual Binding Antibodies

Objective: Screen engineered dual binding antibodies to identify those with highest binding for IL-13 and TSLP.

Results: Following screening and selection of the libraries to bind both IL-13 and TSLP, 45 clones were selected, isolated, and sequenced resulting in 26 unique Heavy chain (VH)—Light chain (VL) pair variable regions, wherein the amino acid sequences of the Heavy chain and Light chain pairs are presented in Table 8 (antibodies 1-26), the nucleotide sequences of the Heavy chain and Light chain scFv for antibodies 1-26, including the encoded linker sequences, are presented in Table 9, and the nucleic acid sequences of the Heavy chain and Light chain pairs for antibodies 1-26, are presented in Table 10. The Clone ID number for antibodies 1-26, is provided as “C #”—of each “Name” provided, for example at row 2, Clone C2 variable region pair comprises C2 VL sequence SEQ ID NO: 112 and C2 VH sequence SEQ ID NO: 113.

Subsequently, an affinity maturation library was screened and additional dual binding antibody clones identified that showed YSD tight binding to both hIL-13 and hTSLP (Ab clone #s: 33.023, 33.025, 38.014, 38.015. 38.018, 38.019, 38.021, 38.026, 38.040). The amino acid sequences of the VH/VL regions of clones 33.023, 33.025, 38.014, 38.015. 38.018, 38.019, 38.021, 38.026, 38.040, are presented in Table 8 and the nucleotide sequences encoding the VH/VL regions are presented in Table 10. The CDR regions of VH/VL pairs from Ab clone #s: 33.023, 33.025, 38.014, 38.015. 38.018, 38.019, 38.021, 38.026, 38.040, are provided in Table 11 and Table 12 below. These clones were selected for IgG production.

TABLE 8
Engineered dual binding antibodies:
Variable Light chain (VL) and
Variable Heavy chain (VH) amino
acid sequences (See also FIGS. 18A and 18B)

| | SEQ | | | SEQ | | |
|---|---|---|---|---|---|---|
| Ab | ID | | | ID | | |
| # | NO: | Name | VL Sequence | NO: | Name | VH Sequence |
| 1 | 112 | C2- | SYVLTQPPSVSVAPG | 113 | C2- | QMQLVESGGGVVQPG |
| | | VL | QTARITCGGNLIGSK | | VH | RSLRLSCAASGFTFR |
| | | | LVHWYQQKPGQAPVL | | | TYGMHWVRQAPGKGL |
| | | | VVYDDSDRPSLIPER | | | EWVAVIWYDGSNKHY |
| | | | FSGSNSGNTATLTIS | | | ADSVKGRFTITRDNS |
| | | | RVEAGDEADYYCQVW | | | KNTLNLQMNSLRAED |
| | | | DSSSDGVVFGGGTKL | | | TAVYYCARAPQWELT |
| | | | TVL | | | AEAFDIWGQGTMVTV |
| | | | | | | SS |
| 2 | 114 | C27- | SYVLTQPPSVSVAPG | 115 | C27- | QMQLVESGGGVVQPG |
| | | VL | QTARITCGGNLIGSK | | VH | RSLRLSCAASGFTFR |
| | | | LVHWYQQKPGQAPVL | | | TYGMHWVRQAPGKGL |
| | | | VVYDDSDRPSLIPER | | | EWVAVIWYDGSNKHY |
| | | | FSGSNSGNTATLTIS | | | ADSVKGRFTITRDNS |
| | | | RVEAGDEADYYCQVW | | | KNTLNLQMNSLRAED |
| | | | DSSSDGVVFGGGTKL | | | TAVYYCARAPQWELV |
| | | | TVL | | | AEAFDLWGQGTMVTV |
| | | | | | | SS |
| 3 | 116 | C19- | SYVLTQPPSVSVAPG | 117 | C19VH | QMQLVESGGGVVQPG |
| | | VL | QTARITCGGNLIGSK | | | RSLRLSCAASGFTFR |
| | | | LVHWYQQKPGQAPVL | | | TYGMHWVRQAPGKGL |
| | | | VVYDDSDRPSLIPER | | | EWVAVIWYDGSNKHY |
| | | | FSGSNSGNTATLTIS | | | ADSVKGRFTITRDNS |
| | | | RVEAGDEADYYCQVW | | | KNTLNLQMNSLRAED |
| | | | DTSSDGVVFGGGTKL | | | TAVYYCARAPQWQLV |
| | | | TVL | | | AEAFDIWGQGTMVTV |
| | | | | | | SS |
| 4 | 118 | C43- | SYVLTQPPSVSVAPG | 119 | C43- | QMQLVESGGGVVQPG |
| | | VL | QTARITCGGNLIGSK | | VH | RSLRLSCAASGFTFR |
| | | | LVHWYQQKPGQAPVL | | | TYGMHWVRQAPGKGL |
| | | | VVYDDSDRPSAIPER | | | EWVAVIWYDGSNKHY |
| | | | FSGSNSGNTATLTIS | | | ADSVKGRFTITRDNS |
| | | | RVEAGDEADYYCQVW | | | KNTLNLQMNSLRAED |
| | | | DTSSDGVVFGGGTKL | | | TAVYYCARAPQWQLV |
| | | | TVL | | | AEAFDIWGQGTMVTV |
| | | | | | | SS |
| 5 | 120 | C5- | SYVLTQPPSVSVAPG | 121 | C5- | QMQLVESGGGVVQPG |
| | | VL | QTARITCGGNLIGSK | | VH | RSLRLSCAASGFTFR |
| | | | LVHWYQQKPGQAPVL | | | TYGMHWVRQAPGKGL |
| | | | VVYDDSDRPSLIPER | | | EWVAVIWYDGSNKHY |
| | | | FSGSNSGNTATLTIS | | | ADSVKGRFTITRDNS |
| | | | RVEAGDEADYYCQVW | | | KNTLNLQMNSLRAED |
| | | | DTSSDGVVFGGGTKL | | | TAVYYCARAPQWELV |
| | | | TVL | | | AEAFDLWGQGTMVTV |
| | | | | | | SS |
| 6 | 122 | C8- | SYVLTQPPSVSVAPG | 123 | C8- | QMQLVESGGGVVQPG |
| | | VL | QTARITCGGNLLGSK | | VH | RSLRLSCAASGFTFR |
| | | | LVHWYQQKPGQAPVL | | | TYGMHWVRQAPGKGL |
| | | | VVYDDSDRPSAIPER | | | EWVAVIWYDGSNKHY |
| | | | FSGSNSGNTATLTIS | | | ADSVKGRFTITRDNS |
| | | | RVEAGDEADYYCQVW | | | KNTLNLQMNSLRAED |
| | | | DSSSDHVVFGGGTKL | | | TAVYYCARAPQWELV |
| | | | TVL | | | AEAFDIWGQGTMVTV |
| | | | | | | SS |
| 7 | 124 | C6- | SYVLTQPPSVSVAPG | 125 | C6- | QMQLVESGGGVVQPG |
| | | VL | QTARITCGGNLIGSK | | VH | RSLRLSCAASGFTFR |
| | | | LVHWYQQKPGQAPVL | | | TYGMHWVRQAPGKGL |
| | | | VVYDDSDRPSRIPER | | | EWVAVIWYDGSNKHY |
| | | | FSGSNSGNTATLTIS | | | ADSVKGRFTITRDNS |
| | | | RVEAGDEADYYCQVW | | | KNTLNLQMNSLRAED |
| | | | DSSSDHVVFGGGTKL | | | TAVYYCARAPQWMLV |
| | | | TVL | | | AEAFDLWGQGTMVTV |
| | | | | | | SS |
| 8 | 126 | C3- | SYVLTQPPSVSVAPG | 127 | C3- | QMQLVESGGGVVQPG |
| | | VL | QTARITCGGNLIGSK | | VH | RSLRLSCAASGFTFR |
| | | | LVHWYQQKPGQAPVL | | | TYGMHWVRQAPGKGL |
| | | | VVYDDSDRPSRIPER | | | EWVAVIWYDGSNKHY |
| | | | FSGSNSGNTATLTIS | | | ADSVKGRFTITRDNS |
| | | | RVEAGDEADYYCQVW | | | KNTLNLQMNSLRAED |
| | | | DSSSDHVVFGGGTKL | | | TAVYYCARAPQWEWV |
| | | | TVL | | | AEAFDLWGQGTMVTV |
| | | | | | | SS |
| 9 | 128 | C37- | SYVLTQPPSVSVAPG | 129 | C37- | QMQLVESGGGVVQPG |
| | | VL | QTARITCGGNLIGSK | | VH | RSLRLSCAASGFTFR |
| | | | LVHWYQQKPGQAPVL | | | TYGMHWVRQAPGKGL |
| | | | VVYDDSDRPSRIPER | | | EWVAVIWYDGSNKHY |
| | | | FSGSNSGNTATLTIS | | | ADSVKGRFTITRDNS |
| | | | RVEAGDEADYYCQVW | | | KNTLNLQMNSLRAED |
| | | | DSSSDHVVFGGGTKL | | | TAVYYCARAPQWELV |
| | | | TVL | | | AEAFDMWGQGTMVTV |
| | | | | | | SS |
| 10 | 130 | C32- | SYVLTQPPSVSVAPG | 131 | C32- | QMQLVESGGGVVQPG |
| | | VL | QTARITCGGNLIGSK | | VH | RSLRLSCAASGFTFR |
| | | | LVHWYQQKPGQAPVL | | | TYGMHWVRQAPGKGL |
| | | | VVYDDSDRPSKIPER | | | EWVAVIWYDGSNKHY |
| | | | FSGSNSGNTATLTIS | | | ADSVKGRFTITRDNS |
| | | | RVEAGDEADYYCQVW | | | KNTLNLQMNSLRAED |
| | | | DSSSDHVVFGGGTKL | | | TAVYYCARAPQWELV |
| | | | TVL | | | AEAFDLWGQGTMVTV |
| | | | | | | SS |
| 11 | 132 | C38- | SYVLTQPPSVSVAPG | 133 | C38- | QMQLVESGGGVVQPG |
| | | VL | QTARITCGGNLIGSK | | VH | RSLRLSCAASGFTFR |
| | | | LVHWYQQKPGQAPVL | | | TYGMHWVRQAPGKGL |
| | | | VVYDDSDRPSDIPER | | | EWVAVIWYDGSNKHY |
| | | | FSGSNSGNTATLTIS | | | ADSVKGRFTITRDNS |
| | | | RVEAGDEADYYCQVW | | | KNTLNLQMNSLRAED |
| | | | DTSSDHVVFGGGTKL | | | TAVYYCARAPQWEYV |
| | | | TVL | | | AEAFDLWGQGTMVTV |
| | | | | | | SS |
| 12 | 134 | C26- | SYVLTQPPSVSVAPG | 135 | C26 | QMQLVESGGGVVQPG |
| | | VL | QTARITCGGNIIGSK | | VH | RSLRLSCAASGFTFR |
| | | | LVHWYQQKPGQAPVL | | | TYGMHWVRQAPGKGL |
| | | | VVYDDGDRPSGIPER | | | EWVAVIWYDGSNKHY |
| | | | FSGSNSGNTATLTIS | | | ADSVKGRFTITRDNS |
| | | | RVEAGDEADYYCQVW | | | KNTLNLQMNSLRAED |
| | | | DTSSDHVVFGGGTKL | | | TAVYYCARAPQWELT |
| | | | TVL | | | AEAFDLWGQGTMVTV |
| | | | | | | SS |
| 13 | 136 | C13- | SYVLTQPPSVSVAPG | 137 | C13 | QMQLVESGGGVVQPG |
| | | VL | QTARITCGGNLLGSK | | VH | RSLRLSCAASGFTFR |
| | | | LVHWYQQKPGQAPVL | | | TYGMHWVRQAPGKGL |
| | | | VVYDDGDRPSLIPER | | | EWVAVIWYDGSNKHY |
| | | | FSGSNSGNTATLTIS | | | ADSVKGRFTITRDNS |
| | | | RVEAGDEADYYCQVW | | | KNTLNLQMNSLRAED |
| | | | DTSSDHVVFGGGTKL | | | TAVYYCARAPQWELV |
| | | | TVL | | | AEAFDLWGQGTMVTV |
| | | | | | | SS |
| 14 | 138 | C23- | SYVLTQPPSVSVAPG | 139 | C23- | QMQLVESGGGVVQPG |
| | | VL | QTARITCGGNLLGSK | | VH | RSLRLSCAASGFTFR |
| | | | LVHWYQQKPGQAPVL | | | TYGMHWVRQAPGKGL |
| | | | VVYDDSDRPSEIPER | | | EWVAVIWYDGSNKHY |
| | | | FSGSNSGNTATLTIS | | | ADSVKGRFTITRDNS |
| | | | RVEAGDEADYYCQVW | | | KNTLNLQMNSLRAED |
| | | | DTSSDGVVFGGGTKL | | | TAVYYCARAPQWELT |
| | | | TVL | | | AEAFDIWGQGTMVTV |
| | | | | | | SS |
| 15 | 140 | C39- | SYVLTQPPSVSVAPG | 141 | C39- | QMQLVESGGGVVQPG |
| | | VL | QTARITCGGNLLGSK | | VH | RSLRLSCAASGFTFR |
| | | | LVHWYQQKPGQAPVL | | | TYGMHWVRQAPGKGL |
| | | | VVYDDSDRPSWIPER | | | EWVAVIWYDGSNKHY |
| | | | FSGSNSGNTATLTIS | | | ADSVKGRFTITRDNS |
| | | | RVEAGDEADYYCQVW | | | KNTLNLQMNSLRAED |
| | | | DTSSDHVVFGGGTKL | | | TAVYYCARAPQWELT |
| | | | TVL | | | AEAFDLWGQGTMVTV |
| | | | | | | SS |
| 16 | 142 | C17- | SYVLTQPPSVSVAPG | 143 | C17- | QMQLVESGGGVVQPG |
| | | VL | QTARITCGGNLIGSK | | VH | RSLRLSCAASGFTFR |
| | | | LVHWYQQKPGQAPVL | | | TYGMHWVRQAPGKGL |
| | | | VVYDDSDRPSAIPER | | | EWVAVIWYDGSNKHY |
| | | | FSGSNSGNTATLTIS | | | ADSVKGRFTITRDNS |
| | | | RVEAGDEADYYCQVW | | | KNTLNLQMNSLRAED |
| | | | DTSSDHVVFGGGTKL | | | TAVYYCARAPQWELT |
| | | | TVL | | | SEAFDLWGQGTMVTV |
| | | | | | | SS |
| 17 | 144 | C45- | SYVLTQPPSVSVAPG | 145 | C45- | QMQLVESGGGVVQPG |
| | | VL | QTARITCGGNLIGSK | | VH | RSLRLSCAASGFTFR |
| | | | LVHWYQQKPGQAPVL | | | TYGMHWVRQAPGKGL |
| | | | VVYDDSDRPSEIPER | | | EWVAVIWYDGSNKHY |
| | | | FSGSNSGNTATLTIS | | | ADSVKGRFTITRDNS |
| | | | RVEAGDEADYYCQVW | | | KNTLNLQMNSLRAED |
| | | | DSSSDGVVFGGGTKL | | | TAVYYCARAPQWELT |
| | | | TVL | | | SEAFDLWGQGTMVTV |
| | | | | | | SS |
| 18 | 146 | C7- | SYVLTQPPSVSVAPG | 147 | C7- | QMQLVESGGGVVQPG |
| | | VL | QTARITCGGNIIGSK | | VH | RSLRLSCAASGFTFR |
| | | | LVHWYQQKPGQAPVL | | | TYGMHWVRQAPGKGL |
| | | | VVYDDSDRPSGIPER | | | EWVAVIWYDGSNKHY |
| | | | FSGSNSGNTATLTIS | | | ADSVKGRFTITRDNS |
| | | | RVEAGDEADYYCQVW | | | KNTLNLQMNSLRAED |
| | | | DSGSDHVVFGGGTKL | | | TAVYYCARAPQWLLV |
| | | | TVL | | | AEAFDLWGQGTMVTV |
| | | | | | | SS |
| 19 | 148 | C31- | SYVLTQPPSVSVAPG | 149 | C31- | QMQLVESGGGVVQPG |
| | | VL | QTARITCGGNIIGSK | | VH | RSLRLSCAASGFTFR |
| | | | LVHWYQQKPGQAPVL | | | TYGMHWVRQAPGKGL |
| | | | VVYDDSDRPSDIPER | | | EWVAVIWYDGSNKHY |
| | | | FSGSNSGNTATLTIS | | | ADSVKGRFTITRDNS |
| | | | RVEAGDEADYYCQVW | | | KNTLNLQMNSLRAED |
| | | | DSGSDGVVFGGGTKL | | | TAVYYCARAPQWELV |
| | | | TVL | | | AEAFDLWGQGTMVTV |
| | | | | | | SS |
| 20 | 150 | C15- | SYVLTQPPSVSVAPG | 151 | C15- | QMQLVESGGGVVQPG |
| | | VL | QTARITCGGNLIGSK | | VH | RSLRLSCAASGFTFR |
| | | | LVHWYQQKPGQAPVL | | | TYGMHWVRQAPGKGL |
| | | | VVYDDGDRPSWIPER | | | EWVAVIWYDGSNKHY |
| | | | FSGSNSGNTATLTIS | | | ADSVKGRFTITRDNS |
| | | | RVEAGDEADYYCQVW | | | KNTLNLQMNSLRAED |
| | | | DSGSDGVVFGGGTKL | | | TAVYYCARAPQWELV |
| | | | TVL | | | AEAFDLWGQGTMVTV |
| | | | | | | SS |
| 21 | 152 | C35- | SYVLTQPPSVSVAPG | 153 | C35- | QMQLVESGGGVVQPG |
| | | VL | QTARITCGGNLIGSK | | VH | RSLRLSCAASGFTFR |
| | | | LVHWYQQKPGQAPVL | | | TYGMHWVRQAPGKGL |
| | | | VVYDDSDRPSRIPER | | | EWVAVIWYDGSNKHY |
| | | | FSGSNSGNTATLTIS | | | ADSVKGRFTITRDNS |
| | | | RVEAGDEADYYCQVW | | | KNTLNLQMNSLRAED |
| | | | DSGSDGVVFGGGTKL | | | TAVYYCARAPQWVLV |
| | | | TVL | | | SEAFDLWGQGTMVTV |
| | | | | | | SS |
| 22 | 154 | C12 - | SYVLTQPPSVSVAPG | 155 | C12- | QMQLVESGGGVVQPG |
| | | VL | QTARITCGGNIIGSK | | VH | RSLRLSCAASGFTFR |
| | | | LVHWYQQKPGQAPVL | | | TYGMHWVRQAPGKGL |
| | | | VVYDDSDRPSAIPER | | | EWVAVIWYDGSNKHY |
| | | | FSGSNSGNTATLTIS | | | ADSVKGRFTITRDNS |
| | | | RVEAGDEADYYCQVW | | | KNTLNLQMNSLRAED |
| | | | DSSSDGVVFGGGTKL | | | TAVYYCARAPQWELV |
| | | | TVL | | | AEAFDLWGQGTMVTV |
| | | | | | | SS |
| 23 | 156 | C4- | SYVLTQPPSVSVAPG | 157 | C4- | QMQLVESGGGVVQPG |
| | | VL | QTARITCGGNNIGSK | | VH | RSLRLSCAASGFTFR |
| | | | LVHWYQQKPGQAPVL | | | TYGMHWVRQAPGKGL |
| | | | VVYDDSDRPSGIPER | | | EWVAVISYDGSNKHY |
| | | | FSGSNSGNTATLTIS | | | ADSVKGRFTITRDNS |
| | | | RVEAGDEADYYCQVW | | | KNTLNLQMNSLRAED |
| | | | DSSSDGVVFGGGTKL | | | TAVYYCARSPQWEWV |
| | | | TVL | | | HEAFDMWGQGTMVTV |
| | | | | | | SS |
| 24 | 158 | C41- | SYVLTQPPSVSVAPG | 159 | C41- | QMQLVESGGGVVQPG |
| | | VL | QTARITCGGNILGSK | | VH | RSLRLSCAASGFTFR |
| | | | LVHWYQQKPGQAPVL | | | TYGMHWVRQAPGKGL |
| | | | VVYDDSDRPSEIPER | | | EWVAVISYDGSNKHY |
| | | | FSGSNSGNTATLTIS | | | ADSVKGRFTITRDNS |
| | | | RVEAGDEADYYCQVW | | | KNTLNLQMNSLRAED |
| | | | DTSSDGVVFGGGTKL | | | TAVYYCARSPQWEWV |
| | | | TVL | | | HEAFDLWGQGTMVTV |
| | | | | | | SS |
| 25 | 160 | C40- | SYVLTQPPSVSVAPG | 161 | C40- | QMQLVESGGGVVQPG |
| | | VL | QTARITCGGNNIGSK | | VH | RSLRLSCAASGFTFR |
| | | | LVHWYQQKPGQAPVL | | | TYGMHWVRQAPGKGL |
| | | | VVYDDSDRPSRIPER | | | EWVAVIWYDGSNKHY |
| | | | FSGSNSGNTATLTIS | | | ADSVKGRFTITRDNS |
| | | | RVEAGDEADYYCQVW | | | KNTLNLQMNSLRAED |
| | | | DTSSDGVVFGGGTKL | | | TAVYYCARSPQWEWV |
| | | | TVL | | | HEAFDLWGQGTMVTV |
| | | | | | | SS |
| 26 | 162 | C9- | SYVLTQPPSVSVAPG | 163 | C9- | QMQLVESGGGVVQPG |
| | | VL | QTARITCGGNLIGSK | | VH | RSLRLSCAASGFTFR |
| | | | LVHWYQQKPGQAPVL | | | TYGMHWVRQAPGKGL |
| | | | VVYDDGDRPSWIPER | | | EWVAVIWYDGSNKHY |
| | | | FSGSNSGNTATLTIS | | | ADSVKGRFTITRDNS |
| | | | RVEAGDEADYYCQVW | | | KNTLNLQMNSLRAED |
| | | | DSSSDGVVFGGGTKL | | | TAVYYCARSPQWEWV |
| | | | TVL | | | HEAFDLWGQGTMVTV |
| | | | | | | SS |
| | 265 | 33.023 | SYVLTQPPSVSVAPG | 264 | 33.023 | QMQLVESGGGVVQPG |
| | | VL | QTARITCGGNLIGSK | | VH | RSLRLSCAASGFAFR |
| | | | LVHWYQQKPGQAPVL | | | TYGMHWVRQAPGKGL |
| | | | VVYDDSDRPSRIPER | | | EWVAVIWYDGSNTHY |
| | | | FSGSNSGNTATLTIS | | | ADSVKGRFTITRDNS |
| | | | RVEAGDEADYYCQVW | | | KNTLNLQMNSLRAED |
| | | | DHSSDHVVFGGGTKL | | | TAVYYCARAPQWYLS |
| | | | TVL | | | AEAFDLWGQGTMVTV |
| | | | | | | SS |
| | 269 | 33.025 | SYVLTQPPSVSVAPG | 268 | 33.025 | QMQLVESGGGVVQPG |
| | | VL | QTARITCGGNLIGSK | | VH | RSLRLSCAASGFTFR |
| | | | LVHWYQQKPGQAPVL | | | TYGMHWVRQAPGKGL |
| | | | VVYDDSDRPSLIPER | | | EWVADIWYDGSNKHY |
| | | | FSGSNSGNTATLTIS | | | ADSVKGRFTITRDNS |
| | | | RVEAGDEADYYCQVW | | | KNTLNLQMNSLRAED |
| | | | DSSSDGVVFGGGTKL | | | TAVYYCARAPQWYLV |
| | | | TVL | | | AEPFDLWGQGTMVTV |
| | | | | | | SS |
| | 279 | 38.014 | SYVLTQPPSVSVAPG | 278 | 38.014 | QMQLVESGGGVVQPG |
| | | VL | QTARITCGGNLIGAK | | VH | RSLRLSCAASGFAFR |
| | | | LVHWYQQKPGQAPVL | | | TYGMHWVRQAPGKGL |
| | | | VVYDDSDRPSRIPER | | | EWVAVIWYDGSNTHY |
| | | | FSGSNSGNTATLTIS | | | ADSVKGRFTITRDNS |
| | | | RVEAGDEADYYCQVW | | | KNTLNLQMNSLRAED |
| | | | DHSSDHVVFGGGTKL | | | TAVYYCVRAPQWYLS |
| | | | TVL | | | AEAFDLWGQGTMVTV |
| | | | | | | SS |
| | 281 | 38.018 | SYVLTQPPSVSVAPG | 280 | 38.018 | QMQLVESGGGVVQPG |
| | | VL | QTARITCGGNLIGSK | | VH | RSLRLSCAASGFAFR |
| | | | LVHWYQQKPGQAPVL | | | TYGMHWVRQAPGKGL |
| | | | VVYDDSDRPSRIPER | | | EWVAVIWDDGSNTHY |
| | | | FSGSNSGNTATLTIS | | | ADSVKGRFTITRDNS |
| | | | RVEAGDEADYYCQVW | | | KNTLNLQMNSLRAED |
| | | | DHSSDHVVFGGGTKL | | | TAVYYCARAPQWYLS |
| | | | TVL | | | AEAFDLWGQGTMVTV |
| | | | | | | SS |
| | 283 | 38.019 | SYVLTQPPSVSVAPG | 282 | 38.019 | QMQLVESGGGVVQPG |
| | | VL | QTARITCGGNLIGSK | | VH | RSLRLSCAASGFAFR |
| | | | LVHWYQQKPGQAPVL | | | TYGMHWVRQAPGKGL |
| | | | VVYDDSDRPSRIPER | | | EWVAVIWYDGSNTHY |
| | | | FSGSNSGNTATLTIS | | | ADSVKGRFTITRDNS |
| | | | RVEAGDEADYYCQVW | | | KNTLNLQMNSLRAED |
| | | | DYSSNHVVFGGGTKL | | | TAVYYCVRAPQWYLS |
| | | | TVL | | | AEAFDLWGQGTMVTV |
| | | | | | | SS |
| | 285 | 38.021 | SYVLTQPPSVSVAPG | 284 | 38.021 | QMQLVESGGGVVQPG |
| | | VL | QTARITCGGNLIGSK | | VH | RSLRLSCAASGFAFD |
| | | | LVHWYQQKPGQAPVL | | | TYGMHWVRQAPGKGL |
| | | | VVYDDSDRPSRIPER | | | EWVAVIWYDGSNTVY |
| | | | FSGSNSGNTATLTIS | | | ADSVKGRFTITRDNS |
| | | | RVEAGDEADYYCQVW | | | KNTLNLQMNSLRAED |
| | | | DHSSDHYVFGGGTKL | | | TAVYYCARAPQWYLS |
| | | | TVL | | | AEAFDLWGQGTMVTV |
| | | | | | | SS |
| | 287 | 38.025 | SYVLTQPPSVSVAPG | 286 | 38.025 | QMQLVESGGGVVQPG |
| | | VL | ETATITCGGNLIGSK | | VH | RSLRLSCAASGFAFR |
| | | | LVHWYQQKPGQAPVL | | | TYGMHWVRQAPGKGL |
| | | | VVYDDSDRPSRIPER | | | EWVAVIWYDGSNTHY |
| | | | FSGSNSGNTATLTIS | | | ADSVKGRFTITRDNS |
| | | | RVEAGDEADYYCQVW | | | KNTLNLQMNSLRAED |
| | | | DHSSDHVVFGGGTKL | | | TAVYYCVRAPQWYLS |
| | | | TVL | | | AEAFDLWGQGTMVTV |
| | | | | | | SS |
| | 289 | 38.026 | SYVLTQPPSVSVAPG | 288 | 38.026 | QMQLVESGGGVVQPG |
| | | VL | QTATITCGGNLIGSK | | VH | RSLRLSCAASGFAFR |
| | | | LVHWYQQKPGQAPVL | | | TYGMHWVRQAPGKGL |
| | | | VVYDDSDRPSRIPER | | | EWVAVIWYDGSATHY |
| | | | FSGSNIGNTATLTIS | | | ADSVKGRFTITRDNS |
| | | | RVEAGDEADYYCQVW | | | KNTLNLQMNSLRAED |
| | | | DHSSDHVVFGGGTKL | | | TAVYYCVRAPQWYLS |
| | | | TVL | | | AEAFDLWGQGTMVTV |
| | | | | | | SS |
| | 291 | 38.040 | SYVLTQPPSVSVAPG | 290 | 38.040 | QMQLVESGGGVVQPG |
| | | VL | QTARITCGGNLIGTK | | VH | RSLRLSCAASGFDFR |
| | | | LVHWYQQKPGQAPVL | | | TYGMHWVRQAPGKGL |
| | | | VVYDDSDRPSRIPER | | | EWVAVIWYDGSITHY |
| | | | FSGSNSGNTATLTIS | | | ADSVKGRFTITRDNS |
| | | | RVEAGDEADYYCQVW | | | KNTLNLQMNSLRAED |
| | | | DHNEDEVVFGGGTKL | | | TAVYYCVRAPQWYLT |
| | | | TVL | | | AEAFDLWGQGTMVTV |
| | | | | | | SS |

TABLE 9
Nucleotide Sequences Encoding Engineered
dual Binding scFv: Variable
Heavy chain (VH)-Linker-Variable
Light chain (VL)

| | SEQ | | |
|---|---|---|---|
| Ab | ID | | |
| # | NO: | Name | VH-Linker-VL Sequence |
| | 218 | Template | CAAATGCAGCTGGTCGAGTCTGGCGGTGGG |
| | | VH-L-VL | GTAGTGCAACCAGGCCGTTCTCTGCGTCTT |
| | | | AGCTGCGCCGCATCTGGTTTTACCTTTCGT |
| | | | ACCTACGGTATGCACTGGGTGCGTCAGGCA |
| | | | CCAGGCAAAGGTCTGGAATGGGTCGCAGTA |
| | | | ATCTGGTATGATGGTAGCAATAAACACTAT |
| | | | GCTGACTCAGTCAAAGGCCGTTTCACCATC |
| | | | ACCCGTGATAACAGCAAGAACACTCTTAAC |
| | | | TTACAGATGAACTCTCTGCGTGCCGAAGAC |
| | | | ACCGCAGTTTATTACTGTGCCCGTGCACCA |
| | | | CAGTGGGAATTAGTACACGAAGCATTCGAT |
| | | | ATCTGGGGTCAGGGTACTATGGTGACCGTT |
| | | | AGCTCTGGCGGTGGTGGTAGCGGAGGCGGA |
| | | | GGATCAGGTGGAGGCGGCAGTTCTTACGTG |
| | | | CTGACTCAACCACCATCAGTGTCTGTAGCA |
| | | | CCAGGCCAGACCGCACGTATTACCTGTGGC |
| | | | GGTAACAATCTGGGCTCTAAGTCTGTTCAC |
| | | | TGGTATCAGCAAAAACCAGGCCAGGCACCA |
| | | | GTACTGGTTGTGTACGATGATTCCGATCGT |
| | | | CCAAGCTGGATTCCAGAGCGTTTCAGCGGC |
| | | | TCTAATTCCGGCAACACCGCTACTCTGACT |
| | | | ATTTCCCGTGGGGAAGCCGGCGATGAAGCC |
| | | | GACTACTATTGCCAGGTCTGGGACTCTTCT |
| | | | TCCGACCATGTAGTCTTTGGCGGGGGCACC |
| | | | AAACTGACCGTTTTG |
| 1 | 219 | C2- | CAAATGCAGCTGGTCGAGTCTGGCGGTGGG |
| | | VH-L-VL | GTAGTGCAACCAGGCCGTTCTCTGCGTCTT |
| | | | AGCTGCGCCGCATCTGGTTTTACCTTTCGT |
| | | | ACCTACGGTATGCACTGGGTGCGTCAGGCA |
| | | | CCAGGCAAAGGTCTGGAATGGGTCGCAGTA |
| | | | ATCTGGTATGATGGTAGCAATAAACACTAT |
| | | | GCTGACTCAGTCAAAGGCCGTTTCACCATC |
| | | | ACCCGTGATAACAGCAAGAACACTCTTAAC |
| | | | TTACAGATGAACTCTCTGCGTGCCGAAGAC |
| | | | ACCGCCGTTTACTACTGTGCCCGTGCACCA |
| | | | CAGTGGGAATTAACGGCGGAAGCATTCGAT |
| | | | ATTTGGGGCCAGGGCACTATGGTGACCGTT |
| | | | AGCTCTGGCGGTGGTGGTAGCGGAGGCGGA |
| | | | GGATCAGGTGGAGGCGGCAGTTCTTACGTG |
| | | | CTGACTCAACCACCATCAGTGTCTGTAGCA |
| | | | CCAGGCCAGACCGCACGTATTACCTGTGGC |
| | | | GGTAACCTGATCGGCTCTAAGCTGGTTCAC |
| | | | TGGTATCAGCAAAAACCAGGCCAGGCACCA |
| | | | GTACTGGTTGTGTACGATGATAGCGATCGT |
| | | | CCAAGCCTGATTCCAGAGCGTTTCAGCGGC |
| | | | TCTAATTCCGGCAACACCGCTACTCTGACT |
| | | | ATTTCCCGTGTTGAAGCCGGCGATGAAGCC |
| | | | GACTACTATTGCCAGGTCTGGGACTCTAGC |
| | | | TCCGACGGTGTAGTCTTTGGCGGGGGCACC |
| | | | AAACTGACCGTTTTG |
| 2 | 220 | C27- | CAAATGCAGCTGGTCGAGTCTGGCGGTGGG |
| | | VH-L-VL | GTAGTGCAACCAGGCCGTTCTCTGCGTCTT |
| | | | AGCTGCGCCGCATCTGGTTTTACCTTTCGT |
| | | | ACCTACGGTATGCACTGGGTGCGTCAGGCA |
| | | | CCAGGCAAAGGTCTGGAATGGGTCGCAGTA |
| | | | ATCTGGTATGATGGTAGCAATAAACACTAT |
| | | | GCTGACTCAGTCAAAGGCCGTTTCACCATC |
| | | | ACCCGTGATAACAGCAAGAACACTCTTAAC |
| | | | TTACAGATGAACTCTCTGCGTGCCGAAGAC |
| | | | ACCGCCGTTTACTACTGTGCCCGTGCACCA |
| | | | CAGTGGGAATTAGTAGCGGAAGCATTCGAT |
| | | | CTGTGGGGCCAGGGCACTATGGTGACCGTT |
| | | | AGCTCTGGCGGTGGTGGTAGCGGAGGCGGA |
| | | | GGATCAGGTGGAGGCGGCAGTTCTTACGTG |
| | | | CTGACTCAACCACCATCAGTGTCTGTAGCA |
| | | | CCAGGCCAGACCGCACGTATTACCTGTGGC |
| | | | GGTAACCTGATCGGCTCTAAGCTGGTTCAC |
| | | | TGGTATCAGCAAAAACCAGGCCAGGCACCA |
| | | | GTACTGGTTGTGTACGATGATAGCGATCGT |
| | | | CCAAGCCTGATTCCAGAGCGTTTCAGCGGC |
| | | | TCTAATTCCGGCAACACCGCTACTCTGACT |
| | | | ATTTCCCGTGTTGAAGCCGGCGATGAAGCC |
| | | | GACTACTATTGCCAGGTCTGGGACTCTAGC |
| | | | TCCGACGGTGTAGTCTTTGGCGGGGGCACC |
| | | | AAACTGACCGTTTTG |
| 3 | 221 | C19- | CAAATGCAGCTGGTCGAGTCTGGCGGTGGG |
| | | VH-L-VL | GTAGTGCAACCAGGCCGTTCTCTGCGTCTT |
| | | | AGCTGCGCCGCATCTGGTTTTACCTTTCGT |
| | | | ACCTACGGTATGCACTGGGTGCGTCAGGCA |
| | | | CCAGGCAAAGGTCTGGAATGGGTCGCAGTA |
| | | | ATCTGGTATGATGGTAGCAATAAACACTAT |
| | | | GCTGACTCAGTCAAAGGCCGTTTCACCATC |
| | | | ACCCGTGATAACAGCAAGAACACTCTTAAC |
| | | | TTACAGATGAACTCTCTGCGTGCCGAAGAC |
| | | | ACCGCCGTTTACTACTGTGCCCGTGCACCA |
| | | | CAGTGGCAGTTAGTAGCGGAAGCATTCGAT |
| | | | ATTTGGGGCCAGGGCACTATGGTGACCGTT |
| | | | AGCTCTGGCGGTGGTGGTAGCGGAGGCGGA |
| | | | GGATCAGGTGGAGGCGGCAGTTCTTACGTG |
| | | | CTGACTCAACCACCATCAGTGTCTGTAGCA |
| | | | CCAGGCCAGACCGCACGTATTACCTGTGGC |
| | | | GGTAACCTGATCGGCTCTAAGCTGGTTCAC |
| | | | TGGTATCAGCAAAAACCAGGCCAGGCACCA |
| | | | GTACTGGTTGTGTACGATGATAGCGATCGT |
| | | | CCAAGCCTTATTCCAGAGCGTTTCAGCGGC |
| | | | TCTAATTCCGGCAACACCGCTACTCTGACT |
| | | | ATTTCCCGTGTTGAAGCCGGCGATGAAGCC |
| | | | GACTACTATTGCCAGGTCTGGGACACCAGC |
| | | | TCCGACGGTGTAGTCTTTGGCGGGGGCACC |
| | | | AAACTGACCGTTTTG |
| 4 | 222 | C43- | CAAATGCAGCTGGTCGAGTCTGGCGGTGGG |
| | | VH-L-VL | GTAGTGCAACCAGGCCGTTCTCTGCGTCTT |
| | | | AGCTGCGCCGCATCTGGTTTTACCTTTCGT |
| | | | ACCTACGGTATGCACTGGGTGCGTCAGGCA |
| | | | CCAGGCAAAGGTCTGGAATGGGTCGCAGTA |
| | | | ATCTGGTATGATGGTAGCAATAAACACTAT |
| | | | GCTGACTCAGTCAAAGGCCGTTTCACCATC |
| | | | ACCCGTGATAACAGCAAGAACACTCTTAAC |
| | | | TTACAGATGAACTCTCTGCGTGCCGAAGAC |
| | | | ACCGCCGTTTACTACTGTGCCCGTGCACCA |
| | | | CAGTGGCAGTTAGTAGCCGAAGCATTCGAT |
| | | | ATATGGGGCCAGGGCACTATGGTGACCGTT |
| | | | AGCTCTGGCGGTGGTGGTAGCGGAGGCGGA |
| | | | GGATCAGGTGGAGGCGGCAGTTCTTACGTG |
| | | | CTGACTCAACCACCATCAGTGTCTGTAGCA |
| | | | CCAGGCCAGACCGCACGTATTACCTGTGGC |
| | | | GGTAACCTGATCGGCTCTAAGCTGGTTCAC |
| | | | TGGTATCAGCAAAAACCAGGCCAGGCACCA |
| | | | GTACTGGTTGTGTACGATGATAGCGATCGT |
| | | | CCAAGCGCTATTCCAGAGCGTTTCAGCGGC |
| | | | TCTAATTCCGGCAACACCGCTACTCTGACT |
| | | | ATTTCCCGTGTTGAAGCCGGCGATGAAGCC |
| | | | GACTACTATTGCCAGGTCTGGGACACTAGC |
| | | | TCCGACGGTGTAGTCTTTGGCGGGGGCACC |
| | | | AAACTGACCGTTTTG |
| 5 | 223 | C5- | CAAATGCAGCTGGTCGAGTCTGGCGGTGGG |
| | | VH-L-VL | GTAGTGCAACCAGGCCGTTCTCTGCGTCTT |
| | | | AGCTGCGCCGCATCTGGTTTTACCTTTCGT |
| | | | ACCTACGGTATGCACTGGGTGCGTCAGGCA |
| | | | CCAGGCAAAGGTCTGGAATGGGTCGCAGTA |
| | | | ATCTGGTATGATGGTAGCAATAAACACTAT |
| | | | GCTGACTCAGTCAAAGGCCGTTTCACCATC |
| | | | ACCCGTGATAACAGCAAGAACACTCTTAAC |
| | | | TTACAGATGAACTCTCTGCGTGCCGAAGAC |
| | | | ACCGCCGTTTACTACTGTGCCCGTGCACCA |
| | | | CAGTGGGAATTAGTGGCGGAAGCATTCGAT |
| | | | CTTTGGGGCCAGGGCACTATGGTGACCGTT |
| | | | AGCTCTGGTGGTGGTGGTAGCGGAGGCGGA |
| | | | GGATCAGGTGGAGGCGGCAGTTCTTACGTG |
| | | | CTGACTCAACCACCATCAGTGTCTGTAGCA |
| | | | CCAGGCCAGACCGCACGTATTACCTGTGGC |
| | | | GGTAACCTGATCGGCTCTAAGCTGGTTCAC |
| | | | TGGTATCAGCAAAAACCAGGCCAGGCACCA |
| | | | GTACTGGTTGTGTACGATGATAGCGATCGT |
| | | | CCAAGCCTTATTCCAGAGCGTTTCAGCGGC |
| | | | TCTAATTCCGGCAACACCGCTACTCTGACT |
| | | | ATTTCCCGTGTTGAAGCCGGCGATGAAGCC |
| | | | GACTACTATTGCCAGGTCTGGGACACCAGC |
| | | | TCCGACGGTGTAGTCTTTGGCGGGGGCACC |
| | | | AAACTGACCGTTTTG |
| 6 | 224 | C8- | CAAATGCAGCTGGTCGAGTCTGGCGGTGGG |
| | | VH-L-VL | GTAGTGCAACCAGGCCGTTCTCTGCGTCTT |
| | | | AGCTGCGCCGCATCTGGTTTTACCTTTCGT |
| | | | ACCTACGGTATGCACTGGGTGCGTCAGGCA |
| | | | CCAGGCAAAGGTCTGGAATGGGTCGCAGTA |
| | | | ATCTGGTATGATGGTAGCAATAAACACTAT |
| | | | GCTGACTCAGTCAAAGGCCGTTTCACCATC |
| | | | ACCCGTGATAACAGCAAGAACACTCTTAAC |
| | | | TTACAGATGAACTCTCTGCGTGCCGAAGAC |
| | | | ACCGCCGTTTACTACTGTGCCCGTGCACCA |
| | | | CAGTGGGAATTGGTAGCCGAAGCATTCGAT |
| | | | ATCTGGGGCCAGGGCACTATGGTGACCGTT |
| | | | AGCTCTGGCGGTGGTGGTAGCGGAGGCGGA |
| | | | GGATCAGGTGGAGGCGGCAGTTCTTACGTG |
| | | | CTGACTCAACCACCATCAGTGTCTGTAGCA |
| | | | CCAGGCCAGACCGCACGTATTACCTGTGGC |
| | | | GGTAACCTGCTGGGCTCTAAGCTGGTTCAC |
| | | | TGGTATCAGCAAAAACCAGGCCAGGCACCA |
| | | | GTACTGGTTGTGTACGATGATAGCGATCGT |
| | | | CCAAGCGCAATTCCAGAGCGTTTCAGCGGC |
| | | | TCTAATTCCGGCAACACCGCTACTCTGACT |
| | | | ATTTCCCGTGTTGAAGCCGGCGATGAAGCC |
| | | | GACTACTATTGCCAGGTCTGGGACTCTAGC |
| | | | TCCGACCACGTAGTCTTTGGCGGGGGCACC |
| | | | AAACTGACCGTTTTG |
| 7 | 225 | C6- | CAAATGCAGCTGGTCGAGTCTGGCGGTGGG |
| | | VH-L-VL | GTAGTGCAACCAGGCCGTTCTCTGCGTCTT |
| | | | AGCTGCGCCGCATCTGGTTTTACCTTTCGT |
| | | | ACCTACGGTATGCACTGGGTGCGTCAGGCA |
| | | | CCAGGCAAAGGTCTGGAATGGGTCGCAGTA |
| | | | ATCTGGTATGATGGTAGCAATAAACACTAT |
| | | | GCTGACTCAGTCAAAGGCCGTTTCACCATC |
| | | | ACCCGTGATAACAGCAAGAACACTCTTAAC |
| | | | TTACAGATGAACTCTCTGCGTGCCGAAGAC |
| | | | ACCGCCGTTTACTACTGTGCCCGTGCACCA |
| | | | CAGTGGATGTTAGTAGCGGAAGCATTCGAT |
| | | | CTATGGGGCCAGGGCACTATGGTGACCGTT |
| | | | AGCTCTGGCGGTGGTGGTAGCGGAGGCGGA |
| | | | GGATCAGGTGGAGGCGGCAGTTCTTACGTG |
| | | | CTGACTCAACCACCATCAGTGTCTGTAGCA |
| | | | CCAGGCCAGACCGCACGTATTACCTGTGGC |
| | | | GGTAACCTGATCGGCTCTAAGCTGGTTCAC |
| | | | TGGTATCAGCAAAAACCAGGGCAGGCACCA |
| | | | GTACTGGTTGTGTACGATGATAGCGATCGT |
| | | | CCAAGCCGGATTCCAGAGCGTTTCAGCGGC |
| | | | TCTAATTCCGGCAACACCGCTACTCTGACT |
| | | | ATTTCCCGTGTTGAAGCCGGCGATGAAGCC |
| | | | GACTACTATTGCCAGGTCTGGGACTCCAGC |
| | | | TCCGACCATGTAGTCTTTGGCGGGGGCACC |
| | | | AAACTGACCGTTTTG |
| 8 | 226 | C3 | CAAATGCAGCTGGTCGAGTCTGGCGGTGGG |
| | | VH-L-VL | GTAGTGCAACCAGGCCGTTCTCTGCGTCTT |
| | | | AGCTGCGCCGCATCTGGTTTTACCTTTCGT |
| | | | ACCTACGGTATGCACTGGGTGCGTCAGGCA |
| | | | CCAGGCAAAGGTCTGGAATGGGTCGCAGTA |
| | | | ATCTGGTATGATGGTAGCAATAAACACTAT |
| | | | GCTGACTCAGTCAAAGGCCGTTTCACCATC |
| | | | ACCCGTGATAACAGCAAGAACACTCTTAAC |
| | | | TTACAGATGAACTCTCTGCGTGCCGAAGAC |
| | | | ACCGCCGTTTACTACTGTGCCCGTGCACCA |
| | | | CAGTGGGAATGGGTAGCCGAAGCATTCGAT |
| | | | CTGTGGGGCCAGGGCACTATGGTGACCGTT |
| | | | AGCTCTGGCGGTGGTGGTAGCGGAGGCGGA |
| | | | GGATCAGGTGGAGGCGGCAGTTCTTACGTG |
| | | | CTGACTCAACCACCATCAGTGTCTGTAGCA |
| | | | CCAGGCCAGACCGCACGTATTACCTGTGGC |
| | | | GGTAACCTGATCGGCTCTAAGCTGGTTCAC |
| | | | TGGTATCAGCAAAAACCAGGCCAGGCACCA |
| | | | GTACTGGTTGTGTACGATGATAGCGATCGT |
| | | | CCAAGCCGGATTCCAGAGCGTTTCAGCGGC |
| | | | TCTAATTCCGGCAACACCGCTACTCTGACT |
| | | | ATTTCCCGTGTTGAAGCCGGCGATGAAGCC |
| | | | GACTACTATTGCCAGGTCTGGGACTCCAGC |
| | | | TCCGACCACGTAGTCTTTGGCGGGGGCACC |
| | | | AAACTGACCGTTTTG |
| 9 | 227 | C37- | CAAATGCAGCTGGTCGAGTCTGGCGGTGGG |
| | | VH-L-VL | GTAGTGCAACCAGGCCGTTCTCTGCGTCTT |
| | | | AGCTGCGCCGCATCTGGTTTTACCTTTCGT |
| | | | ACCTACGGTATGCACTGGGTGCGTCAGGCA |
| | | | CCAGGCAAAGGTCTGGAATGGGTCGCAGTA |
| | | | ATCTGGTATGATGGTAGCAATAAACACTAT |
| | | | GCTGACTCAGTCAAAGGCCGTTTCACCATC |
| | | | ACCCGTGATAACAGCAAGAACACTCTTAAC |
| | | | TTACAGATGAACTCTCTGCGTGCCGAAGAC |
| | | | ACCGCCGTTTACTACTGTGCCCGTGCACCA |
| | | | CAGTGGGAATTAGTAGCCGAGGCATTCGAT |
| | | | ATGTGGGGCCAGGGCACTATGGTGACCGTT |
| | | | AGCTCTGGCGGTGGTGGTAGCGGAGGCGGA |
| | | | GGATCAGGTGGAGGCGGCAGTTCTTACGTG |
| | | | CTGACTCAACCACCATCAGTGTCTGTAGCA |
| | | | CCAGGCCAGACCGCACGTATTACCTGTGGC |
| | | | GGTAACCTGATCGGCTCTAAGCTGGTTCAC |
| | | | TGGTATCAGCAAAAACCAGGCCAGGCACCA |
| | | | GTACTGGTTGTGTACGATGATAGCGATCGT |
| | | | CCAAGCCGGATTCCAGAGCGTTTCAGCGGC |
| | | | TCTAATTCCGGCAACACCGCTACTCTGACT |
| | | | ATTTCCCGTGTTGAAGCCGGCGATGAAGCC |
| | | | GACTACTATTGCCAGGTCTGGGACTCTAGC |
| | | | TCCGACCATGTAGTCTTTGGCGGGGGCACC |
| | | | AAACTGACCGTTTTG |
| 10 | 228 | C32- | CAAATGCAGCTGGTCGAGTCTGGCGGTGGG |
| | | VH-L-VL | GTAGTGCAACCAGGCCGTTCTCTGCGTCTT |
| | | | AGCTGCGCCGCATCTGGTTTTACCTTTCGT |
| | | | ACCTACGGTATGCACTGGGTGCGTCAGGCA |
| | | | CCAGGCAAAGGTCTGGAATGGGTCGCAGTA |
| | | | ATCTGGTATGATGGTAGCAATAAACACTAT |
| | | | GCTGACTCAGTCAAAGGCCGTTTCACCATC |
| | | | ACCCGTGATAACAGCAAGAACACTCTTAAC |
| | | | TTACAGATGAACTCTCTGCGTGCCGAAGAC |
| | | | ACCGCCGTTTACTACTGTGCCCGTGCACCA |
| | | | CAGTGGGAATTAGTAGCCGAAGCATTCGAT |
| | | | CTGTGGGGCCAGGGCACTATGGTGACCGTT |
| | | | AGCTCTGGCGGTGGTGGTAGCGGAGGCGGA |
| | | | GGATCAGGTGGAGGCGGCAGTTCTTACGTG |
| | | | CTGACTCAACCACCATCAGTGTCTGTAGCA |
| | | | CCAGGCCAGACCGCACGTATTACCTGTGGC |
| | | | GGTAACCTGATCGGCTCTAAGCTGGTTCAC |
| | | | TGGTATCAGCAAAAACCAGGCCAGGCACCA |
| | | | GTACTGGTTGTGTACGATGATAGCGATCGT |
| | | | CCAAGCAAAATTCCAGAGCGTTTCAGCGGC |
| | | | TCTAATTCCGGCAACACCGCTACTCTGACT |
| | | | ATTTCCCGTGTTGAAGCCGGCGATGAAGCC |
| | | | GACTACTATTGCCAGGTCTGGGACTCCAGC |
| | | | TCCGACCATGTAGTCTTTGGCGGGGGCACC |
| | | | AAACTGACCGTTTTG |
| 11 | 229 | C38- | CAAATGCAGCTGGTCGAGTCTGGCGGTGGG |
| | | VH-L-VL | GTAGTGCAACCAGGCCGTTCTCTGCGTCTT |
| | | | AGCTGCGCCGCATCTGGTTTTACCTTTCGT |
| | | | ACCTACGGTATGCACTGGGTGCGTCAGGCA |
| | | | CCAGGCAAAGGTCTGGAATGGGTCGCAGTA |
| | | | ATCTGGTATGATGGTAGCAATAAACACTAT |
| | | | GCTGACTCAGTCAAAGGCCGTTTCACCATC |
| | | | ACCCGTGATAACAGCAAGAACACTCTTAAC |
| | | | TTACAGATGAACTCTCTGCGTGCCGAAGAC |
| | | | ACCGCCGTTTACTACTGTGCCCGTGCACCA |
| | | | CAGTGGGAATACGTAGCGGAAGCATTCGAT |
| | | | CTGTGGGGCCAGGGCACTATGGTGACCGTT |
| | | | AGCTCTGGCGGTGGTGGTAGCGGAGGCGGA |
| | | | GGATCAGGTGGAGGCGGCAGTTCTTACGTG |
| | | | CTGACTCAACCACCATCAGTGTCTGTAGCA |
| | | | CCAGGCCAGACCGCACGTATTACCTGTGGC |
| | | | GGTAACCTGATCGGCTCTAAGCTGGTTCAC |
| | | | TGGTATCAGCAAAAACCAGGCCAGGCACCA |
| | | | GTACTGGTTGTGTACGATGATAGCGATCGT |
| | | | CCAAGCGATATTCCAGAGCGTTTCAGCGGC |
| | | | TCTAATTCCGGCAACACCGCTACTCTGACT |
| | | | ATTTCCCGTGTTGAAGCCGGCGATGAAGCC |
| | | | GACTACTATTGCCAGGTCTGGGACACCAGC |
| | | | TCCGACCATGTAGTCTTTGGCGGGGGCACC |
| | | | AAACTGACCGTTTTG |
| 12 | 230 | C26- | CAAATGCAGCTGGTCGAGTCTGGCGGTGGG |
| | | VH-L-VL | GTAGTGCAACCAGGCCGTTCTCTGCGTCTT |
| | | | AGCTGCGCCGCATCTGGTTTTACCTTTCGT |
| | | | ACCTACGGTATGCACTGGGTGCGTCAGGCA |
| | | | CCAGGCAAAGGTCTGGAATGGGTCGCAGTA |
| | | | ATCTGGTATGATGGTAGCAATAAACACTAT |
| | | | GCTGACTCAGTCAAAGGCCGTTTCACCATC |
| | | | ACCCGTGATAACAGCAAGAACACTCTTAAC |
| | | | TTACAGATGAACTCTCTGCGTGCCGAAGAC |
| | | | ACCGCCGTTTACTACTGTGCCCGTGCACCA |
| | | | CAGTGGGAATTAACGGCCGAAGCATTCGAT |
| | | | CTTTGGGGCCAGGGCACTATGGTGACCGTT |
| | | | AGCTCTGGCGGTGGTGGTAGCGGAGGCGGA |
| | | | GGATCAGGTGGAGGCGGCAGTTCTTACGTG |
| | | | CTGACTCAACCACCATCAGTGTCTGTAGCA |
| | | | CCAGGCCAGACCGCACGTATTACCTGTGGC |
| | | | GGTAACATCATCGGCTCTAAGCTGGTTCAC |
| | | | TGGTATCAGCAAAAACCAGGCCAGGCACCA |
| | | | GTACTGGTTGTGTACGATGATGGCGATCGT |
| | | | CCAAGCGGTATTCCAGAGCGTTTCAGCGGC |
| | | | TCTAATTCCGGCAACACCGCTACTCTGACT |
| | | | ATTTCCCGTGTTGAAGCCGGCGATGAAGCC |
| | | | GACTACTATTGCCAGGTCTGGGACACTAGC |
| | | | TCCGACCACGTAGTCTTTGGCGGGGGCACC |
| | | | AAACTGACCGTTTTG |
| 13 | 231 | C13- | CAAATGCAGCTGGTCGAGTCTGGCGGTGGG |
| | | VH-L-VL | GTAGTGCAACCAGGCCGTTCTCTGCGTCTT |
| | | | AGCTGCGCCGCATCTGGTTTTACCTTTCGT |
| | | | ACCTACGGTATGCACTGGGTGCGTCAGGCA |
| | | | CCAGGCAAAGGTCTGGAATGGGTCGCAGTA |
| | | | ATCTGGTATGATGGTAGCAATAAACACTAT |
| | | | GCTGACTCAGTCAAAGGCCGTTTCACCATC |
| | | | ACCCGTGATAACAGCAAGAACACTCTTAAC |
| | | | TTACAGATGAACTCTCTGCGTGCCGAAGAC |
| | | | ACCGCCGTTTACTACTGTGCCCGTGCACCA |
| | | | CAGTGGGAATTAGTGGCGGAAGCATTCGAT |
| | | | CTTTGGGGCCAGGGCACTATGGTGACCGTT |
| | | | AGCTCTGGCGGTGGTGGTAGCGGAGGCGGA |
| | | | GGATCAGGTGGAGGCGGCAGTTCTTACGTG |
| | | | CTGACTCAACCACCATCAGTGTCTGTAGCA |
| | | | CCAGGCCAGACCGCACGTATTACCTGTGGC |
| | | | GGTAACCTGCTGGGCTCTAAGCTGGTTCAC |
| | | | TGGTATCAGCAAAAACCAGGCCAGGCACCA |
| | | | GTACTGGTTGTGTACGATGATGGCGATCGT |
| | | | CCAAGCCTGATTCCAGAGCGTTTCAGCGGC |
| | | | TCTAATTCCGGCAACACCGCTACTCTGACT |
| | | | ATTTCCCGTGTTGAAGCCGGCGATGAAGCC |
| | | | GACTACTATTGCCAGGTCTGGGACACTAGC |
| | | | TCCGACCACGTAGTCTTTGGCGGGGGCACC |
| | | | AAACTGACCGTTTTG |
| 14 | 232 | C23- | CAAATGCAGCTGGTCGAGTCTGGCGGTGGG |
| | | VH-L-VL | GTAGTGCAACCAGGCCGTTCTCTGCGTCTT |
| | | | AGCTGCGCCGCATCTGGTTTTACCTTTCGT |
| | | | ACCTACGGTATGCACTGGGTGCGTCAGGCA |
| | | | CCAGGCAAAGGTCTGGAATGGGTCGCAGTA |
| | | | ATCTGGTATGATGGTAGCAATAAACACTAT |
| | | | GCTGACTCAGTCAAAGGCCGTTTCACCATC |
| | | | ACCCGTGATAACAGCAAGAACACTCTTAAC |
| | | | TTACAGATGAACTCTCTGCGTGCCGAAGAC |
| | | | ACCGCCGTTTACTACTGTGCCCGTGCACCA |
| | | | CAGTGGGAATTAACGGCGGAAGCATTCGAT |
| | | | ATTTGGGGCCAGGGCACTATGGTGACCGTT |
| | | | AGCTCTGGCGGTGGTGGTAGCGGAGGCGGA |
| | | | GGATCAGGTGGAGGCGGCAGTTCTTACGTG |
| | | | CTGACTCAACCACCATCAGTGTCTGTAGCA |
| | | | CCAGGCCAGACCGCACGTATTACCTGTGGC |
| | | | GGTAACCTGCTGGGCTCTAAGCTGGTTCAC |
| | | | TGGTATCAGCAAAAACCAGGCCAGGCACCA |
| | | | GTACTGGTTGTGTACGATGATAGCGATCGT |
| | | | CCAAGCGAAATTCCAGAGCGTTTCAGCGGC |
| | | | TCTAATTCCGGCAACACCGCTACTCTGACT |
| | | | ATTTCCCGTGTTGAAGCCGGCGATGAAGCC |
| | | | GACTACTATTGCCAGGTCTGGGACACCAGC |
| | | | TCCGACGGTGTAGTCTTTGGCGGGGGCACC |
| | | | AAACTGACCGTTTTG |
| 15 | 233 | C39- | CAAATGCAGCTGGTCGAGTCTGGCGGTGGG |
| | | VH-L-VL | GTAGTGCAACCAGGCCGTTCTCTGCGTCTT |
| | | | AGCTGCGCCGCATCTGGTTTTACCTTTCGT |
| | | | ACCTACGGTATGCACTGGGTGCGTCAGGCA |
| | | | CCAGGCAAAGGTCTGGAATGGGTCGCAGTA |
| | | | ATCTGGTATGATGGTAGCAATAAACACTAT |
| | | | GCTGACTCAGTCAAAGGCCGTTTCACCATC |
| | | | ACCCGTGATAACAGCAAGAACACTCTTAAC |
| | | | TTACAGATGAACTCTCTGCGTGCCGAAGAC |
| | | | ACCGCCGTTTACTACTGTGCCCGTGCACCA |
| | | | CAGTGGGAATTAACGGCCGAAGCATTCGAT |
| | | | CTTTGGGGCCAGGGCACTATGGTGACCGTT |
| | | | AGCTCTGGCGGTGGTGGTAGCGGAGGCGGA |
| | | | GGATCAGGTGGAGGCGGCAGTTCTTACGTG |
| | | | CTGACTCAACCACCATCAGTGTCTGTAGCA |
| | | | CCAGGCCAGACCGCACGTATTACCTGTGGC |
| | | | GGTAACCTGCTGGGCTCTAAGCTGGTTCAC |
| | | | TGGTATCAGCAAAAACCAGGCCAGGCACCA |
| | | | GTACTGGTTGTGTACGATGATAGCGATCGT |
| | | | CCAAGCTGGATTCCAGAGCGTTTCAGCGGC |
| | | | TCTAATTCCGGCAACACCGCTACTCTGACT |
| | | | ATTTCCCGTGTTGAAGCCGGCGATGAAGCC |
| | | | GACTACTATTGCCAGGTCTGGGACACTAGC |
| | | | TCCGACCATGTAGTCTTTGGCGGGGGCACC |
| | | | AAACTGACCGTTTTG |
| 16 | 234 | C17- | CAAATGCAGCTGGTCGAGTCTGGCGGTGGG |
| | | VH-L-VL | GTAGTGCAACCAGGCCGTTCTCTGCGTCTT |
| | | | AGCTGCGCCGCATCTGGTTTTACCTTTCGT |
| | | | ACCTACGGTATGCACTGGGTGCGTCAGGCA |
| | | | CCAGGCAAAGGTCTGGAATGGGTCGCAGTA |
| | | | ATCTGGTATGATGGTAGCAATAAACACTAT |
| | | | GCTGACTCAGTCAAAGGCCGTTTCACCATC |
| | | | ACCCGTGATAACAGCAAGAACACTCTTAAC |
| | | | TTACAGATGAACTCTCTGCGTGCCGAAGAC |
| | | | ACCGCCGTTTACTACTGTGCCCGTGCACCA |
| | | | CAGTGGGAATTAACGTCGGAAGCATTCGAT |
| | | | CTTTGGGGCCAGGGCACTATGGTGACCGTT |
| | | | AGCTCTGGCGGTGGTGGTAGCGGAGGCGGA |
| | | | GGATCAGGTGGAGGCGGCAGTTCTTACGTG |
| | | | CTGACTCAACCACCATCAGTGTCTGTAGCA |
| | | | CCAGGCCAGACCGCACGTATTACCTGTGGC |
| | | | GGTAACCTGATCGGCTCTAAGCTGGTTCAC |
| | | | TGGTATCAGCAAAAACCAGGCCAGGCACCA |
| | | | GTACTGGTTGTGTACGATGATAGCGATCGT |
| | | | CCAAGCGCAATTCCAGAGCGTTTCAGCGGC |
| | | | TCTAATTCCGGCAACACCGCTACTCTGACT |
| | | | ATTTCCCGTGTTGAAGCCGGCGATGAAGCC |
| | | | GACTACTATTGCCAGGTCTGGGACACCAGC |
| | | | TCCGACCACGTAGTCTTTGGCGGGGGCACC |
| | | | AAACTGACCGTTTTG |
| 17 | 235 | C45- | CAAATGCAGCTGGTCGAGTCTGGCGGTGGG |
| | | VH-L-VL | GTAGTGCAACCAGGCCGTTCTCTGCGTCTT |
| | | | AGCTGCGCCGCATCTGGTTTTACCTTTCGT |
| | | | ACCTACGGTATGCACTGGGTGCGTCAGGCA |
| | | | CCAGGCAAAGGTCTGGAATGGGTCGCAGTA |
| | | | ATCTGGTATGATGGTAGCAATAAACACTAT |
| | | | GCTGACTCAGTCAAAGGCCGTTTCACCATC |
| | | | ACCCGTGATAACAGCAAGAACACTCTTAAC |
| | | | TTACAGATGAACTCTCTGCGTGCCGAAGAC |
| | | | ACCGCCGTTTACTACTGTGCCCGTGCACCA |
| | | | CAGTGGGAATTAACCTCCGAAGCATTCGAT |
| | | | CTTTGGGGCCAGGGCACTATGGTGACCGTT |
| | | | AGCTCTGGCGGTGGTGGTAGCGGAGGCGGA |
| | | | GGATCAGGTGGAGGCGGCAGTTCTTACGTG |
| | | | CTGACTCAACCACCATCAGTGTCTGTAGCA |
| | | | CCAGGCCAGACCGCACGTATTACCTGTGGC |
| | | | GGTAACCTGATCGGCTCTAAGCTGGTTCAC |
| | | | TGGTATCAGCAAAAACCAGGCCAGGCACCA |
| | | | GTACTGGTTGTGTACGATGATAGCGATCGT |
| | | | CCAAGCGAAATTCCAGAGCGTTTCAGCGGC |
| | | | TCTAATTCCGGCAACACCGCTACTCTGACT |
| | | | ATTTCCCGTGTTGAAGCCGGCGATGAAGCC |
| | | | GACTACTATTGCCAGGTCTGGGACTCCAGC |
| | | | TCCGACGGTGTAGTCTTTGGCGGGGGCACC |
| | | | AAACTGACCGTTTTG |
| 18 | 236 | C7- | CAAATGCAGCTGGTCGAGTCTGGCGGTGGG |
| | | VH-L-VL | GTAGTGCAACCAGGCCGTTCTCTGCGTCTT |
| | | | AGCTGCGCCGCATCTGGTTTTACCTTTCGT |
| | | | ACCTACGGTATGCACTGGGTGCGTCAGGCA |
| | | | CCAGGCAAAGGTCTGGAATGGGTCGCAGTA |
| | | | ATCTGGTATGATGGTAGCAATAAACACTAT |
| | | | GCTGACTCAGTCAAAGGCCGTTTCACCATC |
| | | | ACCCGTGATAACAGCAAGAACACTCTTAAC |
| | | | TTACAGATGAACTCTCTGCGTGCCGAAGAC |
| | | | ACCGCCGTTTACTACTGTGCCCGTGCACCA |
| | | | CAGTGGCTGTTAGTAGCGGAAGCATTCGAT |
| | | | CTCTGGGGCCAGGGCACTATGGTGACCGTT |
| | | | AGCTCTGGCGGTGGTGGTAGCGGAGGCGGA |
| | | | GGATCAGGTGGAGGCGGCAGTTCTTACGTG |
| | | | CTGACTCAACCACCATCAGTGTCTGTAGCA |
| | | | CCAGGCCAGACCGCACGTATTACCTGTGGC |
| | | | GGTAACATCATCGGCTCTAAGCTGGTTCAC |
| | | | TGGTATCAGCAAAAACCAGGCCAGGCACCA |
| | | | GTACTGGTTGTGTACGATGATAGCGATCGT |
| | | | CCAAGCGGTATTCCAGAGCGTTTCAGCGGC |
| | | | TCTAATTCCGGCAACACCGCTACTCTGACT |
| | | | ATTTCCCGTGTTGAAGCCGGCGATGAAGCC |
| | | | GACTACTATTGCCAGGTCTGGGACTCTGGC |
| | | | TCCGACCACGTAGTCTTTGGCGGGGGCACC |
| | | | AAACTGACCGTTTTG |
| 19 | 237 | C31- | CAAATGCAGCTGGTCGAGTCTGGCGGTGGG |
| | | VH-L-VL | GTAGTGCAACCAGGCCGTTCTCTGCGTCTT |
| | | | AGCTGCGCCGCATCTGGTTTTACCTTTCGT |
| | | | ACCTACGGTATGCACTGGGTGCGTCAGGCA |
| | | | CCAGGCAAAGGTCTGGAATGGGTCGCAGTA |
| | | | ATCTGGTATGATGGTAGCAATAAACACTAT |
| | | | GCTGACTCAGTCAAAGGCCGTTTCACCATC |
| | | | ACCCGTGATAACAGCAAGAACACTCTTAAC |
| | | | TTACAGATGAACTCTCTGCGTGCCGAAGAC |
| | | | ACCGCCGTTTACTACTGTGCCCGTGCACCA |
| | | | CAGTGGGAATTAGTAGCGGAAGCATTCGAT |
| | | | CTTTGGGGCCAGGGCACTATGGTGACCGTT |
| | | | AGCTCTGGCGGTGGTGGTAGCGGAGGCGGA |
| | | | GGATCAGGTGGAGGCGGCAGTTCTTACGTG |
| | | | CTGACTCAACCACCATCAGTGTCTGTAGCA |
| | | | CCAGGCCAGACCGCACGTATTACCTGTGGC |
| | | | GGTAACATCATCGGCTCTAAGCTGGTTCAC |
| | | | TGGTATCAGCAAAAACCAGGCCAGGCACCA |
| | | | GTACTGGTTGTGTACGATGATAGCGATCGT |
| | | | CCAAGCGATATTCCAGAGCGTTTCAGCGGC |
| | | | TCTAATTCCGGCAACACCGCTACTCTGACT |
| | | | ATTTCCCGTGTTGAAGCCGGCGATGAAGCC |
| | | | GACTACTATTGCCAGGTCTGGGACTCCGGC |
| | | | TCCGACGGTGTAGTCTTTGGCGGGGGCACC |
| | | | AAACTGACCGTTTTG |
| 20 | 238 | C15- | CAAATGCAGCTGGTCGAGTCTGGCGGTGGG |
| | | VH-L-VL | GTAGTGCAACCAGGCCGTTCTCTGCGTCTT |
| | | | AGCTGCGCCGCATCTGGTTTTACCTTTCGT |
| | | | ACCTACGGTATGCACTGGGTGCGTCAGGCA |
| | | | CCAGGCAAAGGTCTGGAATGGGTCGCAGTA |
| | | | ATCTGGTATGATGGTAGCAATAAACACTAT |
| | | | GCTGACTCAGTCAAAGGCCGTTTCACCATC |
| | | | ACCCGTGATAACAGCAAGAACACTCTTAAC |
| | | | TTACAGATGAACTCTCTGCGTGCCGAAGAC |
| | | | ACCGCCGTTTACTACTGTGCCCGTGCACCA |
| | | | CAGTGGGAATTAGTAGCGGAAGCATTCGAT |
| | | | CTGTGGGGCCAGGGCACTATGGTGACCGTT |
| | | | AGCTCTGGCGGTGGTGGTAGCGGAGGCGGA |
| | | | GGATCAGGTGGAGGCGGCAGTTCTTACGTG |
| | | | CTGACTCAACCACCATCAGTGTCTGTAGCA |
| | | | CCAGGCCAGACCGCACGTATTACCTGTGGC |
| | | | GGTAACCTGATCGGCTCTAAGCTGGTTCAC |
| | | | TGGTATCAGCAAAAACCAGGCCAGGCACCA |
| | | | GTACTGGTTGTGTACGATGATGGCGATCGT |
| | | | CCAAGCTGGATTCCAGAGCGTTTCAGCGGC |
| | | | TCTAATTCCGGCAACACCGCTACTCTGACT |
| | | | ATTTCCCGTGTTGAAGCCGGCGATGAAGCC |
| | | | GACTACTATTGCCAGGTCTGGGACTCTGGC |
| | | | TCCGACGGTGTAGTCTTTGGCGGGGGCACC |
| | | | AAACTGACCGTTTTG |
| 21 | 239 | C35- | CAAATGCAGCTGGTCGAGTCTGGCGGTGGG |
| | | VH-L-VL | GTAGTGCAACCAGGCCGTTCTCTGCGTCTT |
| | | | AGCTGCGCCGCATCTGGTTTTACCTTTCGT |
| | | | ACCTACGGTATGCACTGGGTGCGTCAGGCA |
| | | | CCAGGCAAAGGTCTGGAATGGGTCGCAGTA |
| | | | ATCTGGTATGATGGTAGCAATAAACACTAT |
| | | | GCTGACTCAGTCAAAGGCCGTTTCACCATC |
| | | | ACCCGTGATAACAGCAAGAACACTCTTAAC |
| | | | TTACAGATGAACTCTCTGCGTGCCGAAGAC |
| | | | ACCGCCGTTTACTACTGTGCCCGTGCACCA |
| | | | CAGTGGGTCTTAGTATCCGAAGCATTCGAT |
| | | | CTTTGGGGCCAGGGCACTATGGTGACCGTT |
| | | | AGCTCTGGCGGTGGTGGTAGCGGAGGCGGA |
| | | | GGATCAGGTGGAGGCGGCAGTTCTTACGTG |
| | | | CTGACTCAACCACCATCAGTGTCTGTAGCA |
| | | | CCAGGCCAGACCGCACGTATTACCTGTGGC |
| | | | GGTAACCTGATCGGCTCTAAGCTGGTTCAC |
| | | | TGGTATCAGCAAAAACCAGGCCAGGCACCA |
| | | | GTACTGGTTGTGTACGATGATAGCGATCGT |
| | | | CCAAGCCGTATTCCAGAGCGTTTCAGCGGC |
| | | | TCTAATTCCGGCAACACCGCTACTCTGACT |
| | | | ATTTCCCGTGTTGAAGCCGGCGATGAAGCC |
| | | | GACTACTATTGCCAGGTCTGGGACTCCGGC |
| | | | TCCGACGGTGTAGTCTTTGGCGGGGGCACC |
| | | | AAACTGACCGTTTTG |
| 22 | 240 | C12 - | CAAATGCAGCTGGTCGAGTCTGGCGGTGGG |
| | | VH-L-VL | GTAGTGCAACCAGGCCGTTCTCTGCGTCTT |
| | | | AGCTGCGCCGCATCTGGTTTTACCTTTCGT |
| | | | ACCTACGGTATGCACTGGGTGCGTCAGGCA |
| | | | CCAGGCAAAGGTCTGGAATGGGTCGCAGTA |
| | | | ATCTGGTATGATGGTAGCAATAAACACTAT |
| | | | GCTGACTCAGTCAAAGGCCGTTTCACCATC |
| | | | ACCCGTGATAACAGCAAGAACACTCTTAAC |
| | | | TTACAGATGAACTCTCTGCGTGCCGAAGAC |
| | | | ACCGCCGTTTACTACTGTGCCCGTGCACCA |
| | | | CAGTGGGAATTAGTAGCGGAGGCATTCGAT |
| | | | CTGTGGGGCCAGGGCACTATGGTGACCGTT |
| | | | AGCTCTGGCGGTGGTGGTAGCGGAGGCGGA |
| | | | GGATCAGGTGGAGGCGGCAGTTCTTACGTG |
| | | | CTGACTCAACCACCATCAGTGTCTGTAGCA |
| | | | CCAGGCCAGACCGCACGTATTACCTGTGGC |
| | | | GGTAACATCATCGGCTCTAAGCTGGTTCAC |
| | | | TGGTATCAGCAAAAACCAGGCCAGGCACCA |
| | | | GTACTGGTTGTGTACGATGATAGCGATCGT |
| | | | CCAAGCGCAATTCCAGAGCGTTTCAGCGGC |
| | | | TCTAATTCCGGCAACACCGCTACTCTGACT |
| | | | ATTTCCCGTGTTGAAGCCGGCGATGAAGCC |
| | | | GACTACTATTGCCAGGTCTGGGACTCCAGC |
| | | | TCCGACGGTGTAGTCTTTGGCGGGGGCACC |
| | | | AAACTGACCGTTTTGCAAATGCAGCTGGTC |
| 23 | 241 | C4- | GAGTCTGGCGGTGGGGTAGTGCAACCAG |
| | | VH-L-VL | GCCGTTCTCTGCGTCTTAGCTGCGCCGCAT |
| | | | CTGGTTTTACCTTTCGTACCTACGGTATGC |
| | | | ACTGGGTGCGTCAGGCACCAGGCAAAGGTC |
| | | | TGGAATGGGTCGCAGTAATCTCTTATGATG |
| | | | GTAGCAATAAACACTATGCTGACTCAGTCA |
| | | | AAGGCCGTTTCACCATCACCCGTGATAACA |
| | | | GCAAGAACACTCTTAACTTACAGATGAACT |
| | | | CTCTGCGTGCCGAAGACACCGCCGTTTACT |
| | | | ACTGTGCCCGTAGCCCACAGTGGGAATGGG |
| | | | TACACGAAGCATTCGATATGTGGGGCCAGG |
| | | | GCACTATGGTGACCGTTAGCTCTGGCGGTG |
| | | | GTGGTAGCGGAGGCGGAGGATCAGGTGGAG |
| | | | GCGGCAGTTCTTACGTGCTGACTCAACCAC |
| | | | CATCAGTGTCTGTAGCACCAGGCCAGACCG |
| | | | CACGTATTACCTGTGGCGGTAACAACATCG |
| | | | GCTCTAAGCTGGTTCACTGGTATCAGCAAA |
| | | | AACCAGGCCAGGCACCAGTACTGGTTGTGT |
| | | | ACGATGATAGCGATCGTCCAAGCGGTATTC |
| | | | CAGAGCGTTTCAGCGGCTCTAATTCCGGCA |
| | | | ACACCGCTACTCTGACTATTTCCCGTGTTG |
| | | | AAGCCGGCGATGAAGCCGACTACTATTGCC |
| | | | AGGTCTGGGACTCTAGCTCCGACGGTGTAG |
| | | | TCTTTGGCGGGGGCACCAAACTGACCGTTT |
| | | | TG |
| 24 | 242 | C41- | CAAATGCAGCTGGTCGAGTCTGGCGGTGGG |
| | | VH-L-VL | GTAGTGCAACCAGGCCGTTCTCTGCGTCTT |
| | | | AGCTGCGCCGCATCTGGTTTTACCTTTCGT |
| | | | ACCTACGGTATGCACTGGGTGCGTCAGGCA |
| | | | CCAGGCAAAGGTCTGGAATGGGTCGCAGTA |
| | | | ATCTCTTATGATGGTAGCAATAAACACTAT |
| | | | GCTGACTCAGTCAAAGGCCGTTTCACCATC |
| | | | ACCCGTGATAACAGCAAGAACACTCTTAAC |
| | | | TTACAGATGAACTCTCTGCGTGCCGAAGAC |
| | | | ACCGCCGTTTACTACTGTGCCCGTTCGCCA |
| | | | CAGTGGGAATGGGTACACGAAGCATTCGAT |
| | | | CTCTGGGGCCAGGGCACTATGGTGACCGTT |
| | | | AGCTCTGGCGGTGGTGGTAGCGGAGGCGGA |
| | | | GGATCAGGTGGAGGCGGCAGTTCTTACGTG |
| | | | CTGACTCAACCACCATCAGTGTCTGTAGCA |
| | | | CCAGGCCAGACCGCACGTATTACCTGTGGC |
| | | | GGTAACATCCTGGGCTCTAAGCTGGTTCAC |
| | | | TGGTATCAGCAAAAACCAGGCCAGGCACCA |
| | | | GTACTGGTTGTGTACGATGATAGCGATCGT |
| | | | CCAAGCGAAATTCCAGAGCGTTTCAGCGGC |
| | | | TCTAATTCCGGCAACACCGCTACTCTGACT |
| | | | ATTTCCCGTGTTGAAGCCGGCGATGAAGCC |
| | | | GACTACTATTGCCAGGTCTGGGACACCAGC |
| | | | TCCGACGGTGTAGTCTTTGGCGGGGGCACC |
| | | | AAACTGACCGTTTTG |
| 25 | 243 | C40- | CAAATGCAGCTGGTCGAGTCTGGCGGTGGG |
| | | VH-L-VL | GTAGTGCAACCAGGCCGTTCTCTGCGTCTT |
| | | | AGCTGCGCCGCATCTGGTTTTACCTTTCGT |
| | | | ACCTACGGTATGCACTGGGTGCGTCAGGCA |
| | | | CCAGGCAAAGGTCTGGAATGGGTCGCAGTA |
| | | | ATCTGGTATGATGGTAGCAATAAACACTAT |
| | | | GCTGACTCAGTCAAAGGCCGTTTCACCATC |
| | | | ACCCGTGATAACAGCAAGAACACTCTTAAC |
| | | | TTACAGATGAACTCTCTGCGTGCCGAAGAC |
| | | | ACCGCCGTTTACTACTGTGCCCGTAGCCCA |
| | | | CAGTGGGAATGGGTACACGAAGCATTCGAT |
| | | | CTATGGGGCCAGGGCACTATGGTGACCGTT |
| | | | AGCTCTGGCGGTGGTGGTAGCGGAGGCGGA |
| | | | GGATCAGGTGGAGGCGGCAGTTCTTACGTG |
| | | | CTGACTCAACCACCATCAGTGTCTGTAGCA |
| | | | CCAGGCCAGACCGCACGTATTACCTGTGGC |
| | | | GGTAACAACATCGGCTCTAAGCTGGTTCAC |
| | | | TGGTATCAGCAAAAACCAGGCCAGGCACCA |
| | | | GTACTGGTTGTGTACGATGATAGCGATCGT |
| | | | CCAAGCCGGATTCCAGAGCGTTTCAGCGGC |
| | | | TCTAATTCCGGCAACACCGCTACTCTGACT |
| | | | ATTTCCCGTGTTGAAGCCGGCGATGAAGCC |
| | | | GACTACTATTGCCAGGTCTGGGACACTAGC |
| | | | TCCGACGGTGTAGTCTTTGGCGGGGGCACC |
| | | | AAACTGACCGTTTTG |
| 26 | 244 | C9- | CAAATGCAGCTGGTCGAGTCTGGCGGTGGG |
| | | VH-L-VL | GTAGTGCAACCAGGCCGTTCTCTGCGTCTT |
| | | | AGCTGCGCCGCATCTGGTTTTACCTTTCGT |
| | | | ACCTACGGTATGCACTGGGTGCGTCAGGCA |
| | | | CCAGGCAAAGGTCTGGAATGGGTCGCAGTA |
| | | | ATCTGGTATGATGGTAGCAATAAACACTAT |
| | | | GCTGACTCAGTCAAAGGCCGTTTCACCATC |
| | | | ACCCGTGATAACAGCAAGAACACTCTTAAC |
| | | | TTACAGATGAACTCTCTGCGTGCCGAAGAC |
| | | | ACCGCCGTTTACTACTGTGCCCGTTCGCCA |
| | | | CAGTGGGAATGGGTACACGAAGCATTCGAT |
| | | | CTCTGGGGCCAGGGCACTATGGTGACCGTT |
| | | | AGCTCTGGCGGTGGTGGTAGCGGAGGCGGA |
| | | | GGATCAGGTGGAGGCGGCAGTTCTTACGTG |
| | | | CTGACTCAACCACCATCAGTGTCTGTAGCA |
| | | | CCAGGCCAGACCGCACGTATTACCTGTGGC |
| | | | GGTAACCTGATCGGCTCTAAGCTGGTTCAC |
| | | | TGGTATCAGCAAAAACCAGGCCAGGCACCA |
| | | | GTACTGGTTGTGTACGATGATGGCGATCGT |
| | | | CCAAGCTGGATTCCAGAGCGTTTCAGCGGC |
| | | | TCTAATTCCGGCAACACCGCTACTCTGACT |
| | | | ATTTCCCGTGTTGAAGCCGGCGATGAAGCC |
| | | | GACTACTATTGCCAGGTCTGGGACTCCAGC |
| | | | TCCGACGGTGTAGTCTTTGGCGGGGGCACC |
| | | | AAACTGACCGTTTTG |

TABLE 10
Nucleotide Sequences Encoding Engineered dual binding
antibodies: Variable Light chain (VL) and
Variable Heavy chain (VH) nucleic acid sequences

| | SEQ | | | SEQ | | |
|---|---|---|---|---|---|---|
| Ab | ID | | | ID | | |
| # | NO: | Name | VL Sequence | NO: | Name | VH Sequence |
| | 165 | Template- | TCTTACGTGCTGACTCAA | 164 | Template- | CAAATGCAGCTGGTCGAGT |
| | | VL | CCACCATCAGTGTCTGTA | | VH | CTGGCGGTGGGGTAGTGCA |
| | | | GCACCAGGCCAGACCGCA | | | ACCAGGCCGTTCTCTGCGT |
| | | | CGTATTACCTGTGGCGGT | | | CTTAGCTGCGCCGCATCTG |
| | | | AACAATCTGGGCTCTAAG | | | GTTTTACCTTTCGTACCTAC |
| | | | TCTGTTCACTGGTATCAG | | | GGTATGCACTGGGTGCGTC |
| | | | CAAAAACCAGGCCAGGC | | | AGGCACCAGGCAAAGGTCT |
| | | | ACCAGTACTGGTTGTGTA | | | GGAATGGGTCGCAGTAATC |
| | | | CGATGATTCCGATCGTCC | | | TGGTATGATGGTAGCAATA |
| | | | AAGCTGGATTCCAGAGCG | | | AACACTATGCTGACTCAGT |
| | | | TTTCAGCGGCTCTAATTC | | | CAAAGGCCGTTTCACCATC |
| | | | CGGCAACACCGCTACTCT | | | ACCCGTGATAACAGCAAGA |
| | | | GACTATTTCCCGTGGGGA | | | ACACTCTTAACTTACAGAT |
| | | | AGCCGGCGATGAAGCCG | | | GAACTCTCTGCGTGCCGAA |
| | | | ACTACTATTGCCAGGTCT | | | GACACCGCAGTTTATTACT |
| | | | GGGACTCTTCTTCCGACC | | | GTGCCCGTGCACCACAGTG |
| | | | ATGTAGTCTTTGGCGGGG | | | GGAATTAGTACACGAAGCA |
| | | | GCACCAAACTGACCGTTT | | | TTCGATATCTGGGGTCAGG |
| | | | TG | | | GTACTATGGTGACCGTT |
| | | | | | | AGCTCT |
| 1 | 167 | C2-VL | TCTTACGTGCTGACTCAA | 166 | C2-VH | CAAATGCAGCTGGTCGAGT |
| | | | CCACCATCAGTGTCTGTA | | | CTGGCGGTGGGGTAGTGCA |
| | | | GCACCAGGCCAGACCGCA | | | ACCAGGCCGTTCTCTGCGT |
| | | | CGTATTACCTGTGGCGGT | | | CTTAGCTGCGCCGCATCTG |
| | | | AACCTGATCGGCTCTAAG | | | GTTTTACCTTTCGTACCTAC |
| | | | CTGGTTCACTGGTATCAG | | | GGTATGCACTGGGTGCGTC |
| | | | CAAAAACCAGGCCAGGC | | | AGGCACCAGGCAAAGGTCT |
| | | | ACCAGTACTGGTTGTGTA | | | GGAATGGGTCGCAGTAATC |
| | | | CGATGATAGCGATCGTCC | | | TGGTATGATGGTAGCAATA |
| | | | AAGCCTGATTCCAGAGCG | | | AACACTAT |
| | | | TTTCAGCGGCTCTAATTC | | | GCTGACTCAGTCAAAGGCC |
| | | | CGGCAACACCGCTACTCT | | | GTTTCACCATCACCCGTGAT |
| | | | GACTATTTCCCGTGTTGA | | | AACAGCAAGAACACTCTTA |
| | | | AGCCGGCGATGAAGCCG | | | ACTTACAGATGAACTCTCT |
| | | | ACTACTATTGCCAGGTCT | | | GCGTGCCGAAGACACCGCC |
| | | | GGGACTCTAGCTCCGACG | | | GTTTACTACTGTGCCCGTGC |
| | | | GTGTAGTCTTTGGCGGGG | | | ACCACAGTGGGAATTAACG |
| | | | GCACCAAACTGACCGTTT | | | GCGGAAGCATTCGATATTT |
| | | | TG | | | GGGGCCAGGGCACTATGGT |
| | | | | | | GACCGTT |
| | | | | | | AGCTCT |
| 2 | 169 | C27-VL | TCTTACGTGCTGACTCAA | 168 | C27-VH | CAAATGCAGCTGGTCGAGT |
| | | | CCACCATCAGTGTCTGTA | | | CTGGCGGTGGGGTAGTGCA |
| | | | GCACCAGGCCAGACCGCA | | | ACCAGGCCGTTCTCTGCGT |
| | | | CGTATTACCTGTGGCGGT | | | CTTAGCTGCGCCGCATCTG |
| | | | AACCTGATCGGCTCTAAG | | | GTTTTACCTTTCGTACCTAC |
| | | | CTGGTTCACTGGTATCAG | | | GGTATGCACTGGGTGCGTC |
| | | | CAAAAACCAGGCCAGGC | | | AGGCACCAGGCAAAGGTCT |
| | | | ACCAGTACTGGTTGTGTA | | | GGAATGGGTCGCAGTAATC |
| | | | CGATGATAGCGATCGTCC | | | TGGTATGATGGTAGCAATA |
| | | | AAGCCTGATTCCAGAGCG | | | AACACTAT |
| | | | TTTCAGCGGCTCTAATTC | | | GCTGACTCAGTCAAAGGCC |
| | | | CGGCAACACCGCTACTCT | | | GTTTCACCATCACCCGTGAT |
| | | | GACTATTTCCCGTGTTGA | | | AACAGCAAGAACACTCTTA |
| | | | AGCCGGCGATGAAGCCG | | | ACTTACAGATGAACTCTCT |
| | | | ACTACTATTGCCAGGTCT | | | GCGTGCCGAAGACACCGCC |
| | | | GGGACTCTAGCTCCGACG | | | GTTTACTACTGTGCCCGTGC |
| | | | GTGTAGTCTTTGGCGGGG | | | ACCACAGTGGGAATTAGTA |
| | | | GCACCAAACTGACCGTTT | | | GCGGAAGCATTCGATCTGT |
| | | | TG | | | GGGGCCAGGGCACTATGGT |
| | | | | | | GACCGTT |
| | | | | | | AGCTCT |
| 3 | 171 | C19-VL | TCTTACGTGCTGACTCAA | 170 | C19-VH | CAAATGCAGCTGGTCGAGT |
| | | | CCACCATCAGTGTCTGTA | | | CTGGCGGTGGGGTAGTGCA |
| | | | GCACCAGGCCAGACCGCA | | | ACCAGGCCGTTCTCTGCGT |
| | | | CGTATTACCTGTGGCGGT | | | CTTAGCTGCGCCGCATCTG |
| | | | AACCTGATCGGCTCTAAG | | | GTTTTACCTTTCGTACCTAC |
| | | | CTGGTTCACTGGTATCAG | | | GGTATGCACTGGGTGCGTC |
| | | | CAAAAACCAGGCCAGGC | | | AGGCACCAGGCAAAGGTCT |
| | | | ACCAGTACTGGTTGTGTA | | | GGAATGGGTCGCAGTAATC |
| | | | CGATGATAGCGATCGTCC | | | TGGTATGATGGTAGCAATA |
| | | | AAGCCTTATTCCAGAGCG | | | AACACTAT |
| | | | TTTCAGCGGCTCTAATTC | | | GCTGACTCAGTCAAAGGCC |
| | | | CGGCAACACCGCTACTCT | | | GTTTCACCATCACCCGTGAT |
| | | | GACTATTTCCCGTGTTGA | | | AACAGCAAGAACACTCTTA |
| | | | AGCCGGCGATGAAGCCG | | | ACTTACAGATGAACTCTCT |
| | | | ACTACTATTGCCAGGTCT | | | GCGTGCCGAAGACACCGCC |
| | | | GGGACACCAGCTCCGACG | | | GTTTACTACTGTGCCCGTGC |
| | | | GTGTAGTCTTTGGCGGGG | | | ACCACAGTGGCAGTTAGTA |
| | | | GCACCAAACTGACCGTTT | | | GCGGAAGCATTCGATATTT |
| | | | TG | | | GGGGCCAGGGCACTATGGT |
| | | | | | | GACCGTT |
| | | | | | | AGCTCT |
| 4 | 173 | C43-VL | TCTTACGTGCTGACTCAA | 172 | C43-VH | CAAATGCAGCTGGTCGAGT |
| | | | CCACCATCAGTGTCTGTA | | | CTGGCGGTGGGGTAGTGCA |
| | | | GCACCAGGCCAGACCGCA | | | ACCAGGCCGTTCTCTGCGT |
| | | | CGTATTACCTGTGGCGGT | | | CTTAGCTGCGCCGCATCTG |
| | | | AACCTGATCGGCTCTAAG | | | GTTTTACCTTTCGTACCTAC |
| | | | CTGGTTCACTGGTATCAG | | | GGTATGCACTGGGTGCGTC |
| | | | CAAAAACCAGGCCAGGC | | | AGGCACCAGGCAAAGGTCT |
| | | | ACCAGTACTGGTTGTGTA | | | GGAATGGGTCGCAGTAATC |
| | | | CGATGATAGCGATCGTCC | | | TGGTATGATGGTAGCAATA |
| | | | AAGCGCTATTCCAGAGCG | | | AACACTAT |
| | | | TTTCAGCGGCTCTAATTC | | | GCTGACTCAGTCAAAGGCC |
| | | | CGGCAACACCGCTACTCT | | | GTTTCACCATCACCCGTGAT |
| | | | GACTATTTCCCGTGTTGA | | | AACAGCAAGAACACTCTTA |
| | | | AGCCGGCGATGAAGCCG | | | ACTTACAGATGAACTCTCT |
| | | | ACTACTATTGCCAGGTCT | | | GCGTGCCGAAGACACCGCC |
| | | | GGGACACTAGCTCCGACG | | | GTTTACTACTGTGCCCGTGC |
| | | | GTGTAGTCTTTGGCGGGG | | | ACCACAGTGGCAGTTAGTA |
| | | | GCACCAAACTGACCGTTT | | | GCCGAAGCATTCGATATAT |
| | | | TG | | | GGGGCCAGGGCACTATGGT |
| | | | | | | GACCGTT |
| | | | | | | AGCTCT |
| 5 | 175 | C5-VL | TCTTACGTGCTGACTCAA | 174 | C5-VH | CAAATGCAGCTGGTCGAGT |
| | | | CCACCATCAGTGTCTGTA | | | CTGGCGGTGGGGTAGTGCA |
| | | | GCACCAGGCCAGACCGCA | | | ACCAGGCCGTTCTCTGCGT |
| | | | CGTATTACCTGTGGCGGT | | | CTTAGCTGCGCCGCATCTG |
| | | | AACCTGATCGGCTCTAAG | | | GTTTTACCTTTCGTACCTAC |
| | | | CTGGTTCACTGGTATCAG | | | GGTATGCACTGGGTGCGTC |
| | | | CAAAAACCAGGCCAGGC | | | AGGCACCAGGCAAAGGTCT |
| | | | ACCAGTACTGGTTGTGTA | | | GGAATGGGTCGCAGTAATC |
| | | | CGATGATAGCGATCGTCC | | | TGGTATGATGGTAGCAATA |
| | | | AAGCCTTATTCCAGAGCG | | | AACACTAT |
| | | | TTTCAGCGGCTCTAATTC | | | GCTGACTCAGTCAAAGGCC |
| | | | CGGCAACACCGCTACTCT | | | GTTTCACCATCACCCGTGAT |
| | | | GACTATTTCCCGTGTTGA | | | AACAGCAAGAACACTCTTA |
| | | | AGCCGGCGATGAAGCCG | | | ACTTACAGATGAACTCTCT |
| | | | ACTACTATTGCCAGGTCT | | | GCGTGCCGAAGACACCGCC |
| | | | GGGACACCAGCTCCGACG | | | GTTTACTACTGTGCCCGTGC |
| | | | GTGTAGTCTTTGGCGGGG | | | ACCACAGTGGGAATTAGTG |
| | | | GCACCAAACTGACCGTTT | | | GCGGAAGCATTCGATCTTT |
| | | | TG | | | GGGGCCAGGGCACTATGGT |
| | | | | | | GACCGTT |
| | | | | | | AGCTCT |
| 6 | 177 | C8-VL | TCTTACGTGCTGACTCAA | 176 | C8-VH | CAAATGCAGCTGGTCGAGT |
| | | | CCACCATCAGTGTCTGTA | | | CTGGCGGTGGGGTAGTGCA |
| | | | GCACCAGGCCAGACCGCA | | | ACCAGGCCGTTCTCTGCGT |
| | | | CGTATTACCTGTGGCGGT | | | CTTAGCTGCGCCGCATCTG |
| | | | AACCTGCTGGGCTCTAAG | | | GTTTTACCTTTCGTACCTAC |
| | | | CTGGTTCACTGGTATCAG | | | GGTATGCACTGGGTGCGTC |
| | | | CAAAAACCAGGCCAGGC | | | AGGCACCAGGCAAAGGTCT |
| | | | ACCAGTACTGGTTGTGTA | | | GGAATGGGTCGCAGTAATC |
| | | | CGATGATAGCGATCGTCC | | | TGGTATGATGGTAGCAATA |
| | | | AAGCGCAATTCCAGAGCG | | | AACACTAT |
| | | | TTTCAGCGGCTCTAATTC | | | GCTGACTCAGTCAAAGGCC |
| | | | CGGCAACACCGCTACTCT | | | GTTTCACCATCACCCGTGAT |
| | | | GACTATTTCCCGTGTTGA | | | AACAGCAAGAACACTCTTA |
| | | | AGCCGGCGATGAAGCCG | | | ACTTACAGATGAACTCTCT |
| | | | ACTACTATTGCCAGGTCT | | | GCGTGCCGAAGACACCGCC |
| | | | GGGACTCTAGCTCCGACC | | | GTTTACTACTGTGCCCGTGC |
| | | | ACGTAGTCTTTGGCGGGG | | | ACCACAGTGGGAATTGGTA |
| | | | GCACCAAACTGACCGTTT | | | GCCGAAGCATTCGATATCT |
| | | | TG | | | GGGGCCAGGGCACTATGGT |
| | | | | | | GACCGTTAGCTCT |
| 7 | 179 | C6-VL | TCTTACGTGCTGACTCAA | 178 | C6-VH | CAAATGCAGCTGGTCGAGT |
| | | | CCACCATCAGTGTCTGTA | | | CTGGCGGTGGGGTAGTGCA |
| | | | GCACCAGGCCAGACCGCA | | | ACCAGGCCGTTCTCTGCGT |
| | | | CGTATTACCTGTGGCGGT | | | CTTAGCTGCGCCGCATCTG |
| | | | AACCTGATCGGCTCTAAG | | | GTTTTACCTTTCGTACCTAC |
| | | | CTGGTTCACTGGTATCAG | | | GGTATGCACTGGGTGCGTC |
| | | | CAAAAACCAGGG | | | AGGCACCAGGCAAAGGTCT |
| | | | CAGGCACCAGTACTGGTT | | | GGAATGGGTCGCAGTAATC |
| | | | GTGTACGATGATAGCGAT | | | TGGTATGATGGTAGCAATA |
| | | | CGTCCAAGCCGGATTCCA | | | AACACTAT |
| | | | GAGCGTTTCAGCGGCTCT | | | GCTGACTCAGTCAAAGGCC |
| | | | AATTCCGGCAACACCGCT | | | GTTTCACCATCACCCGTGAT |
| | | | ACTCTGACTATTTCCCGT | | | AACAGCAAGAACACTCTTA |
| | | | GTTGAAGCCGGC | | | ACTTACAGATGAACTCTCT |
| | | | GATGAAGCCGACTACTAT | | | GCGTGCCGAAGACACCGCC |
| | | | TGCCAGGTCTGGGACTCC | | | GTTTACTACTGTGCCCGTGC |
| | | | AGCTCCGACCATGTAGTC | | | ACCACAGTGGATGTTAGTA |
| | | | TTTGGCGGGGGCACCAAA | | | GCGGAAGCATTCGATCTAT |
| | | | CTGACCGTTTTG | | | GGGGCCAGGGCACTATGGT |
| | | | | | | GACCGTT |
| | | | | | | AGCTCT |
| 8 | 181 | C3-VL | TCTTACGTGCTGACTCAA | 180 | C3-VH | CAAATGCAGCTGGTCGAGT |
| | | | CCACCATCAGTGTCTGTA | | | CTGGCGGTGGGGTAGTGCA |
| | | | GCACCAGGCCAGACCGCA | | | ACCAGGCCGTTCTCTGCGT |
| | | | CGTATTACCTGTGGCGGT | | | CTTAGCTGCGCCGCATCTG |
| | | | AACCTGATCGGCTCTAAG | | | GTTTTACCTTTCGTACCTAC |
| | | | CTGGTTCACTGGTATCAG | | | GGTATGCACTGGGTGCGTC |
| | | | CAAAAACCAGGC | | | AGGCACCAGGCAAAGGTCT |
| | | | CAGGCACCAGTACTGGTT | | | GGAATGGGTCGCAGTAATC |
| | | | GTGTACGATGATAGCGAT | | | TGGTATGATGGTAGCAATA |
| | | | CGTCCAAGCCGGATTCCA | | | AACACTAT |
| | | | GAGCGTTTCAGCGGCTCT | | | GCTGACTCAGTCAAAGGCC |
| | | | AATTCCGGCAACACCGCT | | | GTTTCACCATCACCCGTGAT |
| | | | ACTCTGACTATTTCCCGT | | | AACAGCAAGAACACTCTTA |
| | | | GTTGAAGCCGGC | | | ACTTACAGATGAACTCTCT |
| | | | GATGAAGCCGACTACTAT | | | GCGTGCCGAAGACACCGCC |
| | | | TGCCAGGTCTGGGACTCC | | | GTTTACTACTGTGCCCGTGC |
| | | | AGCTCCGACCACGTAGTC | | | ACCACAGTGGGAATGGGTA |
| | | | TTTGGCGGGGGCACCAAA | | | GCCGAAGCATTCGATCTGT |
| | | | CTGACCGTTTTG | | | GGGGCCAGGGCACTATGGT |
| | | | | | | GACCGTT |
| | | | | | | AGCTCT |
| 9 | 183 | C37-VL | TCTTACGTGCTGACTCAA | 182 | C37-VH | CAAATGCAGCTGGTCGAGT |
| | | | CCACCATCAGTGTCTGTA | | | CTGGCGGTGGGGTAGTGCA |
| | | | GCACCAGGCCAGACCGCA | | | ACCAGGCCGTTCTCTGCGT |
| | | | CGTATTACCTGTGGCGGT | | | CTTAGCTGCGCCGCATCTG |
| | | | AACCTGATCGGCTCTAAG | | | GTTTTACCTTTCGTACCTAC |
| | | | CTGGTTCACTGGTATCAG | | | GGTATGCACTGGGTGCGTC |
| | | | CAAAAACCAGGCCAGGC | | | AGGCACCAGGCAAAGGTCT |
| | | | ACCAGTACTGGTTGTGTA | | | GGAATGGGTCGCAGTAATC |
| | | | CGATGATAGCGATCGTCC | | | TGGTATGATGGTAGCAATA |
| | | | AAGCCGGATTCCAGAGCG | | | AACACTATGCTGACTCAGT |
| | | | TTTCAGCGGCTCTAATTC | | | CAAAGGCCGTTTCACCATC |
| | | | CGGCAACACCGCTACTCT | | | ACCCGTGATAACAGCAAGA |
| | | | GACTATTTCCCGTGTTGA | | | ACACTCTTAACTTACAGAT |
| | | | AGCCGGCGATGAAGCCG | | | GAACTCTCTGCGTGCCGAA |
| | | | ACTACTATTGCCAGGTCT | | | GACACCGCCGTTTACTACT |
| | | | GGGACTCTAGCTCCGACC | | | GTGCCCGTGCACCACAGTG |
| | | | ATGTAGTCTTTGGCGGGG | | | GGAATTAGTAGCCGAGGCA |
| | | | GCACCAAACTGACCGTTT | | | TTCGATATGTGGGGCCAGG |
| | | | TG | | | GCACTATGGTGACCGTT |
| | | | | | | AGCTCT |
| 10 | 185 | C32-VL | TCTTACGTGCTGACTCAA | 184 | C32-VH | CAAATGCAGCTGGTCGAGT |
| | | | CCACCATCAGTGTCTGTA | | | CTGGCGGTGGGGTAGTGCA |
| | | | GCACCAGGCCAGACCGCA | | | ACCAGGCCGTTCTCTGCGT |
| | | | CGTATTACCTGTGGCGGT | | | CTTAGCTGCGCCGCATCTG |
| | | | AACCTGATCGGCTCTAAG | | | GTTTTACCTTTCGTACCTAC |
| | | | CTGGTTCACTGGTATCAG | | | GGTATGCACTGGGTGCGTC |
| | | | CAAAAACCAGGC | | | AGGCACCAGGCAAAGGTCT |
| | | | CAGGCACCAGTACTGGTT | | | GGAATGGGTCGCAGTAATC |
| | | | GTGTACGATGATAGCGAT | | | TGGTATGATGGTAGCAATA |
| | | | CGTCCAAGCAAAATTCCA | | | AACACTAT |
| | | | GAGCGTTTCAGCGGCTCT | | | GCTGACTCAGTCAAAGGCC |
| | | | AATTCCGGCAACACCGCT | | | GTTTCACCATCACCCGTGAT |
| | | | ACTCTGACTATTTCCCGT | | | AACAGCAAGAACACTCTTA |
| | | | GTTGAAGCCGGC | | | ACTTACAGATGAACTCTCT |
| | | | GATGAAGCCGACTACTAT | | | GCGTGCCGAAGACACCGCC |
| | | | TGCCAGGTCTGGGACTCC | | | GTTTACTACTGTGCCCGTGC |
| | | | AGCTCCGACCATGTAGTC | | | ACCACAGTGGGAATTAGTA |
| | | | TTTGGCGGGGGCACCAAA | | | GCCGAAGCATTCGATCTGT |
| | | | CTGACCGTTTTG | | | GGGGCCAGGGCACTATGGT |
| | | | | | | GACCGTT |
| | | | | | | AGCTCT |
| 11 | 187 | C38-VL | TCTTACGTGCTGACTCAA | 186 | C38-VH | CAAATGCAGCTGGTCGAGT |
| | | | CCACCATCAGTGTCTGTA | | | CTGGCGGTGGGGTAGTGCA |
| | | | GCACCAGGCCAGACCGCA | | | ACCAGGCCGTTCTCTGCGT |
| | | | CGTATTACCTGTGGCGGT | | | CTTAGCTGCGCCGCATCTG |
| | | | AACCTGATCGGCTCTAAG | | | GTTTTACCTTTCGTACCTAC |
| | | | CTGGTTCACTGGTATCAG | | | GGTATGCACTGGGTGCGTC |
| | | | CAAAAACCAGGC | | | AGGCACCAGGCAAAGGTCT |
| | | | CAGGCACCAGTACTGGTT | | | GGAATGGGTCGCAGTAATC |
| | | | GTGTACGATGATAGCGAT | | | TGGTATGATGGTAGCAATA |
| | | | CGTCCAAGCGATATTCCA | | | AACACTAT |
| | | | GAGCGTTTCAGCGGCTCT | | | GCTGACTCAGTCAAAGGCC |
| | | | AATTCCGGCAACACCGCT | | | GTTTCACCATCACCCGTGAT |
| | | | ACTCTGACTATTTCCCGT | | | AACAGCAAGAACACTCTTA |
| | | | GTTGAAGCCGGC | | | ACTTACAGATGAACTCTCT |
| | | | GATGAAGCCGACTACTAT | | | GCGTGCCGAAGACACCGCC |
| | | | TGCCAGGTCTGGGACACC | | | GTTTACTACTGTGCCCGTGC |
| | | | AGCTCCGACCATGTAGTC | | | ACCACAGTGGGAATACGTA |
| | | | TTTGGCGGGGGCACCAAA | | | GCGGAAGCATTCGATCTGT |
| | | | CTGACCGTTTTG | | | GGGGCCAGGGCACTATGGT |
| | | | | | | GACCGTT |
| | | | | | | AGCTCT |
| 12 | 189 | C26-VL | TCTTACGTGCTGACTCAA | 188 | C26-VH | CAAATGCAGCTGGTCGAGT |
| | | | CCACCATCAGTGTCTGTA | | | CTGGCGGTGGGGTAGTGCA |
| | | | GCACCAGGCCAGACCGCA | | | ACCAGGCCGTTCTCTGCGT |
| | | | CGTATTACCTGTGGCGGT | | | CTTAGCTGCGCCGCATCTG |
| | | | AACATCATCGGCTCTAAG | | | GTTTTACCTTTCGTACCTAC |
| | | | CTGGTTCACTGGTATCAG | | | GGTATGCACTGGGTGCGTC |
| | | | CAAAAACCAGGC | | | AGGCACCAGGCAAAGGTCT |
| | | | CAGGCACCAGTACTGGTT | | | GGAATGGGTCGCAGTAATC |
| | | | GTGTACGATGATGGCGAT | | | TGGTATGATGGTAGCAATA |
| | | | CGTCCAAGCGGTATTCCA | | | AACACTAT |
| | | | GAGCGTTTCAGCGGCTCT | | | GCTGACTCAGTCAAAGGCC |
| | | | AATTCCGGCAACACCGCT | | | GTTTCACCATCACCCGTGAT |
| | | | ACTCTGACTATTTCCCGT | | | AACAGCAAGAACACTCTTA |
| | | | GTTGAAGCCGGCGATGAA | | | ACTTACAGATGAACTCTCT |
| | | | GCCGACTACTATTGCCAG | | | GCGTGCCGAAGACACCGCC |
| | | | GTCTGGGACACTAGCTCC | | | GTTTACTACTGTGCCCGTGC |
| | | | GACCACGTAGTCTTTGGC | | | ACCACAGTGGGAATTAACG |
| | | | GGGGGCACCAAACTGACC | | | GCCGAAGCATTCGATCTTT |
| | | | GTTTTG | | | GGGGCCAGGGCACTATGGT |
| | | | | | | GACCGTT |
| | | | | | | AGCTCT |
| 13 | 191 | C13-VL | TCTTACGTGCTGACTCAA | 190 | C13-VH | CAAATGCAGCTGGTCGAGT |
| | | | CCACCATCAGTGTCTGTA | | | CTGGCGGTGGGGTAGTGCA |
| | | | GCACCAGGCCAGACCGCA | | | ACCAGGCCGTTCTCTGCGT |
| | | | CGTATTACCTGTGGCGGT | | | CTTAGCTGCGCCGCATCTG |
| | | | AACCTGCTGGGCTCTAAG | | | GTTTTACCTTTCGTACCTAC |
| | | | CTGGTTCACTGGTATCAG | | | GGTATGCACTGGGTGCGTC |
| | | | CAAAAACCAGGC | | | AGGCACCAGGCAAAGGTCT |
| | | | CAGGCACCAGTACTGGTT | | | GGAATGGGTCGCAGTAATC |
| | | | GTGTACGATGATGGCGAT | | | TGGTATGATGGTAGCAATA |
| | | | CGTCCAAGCCTGATTCCA | | | AACACTAT |
| | | | GAGCGTTTCAGCGGCTCT | | | GCTGACTCAGTCAAAGGCC |
| | | | AATTCCGGCAACACCGCT | | | GTTTCACCATCACCCGTGAT |
| | | | ACTCTGACTATTTCCCGT | | | AACAGCAAGAACACTCTTA |
| | | | GTTGAAGCCGGC | | | ACTTACAGATGAACTCTCT |
| | | | GATGAAGCCGACTACTAT | | | GCGTGCCGAAGACACCGCC |
| | | | TGCCAGGTCTGGGACACT | | | GTTTACTACTGTGCCCGTGC |
| | | | AGCTCCGACCACGTAGTC | | | ACCACAGTGGGAATTAGTG |
| | | | TTTGGCGGGGGCACCAAA | | | GCGGAAGCATTCGATCTTT |
| | | | CTGACCGTTTTG | | | GGGGCCAGGGCACTATGGT |
| | | | | | | GACCGTT |
| | | | | | | AGCTCT |
| 14 | 193 | C23-VL | TCTTACGTGCTGACTCAA | 192 | C23-VH | CAAATGCAGCTGGTCGAGT |
| | | | CCACCATCAGTGTCTGTA | | | CTGGCGGTGGGGTAGTGCA |
| | | | GCACCAGGCCAGACCGCA | | | ACCAGGCCGTTCTCTGCGT |
| | | | CGTATTACCTGTGGCGGT | | | CTTAGCTGCGCCGCATCTG |
| | | | AACCTGCTGGGCTCTAAG | | | GTTTTACCTTTCGTACCTAC |
| | | | CTGGTTCACTGGTATCAG | | | GGTATGCACTGGGTGCGTC |
| | | | CAAAAACCAGGC | | | AGGCACCAGGCAAAGGTCT |
| | | | CAGGCACCAGTACTGGTT | | | GGAATGGGTCGCAGTAATC |
| | | | GTGTACGATGATAGCGAT | | | TGGTATGATGGTAGCAATA |
| | | | CGTCCAAGCGAAATTCCA | | | AACACTAT |
| | | | GAGCGTTTCAGCGGCTCT | | | GCTGACTCAGTCAAAGGCC |
| | | | AATTCCGGCAACACCGCT | | | GTTTCACCATCACCCGTGAT |
| | | | ACTCTGACTATTTCCCGT | | | AACAGCAAGAACACTCTTA |
| | | | GTTGAAGCCGGC | | | ACTTACAGATGAACTCTCT |
| | | | GATGAAGCCGACTACTAT | | | GCGTGCCGAAGACACCGCC |
| | | | TGCCAGGTCTGGGACACC | | | GTTTACTACTGTGCCCGTGC |
| | | | AGCTCCGACGGTGTAGTC | | | ACCACAGTGGGAATTAACG |
| | | | TTTGGCGGGGGCACCAAA | | | GCGGAAGCATTCGATATTT |
| | | | CTGACCGTTTTG | | | GGGGCCAGGGCACTATGGT |
| | | | | | | GACCGTT |
| | | | | | | AGCTCT |
| 15 | 195 | C39-VL | TCTTACGTGCTGACTCAA | 194 | C39-VH | CAAATGCAGCTGGTCGAGT |
| | | | CCACCATCAGTGTCTGTA | | | CTGGCGGTGGGGTAGTGCA |
| | | | GCACCAGGCCAGACCGCA | | | ACCAGGCCGTTCTCTGCGT |
| | | | CGTATTACCTGTGGCGGT | | | CTTAGCTGCGCCGCATCTG |
| | | | AACCTGCTGGGCTCTAAG | | | GTTTTACCTTTCGTACCTAC |
| | | | CTGGTTCACTGGTATCAG | | | GGTATGCACTGGGTGCGTC |
| | | | CAAAAACCAGGC | | | AGGCACCAGGCAAAGGTCT |
| | | | CAGGCACCAGTACTGGTT | | | GGAATGGGTCGCAGTAATC |
| | | | GTGTACGATGATAGCGAT | | | TGGTATGATGGTAGCAATA |
| | | | CGTCCAAGCTGGATTCCA | | | AACACTAT |
| | | | GAGCGTTTCAGCGGCTCT | | | GCTGACTCAGTCAAAGGCC |
| | | | AATTCCGGCAACACCGCT | | | GTTTCACCATCACCCGTGAT |
| | | | ACTCTGACTATTTCCCGT | | | AACAGCAAGAACACTCTTA |
| | | | GTTGAAGCCGGC | | | ACTTACAGATGAACTCTCT |
| | | | GATGAAGCCGACTACTAT | | | GCGTGCCGAAGACACCGCC |
| | | | TGCCAGGTCTGGGACACT | | | GTTTACTACTGTGCCCGTGC |
| | | | AGCTCCGACCATGTAGTC | | | ACCACAGTGGGAATTAACG |
| | | | TTTGGCGGGGGCACCAAA | | | GCCGAAGCATTCGATCTTT |
| | | | CTGACCGTTTTG | | | GGGGCCAGGGCACTATGGT |
| | | | | | | GACCGTT |
| | | | | | | AGCTCT |
| 16 | 197 | C17-VL | TCTTACGTGCTGACTCAA | 196 | C17-VH | CAAATGCAGCTGGTCGAGT |
| | | | CCACCATCAGTGTCTGTA | | | CTGGCGGTGGGGTAGTGCA |
| | | | GCACCAGGCCAGACCGCA | | | ACCAGGCCGTTCTCTGCGT |
| | | | CGTATTACCTGTGGCGGT | | | CTTAGCTGCGCCGCATCTG |
| | | | AACCTGATCGGCTCTAAG | | | GTTTTACCTTTCGTACCTAC |
| | | | CTGGTTCACTGGTATCAG | | | GGTATGCACTGGGTGCGTC |
| | | | CAAAAACCAGGC | | | AGGCA |
| | | | CAGGCACCAGTACTGGTT | | | CCAGGCAAAGGTCTGGAAT |
| | | | GTGTACGATGATAGCGAT | | | GGGTCGCAGTAATCTGGTA |
| | | | CGTCCAAGCGCAATTCCA | | | TGATGGTAGCAATAAACAC |
| | | | GAGCGTTTCAGCGGCTCT | | | TATGCTGACTCAGTCAAAG |
| | | | AATTCCGGCAACACCGCT | | | GCCGTTTCACCATCACCCGT |
| | | | ACTCTGACTATTTCCCGT | | | GATAACAGCAAGAACACTC |
| | | | GTTGAAGCCGGC | | | TTAACTTACAGATGAACTC |
| | | | GATGAAGCCGACTACTAT | | | TCTGCGTGCCGAAGACACC |
| | | | TGCCAGGTCTGGGACACC | | | GCCGTTTACTACTGTGCCCG |
| | | | AGCTCCGACCACGTAGTC | | | TGCACCA |
| | | | TTTGGCGGGGGCACCAAA | | | CAGTGGGAATTAACGTCGG |
| | | | CTGACCGTTTTG | | | AAGCATTCGATCTTTGGGG |
| | | | | | | CCAGGGCACTATGGTGACC |
| | | | | | | GTTAGCTCT |
| 17 | 199 | C45-VL | TCTTACGTGCTGACTCAA | 198 | C45-VH | CAAATGCAGCTGGTCGAGT |
| | | | CCACCATCAGTGTCTGTA | | | CTGGCGGTGGGGTAGTGCA |
| | | | GCACCAGGCCAGACCGCA | | | ACCAGGCCGTTCTCTGCGT |
| | | | CGTATTACCTGTGGCGGT | | | CTTAGCTGCGCCGCATCTG |
| | | | AACCTGATCGGCTCTAAG | | | GTTTTACCTTTCGTACCTAC |
| | | | CTGGTTCACTGGTATCAG | | | GGTATGCACTGGGTGCGTC |
| | | | CAAAAACCAGGC | | | AGGCACCAGGCAAAGGTCT |
| | | | CAGGCACCAGTACTGGTT | | | GGAATGGGTCGCAGTAATC |
| | | | GTGTACGATGATAGCGAT | | | TGGTATGATGGTAGCAATA |
| | | | CGTCCAAGCGAAATTCCA | | | AACACTAT |
| | | | GAGCGTTTCAGCGGCTCT | | | GCTGACTCAGTCAAAGGCC |
| | | | AATTCCGGCAACACCGCT | | | GTTTCACCATCACCCGTGAT |
| | | | ACTCTGACTATTTCCCGT | | | AACAGCAAGAACACTCTTA |
| | | | GTTGAAGCCGGC | | | ACTTACAGATGAACTCTCT |
| | | | GATGAAGCCGACTACTAT | | | GCGTGCCGAAGACACCGCC |
| | | | TGCCAGGTCTGGGACTCC | | | GTTTACTACTGTGCCCGTGC |
| | | | AGCTCCGACGGTGTAGTC | | | ACCACAGTGGGAATTAACC |
| | | | TTTGGCGGGGGCACCAAA | | | TCCGAAGCATTCGATCTTTG |
| | | | CTGACCGTTTTG | | | GGGCCAGGGCACTATGGTG |
| | | | | | | ACCGTT |
| | | | | | | AGCTCT |
| 18 | 201 | C7-VL | TCTTACGTGCTGACTCAA | 200 | C7-VH | CAAATGCAGCTGGTCGAGT |
| | | | CCACCATCAGTGTCTGTA | | | CTGGCGGTGGGGTAGTGCA |
| | | | GCACCAGGCCAGACCGCA | | | ACCAGGCCGTTCTCTGCGT |
| | | | CGTATTACCTGTGGCGGT | | | CTTAGCTGCGCCGCATCTG |
| | | | AACATCATCGGCTCTAAG | | | GTTTTACCTTTCGTACCTAC |
| | | | CTGGTTCACTGGTATCAG | | | GGTATGCACTGGGTGCGTC |
| | | | CAAAAACCAGGC | | | AGGCACCAGGCAAAGGTCT |
| | | | CAGGCACCAGTACTGGTT | | | GGAATGGGTCGCAGTAATC |
| | | | GTGTACGATGATAGCGAT | | | TGGTATGATGGTAGCAATA |
| | | | CGTCCAAGCGGTATTCCA | | | AACACTAT |
| | | | GAGCGTTTCAGCGGCTCT | | | GCTGACTCAGTCAAAGGCC |
| | | | AATTCCGGCAACACCGCT | | | GTTTCACCATCACCCGTGAT |
| | | | ACTCTGACTATTTCCCGT | | | AACAGCAAGAACACTCTTA |
| | | | GTTGAAGCCGGC | | | ACTTACAGATGAACTCTCT |
| | | | GATGAAGCCGACTACTAT | | | GCGTGCCGAAGACACCGCC |
| | | | TGCCAGGTCTGGGACTCT | | | GTTTACTACTGTGCCCGTGC |
| | | | GGCTCCGACCACGTAGTC | | | ACCACAGTGGCTGTTAGTA |
| | | | TTTGGCGGGGGCACCAAA | | | GCGGAAGCATTCGATCTCT |
| | | | CTGACCGTTTTG | | | GGGGCCAGGGCACTATGGT |
| | | | | | | GACCGTT |
| | | | | | | AGCTCT |
| 19 | 203 | C31-VL | TCTTACGTGCTGACTCAA | 202 | C31-VH | CAAATGCAGCTGGTCGAGT |
| | | | CCACCATCAGTGTCTGTA | | | CTGGCGGTGGGGTAGTGCA |
| | | | GCACCAGGCCAGACCGCA | | | ACCAGGCCGTTCTCTGCGT |
| | | | CGTATTACCTGTGGCGGT | | | CTTAGCTGCGCCGCATCTG |
| | | | AACATCATCGGCTCTAAG | | | GTTTTACCTTTCGTACCTAC |
| | | | CTGGTTCACTGGTATCAG | | | GGTATGCACTGGGTGCGTC |
| | | | CAAAAACCAGGC | | | AGGCACCAGGCAAAGGTCT |
| | | | CAGGCACCAGTACTGGTT | | | GGAATGGGTCGCAGTAATC |
| | | | GTGTACGATGATAGCGAT | | | TGGTATGATGGTAGCAATA |
| | | | CGTCCAAGCGATATTCCA | | | AACACTAT |
| | | | GAGCGTTTCAGCGGCTCT | | | GCTGACTCAGTCAAAGGCC |
| | | | AATTCCGGCAACACCGCT | | | GTTTCACCATCACCCGTGAT |
| | | | ACTCTGACTATTTCCCGT | | | AACAGCAAGAACACTCTTA |
| | | | GTTGAAGCCGGC | | | ACTTACAGATGAACTCTCT |
| | | | GATGAAGCCGACTACTAT | | | GCGTGCCGAAGACACCGCC |
| | | | TGCCAGGTCTGGGACTCC | | | GTTTACTACTGTGCCCGTGC |
| | | | GGCTCCGACGGTGTAGTC | | | ACCACAGTGGGAATTAGTA |
| | | | TTTGGCGGGGGCACCAAA | | | GCGGAAGCATTCGATCTTT |
| | | | CTGACCGTTTTG | | | GGGGCCAGGGCACTATGGT |
| | | | | | | GACCGTT |
| | | | | | | AGCTCT |
| 20 | 205 | C15-VL | TCTTACGTGCTGACTCAA | 204 | C15-VH | CAAATGCAGCTGGTCGAGT |
| | | | CCACCATCAGTGTCTGTA | | | CTGGCGGTGGGGTAGTGCA |
| | | | GCACCAGGCCAGACCGCA | | | ACCAGGCCGTTCTCTGCGT |
| | | | CGTATTACCTGTGGCGGT | | | CTTAGCTGCGCCGCATCTG |
| | | | AACCTGATCGGCTCTAAG | | | GTTTTACCTTTCGTACCTAC |
| | | | CTGGTTCACTGGTATCAG | | | GGTATGCACTGGGTGCGTC |
| | | | CAAAAACCAGGC | | | AGGCACCAGGCAAAGGTCT |
| | | | CAGGCACCAGTACTGGTT | | | GGAATGGGTCGCAGTAATC |
| | | | GTGTACGATGATGGCGAT | | | TGGTATGATGGTAGCAATA |
| | | | CGTCCAAGCTGGATTCCA | | | AACACTAT |
| | | | GAGCGTTTCAGCGGCTCT | | | GCTGACTCAGTCAAAGGCC |
| | | | AATTCCGGCAACACCGCT | | | GTTTCACCATCACCCGTGAT |
| | | | ACTCTGACTATTTCCCGT | | | AACAGCAAGAACACTCTTA |
| | | | GTTGAAGCCGGC | | | ACTTACAGATGAACTCTCT |
| | | | GATGAAGCCGACTACTAT | | | GCGTGCCGAAGACACCGCC |
| | | | TGCCAGGTCTGGGACTCT | | | GTTTACTACTGTGCCCGTGC |
| | | | GGCTCCGACGGTGTAGTC | | | ACCACAGTGGGAATTAGTA |
| | | | TTTGGCGGGGGCACCAAA | | | GCGGAAGCATTCGATCTGT |
| | | | CTGACCGTTTTG | | | GGGGCCAGGGCACTATGGT |
| | | | | | | GACCGTT |
| | | | | | | AGCTCT |
| 21 | 207 | C35-VL | TCTTACGTGCTGACTCAA | 206 | C35-VH | CAAATGCAGCTGGTCGAGT |
| | | | CCACCATCAGTGTCTGTA | | | CTGGCGGTGGGGTAGTGCA |
| | | | GCACCAGGCCAGACCGCA | | | ACCAGGCCGTTCTCTGCGT |
| | | | CGTATTACCTGTGGCGGT | | | CTTAGCTGCGCCGCATCTG |
| | | | AACCTGATCGGCTCTAAG | | | GTTTTACCTTTCGTACCTAC |
| | | | CTGGTTCACTGGTATCAG | | | GGTATGCACTGGGTGCGTC |
| | | | CAAAAACCAGGC | | | AGGCACCAGGCAAAGGTCT |
| | | | CAGGCACCAGTACTGGTT | | | GGAATGGGTCGCAGTAATC |
| | | | GTGTACGATGATAGCGAT | | | TGGTATGATGGTAGCAATA |
| | | | CGTCCAAGCCGTATTCCA | | | AACACTAT |
| | | | GAGCGTTTCAGCGGCTCT | | | GCTGACTCAGTCAAAGGCC |
| | | | AATTCCGGCAACACCGCT | | | GTTTCACCATCACCCGTGAT |
| | | | ACTCTGACTATTTCCCGT | | | AACAGCAAGAACACTCTTA |
| | | | GTTGAAGCCGGC | | | ACTTACAGATGAACTCTCT |
| | | | GATGAAGCCGACTACTAT | | | GCGTGCCGAAGACACCGCC |
| | | | TGCCAGGTCTGGGACTCC | | | GTTTACTACTGTGCCCGTGC |
| | | | GGCTCCGACGGTGTAGTC | | | ACCACAGTGGGTCTTAGTA |
| | | | TTTGGCGGGGGCACCAAA | | | TCCGAAGCATTCGATCTTTG |
| | | | CTGACCGTTTTG | | | GGGCCAGGGCACTATGGTG |
| | | | | | | ACCGTT |
| | | | | | | AGCTCT |
| 22 | 209 | C12-VL | TCTTACGTGCTGACTCAA | 208 | C12-VH | CAAATGCAGCTGGTCGAGT |
| | | | CCACCATCAGTGTCTGTA | | | CTGGCGGTGGGGTAGTGCA |
| | | | GCACCAGGCCAGACCGCA | | | ACCAGGCCGTTCTCTGCGT |
| | | | CGTATTACCTGTGGCGGT | | | CTTAGCTGCGCCGCATCTG |
| | | | AACATCATCGGCTCTAAG | | | GTTTTACCTTTCGTACCTAC |
| | | | CTGGTTCACTGGTATCAG | | | GGTATGCACTGGGTGCGTC |
| | | | CAAAAACCAGGC | | | AGGCACCAGGCAAAGGTCT |
| | | | CAGGCACCAGTACTGGTT | | | GGAATGGGTCGCAGTAATC |
| | | | GTGTACGATGATAGCGAT | | | TGGTATGATGGTAGCAATA |
| | | | CGTCCAAGCGCAATTCCA | | | AACACTAT |
| | | | GAGCGTTTCAGCGGCTCT | | | GCTGACTCAGTCAAAGGCC |
| | | | AATTCCGGCAACACCGCT | | | GTTTCACCATCACCCGTGAT |
| | | | ACTCTGACTATTTCCCGT | | | AACAGCAAGAACACTCTTA |
| | | | GTTGAAGCCGGC | | | ACTTACAGATGAACTCTCT |
| | | | GATGAAGCCGACTACTAT | | | GCGTGCCGAAGACACCGCC |
| | | | TGCCAGGTCTGGGACTCC | | | GTTTACTACTGTGCCCGTGC |
| | | | AGCTCCGACGGTGTAGTC | | | ACCACAGTGGGAATTAGTA |
| | | | TTTGGCGGGGGCACCAAA | | | GCGGAGGCATTCGATCTGT |
| | | | CTGACCGTTTTG | | | GGGGCCAGGGCACTATGGT |
| | | | | | | GACCGTT |
| | | | | | | AGCTCT |
| 23 | 211 | C4-VL | TCTTACGTGCTGACTCAA | 210 | C4-VH | CAAATGCAGCTGGTCGAGT |
| | | | CCACCATCAGTGTCTGTA | | | CTGGCGGTGGGGTAGTGCA |
| | | | GCACCAGGCCAGACCGCA | | | ACCAGGCCGTTCTCTGCGT |
| | | | CGTATTACCTGTGGCGGT | | | CTTAGCTGCGCCGCATCTG |
| | | | AACAACATCGGCTCTAAG | | | GTTTTACCTTTCGTACCTAC |
| | | | CTGGTTCACTGGTATCAG | | | GGTATGCACTGGGTGCGTC |
| | | | CAAAAACCAGGC | | | AGGCACCAGGCAAAGGTCT |
| | | | CAGGCACCAGTACTGGTT | | | GGAATGGGTCGCAGTAATC |
| | | | GTGTACGATGATAGCGAT | | | TCTTATGATGGTAGCAATA |
| | | | CGTCCAAGCGGTATTCCA | | | AACACTAT |
| | | | GAGCGTTTCAGCGGCTCT | | | GCTGACTCAGTCAAAGGCC |
| | | | AATTCCGGCAACACCGCT | | | GTTTCACCATCACCCGTGAT |
| | | | ACTCTGACTATTTCCCGT | | | AACAGCAAGAACACTCTTA |
| | | | GTTGAAGCCGGC | | | ACTTACAGATGAACTCTCT |
| | | | GATGAAGCCGACTACTAT | | | GCGTGCCGAAGACACCGCC |
| | | | TGCCAGGTCTGGGACTCT | | | GTTTACTACTGTGCCCGTAG |
| | | | AGCTCCGACGGTGTAGTC | | | CCCACAGTGGGAATGGGTA |
| | | | TTTGGCGGGGGCACCAAA | | | CACGAAGCATTCGATATGT |
| | | | CTGACCGTTTTG | | | GGGGCCAGGGCACTATGGT |
| | | | | | | GACCGTT |
| | | | | | | AGCTCT |
| 24 | 213 | C41-VL | TCTTACGTGCTGACTCAA | 212 | C41-VH | CAAATGCAGCTGGTCGAGT |
| | | | CCACCATCAGTGTCTGTA | | | CTGGCGGTGGGGTAGTGCA |
| | | | GCACCAGGCCAGACCGCA | | | ACCAGGCCGTTCTCTGCGT |
| | | | CGTATTACCTGTGGCGGT | | | CTTAGCTGCGCCGCATCTG |
| | | | AACATCCTGGGCTCTAAG | | | GTTTTACCTTTCGTACCTAC |
| | | | CTGGTTCACTGGTATCAG | | | GGTATGCACTGGGTGCGTC |
| | | | CAAAAACCAGGC | | | AGGCACCAGGCAAAGGTCT |
| | | | CAGGCACCAGTACTGGTT | | | GGAATGGGTCGCAGTAATC |
| | | | GTGTACGATGATAGCGAT | | | TCTTATGATGGTAGCAATA |
| | | | CGTCCAAGCGAAATTCCA | | | AACACTAT |
| | | | GAGCGTTTCAGCGGCTCT | | | GCTGACTCAGTCAAAGGCC |
| | | | AATTCCGGCAACACCGCT | | | GTTTCACCATCACCCGTGAT |
| | | | ACTCTGACTATTTCCCGT | | | AACAGCAAGAACACTCTTA |
| | | | GTTGAAGCCGGC | | | ACTTACAGATGAACTCTCT |
| | | | GATGAAGCCGACTACTAT | | | GCGTGCCGAAGACACCGCC |
| | | | TGCCAGGTCTGGGACACC | | | GTTTACTACTGTGCCCGTTC |
| | | | AGCTCCGACGGTGTAGTC | | | GCCACAGTGGGAATGGGTA |
| | | | TTTGGCGGGGGCACCAAA | | | CACGAAGCATTCGATCTCT |
| | | | CTGACCGTTTTG | | | GGGGCCAGGGCACTATGGT |
| | | | | | | GACCGTT |
| | | | | | | AGCTCT |
| 25 | 215 | C40-VL | TCTTACGTGCTGACTCAA | 214 | C40-VH | CAAATGCAGCTGGTCGAGT |
| | | | CCACCATCAGTGTCTGTA | | | CTGGCGGTGGGGTAGTGCA |
| | | | GCACCAGGCCAGACCGCA | | | ACCAGGCCGTTCTCTGCGT |
| | | | CGTATTACCTGTGGCGGT | | | CTTAGCTGCGCCGCATCTG |
| | | | AACAACATCGGCTCTAAG | | | GTTTTACCTTTCGTACCTAC |
| | | | CTGGTTCACTGGTATCAG | | | GGTATGCACTGGGTGCGTC |
| | | | CAAAAACCAGGC | | | AGGCACCAGGCAAAGGTCT |
| | | | CAGGCACCAGTACTGGTT | | | GGAATGGGTCGCAGTAATC |
| | | | GTGTACGATGATAGCGAT | | | TGGTATGATGGTAGCAATA |
| | | | CGTCCAAGCCGGATTCCA | | | AACACTAT |
| | | | GAGCGTTTCAGCGGCTCT | | | GCTGACTCAGTCAAAGGCC |
| | | | AATTCCGGCAACACCGCT | | | GTTTCACCATCACCCGTGAT |
| | | | ACTCTGACTATTTCCCGT | | | AACAGCAAGAACACTCTTA |
| | | | GTTGAAGCCGGC | | | ACTTACAGATGAACTCTCT |
| | | | GATGAAGCCGACTACTAT | | | GCGTGCCGAAGACACCGCC |
| | | | TGCCAGGTCTGGGACACT | | | GTTTACTACTGTGCCCGTAG |
| | | | AGCTCCGACGGTGTAGTC | | | CCCACAGTGGGAATGGGTA |
| | | | TTTGGCGGGGGCACCAAA | | | CACGAAGCATTCGATCTAT |
| | | | CTGACCGTTTTG | | | GGGGCCAGGGCACTATGGT |
| | | | | | | GACCGTT |
| | | | | | | AGCTCT |
| 26 | 217 | C9-VL | TCTTACGTGCTGACTCAA | 216 | C9-VH | CAAATGCAGCTGGTCGAGT |
| | | | CCACCATCAGTGTCTGTA | | | CTGGCGGTGGGGTAGTGCA |
| | | | GCACCAGGCCAGACCGCA | | | ACCAGGCCGTTCTCTGCGT |
| | | | CGTATTACCTGTGGCGGT | | | CTTAGCTGCGCCGCATCTG |
| | | | AACCTGATCGGCTCTAAG | | | GTTTTACCTTTCGTACCTAC |
| | | | CTGGTTCACTGGTATCAG | | | GGTATGCACTGGGTGCGTC |
| | | | CAAAAACCAGGC | | | AGGCACCAGGCAAAGGTCT |
| | | | CAGGCACCAGTACTGGTT | | | GGAATGGGTCGCAGTAATC |
| | | | GTGTACGATGATGGCGAT | | | TGGTATGATGGTAGCAATA |
| | | | CGTCCAAGCTGGATTCCA | | | AACACTAT |
| | | | GAGCGTTTCAGCGGCTCT | | | GCTGACTCAGTCAAAGGCC |
| | | | AATTCCGGCAACACCGCT | | | GTTTCACCATCACCCGTGAT |
| | | | ACTCTGACTATTTCCCGT | | | AACAGCAAGAACACTCTTA |
| | | | GTTGAAGCCGGCGATGAA | | | ACTTACAGATGAACTCTCT |
| | | | GCCGACTACTATTGCCAG | | | GCGTGCCGAAGACACCGCC |
| | | | GTCTGGGACTCCAGCTCC | | | GTTTACTACTGTGCCCGTTC |
| | | | GACGGTGTAGTCTTTGGC | | | GCCACAGTGGGAATGGGTA |
| | | | GGGGGCACCAAACTGACC | | | CACGAAGCATTCGATCTCT |
| | | | GTTTTG | | | GGGGCCAGGGCACTATGGT |
| | | | | | | GACCGTT |
| | | | | | | AGCTCT |
| | 271 | 33.023 | TCTTACGTGCTGACTCAA | 270 | 33.023 | CAAATGCAGCTGGTCGAGT |
| | | VL | CCACCATCAGTGTCTGTA | | VH | CTGGCGGTGGGGTAGTGCA |
| | | | GCACCAGGCCAGACCGCA | | | ACCAGGCCGTTCTCTGCGT |
| | | | CGTATTACCTGTGGCGGT | | | CTTAGCTGCGCCGCATCTG |
| | | | AACCTGATCGGCTCTAAG | | | GTTTTGCGTTTCGTACCTAC |
| | | | CTGGTTCACTGGTATCAG | | | GGTATGCACTGGGTGCGTC |
| | | | CAAAAACCAGGCCAGGC | | | AGGCACCAGGCAAAGGTCT |
| | | | ACCAGTACTGGTTGTGTA | | | GGAATGGGTCGCAGTAATC |
| | | | CGATGATAGCGATCGTCC | | | TGGTATGATGGTAGCAATA |
| | | | AAGCCGTATTCCAGAGCG | | | CCCACTATGCTGACTCAGT |
| | | | TTTCAGCGGCTCTAATTC | | | CAAAGGCCGTTTCACCATC |
| | | | CGGCAACACCGCTACTCT | | | ACCCGTGATAACAGCAAGA |
| | | | GACTATTTCCCGTGTTGA | | | ACACTCTTAACTTACAGAT |
| | | | AGCCGGCGATGAAGCCG | | | GAACTCTCTGCGTGCCGAA |
| | | | ACTACTATTGCCAGGTCT | | | GACACCGCCGTTTACTACT |
| | | | GGGACCACAGCTCCGACC | | | GTGCCCGTGCACCACAGTG |
| | | | ATGTAGTCTTTGGCGGGG | | | GTACTTAAGCGCGGAAGCA |
| | | | GCACCAAACTGACCGTTT | | | TTCGATCTATGGGGCCAGG |
| | | | TG | | | GCACTATGGTGACCGTTAG |
| | | | | | | CTCT |
| | 273 | 33.025 | TCTTACGTGCTGACTCAA | 272 | 33.025 | CAAATGCAGCTGGTCGAGT |
| | | VL | CCACCATCAGTGTCTGTA | | VH | CTGGCGGTGGGGTAGTGCA |
| | | | GCACCAGGCCAGACCGCA | | | ACCAGGCCGTTCTCTGCGT |
| | | | CGTATTACCTGTGGCGGT | | | CTTAGCTGCGCCGCATCTG |
| | | | AACCTGATCGGCTCTAAG | | | GTTTTACCTTTCGTACCTAC |
| | | | CTGGTTCACTGGTATCAG | | | GGTATGCACTGGGTGCGTC |
| | | | CAAAAACCAGGCCAGGC | | | AGGCACCAGGCAAAGGTCT |
| | | | ACCAGTACTGGTTGTGTA | | | GGAATGGGTCGCAGACATC |
| | | | CGATGATAGCGATCGTCC | | | TGGTATGATGGTAGCAATA |
| | | | AAGCCTGATTCCAGAGCG | | | AACACTATGCTGACTCAGT |
| | | | TTTCAGCGGCTCTAATTC | | | CAAAGGCCGTTTCACCATC |
| | | | CGGCAACACCGCTACTCT | | | ACCCGTGATAACAGCAAGA |
| | | | GACTATTTCCCGTGTTGA | | | ACACTCTTAACTTACAGAT |
| | | | AGCCGGCGATGAAGCCG | | | GAACTCTCTGCGTGCCGAA |
| | | | ACTACTATTGCCAGGTCT | | | GACACCGCCGTTTACTACT |
| | | | GGGACTCTAGCTCCGACG | | | GTGCCCGTGCACCACAGTG |
| | | | GTGTAGTCTTTGGCGGGG | | | GTACTTAGTAGCGGAACCG |
| | | | GCACCAAACTGACCGTTT | | | TTCGATCTATGGGGCCAGG |
| | | | TG | | | GCACTATGGTGACCGTTAG |
| | | | | | | CTCT |
| | 293 | 38.014 | AGTTACGTGCTGACACAA | 292 | 38.014 | CAGATGCAGTTGGTGGAGT |
| | | VL | CCTCCAAGTGTTAGTGTC | | VH | CCGGAGGTGGAGTGGTGCA |
| | | | GCACCAGGACAAACAGC | | | ACCAGGGCGTTCCTTGCGT |
| | | | ACGTATTACATGTGGAGG | | | TTGTCTTGTGCTGCTTCCGG |
| | | | AAATCTTATCGGTGCCAA | | | ATTCGCCTTTCGTACATATG |
| | | | GCTGGTGCACTGGTACCA | | | GCATGCATTGGGTGCGTCA |
| | | | GCAGAAACCTGGTCAGGC | | | AGCACCTGGTAAGGGCCTG |
| | | | CCCAGTACTGGTTGTGTA | | | GAGTGGGTTGCCGTTATTT |
| | | | TGATGACAGCGACCGTCC | | | GGTACGACGGCTCCAACAC |
| | | | AAGCCGTATCCCAGAACG | | | CCACTACGCAGATAGCGTG |
| | | | TTTTTCTGGGAGCAACTC | | | AAAGGACGTTTCACTATTA |
| | | | AGGTAATACAGCCACTCT | | | CCCGTGATAACTCCAAGAA |
| | | | GACCATTTCACGTGTTGA | | | TACCCTTAACCTGCAGATG |
| | | | GGCAGGAGATGAGGCCG | | | AATAGCTTGCGTGCTGAGG |
| | | | ATTATTATTGCCAAGTAT | | | ACACAGCAGTATATTACTG |
| | | | GGGACCACAGCTCTGACC | | | CGTCCGTGCACCACAATGG |
| | | | ATGTTGTTTTTGGCGGAG | | | TACCTGAGCGCCGAGGCCT |
| | | | GGACTAAGCTGACCGTGC | | | TTGATCTGTGGGGGCAGGG |
| | | | TT | | | CACAATGGTGACCGTTTCC |
| | | | | | | TCA |
| | 295 | 38.018 | TCCTATGTGCTGACACAG | 294 | 38.018 | CAGATGCAACTGGTGGAGT |
| | | VL | CCACCTAGCGTGAGCGTC | | VH | CAGGAGGCGGCGTGGTGCA |
| | | | GCCCCAGGTCAGACCGCT | | | GCCAGGACGTTCTCTGCGT |
| | | | CGTATCACTTGTGGCGGG | | | CTGTCTTGCGCAGCTTCCGG |
| | | | AACCTTATCGGCAGCAAG | | | GTTCGCCTTTCGTACCTATG |
| | | | CTGGTGCACTGGTACCAG | | | GGATGCATTGGGTGCGTCA |
| | | | CAGAAGCCTGGCCAAGCA | | | GGCTCCAGGTAAGGGACTG |
| | | | CCTGTGCTGGTCGTTTAT | | | GAGTGGGTCGCTGTTATTT |
| | | | GACGACTCTGACCGTCCA | | | GGGACGACGGAAGTAACAC |
| | | | TCCCGTATCCCAGAACGT | | | TCATTACGCCGACAGCGTG |
| | | | TTCTCTGGCTCTAACTCTG | | | AAGGGCCGTTTCACAATTA |
| | | | GGAATACCGCTACCCTGA | | | CCCGTGACAATTCCAAGAA |
| | | | CAATCTCACGTGTTGAAG | | | TACCTTGAACCTGCAGATG |
| | | | CTGGCGACGAGGCAGATT | | | AACTCTCTTCGTGCTGAAG |
| | | | ATTATTGCCAAGTCTGGG | | | ATACCGCCGTGTACTATTG |
| | | | ATCACTCCAGCGATCACG | | | CGCCCGTGCTCCACAGTGG |
| | | | TCGTGTTCGGAGGCGGAA | | | TATCTGTCAGCAGAGGCCT |
| | | | CAAAATTGACTGTCCTG | | | TCGACCTGTGGGGACAGGG |
| | | | | | | AACAATGGTGACCGTATCT |
| | | | | | | TCA |
| | 297 | 38.019 | TCTTACGTGTTGACACAA | 296 | 38.019 | CAAATGCAGCTGGTGGAAT |
| | | VL | CCACCAAGTGTTAGTGTC | | VH | CCGGGGGTGGGGTCGTCCA |
| | | | GCACCTGGCCAAACCGCT | | | GCCTGGCCGTAGTCTGCGT |
| | | | CGTATCACCTGTGGTGGG | | | CTTTCCTGTGCCGCATCAGG |
| | | | AATCTTATTGGCTCTAAG | | | CTTTGCTTTCCGTACCTACG |
| | | | CTGGTGCACTGGTATCAG | | | GGATGCACTGGGTGCGTCA |
| | | | CAGAAACCAGGCCAGGCT | | | GGCCCCAGGAAAGGGACTT |
| | | | CCAGTACTGGTGGTGTAC | | | GAATGGGTGGCTGTCATCT |
| | | | GACGACTCTGACCGTCCA | | | GGTACGATGGTTCCAACAC |
| | | | AGCCGTATCCCAGAGCGT | | | ACACTATGCCGATTCAGTG |
| | | | TTCAGTGGCTCTAACTCC | | | AAAGGGCGTTTCACCATTA |
| | | | GGGAACACAGCAACTCTT | | | CTCGTGACAATAGTAAGAA |
| | | | ACAATTTCACGTGTGGAG | | | TACTCTGAATCTGCAAATG |
| | | | GCCGGTGATGAAGCCGAC | | | AATTCACTGCGTGCTGAGG |
| | | | TACTATTGCCAGGTTTGG | | | ACACCGCTGTTTATTACTGT |
| | | | GACTACAGTAGTAATCAC | | | GTGCGTGCTCCTCAGTGGT |
| | | | GTGGTTTTCGGTGGTGGT | | | ACCTGAGTGCCGAAGCTTT |
| | | | ACCAAGCTGACTGTGTTG | | | CGATTTGTGGGGACAGGGC |
| | | | | | | ACAATGGTGACAGTCAGTT |
| | | | | | | CT |
| | 299 | 38.021 | AGCTATGTTCTGACTCAA | 298 | 38.021 | CAGATGCAGCTGGTTGAAT |
| | | VL | CCACCTAGTGTGAGTGTG | | VH | CTGGCGGCGGTGTGGTCCA |
| | | | GCCCCTGGTCAGACTGCA | | | GCCTGGTCGTAGCCTGCGT |
| | | | CGTATTACCTGTGGCGGA | | | CTGTCCTGTGCTGCAAGCG |
| | | | AACCTTATCGGCAGTAAG | | | GATTTGCCTTTGACACCTAT |
| | | | CTGGTTCATTGGTATCAG | | | GGGATGCACTGGGTACGTC |
| | | | CAGAAGCCAGGACAGGC | | | AGGCCCCAGGAAAGGGCCT |
| | | | ACCAGTGCTGGTCGTTTA | | | GGAATGGGTGGCAGTTATC |
| | | | CGACGATAGTGACCGTCC | | | TGGTATGATGGTTCTAATA |
| | | | ATCACGTATCCCAGAGCG | | | CCGTGTATGCCGACTCCGTT |
| | | | TTTTAGCGGGTCCAATTC | | | AAAGGCCGTTTCACTATCA |
| | | | CGGAAATACAGCAACCTT | | | CCCGTGATAATAGTAAAAA |
| | | | GACCATTAGCCGTGTGGA | | | CACACTGAACCTGCAGATG |
| | | | AGCCGGCGATGAAGCTGA | | | AATAGCTTGCGTGCTGAGG |
| | | | TTATTACTGCCAGGTATG | | | ACACCGCAGTGTACTACTG |
| | | | GGACCATTCCTCCGACCA | | | TGCCCGTGCTCCTCAGTGGT |
| | | | CTACGTTTTTGGTGGCGG | | | ATCTGTCAGCAGAGGCCTT |
| | | | AACTAAGCTGACAGTCTT | | | CGATCTGTGGGGCCAAGGG |
| | | | G | | | ACAATGGTGACCGTGTCTT |
| | | | | | | CC |
| | 301 | 38.025 | TCTTACGTGCTTACTCAG | 300 | 38.025 | CAGATGCAGCTTGTTGAGA |
| | | VL | CCTCCTAGCGTCTCAGTG | | VH | GCGGCGGAGGCGTGGTGCA |
| | | | GCCCCAGGCGAGACAGC | | | ACCAGGCCGTTCATTGCGT |
| | | | AACCATTACATGCGGGGG | | | CTGTCCTGCGCCGCCAGCG |
| | | | TAATTTGATCGGTAGCAA | | | GCTTTGCTTTTCGTACATAC |
| | | | GCTGGTGCATTGGTATCA | | | GGCATGCACTGGGTGCGTC |
| | | | GCAGAAGCCTGGCCAGGC | | | AGGCCCCTGGCAAGGGGCT |
| | | | CCCAGTGCTGGTTGTATA | | | GGAATGGGTCGCCGTGATT |
| | | | TGACGATAGTGATCGTCC | | | TGGTATGACGGTAGTAACA |
| | | | AAGTCGTATCCCTGAGCG | | | CCCATTATGCTGATTCCGTC |
| | | | TTTTAGCGGATCTAACTC | | | AAGGGACGTTTCACTATCA |
| | | | CGGCAACACAGCCACATT | | | CCCGTGACAATAGCAAAAA |
| | | | GACAATCAGCCGTGTGGA | | | TACACTGAATCTGCAAATG |
| | | | GGCAGGCGATGAGGCCG | | | AATTCATTGCGTGCCGAAG |
| | | | ACTACTACTGCCAAGTTT | | | ACACCGCCGTATATTACTG |
| | | | GGGACCACTCCTCTGACC | | | TGTCCGTGCCCCACAGTGG |
| | | | ACGTGGTATTTGGCGGAG | | | TACCTGAGCGCTGAGGCCT |
| | | | GAACAAAGCTTACAGTTT | | | TCGATCTGTGGGGTCAGGG |
| | | | TG | | | GACTATGGTGACCGTATCA |
| | | | | | | TCC |
| | 303 | 38.026 | TCTTATGTTTTGACCCAAC | 302 | 38.026 | CAGATGCAGCTGGTGGAGA |
| | | VL | CTCCATCCGTTAGCGTGG | | VH | GTGGAGGTGGTGTGGTGCA |
| | | | CTCCAGGTCAAACAGCTA | | | ACCTGGGCGTAGCCTGCGT |
| | | | CCATCACATGTGGCGGTA | | | TTGAGCTGCGCTGCCTCTG |
| | | | ACCTTATTGGCTCAAAGC | | | GATTTGCCTTCCGTACCTAT |
| | | | TGGTTCATTGGTATCAAC | | | GGCATGCACTGGGTGCGTC |
| | | | AGAAACCAGGCCAAGCC | | | AGGCTCCAGGAAAGGGGTT |
| | | | CCAGTGCTGGTGGTGTAT | | | GGAATGGGTGGCTGTGATT |
| | | | GACGACAGTGACCGTCCT | | | TGGTACGACGGGAGCGCCA |
| | | | TCTCGTATTCCTGAGCGTT | | | CACATTACGCAGACAGCGT |
| | | | TTTCCGGCTCTAATATTG | | | TAAGGGCCGTTTCACAATT |
| | | | GCAACACTGCCACCCTGA | | | ACCCGTGACAATAGCAAAA |
| | | | CCATTTCTCGTGTGGAAG | | | ATACATTGAACCTGCAGAT |
| | | | CAGGAGATGAGGCAGAC | | | GAATTCCCTGCGTGCAGAG |
| | | | TATTATTGTCAGGTTTGG | | | GATACTGCAGTGTACTATT |
| | | | GATCACTCCAGCGATCAT | | | GCGTCCGTGCCCCACAGTG |
| | | | GTGGTATTCGGAGGTGGG | | | GTATCTGTCAGCCGAAGCC |
| | | | ACAAAACTTACTGTTCTT | | | TTCGATCTGTGGGGGCAGG |
| | | | | | | GTACTATGGTCACCGTAAG |
| | | | | | | TTCC |
| | 305 | 38.040 | AGCTACGTGCTTACCCAG | 304 | 38.040 | CAGATGCAGCTGGTGGAAA |
| | | VL | CCACCATCAGTCAGTGTG | | VH | GTGGTGGGGGAGTCGTGCA |
| | | | GCTCCAGGCCAAACTGCC | | | ACCAGGACGTTCCTTGCGT |
| | | | CGTATCACCTGCGGCGGC | | | CTGTCATGCGCTGCTTCAG |
| | | | AATTTGATTGGCACCAAG | | | GTTTCGACTTTCGTACCTAC |
| | | | CTTGTGCACTGGTACCAA | | | GGCATGCATTGGGTGCGTC |
| | | | CAGAAGCCAGGGCAGGC | | | AGGCTCCAGGTAAAGGACT |
| | | | CCCTGTGCTGGTTGTCTA | | | TGAGTGGGTCGCAGTGATC |
| | | | CGACGATAGTGATCGTCC | | | TGGTACGACGGATCAATTA |
| | | | TTCCCGTATTCCTGAACG | | | CTCACTACGCCGATAGCGT |
| | | | TTTCTCTGGAAGCAATTC | | | GAAAGGCCGTTTCACCATC |
| | | | CGGAAACACAGCCACACT | | | ACCCGTGACAACTCCAAGA |
| | | | TACCATTTCTCGTGTTGA | | | ACACCCTGAACTTGCAGAT |
| | | | GGCTGGGGATGAAGCCG | | | GAACAGTCTGCGTGCAGAA |
| | | | ACTACTATTGCCAGGTTT | | | GACACTGCAGTATATTATT |
| | | | GGGACCACAATGAAGAC | | | GTGTCCGTGCCCCACAGTG |
| | | | GAAGTTGTTTTTGGAGGA | | | GTACTTGACCGCCGAGGCT |
| | | | GGAACTAAGCTGACAGTT | | | TTTGATCTGTGGGGACAGG |
| | | | CTG | | | GCACAATGGTGACCGTATC |
| | | | | | | TAGC |

TABLE 11
Amino Acid Sequences of Heavy-chain CDR Regions
for Antibody Clone #s. 33.023, 33.025, 38.014, 38.015.
38.018, 38.019, 38.021, 38.026, 38.040

| | | SEQ | | SEQ | | SEQ |
|---|---|---|---|---|---|---|
| CLONE | HCDR1 | ID | HCDR2 | ID | HCDR3 | ID |
| BDG33_023_VH | GFAFRTYG | 258 | IWYDGSNT | 259 | ARAPQWYLSAEAFDL | 260 |
| BDG33_025_VH | GFTFRTYG | 274 | IWYDGSNK | 275 | ARAPQWYLVAEPFDL | 276 |
| BDG38_014_VH | GFAFRTYG | 258 | IWYDGSNT | 259 | VRAPQWYLSAEAFDL | 310 |
| BDG38_018_VH | GFAFRTYG | 258 | IWDDGSNT | 317 | ARAPQWYLSAEAFDL | 260 |
| BDG38_019_VH | GFAFRTYG | 258 | IWYDGSNT | 259 | VRAPQWYLSAEAFDL | 310 |
| BDG38_021_VH | GFAFDTYG | 306 | IWYDGSNT | 259 | ARAPQWYLSAEAFDL | 260 |
| BDG38_025_VH | GFAFRTYG | 258 | IWYDGSNT | 259 | VRAPQWYLSAEAFDL | 310 |
| BDG38_026_VH | GFAFRTYG | 258 | IWYDGSAT | 308 | VRAPQWYLSAEAFDL | 310 |
| BDG38_040_VH | GFDFRTYG | 307 | IWYDGSIT | 309 | VRAPQWYLTAEAFDL | 310 |

TABLE 12
Amino Acid Sequences of Light-chain CDR Regions
for Antibody Clone #s. 33.023, 33.025, 38.014,
38.015. 38.018, 38.019, 38.021, 38.026, 38.040

| | | SEQ | | SEQ | | SEQ |
|---|---|---|---|---|---|---|
| CLONE | LCDR1 | ID | LCDR2 | ID | LCDR3 | ID |
| BDG33_023_VL | LIGSKL | 261 | DDS | 262 | QVWDHSSDHVV | 263 |
| BDG33_025_VL | LIGSKL | 261 | DDS | 262 | QVWDSSSDGVV | 277 |
| BDG38_014_VL | LIGAKL | 312 | DDS | 262 | QVWDHSSDHVV | 263 |
| BDG38_018_VL | LIGSKL | 261 | DDS | 262 | QVWDHSSDHVV | 263 |
| BDG38_019_VL | LIGSKL | 261 | DDS | 262 | QVWDYSSNHVV | 314 |
| BDG38_021_VL | LIGSKL | 261 | DDS | 262 | QVWDHSSDHYV | 315 |
| BDG38_025_VL | LIGSKL | 261 | DDS | 262 | QVWDHSSDHVV | 263 |
| BDG38_026_VL | LIGSKL | 261 | DDS | 262 | QVWDHSSDHVV | 263 |
| BDG38_040_VL | LIGTKL | 313 | DDS | 262 | QVWDHNEDEVV | 316 |

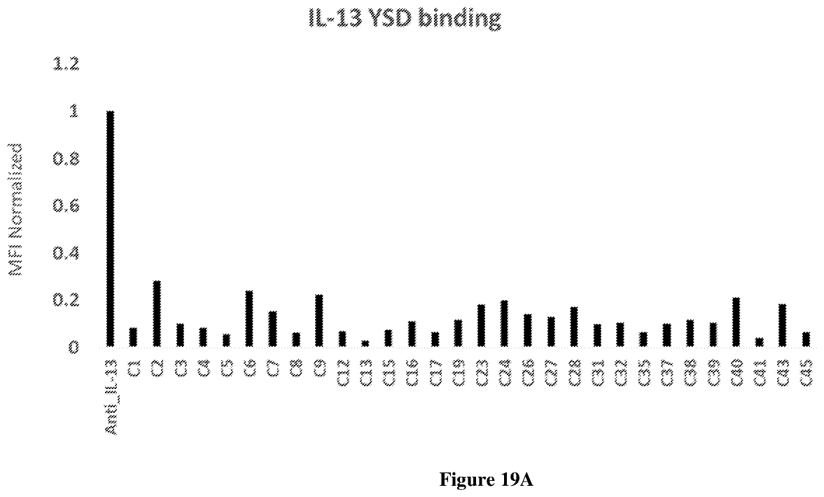

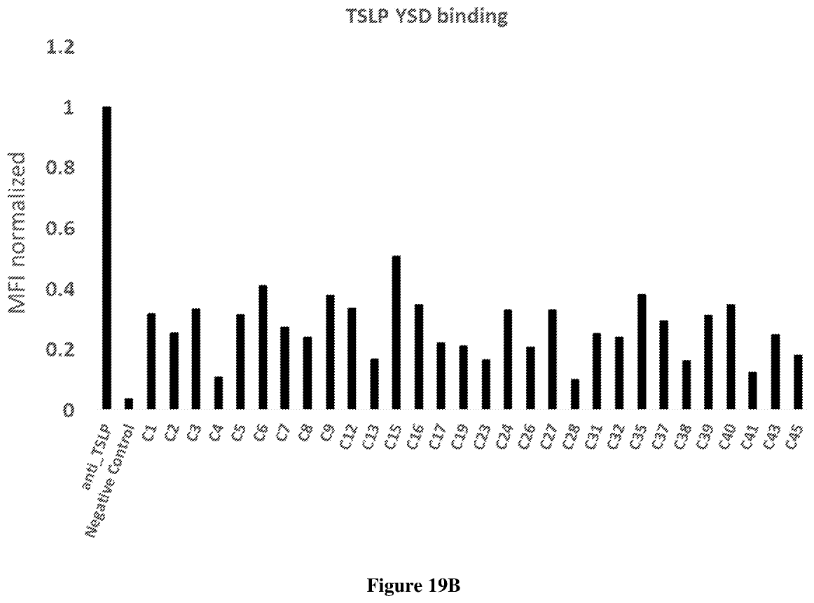

The clones were tested for their binding to 10 nM rh-IL-13 in yeast scFV format and were compared to a positive rh-IL-13 binder displayed on yeast as well. The affinity was normalized based on the mean fluorescence values of the positive control (normalized MFI). Relative affinity of the isolated clones for rh-IL-13 was between 3% and 30% of the affinity displayed by the positive control (FIG. 19A). C2, C6, C9, and C40 clones that exhibited above 20% of the relative affinity for rh-IL-13 and were shown to bind 10 nM TSLP in YSD (FIG. 19B) were chosen to be expressed as human IgG1.

### Example 6

### TSLP/IL-13Ab Production and Biochemical Characterization

Objective: To reformat the selected clones to a human IgG1 format and analyze the IgG1 antibodies for dual IL-13 and TSLP binding.

Results: Subsequent to characterization in the yeast surface display format described in Example 2, the selected clones C2, C6, and C9, were reformatted to human IgG1 by subcloning the variable domain into two separate expression vectors, pSF-CMV-HuIgG1_HC and pSF-CMV-HuLambda_LC, as described in Example 1 (Methods).

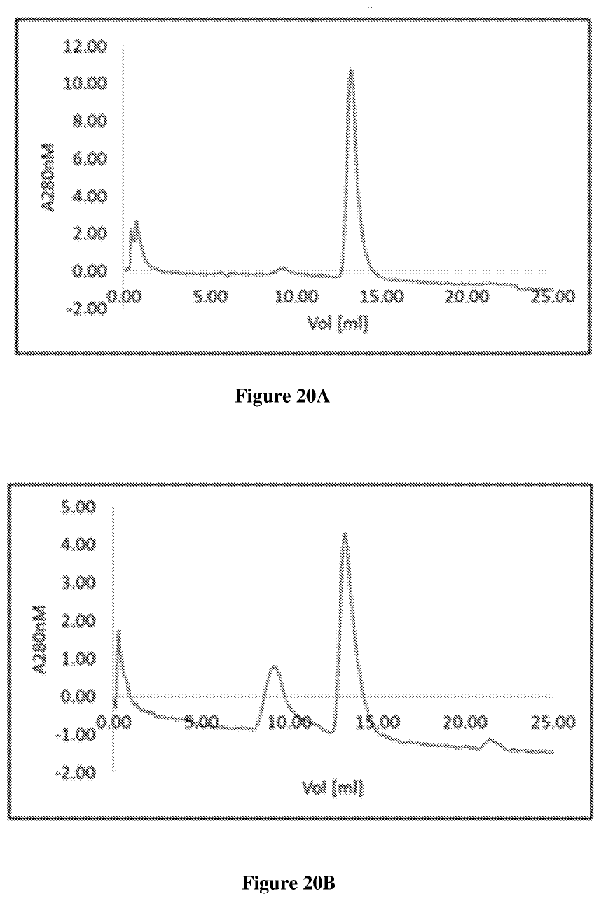

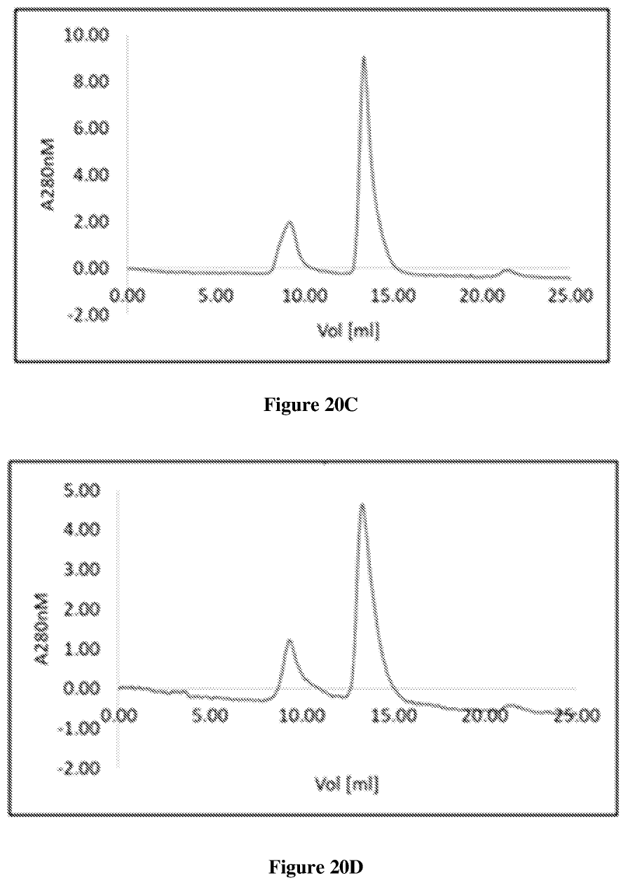

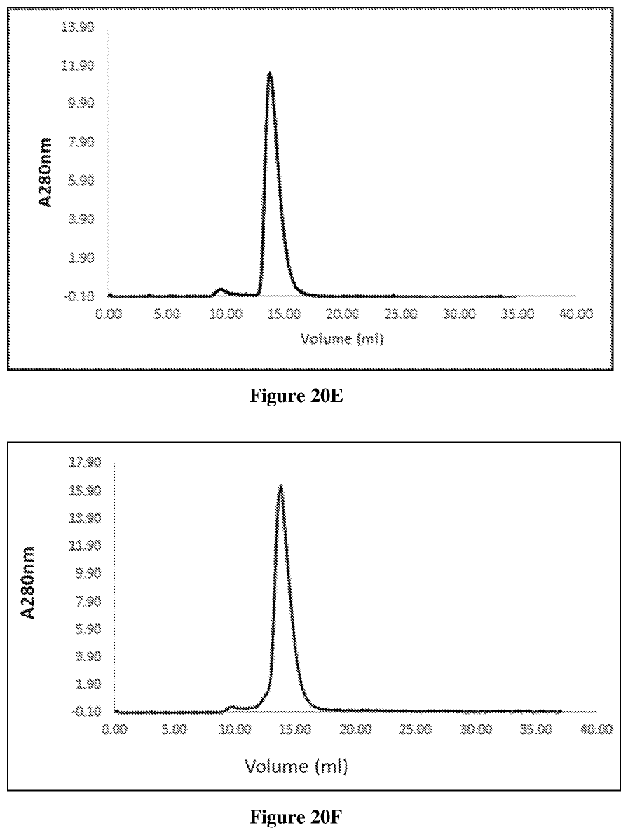

Clones BDG 33.003, BDG 33.004, and BDG 33.005 (Clones C2, C6, and C9, respectively) were expressed and purified as described in Example 4 (Methods), following protein A purification, the IgGs were >95% pure as evident from an SDS PAGE analysis (data not shown). Size exclusion chromatography of BDG 33.003 (clone C2), BDG 33.004 (clone C6), and BDG 33.005 (clone C9) on Superdex® 200 10/300, showed two main peaks the first with a retention time of 9.2 ml (0.36 CV), typical of large aggregate and a second peak with retention of approximately 13.2 ml (0.528 CV), typical of an ordinary human IgG1 (hIgG1). The integrated area under the curve of these two peaks showed a ratio of 22% and 78% respectively (FIGS. 20A-20D).

Both BDG33.0023 and BDG33.025 migrated on Superdex® 200 10/300 with small leading peak corresponds to (0.36 CV) that is typical of a large diameter aggregate, and a second peak with retention of approximately 13.8 ml (0.55 CV) that is typical of an ordinary human IgG. Area Under the Curve (AUC) peak ratio is 97.3% folded/2.8% misfolded and 98.5% folded/1.5% misfolded for BDG33.023 and BDG33.025 respectively (FIGS. 20E-20F).

To test the thermostability of clones BDG 33.003, BDG 30.004, and BDG 30.005, the clones thermal melting was monitored by differential scanning fluorescence (DSF) as described in Example 4. As was evident from the first derivative of the fluorescence thermal shift graph, BDG 33.004 had one distinct transition at point at 62° C. which could possibly correspond to both Tm1 and Tm2. (Data not shown). BDG 33.003 and BDG33.005 each had two transition points, a major one at 62° C. (BDG 33.003) and 64.5° C. (BDG 33.005), respectively, and a minor one at 73° C. (BDG 33.003) and 74.5° C. (BDG 33.005), respectively.

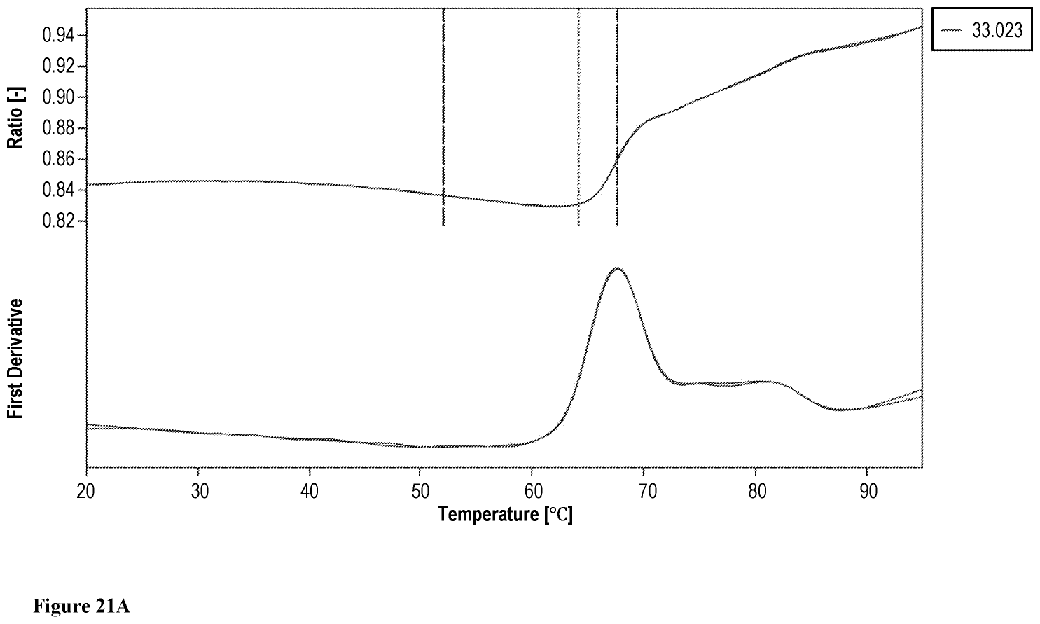

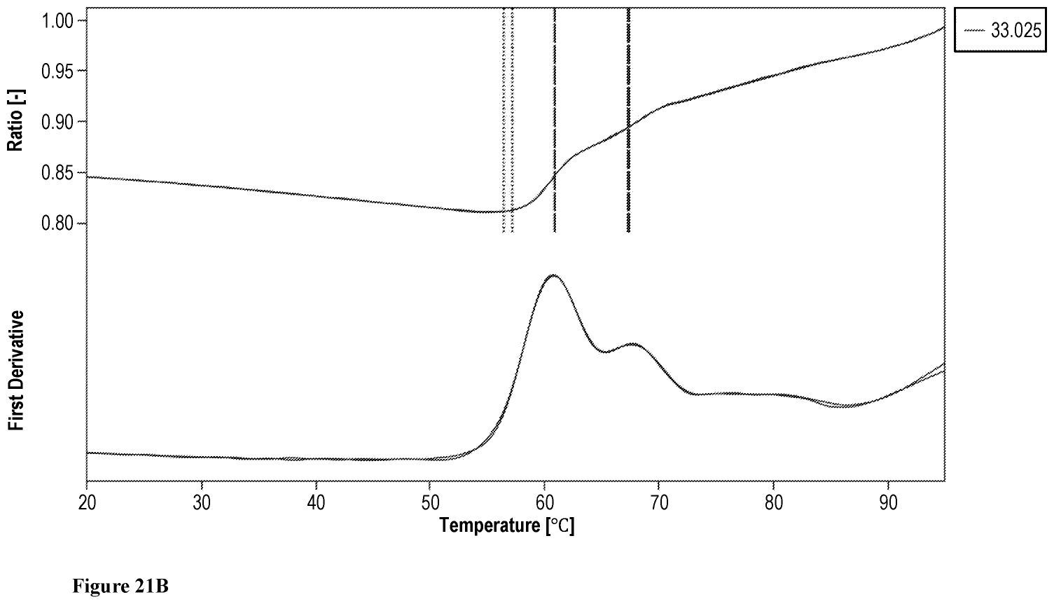

BDG 33.023 and BDG33.025 were tested using NanoDSF Prometheus NT.48 (NanoTemper Technologies, Germany). BDG33.0023 had a T-onset of 64.2° C. and first transition point at 67.7° C., BDG33.0025 had a T-onset of 56.4° C. and first transition point at 60.9° C. and second transition point at 67.4° C. (FIGS. 21A-21B)

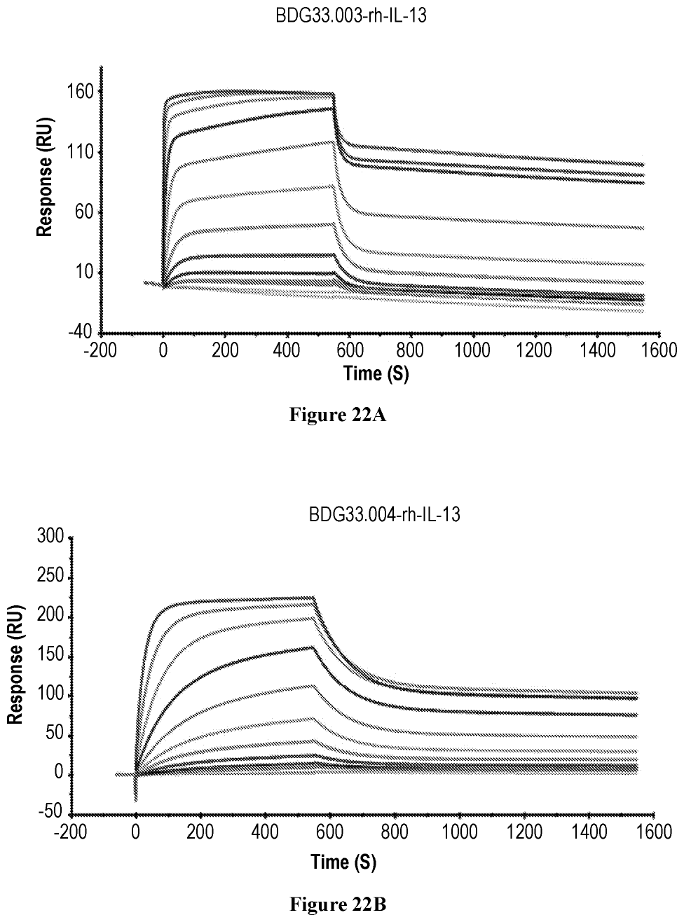

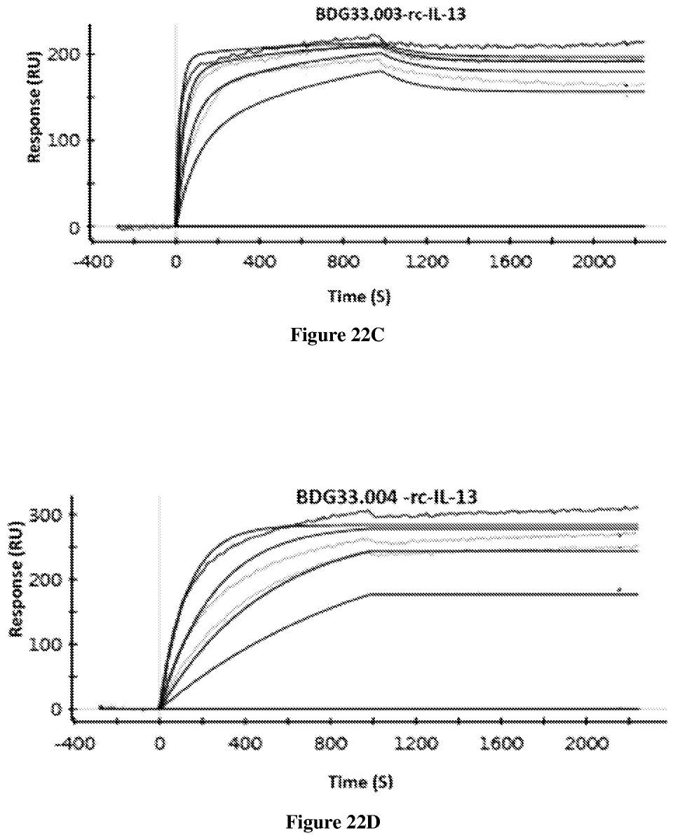

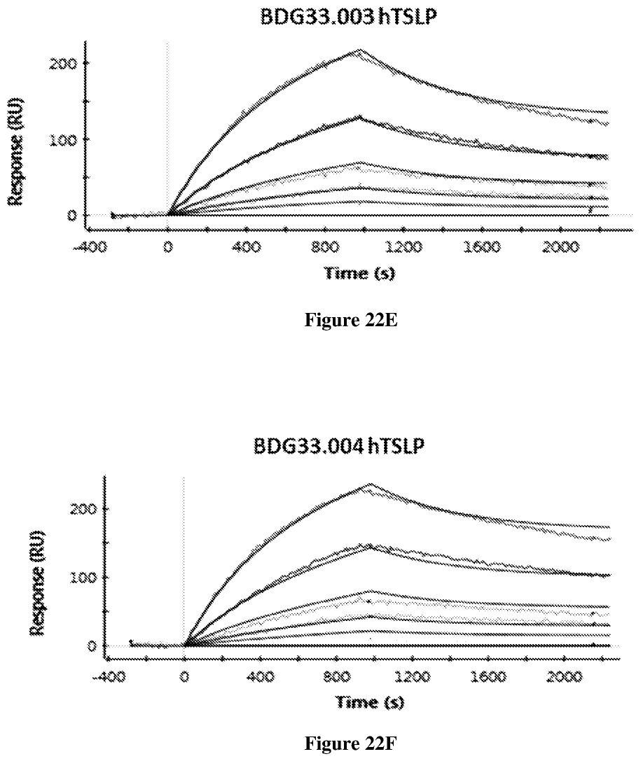

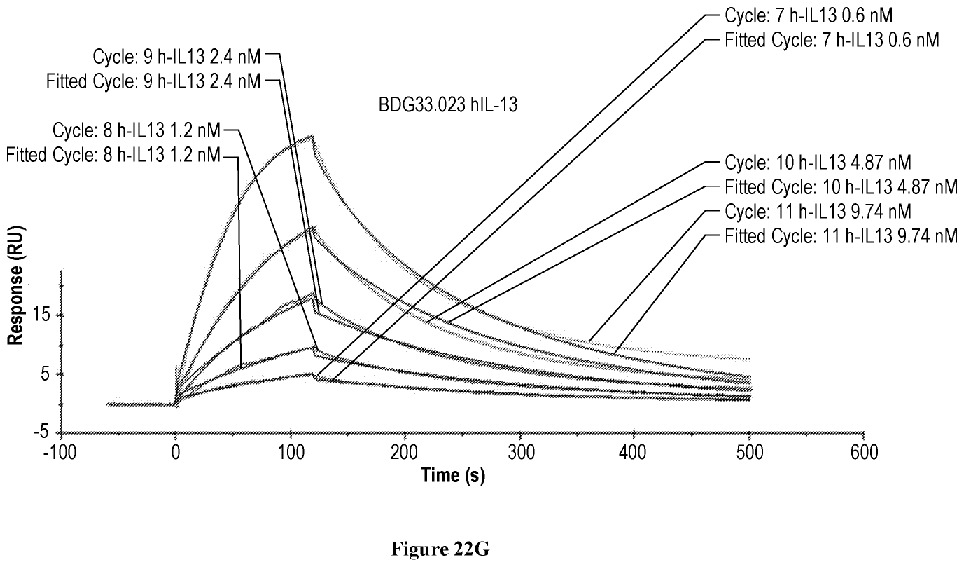

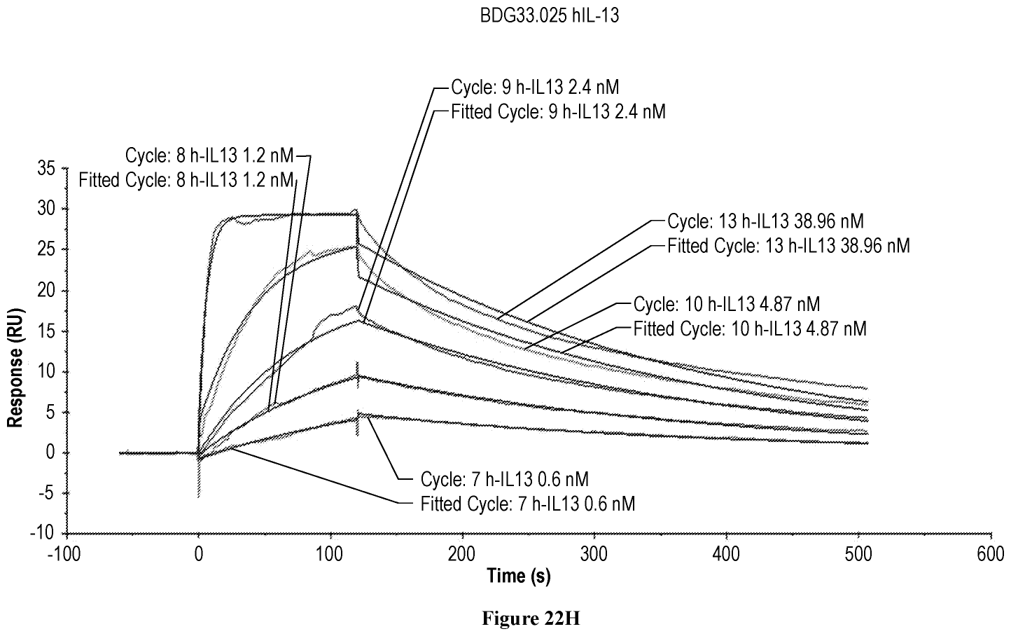

The affinities of the IgGs to human TSLP, human IL-13, and cynomolgus monkey IL-13 were tested. Binding kinetics of hIL-13 to BDG33.023 and BDG33.025 was tested on BIAcoreT200 as described herein, (FIG. 22G-22H). BDG33.003 and BDG33.004 clones were tested by SPR analysis on BiacoreT200 and ProteOn™ XPR36, respectively, using the GE capture antibody kit. While it was not possible to obtain kinetics parameters for binding the human IL-13, steady-state binding measurement resulted in an apparent KD of 21.6 nM and 57.4 nM for BDG33.003 and BDG33.004, respectively (FIGS. 22A-22B) For all other measurements of BDG33.003, BDG 33.004, BDG33.023, and BDG33.025, kinetics of binding to hTSLP and hIL-13 are presented in Tables 13A and 13B, and FIGS. 22E and 22H.

The antibodies were also tested for binding of recombinant cynomolgus monkey IL-13 (rc-IL-13), which shares 85% identity and 88% homology with the human IL-13, as can be seen in FIGS. 5C-5D, injection of cIL-13 as analyte, resulted in strong, dose dependent response indicating that BDG33.003 and BDG33.004 bind to rc-IL-13. Although kinetics for rc-IL-13 could not be obtained, the binding and dissociation slopes had similar profile for rh-IL-13 and rc-IL-13, suggesting that the binding mode for recombinant h-IL-13 and rc-IL-13 is likely similar (FIGS. 22A-22B for human IL-13 and FIGS. 22C-22D for cyno IL-13).

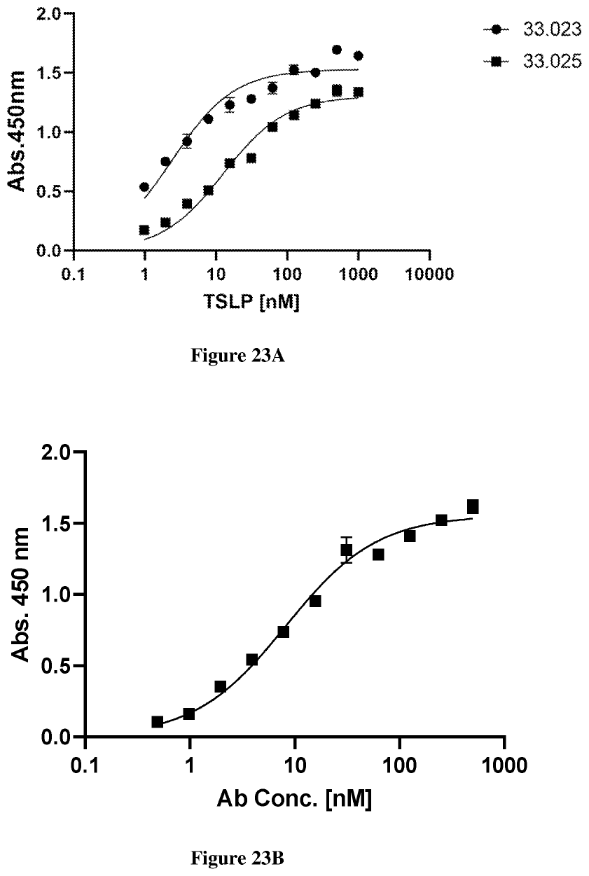

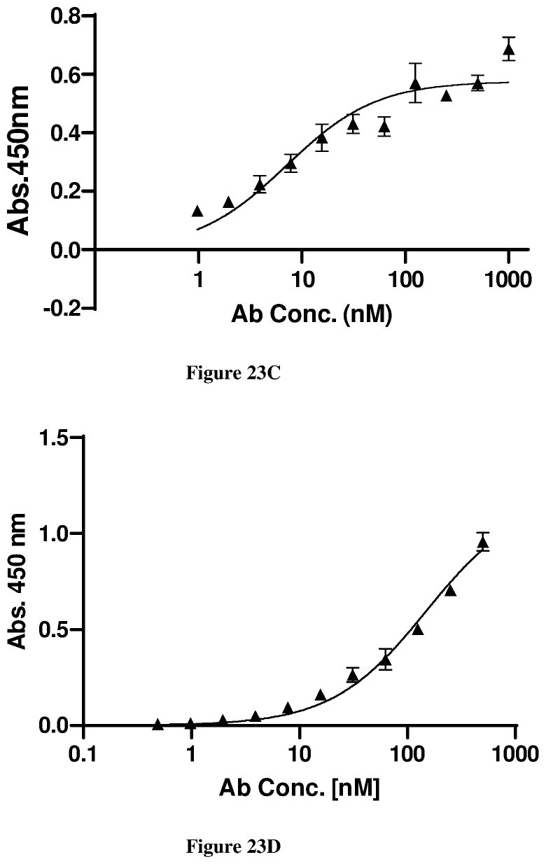

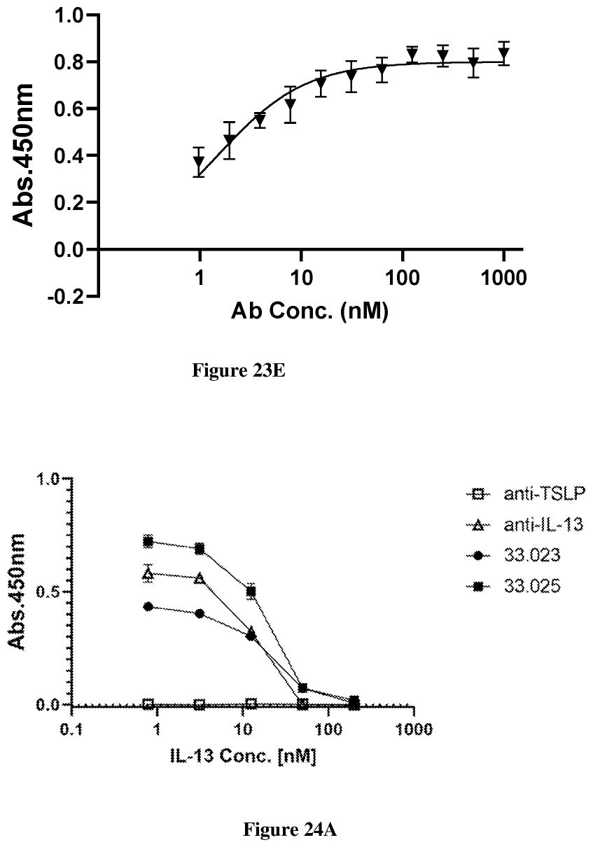

To further test the IgGs affinity to human TSLP, cynomolgus monkey TSLP and cynomolgus monkey IL-13, an ELISA EC50 experiment was done as described herein. Briefly, wells were coated with the respective ligand, then incubated with clones BDG33.003, BDG33.004,BDG33.023, or BDG33.025 at a concentration range of 1 nM to 1000 nM, washed and developed using HRP conjugated secondary antibody. EC50 values are presented in Table 14. Since the IgGs mentioned in the above sections are symmetrical IgGs, and since these same IgGs bind both hIL-13 and hTSLP this data demonstrates that BGD33.003, BGD33.004, BGD33.023, and BGD33.025 antibodies bind the two unrelated targets—TSLP and/or IL-13 from the same standard IgG CDRs, as appose to bi-specific antibody where the Light chain variable domain binds one target and heavy chain binds the other target (FIGS. 23A-23E).

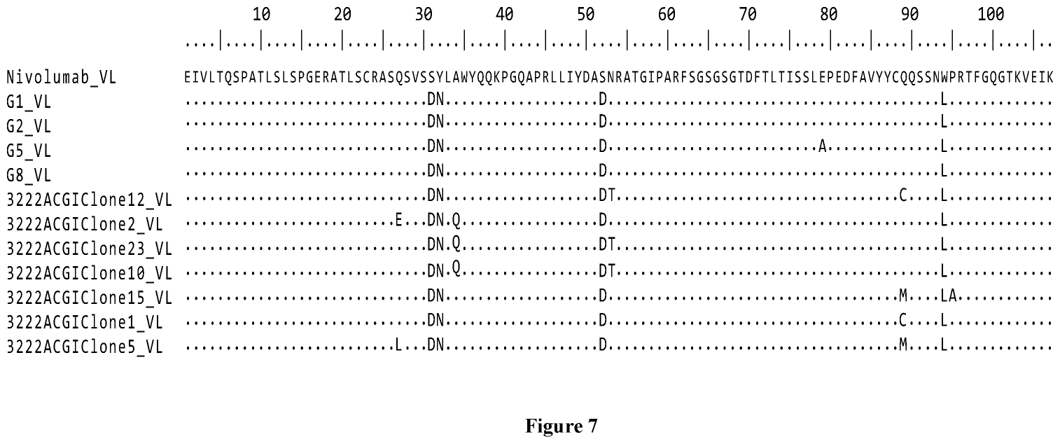

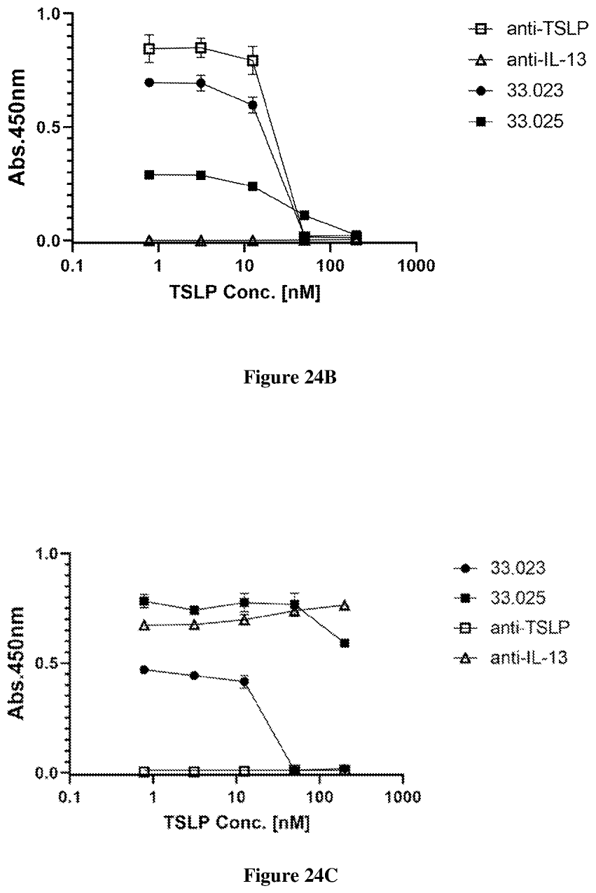

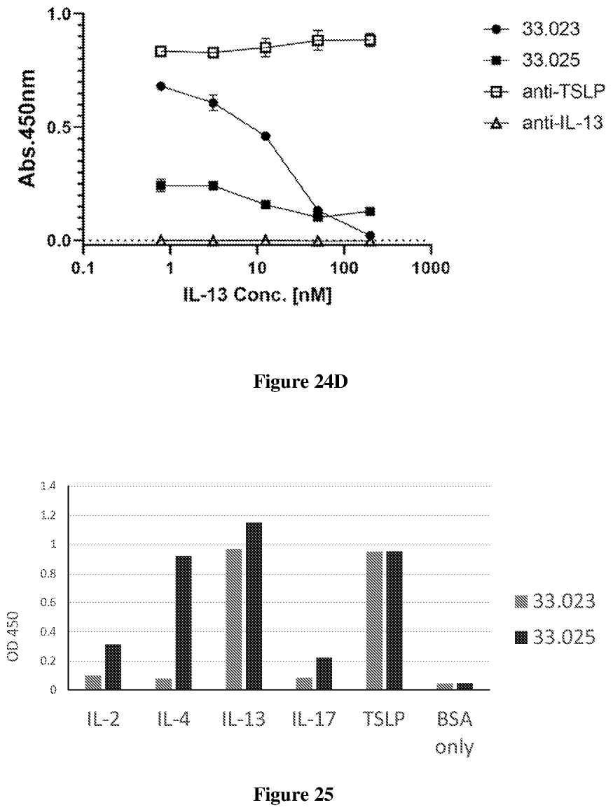

To test whether the IgGs are binding IL-13 and TSLP with overlapping paratopes, a competition assay was done as described herein. Briefly, BDG 33.023 or BDG33.025 were incubated with hIL-13 or hTSLP in concentration range of 0.78 nM to 200 nM and then tested for binding to either IL-13 or TSLP that were pre-coated on an ELISA plate. As can be seen in FIGS. 7A-7D, IL-13 blocks BDG33.023 from binding to IL-13 coated wells, and TSLP blocks BDG33.023 and BDG33.025 from binding to TSLP coated wells (FIGS. 24A-24B). In addition, IL-13 blocked binding of BDG33.023 from binding to TSLP coated wells and reciprocally TSLP blocked binding of BDG33.023 from binding to IL-13 coated wells (FIGS. 24C-24D). This experiment indicates that each of IL-13 and TSLP share at least partially BDG33.023 binding paratope.

IL-13 and TSLP are sequence and structurally unrelated. To test whether binding to these ligands by BDG33.023 and BDG33.025 is specific and not a result of non-specific binding or “stickiness”, BGD33.0023 and BGD33.025 binding to IL-4, IL-2, IL-17, BSA IL-13, and TSLP was tested by ELISA as described herein. As can be seen in FIG. 25, while BDG33.025 shows strong binding to IL-13, TSLP and IL-4, but not to IL-2 and IL-17. BDG33.023 bind strongly to TSLP and IL-13 but shows no binding to the other ligands, indicating that its binding to IL-13 and TSLP is specific.

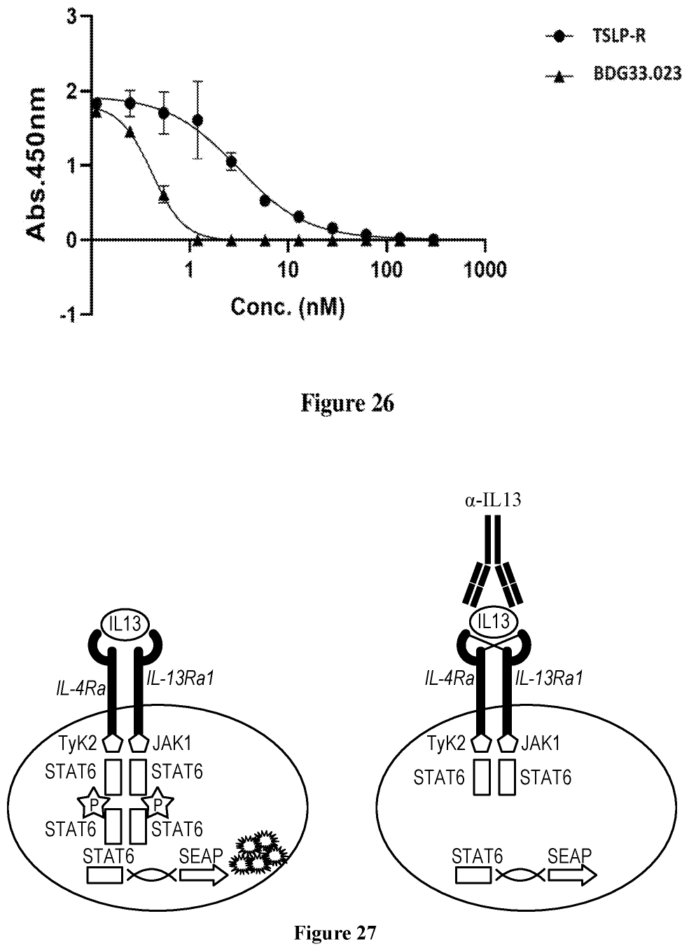

To test if BDG33.023 binds TSLP at a functional epitope, the ability of BDG33.023 to cross-block TSLP from binding to a TSLP receptor was tested. Briefly TSLP-R was coated on ELISA plate wells, and its ability to bind hTSLP in the presence of 0 nM to 500 nM BDG33.023 was tested. As can be seen in FIG. 26, BDG 33.023 can cross block TSLP binding to TSLP-R with an IC 50 of 0.41 nM indicating that BDG33.023 binds tightly TSLP at a biologically functional site.

Tables 13A, and 13B: KD values of antibody clones for human IL-13 and TSLP.

TABLE 13A

| | Human IL-13 | | |
|---|---|---|---|
| Antibody | K_{D (Steady state) (M)} | SE(K_{D}) (M) | |
| BDG33.003 | 2.16E−08 | 2.5E−09 | |
| BDG33.004 | 5.74E−08 | 4.1E−09 | |
| | Human TSLP | | |
| | ka (1/Ms) | kd (1/s) | KD (M) |
| BDG33.003 | 2.87E+05 | 1.29E−3 | 4.5E−9 |
| BDG33.004 | 3.44E+05 | 1.14E−3 | 3.33E−9 |

TABLE 13B

| | Human IL-13 | | |
|---|---|---|---|
| Antibody | ka (1/Ms) | kd (1/s) | KD (M) |
| BDG33.023 | 4.31E+06 | 0.01573 | 3.65E−09 |
| BDG33.025 | 4.07E+6 | 0.0036 | 9.03E−10 |

TABLE 14
EC50 values for human and cyno TSLP, and cyno IL-13

| | EC50 value for ligand | | |
|---|---|---|---|
| IgG | hTSLP | cTSLP | cIL-13 |
| BDG33.023 | 2.5 nM | 8.2 nM | 7.1 nM |
| BDG33.025 | 12.8 nM | 148 nM | 1.5 nM |

### Example 7

### Cell Based Assays for the Inhibitor Antibody BDG33.003 (Clone C2)

Objective: Analyze the IgG1 antibodies for the ability to inhibit IL-13 activity.

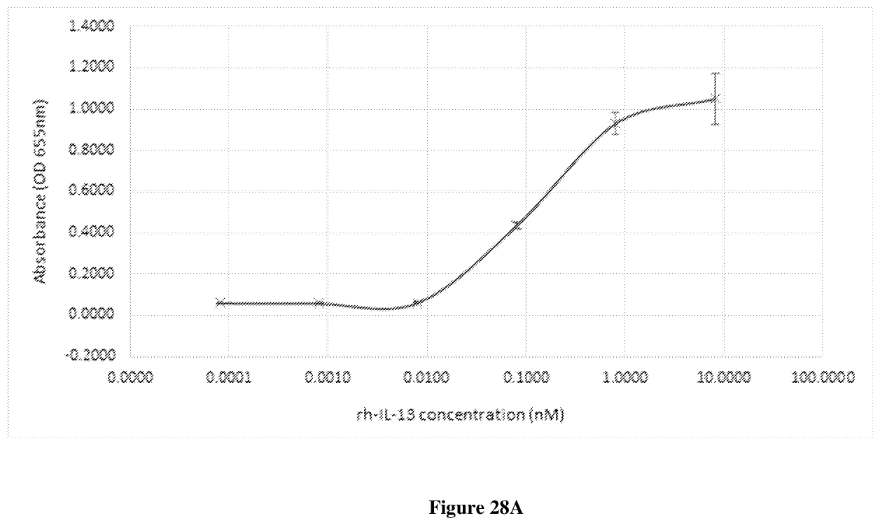

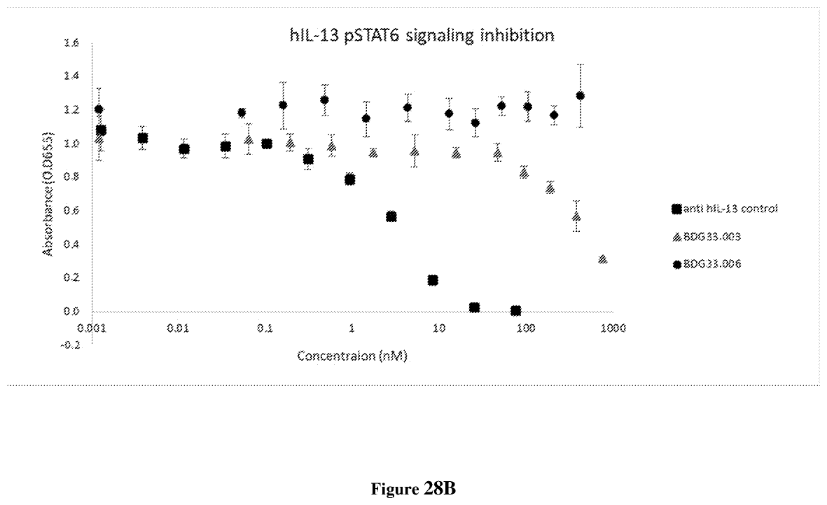

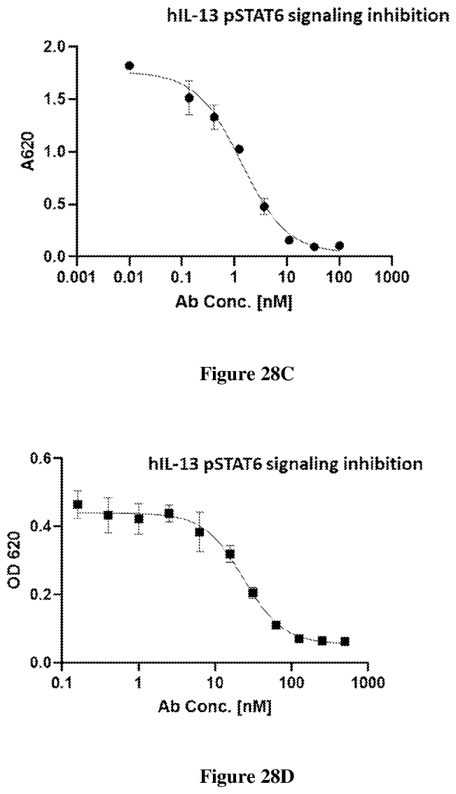

Results: To evaluate the capability of the antibody to inhibit rh-IL-13, the HEK-Blue IL-4/IL-13 system was used. The system uses HEK293 cells, which were stably transfected with human STAT6 gene and the reporter gene secreted embryonic alkaline phosphatase (SEAP) under the control of the IFNβ minimal promoter fused to four STAT6 binding sites (Example 4 (Methods), and FIG. 27). The system was initially tested by introducing rh-IL-13 to the cells and following the cell signaling cascade resulting in IL-13R (IL-13 receptor) activation by rh-IL-13. The results showed that IL-13 had an EC50 of about 0.12 nM to the cells (FIG. 28). Next, the engineered BDG33.003 (clone C2), BDG33.023 and BDG33.025 antibodies were tested to determine if they could inhibit IL-13 mediated activation of the cell's signaling cascade. The antibody was incubated with 0.4 nM rh-IL-13, which was shown to activate IL-13R to approximately 70% of the saturation level, and the IgG/IL-13 mixture was introduced to the cells for 24 hrs. The results obtained showed that the antibodies were able to inhibit IL-13 from binding to the IL-13R/IL-4R receptor complex, thus interfering with the signaling cascade. While for BDG33.003 the exact IC50 value was hard to determine, it is clear that BDG33.003 is inhibiting IL-13 signaling cascade. In addition, BDG33.023 and BDG33.025 inhibited IL-13 signaling cascade with an IC50 of 1.3 nM and 25 nM respectively, indicating that the IgGs are functionally blocking IL-13 in a biologically relevant setting. (FIGS. 28B-28D).

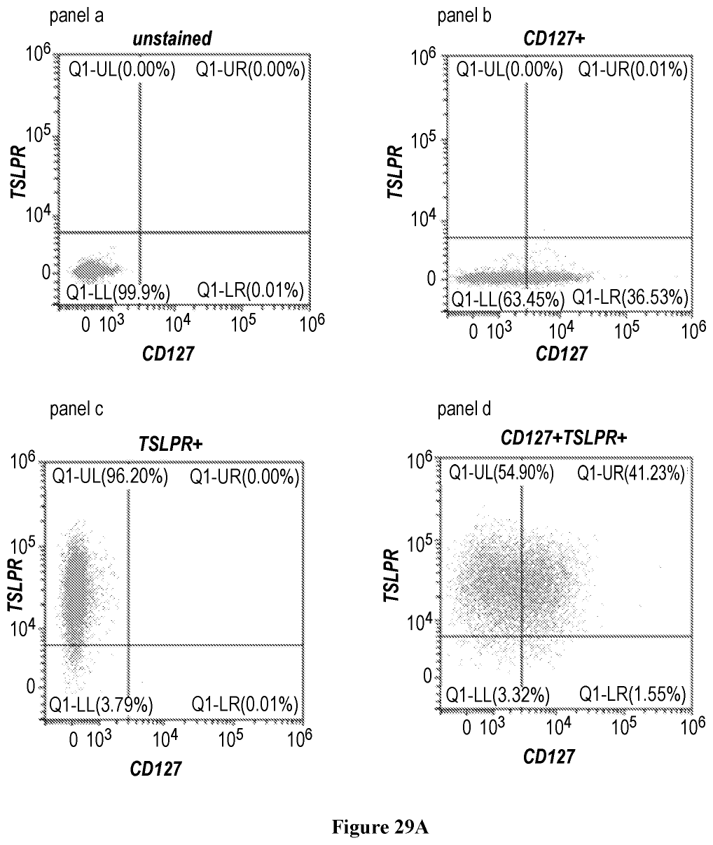

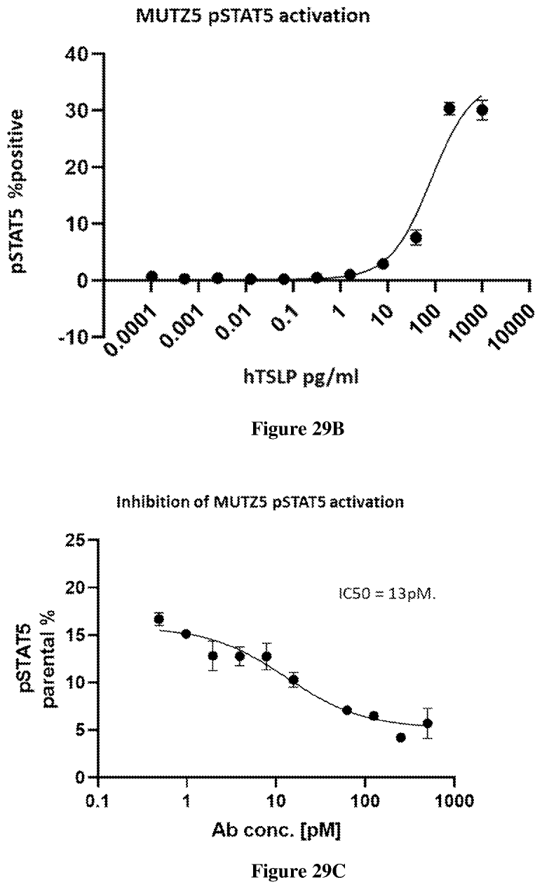

To evaluate the capability of the antibodies to inhibit human TSLP in cells, MUTZ5 cells were used to test pSTAT5 TSLP dependent activation in a similar manner reported by Francis O L, Milford T A, Martinez S R, et al. A novel xenograft model to study the role of TSLP-induced CRLF2 signals in normal and malignant human B lymphopoiesis. Haematologica. 2016; 101(4):417-426. TSLP induced phospho-STAT5 (pSTAT5) cellular activation cascade requires IL-7 receptor and TSLP-R receptor to function, as can be seen in FIG. 29A both are expressed on the MUTZ5 cell line surface indicating that these cells have the necessary receptors for this assay. To establish the cellular response to TSLP cells were incubated with TSLP at a concentration range of 0.0001 to 1000 pg/ml and their pSTAT5 activation levels were determined by flow cytometry. As can be seen in FIG. 29B treatment of MUTZ 5 cells with TSLP activates pSTAT5 in a dose dependent manner, indicating that these cells respond to TSLP via the pSTAT5 pathway. To test BDG33.023 inhibition of TSLP dependent STAT5 activation, TSLP at a concentration of 14 pM was mixed with 0.48 pM to 500 pM BDG33.023 and incubated with MUTZ cells, as can be seen in FIG. 29C BDG33.023 inhibit TSLP pSTAT5 activation with an IC50 value of 13 pM. These experiments demonstrate that BDG33.023 is functionally blocking TSLP in a biologically relevant cell-based setting.

Summary: The “re-epitoped” engineered BDG33.003 (clone 2), BDG33.023, and BDG33.025 antibodies were shown to bind both TSLP and IL-13 In contrast to the bispecific antibody format where each Fv has a specificity to a single antigen, these three antibodies are a standard IgG format, and each Fv has specificity to both IL-13 and TSLP In addition BDG33.023's paratopes for IL-13 and TSLP was shown to be at least partly overlapping. All three IgGs interfere with the IL-13R/IL4R and TSLPR/IL-7R signaling cascade. Such antibodies could be used as a component of a therapeutic treatment, for example but not limited to, severe asthma, atopic dermatitis, and other allergic and respiratory conditions.

While certain features of the engineered dual antibodies disclosed have been illustrated and described herein, many modifications, substitutions, changes, and equivalents will now occur to those of ordinary skill in the art. It is, therefore, to be understood that the appended claims are intended to cover all such modifications and changes as fall within the true spirit of what has been described herein.

1.-22. (canceled)

23. An isolated antibody with dual binding specificity comprising a first binding-site for a first antigen and a second binding-site for a second antigen, said antibody comprising a heavy chain variable region (VH) and a light chain variable region (VL), said VH and VL comprising said first binding-site for said first antigen and said second binding-site for said second antigen, wherein said second binding-site comprises amino acid variations of native amino acid sequences of an antibody that binds to said first antigen, wherein the variations do not abrogate binding to said first antigen.

24. The antibody of claim 23, wherein the antibody is in the form of an IgG, a single-chain fragment variable (scFv), an Fab, an F(ab′)2, a minibody, a diabody, a triabody, a nanobody, or a single domain antibody, and wherein said IgG is of the subclass of IgG1, IgG2, IgG3, or IgG4.

25. The antibody of claim 23, wherein said dual binding specificity comprises binding to said first antigen and binding to said second antigen at the same time.

26. The antibody of claim 23, wherein said antibody cannot bind both said first antigen and said second antigen at the same time.

27. The antibody of claim 23, wherein said antibody is generated by a method comprising the steps of:
(a) identifying and providing a first plurality of amino acid sequences from antibodies that bind to a first antigen, said amino acid sequences comprising an identified antigen-binding site binding to said first antigen, said first antigen-binding site comprising variable heavy chain (VH) and variable light chain (VL) domains, each VH and VL domain comprising complementarity determining regions (CDRs) and framework regions (FR), wherein greater than 75% of said CDR positions of the VH or VL domains are non-paratope CDR residues;
(b) identifying continuous surface patches comprising amino acid residues that do not form specific interactions with said first antigen, wherein said amino acid residues provide favorable sites for variant amino acids and wherein each patch comprises at least one subgroup of amino acid residues forming a continuous surface, wherein said patches identified within said first antigen-binding site comprises amino acid residues on a heavy chain variable (VH) region, or a light chain variable (VL) region, or both, and wherein said identification of the first antigen-binding site comprises one or more of amino acid sequence analysis, structural analysis, mutational analysis, hydrogen-deuterium exchange analysis, computational analysis, or any combination thereof;
(c) selecting at least one subgroup of amino acid residues comprised within at least one of said patches for introducing one or more amino acid variants;
(d) introducing amino acid variants to one or more residues within said one or more selected subgroups, thereby generating a second plurality of amino acid sequences, each of which comprises the antigen-binding site to said first antigen and said amino acid variants;
(e) generating a high-throughput screening (HTS) library comprising said second plurality of amino acid sequences;
(f) screening said HTS library for binding to said first antigen and binding to said second antigen; and
(g) selecting from said HTS library, candidate antibodies that preserved binding to said first antigen and confer binding to said second antigen, thereby generating the antibody with dual binding specificity.

28. The antibody of claim 27, wherein said one or more amino acid variations is in a CDR region or a framework region or a combination thereof.

29. The antibody of claim 27, wherein said patches comprise a set of solvent accessible amino acid residues that are in close proximity.

30. The antibody of claim 29, wherein said set of solvent accessible amino acid residues that are in close proximity has a length of about 2 to 15 amino acid residues.

31. The antibody of claim 27, wherein binding specificity, binding affinity, or binding avidity of said candidate antibodies to said first antigen is not reduced by more than about one to three-orders of magnitude after said introduction of the amino acid variations.

32. The antibody of claim 27, wherein said method further comprises a maturation affinity step of said candidate antibodies following step (g), followed by at least one additional screening step and selecting step.

33. The antibody of claim 27, wherein said first plurality of amino acid sequences comprises one or more sequences set forth in SEQ ID NOs: 3-28.

34. The antibody of claim 27, wherein candidate antibodies of step (g) comprise antibodies with dual binding specificity and having at least 800 μM binding affinity for each antigen.

35. The antibody of claim 23, wherein said first antigen is selected from the group consisting of PD1, tumor necrosis factor alpha, 0-amyloid peptide, CD11a, immunoglobulin E, human epidermal growth factor receptor 2, vascular endothelial growth factor A, CD20, nerve growth factor, IL-13, programmed death ligand 1 (PD-L1), and epidermal growth factor receptor.

36. The antibody of claim 35, wherein said first antigen is said PD1 (Programmed cell death protein 1) antigen.

37. The antibody of claim 36, wherein said second antigen is selected from the group consisting of OX40, a glucocorticoid-Induced TNFR-Related (GITR) antigen, CTLA4, PDL-1, PD-1, CD25, tumor necrosis factor receptor 2 (TNFR2), VISTA (B7-H5), T cell immunoglobulin and mucin domain-containing protein 3 (TIM3), vascular endothelial growth factor (VEGF), Lymphocyte-activation gene 3 (LAG3), 4-1BB (CD137), DR3 (TNFRSF25), IL-2, and CD3.

38. The antibody of claim 37, wherein when said first antigen is PD1 and said second antigen is OX40, the amino acid sequences of the VH and VL pair of said antibody comprise the pair of amino acid sequences set forth in any of SEQ ID NOs: 63 and 64, 54 and 55, 57 and 58, 60 and 61, 66 and 67, 69 and 70, 72 and 73, 75 and 76, 78 and 79, 81 and 82, 84 and 85, and 87 and 88, respectively.

39. The antibody of claim 37, wherein when said first antigen is PD1 and said second antigen is GITR, the amino acid sequences of the VH and VL pair of said antibody comprise the pair of amino acid sequences set forth in any of SEQ ID NOs: 102 and 103, 99 and 100, 96 and 97, and 108 and 109, respectively.


SEQUENCE LISTING (317 sequences)

SEQ ID NO: 1 - protein, 121 aa, Artificial Sequence [sequence omitted]

SEQ ID NO: 2 - protein, 107 aa, Artificial Sequence [sequence omitted]

SEQ ID NO: 3 - protein, 119 aa, Artificial Sequence [sequence omitted]

SEQ ID NO: 4 - protein, 112 aa, Artificial Sequence [sequence omitted]

SEQ ID NO: 5 - protein, 121 aa, Artificial Sequence
  Description of Artificial Sequence Synthetic polypeptide
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSS

SEQ ID NO: 6 - protein, 108 aa, Artificial Sequence
  Description of Artificial Sequence Synthetic polypeptide
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIK

SEQ ID NO: 7 - protein, 121 aa, Artificial Sequence [sequence omitted]

SEQ ID NO: 8 - protein, 107 aa, Artificial Sequence [sequence omitted]

SEQ ID NO: 9 - protein, 126 aa, Artificial Sequence
  Description of Artificial Sequence Synthetic polypeptide
QVQLVESGGGVVQPGRSLRLSCAASGFIFSSYAMHWVRQAPGNGLEWVAFMSYDGSNKKYADSVKGRFTISRDNSKNTLYLQMNSLRAEDTAVYYCARDRGIAAGGNYYYYGMDVWGQGTTVTVSS

SEQ ID NO: 10 - protein, 108 aa, Artificial Sequence
  Description of Artificial Sequence Synthetic polypeptide
EIVLTQSPATLSLSPGERATLSCRASQSVYSYLAWYQQKPGQAPRLLIYDASNRATGIPARFSGSGSGTDFTLTISSLEPEDFAVYYCQQRSNWPPFTFGPGTKVDIK

SEQ ID NO: 11 - protein, 118 aa, Artificial Sequence [sequence omitted]

SEQ ID NO: 12 - protein, 111 aa, Artificial Sequence [sequence omitted]

SEQ ID NO: 13 - protein, 121 aa, Artificial Sequence [sequence omitted]

SEQ ID NO: 14 - protein, 106 aa, Artificial Sequence [sequence omitted]

SEQ ID NO: 15 - protein, 113 aa, Artificial Sequence [sequence omitted]

SEQ ID NO: 16 - protein, 107 aa, Artificial Sequence [sequence omitted]

SEQ ID NO: 17 - protein, 121 aa, Artificial Sequence [sequence omitted]

SEQ ID NO: 18 - protein, 111 aa, Artificial Sequence [sequence omitted]

SEQ ID NO: 19 - protein, 119 aa, Artificial Sequence [sequence omitted]

SEQ ID NO: 20 - protein, 107 aa, Artificial Sequence [sequence omitted]

SEQ ID NO: 21 - protein, 123 aa, Artificial Sequence [sequence omitted]

SEQ ID NO: 22 - protein, 107 aa, Artificial Sequence [sequence omitted]

SEQ ID NO: 23 - protein, 121 aa, Artificial Sequence [sequence omitted]

SEQ ID NO: 24 - protein, 106 aa, Artificial Sequence [sequence omitted]

SEQ ID NO: 25 - protein, 121 aa, Artificial Sequence [sequence omitted]

SEQ ID NO: 26 - protein, 107 aa, Artificial Sequence [sequence omitted]

SEQ ID NO: 27 - protein, 122 aa, Artificial Sequence
  Description of Artificial Sequence Synthetic polypeptide
QVQLVQSGAEVKKPGASVKVSCKASGYTFTNYGLSWVRQAPGQGLEWMGWISANNGDTNYGQEFQGRVTMTTDTSTSTAYMELRSLRSDDTAVYYCARDSSSSWARWFFDLWGRGTLVTVSS

SEQ ID NO: 28 - protein, 108 aa, Artificial Sequence
  Description of Artificial Sequence Synthetic polypeptide
SYVLTQPPSVSVAPGKTARITCGGNIIGSKLVHWYQQKPGQAPVLVIYDDGDRPSGIPERFSGSNSGNTATLTISRVEAGDEADYYCQVWDTGSDPVVFGGGTKLTVL

SEQ ID NO: 29 - DNA, 705 bp, Artificial Sequence
  Description of Artificial Sequence Synthetic polynucleotide
caggtacaattggtagagtccggtggtggcgttgtccagccaggccgttccctgcgtctggactgcaaagcatctggcatcaccttctccaacagcggtatgcactgggtgcgtcaggccccaggcaaagggctggagtgggtcgccgtgatctggtatgatgggtctaaacgttactatgctgactctgtaaaaggccgtttcaccatcagccgtgacaatagcaagaacaccctgttcttacagatgaattccctgcgtgctgaagataccgccgtatactactgcgctactaacgacgattattggggccaaggtaccctggtcaccgtctcctctggcggtggtggtagcggaggcggaggatcaggtggaggcggcagtgaaattgtgctgacccaaagcccagcaaccctgagcctgtctccaggcgaacgtgccactctgtcctgccgtgcaagccagtctgtctcttcttacctggcatggtaccagcagaagccagggcaagccccacgtctgttaatctacgacgcaagcaatcgtgccaccggtattccagcccgtttttctggcagcggttccggcaccgatttcactctgaccatctcctccctggaaccagaagacttcgccgtttactactgtcaacagtcatcaaactggccacgtacttttggtcagggcaccaaagttgagattaaa

SEQ ID NO: 30 - protein, 113 aa, Artificial Sequence [sequence omitted]

SEQ ID NO: 31 - protein, 107 aa, Artificial Sequence [sequence omitted]

SEQ ID NO: 32 - DNA, 705 bp, Artificial Sequence
  Description of Artificial Sequence Synthetic polynucleotide
caggtacaattggtagagtccggtggtggcgttgtccagccaggccgttccctgcgtctggactgcaaagcatctggcatcaccttctccaacagcggtatgcactgggtgcgtcaggccccaggcaaagggctggagtgggtcgccgtgatcggttatgatgggtctagagtcaagtatgctgactctgtaaaaggccgtttcaccatcagccgtgacaatagcaagaacaccctgttcttacagatgaattccctgcgtgctgaagataccgccgtatactactgcgctactaacgacgattattggggccaaggtaccctggtcaccgtctcctctggcggtggtggtagcggaggcggaggatcaggtggaggcggcagtgaaattgtgctgacccaaagcccagcaaccctgagcctgtctccaggcgaacgtgccactctgtcctgccgtgcaagcagacctgtctcttctaatctggcatggtaccagcagaagccagggcaagccccacgtctgttaatctaccgtgcaagcaatcgtgccaccggtattccagcccgtttttctggcagcggttccggcaccgatttcactctgaccatctcctccctggaaccagaagacttcgccgtttactactgtcaacagtcaagaaagtggccacgtacttttggtcagggcaccaaagttgagattaaa

SEQ ID NO: 33 - protein, 113 aa, Artificial Sequence
  Description of Artificial Sequence Synthetic polypeptide
QVQLVESGGGVVQPGRSLRLDCKASGITFSNSGMHWVRQAPGKGLEWVAVIGYDGSRVKYADSVKGRFTISRDNSKNTLFLQMNSLRAEDTAVYYCATNDDYWGQGTLVTVSS

SEQ ID NO: 34 - protein, 107 aa, Artificial Sequence
  Description of Artificial Sequence Synthetic polypeptide
EIVLTQSPATLSLSPGERATLSCRASRPVSSNLAWYQQKPGQAPRLLIYRASNRATGIPARFSGSGSGTDFTLTISSLEPEDFAVYYCQQSRKWPRTFGQGTKVEIK

SEQ ID NO: 35 - DNA, 705 bp, Artificial Sequence
  Description of Artificial Sequence Synthetic polynucleotide
caggtacaattggtagagtccggtggtggcgttgtccagccaggccgttccctgcgtctggactgcaaagcatctggcatcaccttctccaacagcggtatgcactgggtgcgtcaggccccaggcaaagtgctggagtgggtcgccgtgatctggtatgattggtctaaacgttactatgctgactctgtaaaaggccgtttcaccatcagccgtgacaatagcaagaacaccctgttcttacagatgaattccctgcgtgctgaagataccgccgtatactactgcgctactaacgacgattattggggccaaggtaccctggtcaccgtctcctctggcggtggtggtagcggaggcggaggatcaggtggaggcggcagtgaaattgtgctgacccaaagcccagcaaccctgagcctgtctccaggcgaacgtgccactctgtcctgccgtgcaagccagatggtcggttctaagctggcatggtaccagcagaagccagggcaagccccacgtctgttaatctacagtgcaagcaatcgtgccaccggtattccagcccgtttttctggcagcggttccggcagagatttcactctgaccatctcctccctggaaccagaagacttcgccgtttactactgtcaacagtcagttaacttgccacgtacttttggtcagggcaccaaagttgagattaaa

SEQ ID NO: 36 - protein, 113 aa, Artificial Sequence
  Description of Artificial Sequence Synthetic polypeptide
QVQLVESGGGVVQPGRSLRLDCKASGITFSNSGMHWVRQAPGKVLEWVAVIWYDWSKRYYADSVKGRFTISRDNSKNTLFLQMNSLRAEDTAVYYCATNDDYWGQGTLVTVSS

SEQ ID NO: 37 - protein, 113 aa, Artificial Sequence; identical to SEQ ID NO: 36
  Description of Artificial Sequence Synthetic polypeptide

SEQ ID NO: 38 - DNA, 705 bp, Artificial Sequence
  Description of Artificial Sequence Synthetic polynucleotide
caggtacaattggtagagtccggtggtggcgttgtccagccaggccgttccctgcgtctggactgcaaagcatctggcatcaccttctccaacagcggtatgcactgggtgcgtcaggccccaggcaaagggctggagtgggtcgccgtgatctggtatgatgggtctaaacgttactatgctgactctgtaaaaggccgtttcaccatcagccgtgacaatagcaagaacaccctgttcttacagatgaattccctgcgtgctgaagataccgccgtatactactgcgctactaacgacgattattggggccaaggtaccctggtcaccgtctcctctggcggtggtggtagcggaggcggaggatcaggtggaggcggcagtgaaattgtgctgacccaaagcccagcaaccctgagcctgtctccaggcgaacgtgccactctgtcctgccgtgcaagccagtctgtcatccactacctggcatggtaccagcagaagccagggcaagccccacgtctgttaatctacgacgcaagcaatcgtgccaccggtattccagcccgtttttctggcagcggttccggcaccgatttcactctgaccatctcctccctggaaccagaagacttcgccgtttactactgtcaacagtattcaaactggccacttacttttggtcagggcaccaaagttgagattaaa

SEQ ID NO: 39 - protein, 113 aa, Artificial Sequence [sequence omitted]

SEQ ID NO: 40 - protein, 107 aa, Artificial Sequence
  Description of Artificial Sequence Synthetic polypeptide
EIVLTQSPATLSLSPGERATLSCRASQSVIHYLAWYQQKPGQAPRLLIYDASNRATGIPARFSGSGSGTDFTLTISSLEPEDFAVYYCQQYSNWPLTFGQGTKVEIK

SEQ ID NO: 41 - DNA, 705 bp, Artificial Sequence
  Description of Artificial Sequence Synthetic polynucleotide
caggcacaattggtagagtccgggggtggcgttgtccaggcaggccgttccctgcgtctggactgcaaagcatctggcatcaccttctccaacaggggtatgcactgggtgcgtcaggccccaggcaaagggctggagtgggtcgccgtgatctggtatgatgggtctctgcgttactatgctgactctgtaaaaggccgtttcaccatcagccgtgacaatagcaagaacaccctgttcttacagatgaattccctgcgtgctgaagataccgccgtatactactgcgctactaacgacgattattggggccaaggtaccctggtcaccgtctcctctggcggtggtggtagcggaggcggaggatcaggtggaggcggcagtgaaattgtgctgacccaaagcccagcaaccctgagcctgtctccaggcgaacgtgccactctgtcctgccgtgcaagccagtctgtctctgacaacctggcatggtaccagcagaagccagggcaagccccacgtctgttaatctacgacgcagacaatcgtgccaccggtattccagcccgtttttctggcagcggttccggcaccgatttcactctgaccatctcctccctggaaccagaagacttcgccgtttactactgtcaacagtcatcaaacttaccacgtacttttggtcagggcaccaaagttgagattaaa

SEQ ID NO: 42 - protein, 113 aa, Artificial Sequence
  Description of Artificial Sequence Synthetic polypeptide
QAQLVESGGGVVQAGRSLRLDCKASGITFSNRGMHWVRQAPGKGLEWVAVIWYDGSLRYYADSVKGRFTISRDNSKNTLFLQMNSLRAEDTAVYYCATNDDYWGQGTLVTVSS

SEQ ID NO: 43 - protein, 107 aa, Artificial Sequence
  Description of Artificial Sequence Synthetic polypeptide
EIVLTQSPATLSLSPGERATLSCRASQSVSDNLAWYQQKPGQAPRLLIYDADNRATGIPARFSGSGSGTDFTLTISSLEPEDFAVYYCQQSSNLPRTFGQGTKVEIK

SEQ ID NO: 44 - DNA, 705 bp, Artificial Sequence
  Description of Artificial Sequence Synthetic polynucleotide
caggtacaattggtagagtccggtggtggcgttgtccagccaggccgttccctgcgtctggactgcaaagcatctggcatcaccttctccaacagcggtatgcactgggtgcgtcaggccccaggcaaagggctggagtgggtcgccgtgatctggtatgatgggtctctgcgttactatgctgactctgtaaaaggccgtttcaccatcagccgtgacaatagcaagaacaccctgttcttacagatgaattccctgcgtgctgaagataccgccgtatactactgcgctactaacgacgattattggggccaaggtaccctggtcaccgtctcctctggcggtggtggtagcggaggcggaggatcaggtggaggcggcagtgaaattgtgctgacccaaagcccagcaaccctgagcctgtctccaggcgaacgtgccactctgtcctgccgtgcaagccagtctgtctctgacaacctggcatggtaccagcagaagccagggcaagccccacgtctgttaatctacgacgcagacaatcgtgccaccggtattccagcccgtttttctggcagcggttccggcaccgatttcactctgaccatctcctccctggaaccagaagacttcgccgtttactactgtcaacagtcatcaaacttaccacgtacttttggtcagggcaccaaagttgagattaaa

SEQ ID NO: 45 - protein, 113 aa, Artificial Sequence
  Description of Artificial Sequence Synthetic polypeptide
QVQLVESGGGVVQPGRSLRLDCKASGITFSNSGMHWVRQAPGKGLEWVAVIWYDGSLRYYADSVKGRFTISRDNSKNTLFLQMNSLRAEDTAVYYCATNDDYWGQGTLVTVSS

SEQ ID NO: 46 - protein, 107 aa, Artificial Sequence; identical to SEQ ID NO: 43
  Description of Artificial Sequence Synthetic polypeptide

SEQ ID NO: 47 - DNA, 705 bp, Artificial Sequence
  Description of Artificial Sequence Synthetic polynucleotide
caggtccaattggtagagtccggtggtggcgttgtccatccaggccgttccctgcgtgtggactgcaaagcatctggcatcaccttctccaacagcggtatgcactgggtgcgtcaggccccaggcaaagggctggagtgggtcgccgtgatctggtatgatgggtctctgcgttactatgctgactctgtaaaaggccgtttcaccatcagccgtgacaatagcaagaacaccctgttcttacagatgaattccctgcgtgctgaagataccgccgtatactactgcgctactaacgacgattattggggccaaggtaccctggtcaccgtctcctctggcggtggtggtagcggaggcggaggatcaggtggaggcggcagtgaaattgtgctgacccaaagcccagcaaccctgagcctgtctccaggcgaacgtgccactctgtcctgccgtgcaagccagtctgtctctgacaacctggcatggtaccagcagaagccagggcaagccccacgtctgttaatctacgacgcagacaatcgtgccaccggtattccagcccgtttttctggcagcggttccggcaccgatttcactctgaccatctcctccctggcaccagaagacttcgccgtttactactgtcaacagtcatcaaacttaccacgtacttttggtcagggcaccaaagttgagattaaa

SEQ ID NO: 48 - protein, 113 aa, Artificial Sequence
  Description of Artificial Sequence Synthetic polypeptide
QVQLVESGGGVVHPGRSLRVDCKASGITFSNSGMHWVRQAPGKGLEWVAVIWYDGSLRYYADSVKGRFTISRDNSKNTLFLQMNSLRAEDTAVYYCATNDDYWGQGTLVTVSS

SEQ ID NO: 49 - protein, 107 aa, Artificial Sequence
  Description of Artificial Sequence Synthetic polypeptide
EIVLTQSPATLSLSPGERATLSCRASQSVSDNLAWYQQKPGQAPRLLIYDADNRATGIPARFSGSGSGTDFTLTISSLAPEDFAVYYCQQSSNLPRTFGQGTKVEIK

SEQ ID NO: 50 - DNA, 705 bp, Artificial Sequence
  Description of Artificial Sequence Synthetic polynucleotide
caggtacaattggtagagtccggtggtggcgttgtccagcctggccgttccctgcgtctggactgcaaagcatctggcatcaccttctccaacagcggtatgcactgggtgcgtcaggccccaggcaaagggctggagtgggtcgccgtgatctggtatgatgggtctctgcgttactatgctgactctgtaaaaggccgtttcaccatcagccgtgacaatagcaagaacaccctgttcttacagatgaattccctgcgtgctgaagataccgccgtatactactgcgcttctaacgacgattattggggccaaggtaccctggtcaccgtctcctctggcggtggtggtagcggaggcggaggatcaggtggaggcggcagtgaaattgtgctgacccaaagcccagcaaccctgagcctgtctccaggcgaacgtgccactctgtcctgccgtgcaagccagtctgtctctgacaacctggcatggtaccagcagaagccagggcaagccccacgtctgttaatctacgacgcagacaatcgtgccaccggtattccagcccgtttttctggcagcggttccggcaccgatttcactctgaccatctcctccctggaaccagaagacttcgccgtttactactgtcaacagtcatcaaacttaccacgtacttttggtcagggcaccaaagttgagattaaa

SEQ ID NO: 51 - protein, 113 aa, Artificial Sequence
  Description of Artificial Sequence Synthetic polypeptide
QVQLVESGGGVVQPGRSLRLDCKASGITFSNSGMHWVRQAPGKGLEWVAVIWYDGSLRYYADSVKGRFTISRDNSKNTLFLQMNSLRAEDTAVYYCASNDDYWGQGTLVTVSS

SEQ ID NO: 52 - protein, 107 aa, Artificial Sequence; identical to SEQ ID NO: 43
  Description of Artificial Sequence Synthetic polypeptide

SEQ ID NO: 53 - DNA, 705 bp, Artificial Sequence
  Description of Artificial Sequence Synthetic polynucleotide
caggtacaattggtagagtccggtggtggcgttgtccagccaggccgttccctgcgtctggactgcaaagcatctggcatcaccttctccaacagcggtatgcactgggtgcgtcaggccccaggcaaagggctggagtgggtcgccgtgatctggtatgatgggtctaaaagctactatgctgactctgtaaaaggccgtttcaccatcagccgtgacaatagcaagaacaccctgttcttacagatgaattccctgcgtgctgaagataccgccgtatactactgcgctactaacgacgattattggggccaaggtaccctggtcaccgtctcctctggcggtggtggtagcggaggcggaggatcaggtggaggcggcagtgaaattgtgctgacccaaagcccagcaaccctgagcctgtctccaggcgaacgtgccactctgtcctgccgtgcaagccagtctgtccgccactacctggcatggtaccagcagaagccagggcaagccccacgtctgttaatctacgacgcaatgaatcgtgccaccggtattccagcccgtttttctggcagcggttccggcaccgatttcactctgaccatctcctccctggaaccagaagacttcgccgtttactactgtcaacagtatgaggactggccacttacttttggtcagggcaccaaagttgagattaaa

SEQ ID NO: 54 - protein, 113 aa, Artificial Sequence
  Description of Artificial Sequence Synthetic polypeptide
QVQLVESGGGVVQPGRSLRLDCKASGITFSNSGMHWVRQAPGKGLEWVAVIWYDGSKSYYADSVKGRFTISRDNSKNTLFLQMNSLRAEDTAVYYCATNDDYWGQGTLVTVSS

SEQ ID NO: 55 - protein, 107 aa, Artificial Sequence
  Description of Artificial Sequence Synthetic polypeptide
EIVLTQSPATLSLSPGERATLSCRASQSVRHYLAWYQQKPGQAPRLLIYDAMNRATGIPARFSGSGSGTDFTLTISSLEPEDFAVYYCQQYEDWPLTFGQGTKVEIK

SEQ ID NO: 56 - DNA, 705 bp, Artificial Sequence
  Description of Artificial Sequence Synthetic polynucleotide
caggtacaattggtagagtccggtggtggcgttgtccagccaggccgttccctgcgtctggactgcaaagcatctggcatcgcgttctccaacagcggtatgcactgggtgcgtcaggccccaggcaaagggctggagtgggtcgccgtgatctggtatgatgggtctaaaacgtactatgctgactctgtaaaaggccgtttcaccatcagccgtgacaatagcaagaacaccctgttcttacagatgaattccctgcgtgctgaagataccgccgtatactactgcgctactaacgacgattattggggccaaggtaccctggtcaccgtctcctctgacggtggtggtagcggaggcggaggatcaggtggaggcggcagtgaaattgtgctgacccaaagcccagcaaccctgagcctgtctccaggcgaacgtgccactctgtcctgccgtgcaagccagtctgtcatccactacctggcatggtaccagcagaagccagggcaagccccacgtctgttaatctacgacgcaagcaatcgtgccaccggtattccagcccgtttttctggcagcggttccggcaccgatttcactctgaccatctcctccctggaaccagaagacttcgccgtttactactgtcaacagtataccaactggccacttacttttggtcagggcaccaaagttgagattaaa

SEQ ID NO: 57 - protein, 113 aa, Artificial Sequence
  Description of Artificial Sequence Synthetic polypeptide
QVQLVESGGGVVQPGRSLRLDCKASGIAFSNSGMHWVRQAPGKGLEWVAVIWYDGSKTYYADSVKGRFTISRDNSKNTLFLQMNSLRAEDTAVYYCATNDDYWGQGTLVTVSS

SEQ ID NO: 58 - protein, 107 aa, Artificial Sequence
  Description of Artificial Sequence Synthetic polypeptide
EIVLTQSPATLSLSPGERATLSCRASQSVIHYLAWYQQKPGQAPRLLIYDASNRATGIPARFSGSGSGTDFTLTISSLEPEDFAVYYCQQYTNWPLTFGQGTKVEIK

SEQ ID NO: 59 - DNA, 705 bp, Artificial Sequence
  Description of Artificial Sequence Synthetic polynucleotide
caggtacaattggtagagtccggtggtggcgttgtccagccaggccgttccctgcgtctggactgcaaagcatctggcatcgacttctccaacagcggtatgcactgggtgcgtcaggccccaggcaaagggctggagtgggtcgccgtgatctggtatgatgggtctacgcgttactatgctgactctgtaaaaggccgtttcaccatcagccgtgacaatagcaagaacaccctgttcttacagatgaattccctgcgtgctgaagataccgccgtatactactgcgctactaacgacgattattggggccaaggtaccctggtcaccgtctcctctggcggtggtggtagcggaggcggaggatcaggtggaggcggcagtgaaattgtgctgacccaaagcccagcaaccctgagcctgtctccaggcgaacgtgccactctgtcctgccgtgcaagccagtctgtcatccactacctggcatggtaccagcagaagccagggcaagccccacgtctgttaatctacgacgcaagcaatcgtgccaccggtattccagcccgtttttctggcagcggttccggcaccgatttcactctgaccatctcctccctggaaccagaagacttcgccgtttactactgtcaacagtatgacaactggccacttacttttggtcagggcaccaaagttgagattaaa

SEQ ID NO: 60 - protein, 113 aa, Artificial Sequence
  Description of Artificial Sequence Synthetic polypeptide
QVQLVESGGGVVQPGRSLRLDCKASGIDFSNSGMHWVRQAPGKGLEWVAVIWYDGSTRYYADSVKGRFTISRDNSKNTLFLQMNSLRAEDTAVYYCATNDDYWGQGTLVTVSS

SEQ ID NO: 61 - protein, 107 aa, Artificial Sequence
  Description of Artificial Sequence Synthetic polypeptide
EIVLTQSPATLSLSPGERATLSCRASQSVIHYLAWYQQKPGQAPRLLIYDASNRATGIPARFSGSGSGTDFTLTISSLEPEDFAVYYCQQYDNWPLTFGQGTKVEIK

SEQ ID NO: 62 - DNA, 705 bp, Artificial Sequence
  Description of Artificial Sequence Synthetic polynucleotide
caggtacaattggtagagtccggtggtggcgttgtccagccaggccgttccctgcgtctggactgcaaagcatctggcatcaccttctccaacagcggtatgcactgggtgcgtcaggccccaggcaaagggctggagtgggtcgccgtgatctggtatgatgggtctaaaacgtactatgctgactctgtaaaaggccgtttcaccatcagccgtgacaatagcaagaacaccctgttcttacagatgaattccctgcgtgctgaagataccgccgtatactactgcgctactaacgacgattattggggccaaggtaccctggtcaccgtctcctctggcggtggtggtagcggaggcggaggatcaggtggaggcggcagtgaaattgtgctgacccaaagcccagcaaccctgagcctgtctccaggcgaacgtgccactctgtcctgccgtgcaagccagtctgtcgagcactacctggcatggtaccagcagaagccagggcaagccccacgtctgttaatctacgacgcagggaatcgtgccaccggtattccagcccgtttttctggcagcggttccggcaccgatttcactctgaccatctcctccctggaaccagaagacttcgccgtttactactgtcaacagtatagcgactggccacttacttttggtcagggcaccaaagttgagattaaa

SEQ ID NO: 63 - protein, 113 aa, Artificial Sequence
  Description of Artificial Sequence Synthetic polypeptide
QVQLVESGGGVVQPGRSLRLDCKASGITFSNSGMHWVRQAPGKGLEWVAVIWYDGSKTYYADSVKGRFTISRDNSKNTLFLQMNSLRAEDTAVYYCATNDDYWGQGTLVTVSS

SEQ ID NO: 64 - protein, 107 aa, Artificial Sequence
  Description of Artificial Sequence Synthetic polypeptide
EIVLTQSPATLSLSPGERATLSCRASQSVEHYLAWYQQKPGQAPRLLIYDAGNRATGIPARFSGSGSGTDFTLTISSLEPEDFAVYYCQQYSDWPLTFGQGTKVEIK

SEQ ID NO: 65 - DNA, 705 bp, Artificial Sequence
  Description of Artificial Sequence Synthetic polynucleotide
caggtacaattggtagagtccggtggtggcgttgtccagccaggccgttccctgcgtctggactgcaaagcatctggcatcgacttctccaacagcggtatgcactgggtgcgtcaggccccaggcaaagggctggagtgggtcgccgtgatctggtatgatgggtctttgcgttactatgctgactctgtaaaaggccgtttcaccatcagccgtgacaatagcaagaacaccctgttcttacagatgaattccctgcgtgctgaagataccgccgtatactactgcgctactaacgacgattattggggccaaggtaccctggtcaccgtctcctctggcggtggtggtagcggaggcggaggatcaggtggaggcggcagtgaaattgtgctgacccaaagcccagcaaccctgagcctgtctccaggcgaacgtgccactctgtcctgccgtgcaagccagtctgtcgagcactacctggcatggtaccagcagaagccagggcaagccccacgtctgttagtctacgacgcaagcaatcgtgccaccggtattccagcccgtttttctggcagcggttccggcaccgatttcactctgaccatctcctccctggaaccagaagacttcgccgtttactactgtcaacagtattcaaactggccacttacttttggtcagggcaccaaagttgagattaaa

SEQ ID NO: 66 - protein, 113 aa, Artificial Sequence
  Description of Artificial Sequence Synthetic polypeptide
QVQLVESGGGVVQPGRSLRLDCKASGIDFSNSGMHWVRQAPGKGLEWVAVIWYDGSLRYYADSVKGRFTISRDNSKNTLFLQMNSLRAEDTAVYYCATNDDYWGQGTLVTVSS

SEQ ID NO: 67 - protein, 107 aa, Artificial Sequence
  Description of Artificial Sequence Synthetic polypeptide
EIVLTQSPATLSLSPGERATLSCRASQSVEHYLAWYQQKPGQAPRLLVYDASNRATGIPARFSGSGSGTDFTLTISSLEPEDFAVYYCQQYSNWPLTFGQGTKVEIK

SEQ ID NO: 68 - DNA, 705 bp, Artificial Sequence
  Description of Artificial Sequence Synthetic polynucleotide
caggtacaattggtagagtccggtggtggcgttgtccagccaggccgttccctgcgtctggactgcaaagcatctggcatcgacttctccaacagcggtatgcactgggtgcgtcaggccccaggcaaagggctggagtgggtcgccgtgatctggtatgatgggtctaaacgtttgtatgctgactctgtaaaaggccgtttcaccatcagccgtgacaatagcaagaacaccctgttcttacagatgaattccctgcgtgctgaagataccgccgtatactactgcgcttccaacgacgattattggggccaaggtaccctggtcaccgtctcctctggcggtggtggtagcggaggcggaggatcaggtggaggcggcagtgaaattgtgctgacccaaagcccagcaaccctgagcctgtctccaggcgaacgtgccactctgtcctgccgtgcaagccagtctgtcatccactacctggcatggtaccagcagaagccagggcaagccccacgtctgttaatctacgacgcaagcaatcgtgccaccggtattccagcccgtttttctggcagcggttccggcaccgatttcactctgaccatctcctccctggaaccagaagacttcgccgtttactactgtcaacagtatgacaactggccacttacttttggtcagggcaccaaagttgagattaaa

SEQ ID NO: 69 - protein, 113 aa, Artificial Sequence
  Description of Artificial Sequence Synthetic polypeptide
QVQLVESGGGVVQPGRSLRLDCKASGIDFSNSGMHWVRQAPGKGLEWVAVIWYDGSKRLYADSVKGRFTISRDNSKNTLFLQMNSLRAEDTAVYYCASNDDYWGQGTLVTVSS

SEQ ID NO: 70 - protein, 107 aa, Artificial Sequence; identical to SEQ ID NO: 61
  Description of Artificial Sequence Synthetic polypeptide

SEQ ID NO: 71 - DNA, 705 bp, Artificial Sequence
  Description of Artificial Sequence Synthetic polynucleotide
caggtacaattggtagagtccggtggtggcgttgtccagccaggccgttccctgcgtctggactgcaaagcatctggcatcgagttctccaacagcggtatgcactgggtgcgtcaggccccaggcaaagggctggagtgggtcgccgtgatctggtatgatgggtctaaacgttactatgctgactctgtaaaaggccgtttcaccatcagccgtgacaatagcaagaacaccctgttcttacagatgaattccctgcgtgctgaagataccgccgtatactactgcgctactaacgacgattattggggccaaggtaccctggtcaccgtctcctctggcggtggtggtagcggaggcggaggatcaggtggaggcggcagtgaaattgtgctgacccaaagcccagcaaccctgagcctgtctccaggcgaacgtgccactctgtcctgccgtgcaagccagtctgtcgagcactacctggcatggtaccagcagaagccagggcaagccccacgtctgttaatctacgacgcaagcaatcgtgccaccggtattccagcccgtttttctggcagcggttccggcaccgatttcactctgaccatctcctccctggaaccagaagacttcgccgtttactactgtcaacagtattcaaactggccacttacttttggtcagggcaccaaagttgagattaaa

SEQ ID NO: 72 - protein, 113 aa, Artificial Sequence
  Description of Artificial Sequence Synthetic polypeptide
QVQLVESGGGVVQPGRSLRLDCKASGIEFSNSGMHWVRQAPGKGLEWVAVIWYDGSKRYYADSVKGRFTISRDNSKNTLFLQMNSLRAEDTAVYYCATNDDYWGQGTLVTVSS

SEQ ID NO: 73 - protein, 107 aa, Artificial Sequence
  Description of Artificial Sequence Synthetic polypeptide
EIVLTQSPATLSLSPGERATLSCRASQSVEHYLAWYQQKPGQAPRLLIYDASNRATGIPARFSGSGSGTDFTLTISSLEPEDFAVYYCQQYSNWPLTFGQGTKVEIK

SEQ ID NO: 74 - DNA, 705 bp, Artificial Sequence
  Description of Artificial Sequence Synthetic polynucleotide
caggtacaattggtagagtccggtggtggcgttgtccagccaggccgttccctgcgtctggactgcaaagcatctggcatcgagttctccaacagcggtatgcactgggtgcgtcaggccccaggcaaagggctggagtgggtcgccgtgatctggtatgatgggtctaaacgttactatgctgactctgtaaaaggccgtttcaccatcagccgtgacaatagcaagaacaccctgttcttacagatgaattccctgcgtgctgaagataccgccgtatactactgcgctagcaacgacgattattggggccaaggtaccctggtcaccgtctcctctggcggtggtggtagcggaggcggaggatcaggtggaggcggcagtgaaattgtgctgacccaaagcccagcaaccctgagcctgtctccaggcgaacgtgccactctgtcctgccgtgcaagccagtctgtcgaccactacctggcatggtaccagcagaagccagggcaagccccacgtctgttaatctacgacgcaagcaatttcgccaccggtattccagcccgtttttctggcagcggttccggcaccgatttcactctgaccatctcctccctggaaccagaagacttcgccgtttactactgtcaacagtattcaaactggccacttacttttggtcagggcaccaaagttgagattaaa

SEQ ID NO: 75 - protein, 113 aa, Artificial Sequence
  Description of Artificial Sequence Synthetic polypeptide
QVQLVESGGGVVQPGRSLRLDCKASGIEFSNSGMHWVRQAPGKGLEWVAVIWYDGSKRYYADSVKGRFTISRDNSKNTLFLQMNSLRAEDTAVYYCASNDDYWGQGTLVTVSS

SEQ ID NO: 76 - protein, 107 aa, Artificial Sequence
  Description of Artificial Sequence Synthetic polypeptide
EIVLTQSPATLSLSPGERATLSCRASQSVDHYLAWYQQKPGQAPRLLIYDASNFATGIPARFSGSGSGTDFTLTISSLEPEDFAVYYCQQYSNWPLTFGQGTKVEIK

SEQ ID NO: 77 - DNA, 705 bp, Artificial Sequence
  Description of Artificial Sequence Synthetic polynucleotide
caggtacaattggtagagtccggtggtggcgttgtccagccaggccgttccctgcgtctggactgcaaagcatctggcatcgacttctccaacagcggtatgcactgggtgcgtcaggccccaggcaaagggctggagtggctggccgtgatctggtatgatgggtctaaacgttactatgctgactctgtaaaaggccgtttcaccatcagccgtgacaatagcaagaacaccctgttcttacagatgaattccctgcgtgctgaagataccgccgtatactactgcgcttccaacgacgattattggggccaaggtaccctggtcaccgtctcctctggcggtggtggtagcggaggcggaggatcaggtggaggcggcagtgaaattgtgctgacccaaagcccagcaaccctgagcctgtctccaggcgaacgtgccactctgtcctgccgtgcaagccagtctgtcatccactacctggcatggtaccagcagaagccagggcaagccccacgtctgttaatctacgacgcaagcaatcgtgccaccggtattccagcccgtttttctggcagcggttccggcaccgatttcactctgaccatctcctccctggaaccagaagacttcgccgtttactactgtcaacagtatgagaactggccacttacttttggtcagggcaccaaagttgagattaaa

SEQ ID NO: 78 - protein, 113 aa, Artificial Sequence
  Description of Artificial Sequence Synthetic polypeptide
QVQLVESGGGVVQPGRSLRLDCKASGIDFSNSGMHWVRQAPGKGLEWLAVIWYDGSKRYYADSVKGRFTISRDNSKNTLFLQMNSLRAEDTAVYYCASNDDYWGQGTLVTVSS

SEQ ID NO: 79 - protein, 107 aa, Artificial Sequence
  Description of Artificial Sequence Synthetic polypeptide
EIVLTQSPATLSLSPGERATLSCRASQSVIHYLAWYQQKPGQAPRLLIYDASNRATGIPARFSGSGSGTDFTLTISSLEPEDFAVYYCQQYENWPLTFGQGTKVEIK

SEQ ID NO: 80 - DNA, 705 bp, Artificial Sequence
  Description of Artificial Sequence Synthetic polynucleotide
caggtacaattggtagagtccggtggtggcgttgtccagccaggccgttccctgcgtctggactgcaaagcatctggcatcaccttctccaacagcggtatgcactgggtgcgtcaggccccaggcaaagggctggagtgggtcgccgtgatctggtatgatgggtctaaagcgtactatgctgactctgtaaaaggccgtttcaccatcagccgtgacaatagcaagaacaccctgttcttacagatgaattccctgcgtgctgaagataccgccgtatactactgcgctactaacgacgattattggggccaaggtaccctggtcaccgtctcctctggcggtggtggtagcggaggcggaggatcaggtggaggcggcagtgaaattgtgctgacccaaagcccagcaaccctgagcctgtctccaggcgaacgtgccactctgtcctgccgtgcaagccagtctgtctaccactacctggcatggtaccagcagaagccagggcaagccccacgtctgttaatctacgacgcagagaatcgtgccaccggtattccagcccgtttttctggcagcggttccggcaccgatttcactctgaccatctcctccctggaaccagaagacttcgccgtttactactgtcaacagtataacgactggccacttacttttggtcagggcaccaaagttgagattaaa

SEQ ID NO: 81 - protein, 113 aa, Artificial Sequence
  Description of Artificial Sequence Synthetic polypeptide
QVQLVESGGGVVQPGRSLRLDCKASGITFSNSGMHWVRQAPGKGLEWVAVIWYDGSKAYYADSVKGRFTISRDNSKNTLFLQMNSLRAEDTAVYYCATNDDYWGQGTLVTVSS

SEQ ID NO: 82 - protein, 107 aa, Artificial Sequence
  Description of Artificial Sequence Synthetic polypeptide
EIVLTQSPATLSLSPGERATLSCRASQSVYHYLAWYQQKPGQAPRLLIYDAENRATGIPARFSGSGSGTDFTLTISSLEPEDFAVYYCQQYNDWPLTFGQGTKVEIK

SEQ ID NO: 83 - DNA, 705 bp, Artificial Sequence
  Description of Artificial Sequence Synthetic polynucleotide
caggtacaattggtagagtccggtggtggcgttgtccagccaggccgttccctgcgtctggactgcaaagcatctggcatcgacttctccaacagcggtatgcactgggtgcgtcaggccccaggcaaagggctggagtgggtcgccgtgatctggtatgatgggtctaaacgttactatgctgactctgtaaaaggccgtttcaccatcagccgtgacaatagcaagaacaccctgttcttacagatgaattccctgcgtgctgaagataccgccgtatactactgcgcttccaacgacgattattggggccaaggtaccctggtcaccgtctcctctggcggtggtggtagcggaggcggaggatcaggtggaggcggcagtgaaattgtgctgacccaaagcccagcaaccctgagcctgtctccaggcgaacgtgccactctgtcctgccgtgcaagccagtctgtcatccactacctggcatggtaccagcagaagccagggcaagccccacgtctgttaatctacgacgcaagcaatcgtgccaccggtattccagcccgtttttctggcagcggttccggcaccgatttcactctgaccatctcctccctggaaccagaagacttcgccgtttactactgtcaacagtatgacaactggccacttacttttggtcagggcaccaaagttgagattaaa

SEQ ID NO: 84 - protein, 113 aa, Artificial Sequence
  Description of Artificial Sequence Synthetic polypeptide
QVQLVESGGGVVQPGRSLRLDCKASGIDFSNSGMHWVRQAPGKGLEWVAVIWYDGSKRYYADSVKGRFTISRDNSKNTLFLQMNSLRAEDTAVYYCASNDDYWGQGTLVTVSS

SEQ ID NO: 85 - protein, 107 aa, Artificial Sequence; identical to SEQ ID NO: 61
  Description of Artificial Sequence Synthetic polypeptide

SEQ ID NO: 86 - DNA, 705 bp, Artificial Sequence
  Description of Artificial Sequence Synthetic polynucleotide
caggtacaattggtagagtccggtggtggcgttgtccagccaggccgttccctgcgtctggactgcaaagcatctggcatcgacttctccaacagcggtatgcactgggtgcgtcaggccccaggcaaagggctggagtgggtcgccgtgatctggtatgatgggtctaaacgttactatgctgactctgtaaaaggccgtttcaccatcagccgtgacaatagcaagaacaccctgttcttacagatgaattccctgcgtgctgaagataccgccgtatactactgcgcttccaacgacgattattggggccaaggtaccctggtcaccgtctcctctggcggtggtggtagcggaggcggaggatcaggtggaggcggcagtgaaattgtgctgacccaaagcccagcaaccctgagcctgtctccaggcgaacgtgccactctgtcctgccgtgcaagccagtctgtcgagcactacctggcatggtaccagcagaagccagggcaagccccacgtctgttaatctacgacgcaagcaatcccgccaccggtattccagcccgtttttctggcagcggttccggcaccgatttcactctgaccatctcctccctggaaccagaagacttcgccgtttactactgtcaacagtattcaaactggccacttacttttggtcagggcaccaaagttgagattaaa

SEQ ID NO: 87 - protein, 113 aa, Artificial Sequence; identical to SEQ ID NO: 84
  Description of Artificial Sequence Synthetic polypeptide

SEQ ID NO: 88 - protein, 107 aa, Artificial Sequence
  Description of Artificial Sequence Synthetic polypeptide
EIVLTQSPATLSLSPGERATLSCRASQSVEHYLAWYQQKPGQAPRLLIYDASNPATGIPARFSGSGSGTDFTLTISSLEPEDFAVYYCQQYSNWPLTFGQGTKVEIK

SEQ ID NO: 89 - DNA, 705 bp, Artificial Sequence
  Description of Artificial Sequence Synthetic polynucleotide
caggtacaattggtagagtccggtggtggcgttgtccagccaggccgttccctgcgtctggactgcaaagcatctggcatcaccttctccaacagcggtatgcactgggtgcgtcaggccccaggcaaagggctggagtgggtcgccgtgatctggtatgatgggtctctgcgttactatgctgactctgtaaaaggccgtttcaccatcagccgtgacaatagcaagaacaccctgttcttacagatgaattccctgcgtgctgaagataccgccgtatactactgcgctactaacgacgattattggggccaaggtaccctggtcaccgtctcctctggcggtggtggtagcggaggcggaggatcaggtggaggcggcagtgaaattgtgctgacccaaagcccagcaaccctgagcctgtctccaggcgaacgtgccactctgtcctgccgtgcaagccagtctgtctctgacaacctggcgtggtaccagcagaagccagggcaagccccacgtctgttaatctacgacgcagacacccgtgccaccggtattccagcccgtttttctggcagcggttccggcaccgatttcactctgaccatctcctccctggaaccagaagacttcgccgtttactactgttgccagtcatcaaacttaccacgtacttttggtcagggcaccaaagttgagattaaa

SEQ ID NO: 90 - protein, 113 aa, Artificial Sequence; identical to SEQ ID NO: 45
  Description of Artificial Sequence Synthetic polypeptide

SEQ ID NO: 91 - protein, 107 aa, Artificial Sequence
  Description of Artificial Sequence Synthetic polypeptide
EIVLTQSPATLSLSPGERATLSCRASQSVSDNLAWYQQKPGQAPRLLIYDADTRATGIPARFSGSGSGTDFTLTISSLEPEDFAVYYCCQSSNLPRTFGQGTKVEIK

SEQ ID NO: 92 - DNA, 705 bp, Artificial Sequence
  Description of Artificial Sequence Synthetic polynucleotide
caggtacaattggtagagtccggtggtggcgttgtccagccaggccgttccctgcgtctggactgcaaagcatctggcatcaccttctccaacagcggtatgcactgggtgcgtcaggccccaggcaaagggctggagtgggtcgccgtgatctggtatgatgggtctctgcgttactatgctgactctgtaaaaggccgtttcaccatcagccgtgacaatagcaagaacaccctgttcttacagatgaattccctgcgtgctgaagataccgccgtatactactgcgctactaacgacgattattggggccaaggtaccctggtcaccgtctcctctggcggtggtggtagcggaggcggaggatcaggtggaggcggcagtgaaattgtgctgacccaaagcccagcaaccctgagcctgtctccaggcgaacgtgccactctgtcctgccgtgcaagcgagtctgtctctgacaacctgcagtggtaccagcagaagccagggcaagccccacgtctgttaatctacgacgcagacaatcgtgccaccggtattccagcccgtttttctggcagcggttccggcaccgatttcactctgaccatctcctccctggaaccagaagacttcgccgtttactactgtcaacagtcatcaaacttaccacgtacttttggtcagggcaccaaagttgagattaaa

SEQ ID NO: 93 - protein, 113 aa, Artificial Sequence; identical to SEQ ID NO: 45
  Description of Artificial Sequence Synthetic polypeptide

SEQ ID NO: 94 - protein, 107 aa, Artificial Sequence
  Description of Artificial Sequence Synthetic polypeptide
EIVLTQSPATLSLSPGERATLSCRASESVSDNLQWYQQKPGQAPRLLIYDADNRATGIPARFSGSGSGTDFTLTISSLEPEDFAVYYCQQSSNLPRTFGQGTKVEIK

SEQ ID NO: 95 - DNA, 705 bp, Artificial Sequence
  Description of Artificial Sequence Synthetic polynucleotide
caggtacaattggtagagtccggtggtggcgttgtccagccaggccgttccctgcgtctggactgcaaagcatctggcatcaccttctccaacagcggtatgcactgggtgcgtcaggccccaggcaaagggctggagtgggtcgccgtgatctggtatgatgggtctctgcgttactatgctgactctgtaaaaggccgtttcaccatcagccgtgacaatagcaagaacaccctgttcttacagatgaattccctgcgtgctgaagataccgccgtatactactgcgctactaacgacgattattggggccaaggtaccctggtcaccgtctcctctggcggtggtggtagcggaggcggaggatcaggtggaggcggcagtgaaattgtgctgacccaaagcccagcaaccctgagcctgtctccaggcgaacgtgccactctgtcctgccgtgcaagccagtctgtctctgacaacctgcagtggtaccagcagaagccagggcaagccccacgtctgttaatctacgacgcagacacccgtgccaccggtattccagcccgtttttctggcagcggttccggcaccgatttcactctgaccatctcctccctggaaccagaagacttcgccgtttactactgtcaacagtcatcaaacttaccacgtacttttggtcagggcaccaaagttgagattaaa

SEQ ID NO: 96 - protein, 113 aa, Artificial Sequence; identical to SEQ ID NO: 45
  Description of Artificial Sequence Synthetic polypeptide

SEQ ID NO: 97 - protein, 107 aa, Artificial Sequence
  Description of Artificial Sequence Synthetic polypeptide
EIVLTQSPATLSLSPGERATLSCRASQSVSDNLQWYQQKPGQAPRLLIYDADTRATGIPARFSGSGSGTDFTLTISSLEPEDFAVYYCQQSSNLPRTFGQGTKVEIK

SEQ ID NO: 98 - DNA, 705 bp, Artificial Sequence
  Description of Artificial Sequence Synthetic polynucleotide
caggtacaattggtagagtccggtggtggcgttgtccagccaggccgttccctgcgtctggactgcaaagcatctggcttcaccttctccaacagcggtatgcactgggtgcgtcaggccccaggcaaagggctggagtgggtcgccgtgatctggtatgatgggtctctgcgttactatgctgactctgtaaaaggccgtttcaccatcagccgtgacaatagcaagaacaccctgttcttacagatgaattccctgcgtgctgaagataccgccgtatactactgcgctactaacgacgattattggggccaaggtaccctggtcaccgtctcctctggcggtggtggtagcggaggcggaggatcaggtggaggcggcagtgaaattgtgctgacccaaagcccagcaaccctgagcctgtctccaggcgaacgtgccactctgtcctgccgtgcaagccagtctgtctctgacaacctgcagtggtaccagcagaagccagggcaagccccacgtctgttaatctacgacgcagacacgcgtgccaccggtattccagcccgtttttctggcagcggttccggcaccgatttcactctgaccatctcctccctggaaccagaagacttcgccgtttactactgtcaacagtcatcaaacttaccacgtacttttggtcagggcaccaaagttgagattaaa

SEQ ID NO: 99 - protein, 113 aa, Artificial Sequence
  Description of Artificial Sequence Synthetic polypeptide
QVQLVESGGGVVQPGRSLRLDCKASGFTFSNSGMHWVRQAPGKGLEWVAVIWYDGSLRYYADSVKGRFTISRDNSKNTLFLQMNSLRAEDTAVYYCATNDDYWGQGTLVTVSS

SEQ ID NO: 100 - protein, 107 aa, Artificial Sequence; identical to SEQ ID NO: 97
  Description of Artificial Sequence Synthetic polypeptide

SEQ ID NO: 101 - DNA, 705 bp, Artificial Sequence
  Description of Artificial Sequence Synthetic polynucleotide
caggtacaattggtagagtccggtggtggcgttgtccagccaggccgttccctgcgtctggactgcaaagcatctggcatcaccttctccaacagcggtatgcactgggtgcgtcaggccccaggcaaagggctggagtgggtcgccgtgatctggtatgatgggtctctgcgttactatgctgactctgtaaaaggccgtttcaccatcagccgtgacaatagcaagaacaccctgttcttacagatgaattccctgcgtgctgaagataccgccgtatactactgcgctactaacgacgattattggggccaaggtaccctggtcaccgtctcctctggcggtggtggtagcggaggcggaggatcaggtggaggcggcagtgaaattgtgctgacccaaagcccagcaaccctgagcctgtctccaggcgaacgtgccactctgtcctgccgtgcaagccagtctgtctctgacaacctggcatggtaccagcagaagccagggcaagccccacgtctgttaatctacgacgcagacaatcgtgccaccggtattccagcccgtttttctggcagcggttccggcaccgatttcactctgaccatctcctccctggaaccagaagacttcgccgtttactactgtatgcagtcatcaaacttagcccgtacttttggtcagggcaccaaagttgagattaaa

SEQ ID NO: 102 - protein, 113 aa, Artificial Sequence; identical to SEQ ID NO: 45
  Description of Artificial Sequence Synthetic polypeptide

SEQ ID NO: 103 - protein, 107 aa, Artificial Sequence
  Description of Artificial Sequence Synthetic polypeptide
EIVLTQSPATLSLSPGERATLSCRASQSVSDNLAWYQQKPGQAPRLLIYDADNRATGIPARFSGSGSGTDFTLTISSLEPEDFAVYYCMQSSNLARTFGQGTKVEIK

SEQ ID NO: 104 - DNA, 705 bp, Artificial Sequence
  Description of Artificial Sequence Synthetic polynucleotide
caggtacaattggtagagtccggtggtggcgttgtccagccaggccgttccctgcgtctggactgcaaagcatctggcatcgccttctccaacagcggtatgcactgggtgcgtcaggccccaggcaaagggctggagtgggtcgccgtgatctggtatgatgggtctctgcgttactatgctgactctgtaaaaggccgtttcaccatcagccgtgacaatagcaagaacaccctgttcttacagatgaattccctgcgtgctgaagataccgccgtatactactgcgctactaacgacgattattggggccaaggtaccctggtcaccgtctcctctggcggtggtggtagcggaggcggaggatcaggtggaggcggcagtgaaattgtgctgacccaaagcccagcaaccctgagcctgtctccaggagaacgtgccactctgtcctgccgtgcaagccagtctgtctctgacaacctggcatggtaccagcagaagccagggcaagccccacgtctgttaatctacgacgcagacaatcgtgccaccggtattccagcccgtttttctggcagcggttccggcaccgatttcactctgaccatctcctccctggaaccagaagacttcgccgtttactactgttgccagtcatcaaacttaccacgtacttttggtcagggcaccaaagttgagattaaa

SEQ ID NO: 105 - protein, 113 aa, Artificial Sequence
  Description of Artificial Sequence Synthetic polypeptide
QVQLVESGGGVVQPGRSLRLDCKASGIAFSNSGMHWVRQAPGKGLEWVAVIWYDGSLRYYADSVKGRFTISRDNSKNTLFLQMNSLRAEDTAVYYCATNDDYWGQGTLVTVSS

SEQ ID NO: 106 - protein, 107 aa, Artificial Sequence
  Description of Artificial Sequence Synthetic polypeptide
EIVLTQSPATLSLSPGERATLSCRASQSVSDNLAWYQQKPGQAPRLLIYDADNRATGIPARFSGSGSGTDFTLTISSLEPEDFAVYYCCQSSNLPRTFGQGTKVEIK

SEQ ID NO: 107 - DNA, 705 bp, Artificial Sequence
  Description of Artificial Sequence Synthetic polynucleotide
caggtacaattggtagagtccggtggtggcgttgtccagccaggccgttccctgcgtctggactgcaaagcatctggcatcaccttctccaacagcggtatgcactgggtgcgtcaggccccaggcaaagggctggagtgggtcgccgtgatctggtatgatgggtctctgcgttactatgctgactctgtaaaaggccgtttcaccatcagccgtgacaatagcaagaacaccctgttcttacagatgaattccctgcgtgctgaagataccgccgtatactactgcgctactaacgacgattattggggccaaggtaccctggtcaccgtctcctctggcggtggtggtagcggaggcggaggatcaggtggaggcggcagtgaaattgtgctgacccaaagcccagcaaccctgagcctgtctccaggcgaacgtgccactctgtcctgccgtgcaagcctgtctgtctctgacaacctggcatggtaccagcagaagccagggcaagccccacgtctgttaatctacgacgcagacaaccgtgccaccggtattccagcccgtttttctggcagcggttccggcaccgatttcactctgaccatctcctccctggaaccagaagacttcgccgtttactactgtatgcagtcatcaaacttaccacgtacttttggtcagggcaccaaagttgagattaaa

SEQ ID NO: 108 - protein, 113 aa, Artificial Sequence; identical to SEQ ID NO: 45
  Description of Artificial Sequence Synthetic polypeptide

SEQ ID NO: 109 - protein, 107 aa, Artificial Sequence
  Description of Artificial Sequence Synthetic polypeptide
EIVLTQSPATLSLSPGERATLSCRASLSVSDNLAWYQQKPGQAPRLLIYDADNRATGIPARFSGSGSGTDFTLTISSLEPEDFAVYYCMQSSNLPRTFGQGTKVEIK

SEQ ID NO: 110 - protein, 122 aa, Artificial Sequence
  Description of Artificial Sequence Synthetic polypeptide
QMQLVESGGGVVQPGRSLRLSCAASGFTFRTYGMHWVRQAPGKGLEWVAVIWYDGSNKHYADSVKGRFTITRDNSKNTLNLQMNSLRAEDTAVYYCARAPQWELVHEAFDIWGQGTMVTVSS

SEQ ID NO: 111 - protein, 108 aa, Artificial Sequence
  Description of Artificial Sequence Synthetic polypeptide
SYVLTQPPSVSVAPGQTARITCGGNNLGSKSVHWYQQKPGQAPVLVVYDDSDRPSWIPERFSGSNSGNTATLTISRGEAGDEADYYCQVWDSSSDHVVFGGGTKLTVL

SEQ ID NO: 112 - protein, 108 aa, Artificial Sequence
  Description of Artificial Sequence Synthetic polypeptide
SYVLTQPPSVSVAPGQTARITCGGNLIGSKLVHWYQQKPGQAPVLVVYDDSDRPSLIPERFSGSNSGNTATLTISRVEAGDEADYYCQVWDSSSDGVVFGGGTKLTVL

SEQ ID NO: 113 - protein, 122 aa, Artificial Sequence
  Description of Artificial Sequence Synthetic polypeptide
QMQLVESGGGVVQPGRSLRLSCAASGFTFRTYGMHWVRQAPGKGLEWVAVIWYDGSNKHYADSVKGRFTITRDNSKNTLNLQMNSLRAEDTAVYYCARAPQWELTAEAFDIWGQGTMVTVSS

SEQ ID NO: 114 - protein, 108 aa, Artificial Sequence; identical to SEQ ID NO: 112
  Description of Artificial Sequence Synthetic polypeptide

SEQ ID NO: 115 - protein, 122 aa, Artificial Sequence
  Description of Artificial Sequence Synthetic polypeptide
QMQLVESGGGVVQPGRSLRLSCAASGFTFRTYGMHWVRQAPGKGLEWVAVIWYDGSNKHYADSVKGRFTITRDNSKNTLNLQMNSLRAEDTAVYYCARAPQWELVAEAFDLWGQGTMVTVSS

SEQ ID NO: 116 - protein, 108 aa, Artificial Sequence
  Description of Artificial Sequence Synthetic polypeptide
SYVLTQPPSVSVAPGQTARITCGGNLIGSKLVHWYQQKPGQAPVLVVYDDSDRPSLIPERFSGSNSGNTATLTISRVEAGDEADYYCQVWDTSSDGVVFGGGTKLTVL

SEQ ID NO: 117 - protein, 122 aa, Artificial Sequence
  Description of Artificial Sequence Synthetic polypeptide
QMQLVESGGGVVQPGRSLRLSCAASGFTFRTYGMHWVRQAPGKGLEWVAVIWYDGSNKHYADSVKGRFTITRDNSKNTLNLQMNSLRAEDTAVYYCARAPQWQLVAEAFDIWGQGTMVTVSS

SEQ ID NO: 118 - protein, 108 aa, Artificial Sequence
  Description of Artificial Sequence Synthetic polypeptide
SYVLTQPPSVSVAPGQTARITCGGNLIGSKLVHWYQQKPGQAPVLVVYDDSDRPSAIPERFSGSNSGNTATLTISRVEAGDEADYYCQVWDTSSDGVVFGGGTKLTVL

SEQ ID NO: 119 - protein, 122 aa, Artificial Sequence; identical to SEQ ID NO: 117
  Description of Artificial Sequence Synthetic polypeptide

SEQ ID NO: 120 - protein, 108 aa, Artificial Sequence; identical to SEQ ID NO: 116
  Description of Artificial Sequence Synthetic polypeptide

SEQ ID NO: 121 - protein, 122 aa, Artificial Sequence; identical to SEQ ID NO: 115
  Description of Artificial Sequence Synthetic polypeptide

SEQ ID NO: 122 - protein, 108 aa, Artificial Sequence
  Description of Artificial Sequence Synthetic polypeptide
SYVLTQPPSVSVAPGQTARITCGGNLLGSKLVHWYQQKPGQAPVLVVYDDSDRPSAIPERFSGSNSGNTATLTISRVEAGDEADYYCQVWDSSSDHVVFGGGTKLTVL

SEQ ID NO: 123 - protein, 122 aa, Artificial Sequence
  Description of Artificial Sequence Synthetic polypeptide
QMQLVESGGGVVQPGRSLRLSCAASGFTFRTYGMHWVRQAPGKGLEWVAVIWYDGSNKHYADSVKGRFTITRDNSKNTLNLQMNSLRAEDTAVYYCARAPQWELVAEAFDIWGQGTMVTVSS

SEQ ID NO: 124 - protein, 108 aa, Artificial Sequence
  Description of Artificial Sequence Synthetic polypeptide
SYVLTQPPSVSVAPGQTARITCGGNLIGSKLVHWYQQKPGQAPVLVVYDDSDRPSRIPERFSGSNSGNTATLTISRVEAGDEADYYCQVWDSSSDHVVFGGGTKLTVL

SEQ ID NO: 125 - protein, 122 aa, Artificial Sequence
  Description of Artificial Sequence Synthetic polypeptide
QMQLVESGGGVVQPGRSLRLSCAASGFTFRTYGMHWVRQAPGKGLEWVAVIWYDGSNKHYADSVKGRFTITRDNSKNTLNLQMNSLRAEDTAVYYCARAPQWMLVAEAFDLWGQGTMVTVSS

SEQ ID NO: 126 - protein, 108 aa, Artificial Sequence; identical to SEQ ID NO: 124
  Description of Artificial Sequence Synthetic polypeptide

SEQ ID NO: 127 - protein, 122 aa, Artificial Sequence
  Description of Artificial Sequence Synthetic polypeptide
QMQLVESGGGVVQPGRSLRLSCAASGFTFRTYGMHWVRQAPGKGLEWVAVIWYDGSNKHYADSVKGRFTITRDNSKNTLNLQMNSLRAEDTAVYYCARAPQWEWVAEAFDLWGQGTMVTVSS

SEQ ID NO: 128 - protein, 108 aa, Artificial Sequence; identical to SEQ ID NO: 124
  Description of Artificial Sequence Synthetic polypeptide

SEQ ID NO: 129 - protein, 122 aa, Artificial Sequence
  Description of Artificial Sequence Synthetic polypeptide
QMQLVESGGGVVQPGRSLRLSCAASGFTFRTYGMHWVRQAPGKGLEWVAVIWYDGSNKHYADSVKGRFTITRDNSKNTLNLQMNSLRAEDTAVYYCARAPQWELVAEAFDMWGQGTMVTVSS

SEQ ID NO: 130 - protein, 108 aa, Artificial Sequence
  Description of Artificial Sequence Synthetic polypeptide
SYVLTQPPSVSVAPGQTARITCGGNLIGSKLVHWYQQKPGQAPVLVVYDDSDRPSKIPERFSGSNSGNTATLTISRVEAGDEADYYCQVWDSSSDHVVFGGGTKLTVL

SEQ ID NO: 131 - protein, 122 aa, Artificial Sequence; identical to SEQ ID NO: 115
  Description of Artificial Sequence Synthetic polypeptide

SEQ ID NO: 132 - protein, 108 aa, Artificial Sequence
  Description of Artificial Sequence Synthetic polypeptide
SYVLTQPPSVSVAPGQTARITCGGNLIGSKLVHWYQQKPGQAPVLVVYDDSDRPSDIPERFSGSNSGNTATLTISRVEAGDEADYYCQVWDTSSDHVVFGGGTKLTVL

SEQ ID NO: 133 - protein, 122 aa, Artificial Sequence
  Description of Artificial Sequence Synthetic polypeptide
QMQLVESGGGVVQPGRSLRLSCAASGFTFRTYGMHWVRQAPGKGLEWVAVIWYDGSNKHYADSVKGRFTITRDNSKNTLNLQMNSLRAEDTAVYYCARAPQWEYVAEAFDLWGQGTMVTVSS

SEQ ID NO: 134 - protein, 108 aa, Artificial Sequence
  Description of Artificial Sequence Synthetic polypeptide
SYVLTQPPSVSVAPGQTARITCGGNIIGSKLVHWYQQKPGQAPVLVVYDDGDRPSGIPERFSGSNSGNTATLTISRVEAGDEADYYCQVWDTSSDHVVFGGGTKLTVL

SEQ ID NO: 135 - protein, 122 aa, Artificial Sequence
  Description of Artificial Sequence Synthetic polypeptide
QMQLVESGGGVVQPGRSLRLSCAASGFTFRTYGMHWVRQAPGKGLEWVAVIWYDGSNKHYADSVKGRFTITRDNSKNTLNLQMNSLRAEDTAVYYCARAPQWELTAEAFDLWGQGTMVTVSS

SEQ ID NO: 136 - protein, 108 aa, Artificial Sequence
  Description of Artificial Sequence Synthetic polypeptide
SYVLTQPPSVSVAPGQTARITCGGNLLGSKLVHWYQQKPGQAPVLVVYDDGDRPSLIPERFSGSNSGNTATLTISRVEAGDEADYYCQVWDTSSDHVVFGGGTKLTVL

SEQ ID NO: 137 - protein, 122 aa, Artificial Sequence; identical to SEQ ID NO: 115
  Description of Artificial Sequence Synthetic polypeptide

SEQ ID NO: 138 - protein, 108 aa, Artificial Sequence
  Description of Artificial Sequence Synthetic polypeptide
SYVLTQPPSVSVAPGQTARITCGGNLLGSKLVHWYQQKPGQAPVLVVYDDSDRPSEIPERFSGSNSGNTATLTISRVEAGDEADYYCQVWDTSSDGVVFGGGTKLTVL

SEQ ID NO: 139 - protein, 122 aa, Artificial Sequence; identical to SEQ ID NO: 113
  Description of Artificial Sequence Synthetic polypeptide

SEQ ID NO: 140 - protein, 108 aa, Artificial Sequence
  Description of Artificial Sequence Synthetic polypeptide
SYVLTQPPSVSVAPGQTARITCGGNLLGSKLVHWYQQKPGQAPVLVVYDDSDRPSWIPERFSGSNSGNTATLTISRVEAGDEADYYCQVWDTSSDHVVFGGGTKLTVL

SEQ ID NO: 141 - protein, 122 aa, Artificial Sequence; identical to SEQ ID NO: 135
  Description of Artificial Sequence Synthetic polypeptide

SEQ ID NO: 142 - protein, 108 aa, Artificial Sequence
  Description of Artificial Sequence Synthetic polypeptide
SYVLTQPPSVSVAPGQTARITCGGNLIGSKLVHWYQQKPGQAPVLVVYDDSDRPSAIPERFSGSNSGNTATLTISRVEAGDEADYYCQVWDTSSDHVVFGGGTKLTVL

SEQ ID NO: 143 - protein, 122 aa, Artificial Sequence
  Description of Artificial Sequence Synthetic polypeptide
QMQLVESGGGVVQPGRSLRLSCAASGFTFRTYGMHWVRQAPGKGLEWVAVIWYDGSNKHYADSVKGRFTITRDNSKNTLNLQMNSLRAEDTAVYYCARAPQWELTSEAFDLWGQGTMVTVSS

SEQ ID NO: 144 - protein, 108 aa, Artificial Sequence
  Description of Artificial Sequence Synthetic polypeptide
SYVLTQPPSVSVAPGQTARITCGGNLIGSKLVHWYQQKPGQAPVLVVYDDSDRPSEIPERFSGSNSGNTATLTISRVEAGDEADYYCQVWDSSSDGVVFGGGTKLTVL

SEQ ID NO: 145 - protein, 122 aa, Artificial Sequence; identical to SEQ ID NO: 143
  Description of Artificial Sequence Synthetic polypeptide

SEQ ID NO: 146 - protein, 108 aa, Artificial Sequence
  Description of Artificial Sequence Synthetic polypeptide
SYVLTQPPSVSVAPGQTARITCGGNIIGSKLVHWYQQKPGQAPVLVVYDDSDRPSGIPERFSGSNSGNTATLTISRVEAGDEADYYCQVWDSGSDHVVFGGGTKLTVL

SEQ ID NO: 147 - protein, 122 aa, Artificial Sequence
  Description of Artificial Sequence Synthetic polypeptide
QMQLVESGGGVVQPGRSLRLSCAASGFTFRTYGMHWVRQAPGKGLEWVAVIWYDGSNKHYADSVKGRFTITRDNSKNTLNLQMNSLRAEDTAVYYCARAPQWLLVAEAFDLWGQGTMVTVSS

SEQ ID NO: 148 - protein, 108 aa, Artificial Sequence
  Description of Artificial Sequence Synthetic polypeptide
SYVLTQPPSVSVAPGQTARITCGGNIIGSKLVHWYQQKPGQAPVLVVYDDSDRPSDIPERFSGSNSGNTATLTISRVEAGDEADYYCQVWDSGSDGVVFGGGTKLTVL

SEQ ID NO: 149 - protein, 122 aa, Artificial Sequence; identical to SEQ ID NO: 115
  Description of Artificial Sequence Synthetic polypeptide

SEQ ID NO: 150 - protein, 108 aa, Artificial Sequence
  Description of Artificial Sequence Synthetic polypeptide
SYVLTQPPSVSVAPGQTARITCGGNLIGSKLVHWYQQKPGQAPVLVVYDDGDRPSWIPERFSGSNSGNTATLTISRVEAGDEADYYCQVWDSGSDGVVFGGGTKLTVL

SEQ ID NO: 151 - protein, 122 aa, Artificial Sequence; identical to SEQ ID NO: 115
  Description of Artificial Sequence Synthetic polypeptide

SEQ ID NO: 152 - protein, 108 aa, Artificial Sequence
  Description of Artificial Sequence Synthetic polypeptide
SYVLTQPPSVSVAPGQTARITCGGNLIGSKLVHWYQQKPGQAPVLVVYDDSDRPSRIPERFSGSNSGNTATLTISRVEAGDEADYYCQVWDSGSDGVVFGGGTKLTVL

SEQ ID NO: 153 - protein, 122 aa, Artificial Sequence
  Description of Artificial Sequence Synthetic polypeptide
QMQLVESGGGVVQPGRSLRLSCAASGFTFRTYGMHWVRQAPGKGLEWVAVIWYDGSNKHYADSVKGRFTITRDNSKNTLNLQMNSLRAEDTAVYYCARAPQWVLVSEAFDLWGQGTMVTVSS

SEQ ID NO: 154 - protein, 108 aa, Artificial Sequence
  Description of Artificial Sequence Synthetic polypeptide
SYVLTQPPSVSVAPGQTARITCGGNIIGSKLVHWYQQKPGQAPVLVVYDDSDRPSAIPERFSGSNSGNTATLTISRVEAGDEADYYCQVWDSSSDGVVFGGGTKLTVL

SEQ ID NO: 155 - protein, 122 aa, Artificial Sequence; identical to SEQ ID NO: 115
  Description of Artificial Sequence Synthetic polypeptide

SEQ ID NO: 156 - protein, 108 aa, Artificial Sequence
  Description of Artificial Sequence Synthetic polypeptide
SYVLTQPPSVSVAPGQTARITCGGNNIGSKLVHWYQQKPGQAPVLVVYDDSDRPSGIPERFSGSNSGNTATLTISRVEAGDEADYYCQVWDSSSDGVVFGGGTKLTVL

SEQ ID NO: 157 - protein, 122 aa, Artificial Sequence
  Description of Artificial Sequence Synthetic polypeptide
QMQLVESGGGVVQPGRSLRLSCAASGFTFRTYGMHWVRQAPGKGLEWVAVISYDGSNKHYADSVKGRFTITRDNSKNTLNLQMNSLRAEDTAVYYCARSPQWEWVHEAFDMWGQGTMVTVSS

SEQ ID NO: 158 - protein, 108 aa, Artificial Sequence
  Description of Artificial Sequence Synthetic polypeptide
SYVLTQPPSVSVAPGQTARITCGGNILGSKLVHWYQQKPGQAPVLVVYDDSDRPSEIPERFSGSNSGNTATLTISRVEAGDEADYYCQVWDTSSDGVVFGGGTKLTVL

SEQ ID NO: 159 - protein, 122 aa, Artificial Sequence
  Description of Artificial Sequence Synthetic polypeptide
QMQLVESGGGVVQPGRSLRLSCAASGFTFRTYGMHWVRQAPGKGLEWVAVISYDGSNKHYADSVKGRFTITRDNSKNTLNLQMNSLRAEDTAVYYCARSPQWEWVHEAFDLWGQGTMVTVSS

SEQ ID NO: 160 - protein, 108 aa, Artificial Sequence
  Description of Artificial Sequence Synthetic polypeptide
SYVLTQPPSVSVAPGQTARITCGGNNIGSKLVHWYQQKPGQAPVLVVYDDSDRPSRIPERFSGSNSGNTATLTISRVEAGDEADYYCQVWDTSSDGVVFGGGTKLTVL

SEQ ID NO: 161 - protein, 122 aa, Artificial Sequence
  Description of Artificial Sequence Synthetic polypeptide
QMQLVESGGGVVQPGRSLRLSCAASGFTFRTYGMHWVRQAPGKGLEWVAVIWYDGSNKHYADSVKGRFTITRDNSKNTLNLQMNSLRAEDTAVYYCARSPQWEWVHEAFDLWGQGTMVTVSS

SEQ ID NO: 162 - protein, 108 aa, Artificial Sequence
  Description of Artificial Sequence Synthetic polypeptide
SYVLTQPPSVSVAPGQTARITCGGNLIGSKLVHWYQQKPGQAPVLVVYDDGDRPSWIPERFSGSNSGNTATLTISRVEAGDEADYYCQVWDSSSDGVVFGGGTKLTVL

SEQ ID NO: 163 - protein, 122 aa, Artificial Sequence; identical to SEQ ID NO: 161
  Description of Artificial Sequence Synthetic polypeptide

SEQ ID NO: 164 - DNA, 366 bp, Artificial Sequence
  Description of Artificial Sequence Synthetic polynucleotide
caaatgcagctggtcgagtctggcggtggggtagtgcaaccaggccgttctctgcgtcttagctgcgccgcatctggttttacctttcgtacctacggtatgcactgggtgcgtcaggcaccaggcaaaggtctggaatgggtcgcagtaatctggtatgatggtagcaataaacactatgctgactcagtcaaaggccgtttcaccatcacccgtgataacagcaagaacactcttaacttacagatgaactctctgcgtgccgaagacaccgcagtttattactgtgcccgtgcaccacagtgggaattagtacacgaagcattcgatatctggggtcagggtactatggtgaccgttagctct

SEQ ID NO: 165 - DNA, 324 bp, Artificial Sequence
  Description of Artificial Sequence Synthetic polynucleotide
tcttacgtgctgactcaaccaccatcagtgtctgtagcaccaggccagaccgcacgtattacctgtggcggtaacaatctgggctctaagtctgttcactggtatcagcaaaaaccaggccaggcaccagtactggttgtgtacgatgattccgatcgtccaagctggattccagagcgtttcagcggctctaattccggcaacaccgctactctgactatttcccgtggggaagccggcgatgaagccgactactattgccaggtctgggactcttcttccgaccatgtagtctttggcgggggcaccaaactgaccgttttg

SEQ ID NO: 166 - DNA, 366 bp, Artificial Sequence
  Description of Artificial Sequence Synthetic polynucleotide
caaatgcagctggtcgagtctggcggtggggtagtgcaaccaggccgttctctgcgtcttagctgcgccgcatctggttttacctttcgtacctacggtatgcactgggtgcgtcaggcaccaggcaaaggtctggaatgggtcgcagtaatctggtatgatggtagcaataaacactatgctgactcagtcaaaggccgtttcaccatcacccgtgataacagcaagaacactcttaacttacagatgaactctctgcgtgccgaagacaccgccgtttactactgtgcccgtgcaccacagtgggaattaacggcggaagcattcgatatttggggccagggcactatggtgaccgttagctct

SEQ ID NO: 167 - DNA, 324 bp, Artificial Sequence
  Description of Artificial Sequence Synthetic polynucleotide
tcttacgtgctgactcaaccaccatcagtgtctgtagcaccaggccagaccgcacgtattacctgtggcggtaacctgatcggctctaagctggttcactggtatcagcaaaaaccaggccaggcaccagtactggttgtgtacgatgatagcgatcgtccaagcctgattccagagcgtttcagcggctctaattccggcaacaccgctactctgactatttcccgtgttgaagccggcgatgaagccgactactattgccaggtctgggactctagctccgacggtgtagtctttggcgggggcaccaaactgaccgttttg

SEQ ID NO: 168 - DNA, 366 bp, Artificial Sequence
  Description of Artificial Sequence Synthetic polynucleotide
caaatgcagctggtcgagtctggcggtggggtagtgcaaccaggccgttctctgcgtcttagctgcgccgcatctggttttacctttcgtacctacggtatgcactgggtgcgtcaggcaccaggcaaaggtctggaatgggtcgcagtaatctggtatgatggtagcaataaacactatgctgactcagtcaaaggccgtttcaccatcacccgtgataacagcaagaacactcttaacttacagatgaactctctgcgtgccgaagacaccgccgtttactactgtgcccgtgcaccacagtgggaattagtagcggaagcattcgatctgtggggccagggcactatggtgaccgttagctct

SEQ ID NO: 169 - DNA, 324 bp, Artificial Sequence; identical to SEQ ID NO: 167
  Description of Artificial Sequence Synthetic polynucleotide

SEQ ID NO: 170 - DNA, 366 bp, Artificial Sequence
  Description of Artificial Sequence Synthetic polynucleotide
caaatgcagctggtcgagtctggcggtggggtagtgcaaccaggccgttctctgcgtcttagctgcgccgcatctggttttacctttcgtacctacggtatgcactgggtgcgtcaggcaccaggcaaaggtctggaatgggtcgcagtaatctggtatgatggtagcaataaacactatgctgactcagtcaaaggccgtttcaccatcacccgtgataacagcaagaacactcttaacttacagatgaactctctgcgtgccgaagacaccgccgtttactactgtgcccgtgcaccacagtggcagttagtagcggaagcattcgatatttggggccagggcactatggtgaccgttagctct

SEQ ID NO: 171 - DNA, 324 bp, Artificial Sequence
  Description of Artificial Sequence Synthetic polynucleotide
tcttacgtgctgactcaaccaccatcagtgtctgtagcaccaggccagaccgcacgtattacctgtggcggtaacctgatcggctctaagctggttcactggtatcagcaaaaaccaggccaggcaccagtactggttgtgtacgatgatagcgatcgtccaagccttattccagagcgtttcagcggctctaattccggcaacaccgctactctgactatttcccgtgttgaagccggcgatgaagccgactactattgccaggtctgggacaccagctccgacggtgtagtctttggcgggggcaccaaactgaccgttttg

SEQ ID NO: 172 - DNA, 366 bp, Artificial Sequence
  Description of Artificial Sequence Synthetic polynucleotide
caaatgcagctggtcgagtctggcggtggggtagtgcaaccaggccgttctctgcgtcttagctgcgccgcatctggttttacctttcgtacctacggtatgcactgggtgcgtcaggcaccaggcaaaggtctggaatgggtcgcagtaatctggtatgatggtagcaataaacactatgctgactcagtcaaaggccgtttcaccatcacccgtgataacagcaagaacactcttaacttacagatgaactctctgcgtgccgaagacaccgccgtttactactgtgcccgtgcaccacagtggcagttagtagccgaagcattcgatatatggggccagggcactatggtgaccgttagctct

SEQ ID NO: 173 - DNA, 324 bp, Artificial Sequence
  Description of Artificial Sequence Synthetic polynucleotide
tcttacgtgctgactcaaccaccatcagtgtctgtagcaccaggccagaccgcacgtattacctgtggcggtaacctgatcggctctaagctggttcactggtatcagcaaaaaccaggccaggcaccagtactggttgtgtacgatgatagcgatcgtccaagcgctattccagagcgtttcagcggctctaattccggcaacaccgctactctgactatttcccgtgttgaagccggcgatgaagccgactactattgccaggtctgggacactagctccgacggtgtagtctttggcgggggcaccaaactgaccgttttg

SEQ ID NO: 174 - DNA, 366 bp, Artificial Sequence
  Description of Artificial Sequence Synthetic polynucleotide
caaatgcagctggtcgagtctggcggtggggtagtgcaaccaggccgttctctgcgtcttagctgcgccgcatctggttttacctttcgtacctacggtatgcactgggtgcgtcaggcaccaggcaaaggtctggaatgggtcgcagtaatctggtatgatggtagcaataaacactatgctgactcagtcaaaggccgtttcaccatcacccgtgataacagcaagaacactcttaacttacagatgaactctctgcgtgccgaagacaccgccgtttactactgtgcccgtgcaccacagtgggaattagtggcggaagcattcgatctttggggccagggcactatggtgaccgttagctct

SEQ ID NO: 175 - DNA, 324 bp, Artificial Sequence; identical to SEQ ID NO: 171
  Description of Artificial Sequence Synthetic polynucleotide

SEQ ID NO: 176 - DNA, 366 bp, Artificial Sequence
  Description of Artificial Sequence Synthetic polynucleotide
caaatgcagctggtcgagtctggcggtggggtagtgcaaccaggccgttctctgcgtcttagctgcgccgcatctggttttacctttcgtacctacggtatgcactgggtgcgtcaggcaccaggcaaaggtctggaatgggtcgcagtaatctggtatgatggtagcaataaacactatgctgactcagtcaaaggccgtttcaccatcacccgtgataacagcaagaacactcttaacttacagatgaactctctgcgtgccgaagacaccgccgtttactactgtgcccgtgcaccacagtgggaattggtagccgaagcattcgatatctggggccagggcactatggtgaccgttagctct

SEQ ID NO: 177 - DNA, 324 bp, Artificial Sequence
  Description of Artificial Sequence Synthetic polynucleotide
tcttacgtgctgactcaaccaccatcagtgtctgtagcaccaggccagaccgcacgtattacctgtggcggtaacctgctgggctctaagctggttcactggtatcagcaaaaaccaggccaggcaccagtactggttgtgtacgatgatagcgatcgtccaagcgcaattccagagcgtttcagcggctctaattccggcaacaccgctactctgactatttcccgtgttgaagccggcgatgaagccgactactattgccaggtctgggactctagctccgaccacgtagtctttggcgggggcaccaaactgaccgttttg

SEQ ID NO: 178 - DNA, 366 bp, Artificial Sequence
  Description of Artificial Sequence Synthetic polynucleotide
caaatgcagctggtcgagtctggcggtggggtagtgcaaccaggccgttctctgcgtcttagctgcgccgcatctggttttacctttcgtacctacggtatgcactgggtgcgtcaggcaccaggcaaaggtctggaatgggtcgcagtaatctggtatgatggtagcaataaacactatgctgactcagtcaaaggccgtttcaccatcacccgtgataacagcaagaacactcttaacttacagatgaactctctgcgtgccgaagacaccgccgtttactactgtgcccgtgcaccacagtggatgttagtagcggaagcattcgatctatggggccagggcactatggtgaccgttagctct

SEQ ID NO: 179 - DNA, 324 bp, Artificial Sequence
  Description of Artificial Sequence Synthetic polynucleotide
tcttacgtgctgactcaaccaccatcagtgtctgtagcaccaggccagaccgcacgtattacctgtggcggtaacctgatcggctctaagctggttcactggtatcagcaaaaaccagggcaggcaccagtactggttgtgtacgatgatagcgatcgtccaagccggattccagagcgtttcagcggctctaattccggcaacaccgctactctgactatttcccgtgttgaagccggcgatgaagccgactactattgccaggtctgggactccagctccgaccatgtagtctttggcgggggcaccaaactgaccgttttg

SEQ ID NO: 180 - DNA, 366 bp, Artificial Sequence
  Description of Artificial Sequence Synthetic polynucleotide
caaatgcagctggtcgagtctggcggtggggtagtgcaaccaggccgttctctgcgtcttagctgcgccgcatctggttttacctttcgtacctacggtatgcactgggtgcgtcaggcaccaggcaaaggtctggaatgggtcgcagtaatctggtatgatggtagcaataaacactatgctgactcagtcaaaggccgtttcaccatcacccgtgataacagcaagaacactcttaacttacagatgaactctctgcgtgccgaagacaccgccgtttactactgtgcccgtgcaccacagtgggaatgggtagccgaagcattcgatctgtggggccagggcactatggtgaccgttagctct

SEQ ID NO: 181 - DNA, 324 bp, Artificial Sequence
  Description of Artificial Sequence Synthetic polynucleotide
tcttacgtgctgactcaaccaccatcagtgtctgtagcaccaggccagaccgcacgtattacctgtggcggtaacctgatcggctctaagctggttcactggtatcagcaaaaaccaggccaggcaccagtactggttgtgtacgatgatagcgatcgtccaagccggattccagagcgtttcagcggctctaattccggcaacaccgctactctgactatttcccgtgttgaagccggcgatgaagccgactactattgccaggtctgggactccagctccgaccacgtagtctttggcgggggcaccaaactgaccgttttg

SEQ ID NO: 182 - DNA, 366 bp, Artificial Sequence
  Description of Artificial Sequence Synthetic polynucleotide
caaatgcagctggtcgagtctggcggtggggtagtgcaaccaggccgttctctgcgtcttagctgcgccgcatctggttttacctttcgtacctacggtatgcactgggtgcgtcaggcaccaggcaaaggtctggaatgggtcgcagtaatctggtatgatggtagcaataaacactatgctgactcagtcaaaggccgtttcaccatcacccgtgataacagcaagaacactcttaacttacagatgaactctctgcgtgccgaagacaccgccgtttactactgtgcccgtgcaccacagtgggaattagtagccgaggcattcgatatgtggggccagggcactatggtgaccgttagctct

SEQ ID NO: 183 - DNA, 324 bp, Artificial Sequence
  Description of Artificial Sequence Synthetic polynucleotide
tcttacgtgctgactcaaccaccatcagtgtctgtagcaccaggccagaccgcacgtattacctgtggcggtaacctgatcggctctaagctggttcactggtatcagcaaaaaccaggccaggcaccagtactggttgtgtacgatgatagcgatcgtccaagccggattccagagcgtttcagcggctctaattccggcaacaccgctactctgactatttcccgtgttgaagccggcgatgaagccgactactattgccaggtctgggactctagctccgaccatgtagtctttggcgggggcaccaaactgaccgttttg

SEQ ID NO: 184 - DNA, 366 bp, Artificial Sequence
  Description of Artificial Sequence Synthetic polynucleotide
caaatgcagctggtcgagtctggcggtggggtagtgcaaccaggccgttctctgcgtcttagctgcgccgcatctggttttacctttcgtacctacggtatgcactgggtgcgtcaggcaccaggcaaaggtctggaatgggtcgcagtaatctggtatgatggtagcaataaacactatgctgactcagtcaaaggccgtttcaccatcacccgtgataacagcaagaacactcttaacttacagatgaactctctgcgtgccgaagacaccgccgtttactactgtgcccgtgcaccacagtgggaattagtagccgaagcattcgatctgtggggccagggcactatggtgaccgttagctct

SEQ ID NO: 185 - DNA, 324 bp, Artificial Sequence
  Description of Artificial Sequence Synthetic polynucleotide
tcttacgtgctgactcaaccaccatcagtgtctgtagcaccaggccagaccgcacgtattacctgtggcggtaacctgatcggctctaagctggttcactggtatcagcaaaaaccaggccaggcaccagtactggttgtgtacgatgatagcgatcgtccaagcaaaattccagagcgtttcagcggctctaattccggcaacaccgctactctgactatttcccgtgttgaagccggcgatgaagccgactactattgccaggtctgggactccagctccgaccatgtagtctttggcgggggcaccaaactgaccgttttg

SEQ ID NO: 186 - DNA, 366 bp, Artificial Sequence
  Description of Artificial Sequence Synthetic polynucleotide
caaatgcagctggtcgagtctggcggtggggtagtgcaaccaggccgttctctgcgtcttagctgcgccgcatctggttttacctttcgtacctacggtatgcactgggtgcgtcaggcaccaggcaaaggtctggaatgggtcgcagtaatctggtatgatggtagcaataaacactatgctgactcagtcaaaggccgtttcaccatcacccgtgataacagcaagaacactcttaacttacagatgaactctctgcgtgccgaagacaccgccgtttactactgtgcccgtgcaccacagtgggaatacgtagcggaagcattcgatctgtggggccagggcactatggtgaccgttagctct

SEQ ID NO: 187 - DNA, 324 bp, Artificial Sequence
  Description of Artificial Sequence Synthetic polynucleotide
tcttacgtgctgactcaaccaccatcagtgtctgtagcaccaggccagaccgcacgtattacctgtggcggtaacctgatcggctctaagctggttcactggtatcagcaaaaaccaggccaggcaccagtactggttgtgtacgatgatagcgatcgtccaagcgatattccagagcgtttcagcggctctaattccggcaacaccgctactctgactatttcccgtgttgaagccggcgatgaagccgactactattgccaggtctgggacaccagctccgaccatgtagtctttggcgggggcaccaaactgaccgttttg

SEQ ID NO: 188 - DNA, 366 bp, Artificial Sequence
  Description of Artificial Sequence Synthetic polynucleotide
caaatgcagctggtcgagtctggcggtggggtagtgcaaccaggccgttctctgcgtcttagctgcgccgcatctggttttacctttcgtacctacggtatgcactgggtgcgtcaggcaccaggcaaaggtctggaatgggtcgcagtaatctggtatgatggtagcaataaacactatgctgactcagtcaaaggccgtttcaccatcacccgtgataacagcaagaacactcttaacttacagatgaactctctgcgtgccgaagacaccgccgtttactactgtgcccgtgcaccacagtgggaattaacggccgaagcattcgatctttggggccagggcactatggtgaccgttagctct

SEQ ID NO: 189 - DNA, 324 bp, Artificial Sequence
  Description of Artificial Sequence Synthetic polynucleotide
tcttacgtgctgactcaaccaccatcagtgtctgtagcaccaggccagaccgcacgtattacctgtggcggtaacatcatcggctctaagctggttcactggtatcagcaaaaaccaggccaggcaccagtactggttgtgtacgatgatggcgatcgtccaagcggtattccagagcgtttcagcggctctaattccggcaacaccgctactctgactatttcccgtgttgaagccggcgatgaagccgactactattgccaggtctgggacactagctccgaccacgtagtctttggcgggggcaccaaactgaccgttttg

SEQ ID NO: 190 - DNA, 366 bp, Artificial Sequence; identical to SEQ ID NO: 174
  Description of Artificial Sequence Synthetic polynucleotide

SEQ ID NO: 191 - DNA, 324 bp, Artificial Sequence
  Description of Artificial Sequence Synthetic polynucleotide
tcttacgtgctgactcaaccaccatcagtgtctgtagcaccaggccagaccgcacgtattacctgtggcggtaacctgctgggctctaagctggttcactggtatcagcaaaaaccaggccaggcaccagtactggttgtgtacgatgatggcgatcgtccaagcctgattccagagcgtttcagcggctctaattccggcaacaccgctactctgactatttcccgtgttgaagccggcgatgaagccgactactattgccaggtctgggacactagctccgaccacgtagtctttggcgggggcaccaaactgaccgttttg

SEQ ID NO: 192 - DNA, 366 bp, Artificial Sequence; identical to SEQ ID NO: 166
  Description of Artificial Sequence Synthetic polynucleotide

SEQ ID NO: 193 - DNA, 324 bp, Artificial Sequence
  Description of Artificial Sequence Synthetic polynucleotide
tcttacgtgctgactcaaccaccatcagtgtctgtagcaccaggccagaccgcacgtattacctgtggcggtaacctgctgggctctaagctggttcactggtatcagcaaaaaccaggccaggcaccagtactggttgtgtacgatgatagcgatcgtccaagcgaaattccagagcgtttcagcggctctaattccggcaacaccgctactctgactatttcccgtgttgaagccggcgatgaagccgactactattgccaggtctgggacaccagctccgacggtgtagtctttggcgggggcaccaaactgaccgttttg

SEQ ID NO: 194 - DNA, 366 bp, Artificial Sequence; identical to SEQ ID NO: 188
  Description of Artificial Sequence Synthetic polynucleotide

SEQ ID NO: 195 - DNA, 324 bp, Artificial Sequence
  Description of Artificial Sequence Synthetic polynucleotide
tcttacgtgctgactcaaccaccatcagtgtctgtagcaccaggccagaccgcacgtattacctgtggcggtaacctgctgggctctaagctggttcactggtatcagcaaaaaccaggccaggcaccagtactggttgtgtacgatgatagcgatcgtccaagctggattccagagcgtttcagcggctctaattccggcaacaccgctactctgactatttcccgtgttgaagccggcgatgaagccgactactattgccaggtctgggacactagctccgaccatgtagtctttggcgggggcaccaaactgaccgttttg

SEQ ID NO: 196 - DNA, 366 bp, Artificial Sequence
  Description of Artificial Sequence Synthetic polynucleotide
caaatgcagctggtcgagtctggcggtggggtagtgcaaccaggccgttctctgcgtcttagctgcgccgcatctggttttacctttcgtacctacggtatgcactgggtgcgtcaggcaccaggcaaaggtctggaatgggtcgcagtaatctggtatgatggtagcaataaacactatgctgactcagtcaaaggccgtttcaccatcacccgtgataacagcaagaacactcttaacttacagatgaactctctgcgtgccgaagacaccgccgtttactactgtgcccgtgcaccacagtgggaattaacgtcggaagcattcgatctttggggccagggcactatggtgaccgttagctct

SEQ ID NO: 197 - DNA, 324 bp, Artificial Sequence
  Description of Artificial Sequence Synthetic polynucleotide
tcttacgtgctgactcaaccaccatcagtgtctgtagcaccaggccagaccgcacgtattacctgtggcggtaacctgatcggctctaagctggttcactggtatcagcaaaaaccaggccaggcaccagtactggttgtgtacgatgatagcgatcgtccaagcgcaattccagagcgtttcagcggctctaattccggcaacaccgctactctgactatttcccgtgttgaagccggcgatgaagccgactactattgccaggtctgggacaccagctccgaccacgtagtctttggcgggggcaccaaactgaccgttttg

SEQ ID NO: 198 - DNA, 366 bp, Artificial Sequence
  Description of Artificial Sequence Synthetic polynucleotide
caaatgcagctggtcgagtctggcggtggggtagtgcaaccaggccgttctctgcgtcttagctgcgccgcatctggttttacctttcgtacctacggtatgcactgggtgcgtcaggcaccaggcaaaggtctggaatgggtcgcagtaatctggtatgatggtagcaataaacactatgctgactcagtcaaaggccgtttcaccatcacccgtgataacagcaagaacactcttaacttacagatgaactctctgcgtgccgaagacaccgccgtttactactgtgcccgtgcaccacagtgggaattaacctccgaagcattcgatctttggggccagggcactatggtgaccgttagctct

SEQ ID NO: 199 - DNA, 324 bp, Artificial Sequence
  Description of Artificial Sequence Synthetic polynucleotide
tcttacgtgctgactcaaccaccatcagtgtctgtagcaccaggccagaccgcacgtattacctgtggcggtaacctgatcggctctaagctggttcactggtatcagcaaaaaccaggccaggcaccagtactggttgtgtacgatgatagcgatcgtccaagcgaaattccagagcgtttcagcggctctaattccggcaacaccgctactctgactatttcccgtgttgaagccggcgatgaagccgactactattgccaggtctgggactccagctccgacggtgtagtctttggcgggggcaccaaactgaccgttttg

SEQ ID NO: 200 - DNA, 366 bp, Artificial Sequence
  Description of Artificial Sequence Synthetic polynucleotide
caaatgcagctggtcgagtctggcggtggggtagtgcaaccaggccgttctctgcgtcttagctgcgccgcatctggttttacctttcgtacctacggtatgcactgggtgcgtcaggcaccaggcaaaggtctggaatgggtcgcagtaatctggtatgatggtagcaataaacactatgctgactcagtcaaaggccgtttcaccatcacccgtgataacagcaagaacactcttaacttacagatgaactctctgcgtgccgaagacaccgccgtttactactgtgcccgtgcaccacagtggctgttagtagcggaagcattcgatctctggggccagggcactatggtgaccgttagctct

SEQ ID NO: 201 - DNA, 324 bp, Artificial Sequence
  Description of Artificial Sequence Synthetic polynucleotide
tcttacgtgctgactcaaccaccatcagtgtctgtagcaccaggccagaccgcacgtattacctgtggcggtaacatcatcggctctaagctggttcactggtatcagcaaaaaccaggccaggcaccagtactggttgtgtacgatgatagcgatcgtccaagcggtattccagagcgtttcagcggctctaattccggcaacaccgctactctgactatttcccgtgttgaagccggcgatgaagccgactactattgccaggtctgggactctggctccgaccacgtagtctttggcgggggcaccaaactgaccgttttg

SEQ ID NO: 202 - DNA, 366 bp, Artificial Sequence
  Description of Artificial Sequence Synthetic polynucleotide
caaatgcagctggtcgagtctggcggtggggtagtgcaaccaggccgttctctgcgtcttagctgcgccgcatctggttttacctttcgtacctacggtatgcactgggtgcgtcaggcaccaggcaaaggtctggaatgggtcgcagtaatctggtatgatggtagcaataaacactatgctgactcagtcaaaggccgtttcaccatcacccgtgataacagcaagaacactcttaacttacagatgaactctctgcgtgccgaagacaccgccgtttactactgtgcccgtgcaccacagtgggaattagtagcggaagcattcgatctttggggccagggcactatggtgaccgttagctct

SEQ ID NO: 203 - DNA, 324 bp, Artificial Sequence
  Description of Artificial Sequence Synthetic polynucleotide
tcttacgtgctgactcaaccaccatcagtgtctgtagcaccaggccagaccgcacgtattacctgtggcggtaacatcatcggctctaagctggttcactggtatcagcaaaaaccaggccaggcaccagtactggttgtgtacgatgatagcgatcgtccaagcgatattccagagcgtttcagcggctctaattccggcaacaccgctactctgactatttcccgtgttgaagccggcgatgaagccgactactattgccaggtctgggactccggctccgacggtgtagtctttggcgggggcaccaaactgaccgttttg

SEQ ID NO: 204 - DNA, 366 bp, Artificial Sequence; identical to SEQ ID NO: 168
  Description of Artificial Sequence Synthetic polynucleotide

SEQ ID NO: 205 - DNA, 324 bp, Artificial Sequence
  Description of Artificial Sequence Synthetic polynucleotide
tcttacgtgctgactcaaccaccatcagtgtctgtagcaccaggccagaccgcacgtattacctgtggcggtaacctgatcggctctaagctggttcactggtatcagcaaaaaccaggccaggcaccagtactggttgtgtacgatgatggcgatcgtccaagctggattccagagcgtttcagcggctctaattccggcaacaccgctactctgactatttcccgtgttgaagccggcgatgaagccgactactattgccaggtctgggactctggctccgacggtgtagtctttggcgggggcaccaaactgaccgttttg

SEQ ID NO: 206 - DNA, 366 bp, Artificial Sequence
  Description of Artificial Sequence Synthetic polynucleotide
caaatgcagctggtcgagtctggcggtggggtagtgcaaccaggccgttctctgcgtcttagctgcgccgcatctggttttacctttcgtacctacggtatgcactgggtgcgtcaggcaccaggcaaaggtctggaatgggtcgcagtaatctggtatgatggtagcaataaacactatgctgactcagtcaaaggccgtttcaccatcacccgtgataacagcaagaacactcttaacttacagatgaactctctgcgtgccgaagacaccgccgtttactactgtgcccgtgcaccacagtgggtcttagtatccgaagcattcgatctttggggccagggcactatggtgaccgttagctct

SEQ ID NO: 207 - DNA, 324 bp, Artificial Sequence
  Description of Artificial Sequence Synthetic polynucleotide
tcttacgtgctgactcaaccaccatcagtgtctgtagcaccaggccagaccgcacgtattacctgtggcggtaacctgatcggctctaagctggttcactggtatcagcaaaaaccaggccaggcaccagtactggttgtgtacgatgatagcgatcgtccaagccgtattccagagcgtttcagcggctctaattccggcaacaccgctactctgactatttcccgtgttgaagccggcgatgaagccgactactattgccaggtctgggactccggctccgacggtgtagtctttggcgggggcaccaaactgaccgttttg

SEQ ID NO: 208 - DNA, 366 bp, Artificial Sequence
  Description of Artificial Sequence Synthetic polynucleotide
caaatgcagctggtcgagtctggcggtggggtagtgcaaccaggccgttctctgcgtcttagctgcgccgcatctggttttacctttcgtacctacggtatgcactgggtgcgtcaggcaccaggcaaaggtctggaatgggtcgcagtaatctggtatgatggtagcaataaacactatgctgactcagtcaaaggccgtttcaccatcacccgtgataacagcaagaacactcttaacttacagatgaactctctgcgtgccgaagacaccgccgtttactactgtgcccgtgcaccacagtgggaattagtagcggaggcattcgatctgtggggccagggcactatggtgaccgttagctct

SEQ ID NO: 209 - DNA, 324 bp, Artificial Sequence
  Description of Artificial Sequence Synthetic polynucleotide
tcttacgtgctgactcaaccaccatcagtgtctgtagcaccaggccagaccgcacgtattacctgtggcggtaacatcatcggctctaagctggttcactggtatcagcaaaaaccaggccaggcaccagtactggttgtgtacgatgatagcgatcgtccaagcgcaattccagagcgtttcagcggctctaattccggcaacaccgctactctgactatttcccgtgttgaagccggcgatgaagccgactactattgccaggtctgggactccagctccgacggtgtagtctttggcgggggcaccaaactgaccgttttg

SEQ ID NO: 210 - DNA, 366 bp, Artificial Sequence
  Description of Artificial Sequence Synthetic polynucleotide
caaatgcagctggtcgagtctggcggtggggtagtgcaaccaggccgttctctgcgtcttagctgcgccgcatctggttttacctttcgtacctacggtatgcactgggtgcgtcaggcaccaggcaaaggtctggaatgggtcgcagtaatctcttatgatggtagcaataaacactatgctgactcagtcaaaggccgtttcaccatcacccgtgataacagcaagaacactcttaacttacagatgaactctctgcgtgccgaagacaccgccgtttactactgtgcccgtagcccacagtgggaatgggtacacgaagcattcgatatgtggggccagggcactatggtgaccgttagctct

SEQ ID NO: 211 - DNA, 324 bp, Artificial Sequence
  Description of Artificial Sequence Synthetic polynucleotide
tcttacgtgctgactcaaccaccatcagtgtctgtagcaccaggccagaccgcacgtattacctgtggcggtaacaacatcggctctaagctggttcactggtatcagcaaaaaccaggccaggcaccagtactggttgtgtacgatgatagcgatcgtccaagcggtattccagagcgtttcagcggctctaattccggcaacaccgctactctgactatttcccgtgttgaagccggcgatgaagccgactactattgccaggtctgggactctagctccgacggtgtagtctttggcgggggcaccaaactgaccgttttg

SEQ ID NO: 212 - DNA, 366 bp, Artificial Sequence
  Description of Artificial Sequence Synthetic polynucleotide
caaatgcagctggtcgagtctggcggtggggtagtgcaaccaggccgttctctgcgtcttagctgcgccgcatctggttttacctttcgtacctacggtatgcactgggtgcgtcaggcaccaggcaaaggtctggaatgggtcgcagtaatctcttatgatggtagcaataaacactatgctgactcagtcaaaggccgtttcaccatcacccgtgataacagcaagaacactcttaacttacagatgaactctctgcgtgccgaagacaccgccgtttactactgtgcccgttcgccacagtgggaatgggtacacgaagcattcgatctctggggccagggcactatggtgaccgttagctct

SEQ ID NO: 213 - DNA, 324 bp, Artificial Sequence
  Description of Artificial Sequence Synthetic polynucleotide
tcttacgtgctgactcaaccaccatcagtgtctgtagcaccaggccagaccgcacgtattacctgtggcggtaacatcctgggctctaagctggttcactggtatcagcaaaaaccaggccaggcaccagtactggttgtgtacgatgatagcgatcgtccaagcgaaattccagagcgtttcagcggctctaattccggcaacaccgctactctgactatttcccgtgttgaagccggcgatgaagccgactactattgccaggtctgggacaccagctccgacggtgtagtctttggcgggggcaccaaactgaccgttttg

SEQ ID NO: 214 - DNA, 366 bp, Artificial Sequence
  Description of Artificial Sequence Synthetic polynucleotide
caaatgcagctggtcgagtctggcggtggggtagtgcaaccaggccgttctctgcgtcttagctgcgccgcatctggttttacctttcgtacctacggtatgcactgggtgcgtcaggcaccaggcaaaggtctggaatgggtcgcagtaatctggtatgatggtagcaataaacactatgctgactcagtcaaaggccgtttcaccatcacccgtgataacagcaagaacactcttaacttacagatgaactctctgcgtgccgaagacaccgccgtttactactgtgcccgtagcccacagtgggaatgggtacacgaagcattcgatctatggggccagggcactatggtgaccgttagctct

SEQ ID NO: 215 - DNA, 324 bp, Artificial Sequence
  Description of Artificial Sequence Synthetic polynucleotide
tcttacgtgctgactcaaccaccatcagtgtctgtagcaccaggccagaccgcacgtattacctgtggcggtaacaacatcggctctaagctggttcactggtatcagcaaaaaccaggccaggcaccagtactggttgtgtacgatgatagcgatcgtccaagccggattccagagcgtttcagcggctctaattccggcaacaccgctactctgactatttcccgtgttgaagccggcgatgaagccgactactattgccaggtctgggacactagctccgacggtgtagtctttggcgggggcaccaaactgaccgttttg

SEQ ID NO: 216 - DNA, 366 bp, Artificial Sequence
  Description of Artificial Sequence Synthetic polynucleotide
caaatgcagctggtcgagtctggcggtggggtagtgcaaccaggccgttctctgcgtcttagctgcgccgcatctggttttacctttcgtacctacggtatgcactgggtgcgtcaggcaccaggcaaaggtctggaatgggtcgcagtaatctggtatgatggtagcaataaacactatgctgactcagtcaaaggccgtttcaccatcacccgtgataacagcaagaacactcttaacttacagatgaactctctgcgtgccgaagacaccgccgtttactactgtgcccgttcgccacagtgggaatgggtacacgaagcattcgatctctggggccagggcactatggtgaccgttagctct

SEQ ID NO: 217 - DNA, 324 bp, Artificial Sequence
  Description of Artificial Sequence Synthetic polynucleotide
tcttacgtgctgactcaaccaccatcagtgtctgtagcaccaggccagaccgcacgtattacctgtggcggtaacctgatcggctctaagctggttcactggtatcagcaaaaaccaggccaggcaccagtactggttgtgtacgatgatggcgatcgtccaagctggattccagagcgtttcagcggctctaattccggcaacaccgctactctgactatttcccgtgttgaagccggcgatgaagccgactactattgccaggtctgggactccagctccgacggtgtagtctttggcgggggcaccaaactgaccgttttg

SEQ ID NO: 218 - DNA, 735 bp, Artificial Sequence
  Description of Artificial Sequence Synthetic polynucleotide
caaatgcagctggtcgagtctggcggtggggtagtgcaaccaggccgttctctgcgtcttagctgcgccgcatctggttttacctttcgtacctacggtatgcactgggtgcgtcaggcaccaggcaaaggtctggaatgggtcgcagtaatctggtatgatggtagcaataaacactatgctgactcagtcaaaggccgtttcaccatcacccgtgataacagcaagaacactcttaacttacagatgaactctctgcgtgccgaagacaccgcagtttattactgtgcccgtgcaccacagtgggaattagtacacgaagcattcgatatctggggtcagggtactatggtgaccgttagctctggcggtggtggtagcggaggcggaggatcaggtggaggcggcagttcttacgtgctgactcaaccaccatcagtgtctgtagcaccaggccagaccgcacgtattacctgtggcggtaacaatctgggctctaagtctgttcactggtatcagcaaaaaccaggccaggcaccagtactggttgtgtacgatgattccgatcgtccaagctggattccagagcgtttcagcggctctaattccggcaacaccgctactctgactatttcccgtggggaagccggcgatgaagccgactactattgccaggtctgggactcttcttccgaccatgtagtctttggcgggggcaccaaactgaccgttttg

SEQ ID NO: 219 - DNA, 735 bp, Artificial Sequence
  Description of Artificial Sequence Synthetic polynucleotide
caaatgcagctggtcgagtctggcggtggggtagtgcaaccaggccgttctctgcgtcttagctgcgccgcatctggttttacctttcgtacctacggtatgcactgggtgcgtcaggcaccaggcaaaggtctggaatgggtcgcagtaatctggtatgatggtagcaataaacactatgctgactcagtcaaaggccgtttcaccatcacccgtgataacagcaagaacactcttaacttacagatgaactctctgcgtgccgaagacaccgccgtttactactgtgcccgtgcaccacagtgggaattaacggcggaagcattcgatatttggggccagggcactatggtgaccgttagctctggcggtggtggtagcggaggcggaggatcaggtggaggcggcagttcttacgtgctgactcaaccaccatcagtgtctgtagcaccaggccagaccgcacgtattacctgtggcggtaacctgatcggctctaagctggttcactggtatcagcaaaaaccaggccaggcaccagtactggttgtgtacgatgatagcgatcgtccaagcctgattccagagcgtttcagcggctctaattccggcaacaccgctactctgactatttcccgtgttgaagccggcgatgaagccgactactattgccaggtctgggactctagctccgacggtgtagtctttggcgggggcaccaaactgaccgttttg

SEQ ID NO: 220 - DNA, 735 bp, Artificial Sequence
  Description of Artificial Sequence Synthetic polynucleotide
caaatgcagctggtcgagtctggcggtggggtagtgcaaccaggccgttctctgcgtcttagctgcgccgcatctggttttacctttcgtacctacggtatgcactgggtgcgtcaggcaccaggcaaaggtctggaatgggtcgcagtaatctggtatgatggtagcaataaacactatgctgactcagtcaaaggccgtttcaccatcacccgtgataacagcaagaacactcttaacttacagatgaactctctgcgtgccgaagacaccgccgtttactactgtgcccgtgcaccacagtgggaattagtagcggaagcattcgatctgtggggccagggcactatggtgaccgttagctctggcggtggtggtagcggaggcggaggatcaggtggaggcggcagttcttacgtgctgactcaaccaccatcagtgtctgtagcaccaggccagaccgcacgtattacctgtggcggtaacctgatcggctctaagctggttcactggtatcagcaaaaaccaggccaggcaccagtactggttgtgtacgatgatagcgatcgtccaagcctgattccagagcgtttcagcggctctaattccggcaacaccgctactctgactatttcccgtgttgaagccggcgatgaagccgactactattgccaggtctgggactctagctccgacggtgtagtctttggcgggggcaccaaactgaccgttttg

SEQ ID NO: 221 - DNA, 735 bp, Artificial Sequence
  Description of Artificial Sequence Synthetic polynucleotide
caaatgcagctggtcgagtctggcggtggggtagtgcaaccaggccgttctctgcgtcttagctgcgccgcatctggttttacctttcgtacctacggtatgcactgggtgcgtcaggcaccaggcaaaggtctggaatgggtcgcagtaatctggtatgatggtagcaataaacactatgctgactcagtcaaaggccgtttcaccatcacccgtgataacagcaagaacactcttaacttacagatgaactctctgcgtgccgaagacaccgccgtttactactgtgcccgtgcaccacagtggcagttagtagcggaagcattcgatatttggggccagggcactatggtgaccgttagctctggcggtggtggtagcggaggcggaggatcaggtggaggcggcagttcttacgtgctgactcaaccaccatcagtgtctgtagcaccaggccagaccgcacgtattacctgtggcggtaacctgatcggctctaagctggttcactggtatcagcaaaaaccaggccaggcaccagtactggttgtgtacgatgatagcgatcgtccaagccttattccagagcgtttcagcggctctaattccggcaacaccgctactctgactatttcccgtgttgaagccggcgatgaagccgactactattgccaggtctgggacaccagctccgacggtgtagtctttggcgggggcaccaaactgaccgttttg

SEQ ID NO: 222 - DNA, 735 bp, Artificial Sequence
  Description of Artificial Sequence Synthetic polynucleotide
caaatgcagctggtcgagtctggcggtggggtagtgcaaccaggccgttctctgcgtcttagctgcgccgcatctggttttacctttcgtacctacggtatgcactgggtgcgtcaggcaccaggcaaaggtctggaatgggtcgcagtaatctggtatgatggtagcaataaacactatgctgactcagtcaaaggccgtttcaccatcacccgtgataacagcaagaacactcttaacttacagatgaactctctgcgtgccgaagacaccgccgtttactactgtgcccgtgcaccacagtggcagttagtagccgaagcattcgatatatggggccagggcactatggtgaccgttagctctggcggtggtggtagcggaggcggaggatcaggtggaggcggcagttcttacgtgctgactcaaccaccatcagtgtctgtagcaccaggccagaccgcacgtattacctgtggcggtaacctgatcggctctaagctggttcactggtatcagcaaaaaccaggccaggcaccagtactggttgtgtacgatgatagcgatcgtccaagcgctattccagagcgtttcagcggctctaattccggcaacaccgctactctgactatttcccgtgttgaagccggcgatgaagccgactactattgccaggtctgggacactagctccgacggtgtagtctttggcgggggcaccaaactgaccgttttg

SEQ ID NO: 223 - DNA, 735 bp, Artificial Sequence
  Description of Artificial Sequence Synthetic polynucleotide
caaatgcagctggtcgagtctggcggtggggtagtgcaaccaggccgttctctgcgtcttagctgcgccgcatctggttttacctttcgtacctacggtatgcactgggtgcgtcaggcaccaggcaaaggtctggaatgggtcgcagtaatctggtatgatggtagcaataaacactatgctgactcagtcaaaggccgtttcaccatcacccgtgataacagcaagaacactcttaacttacagatgaactctctgcgtgccgaagacaccgccgtttactactgtgcccgtgcaccacagtgggaattagtggcggaagcattcgatctttggggccagggcactatggtgaccgttagctctggtggtggtggtagcggaggcggaggatcaggtggaggcggcagttcttacgtgctgactcaaccaccatcagtgtctgtagcaccaggccagaccgcacgtattacctgtggcggtaacctgatcggctctaagctggttcactggtatcagcaaaaaccaggccaggcaccagtactggttgtgtacgatgatagcgatcgtccaagccttattccagagcgtttcagcggctctaattccggcaacaccgctactctgactatttcccgtgttgaagccggcgatgaagccgactactattgccaggtctgggacaccagctccgacggtgtagtctttggcgggggcaccaaactgaccgttttg

SEQ ID NO: 224 - DNA, 735 bp, Artificial Sequence
  Description of Artificial Sequence Synthetic polynucleotide
caaatgcagctggtcgagtctggcggtggggtagtgcaaccaggccgttctctgcgtcttagctgcgccgcatctggttttacctttcgtacctacggtatgcactgggtgcgtcaggcaccaggcaaaggtctggaatgggtcgcagtaatctggtatgatggtagcaataaacactatgctgactcagtcaaaggccgtttcaccatcacccgtgataacagcaagaacactcttaacttacagatgaactctctgcgtgccgaagacaccgccgtttactactgtgcccgtgcaccacagtgggaattggtagccgaagcattcgatatctggggccagggcactatggtgaccgttagctctggcggtggtggtagcggaggcggaggatcaggtggaggcggcagttcttacgtgctgactcaaccaccatcagtgtctgtagcaccaggccagaccgcacgtattacctgtggcggtaacctgctgggctctaagctggttcactggtatcagcaaaaaccaggccaggcaccagtactggttgtgtacgatgatagcgatcgtccaagcgcaattccagagcgtttcagcggctctaattccggcaacaccgctactctgactatttcccgtgttgaagccggcgatgaagccgactactattgccaggtctgggactctagctccgaccacgtagtctttggcgggggcaccaaactgaccgttttg

SEQ ID NO: 225 - DNA, 735 bp, Artificial Sequence
  Description of Artificial Sequence Synthetic polynucleotide
caaatgcagctggtcgagtctggcggtggggtagtgcaaccaggccgttctctgcgtcttagctgcgccgcatctggttttacctttcgtacctacggtatgcactgggtgcgtcaggcaccaggcaaaggtctggaatgggtcgcagtaatctggtatgatggtagcaataaacactatgctgactcagtcaaaggccgtttcaccatcacccgtgataacagcaagaacactcttaacttacagatgaactctctgcgtgccgaagacaccgccgtttactactgtgcccgtgcaccacagtggatgttagtagcggaagcattcgatctatggggccagggcactatggtgaccgttagctctggcggtggtggtagcggaggcggaggatcaggtggaggcggcagttcttacgtgctgactcaaccaccatcagtgtctgtagcaccaggccagaccgcacgtattacctgtggcggtaacctgatcggctctaagctggttcactggtatcagcaaaaaccagggcaggcaccagtactggttgtgtacgatgatagcgatcgtccaagccggattccagagcgtttcagcggctctaattccggcaacaccgctactctgactatttcccgtgttgaagccggcgatgaagccgactactattgccaggtctgggactccagctccgaccatgtagtctttggcgggggcaccaaactgaccgttttg

SEQ ID NO: 226 - DNA, 735 bp, Artificial Sequence
  Description of Artificial Sequence Synthetic polynucleotide
caaatgcagctggtcgagtctggcggtggggtagtgcaaccaggccgttctctgcgtcttagctgcgccgcatctggttttacctttcgtacctacggtatgcactgggtgcgtcaggcaccaggcaaaggtctggaatgggtcgcagtaatctggtatgatggtagcaataaacactatgctgactcagtcaaaggccgtttcaccatcacccgtgataacagcaagaacactcttaacttacagatgaactctctgcgtgccgaagacaccgccgtttactactgtgcccgtgcaccacagtgggaatgggtagccgaagcattcgatctgtggggccagggcactatggtgaccgttagctctggcggtggtggtagcggaggcggaggatcaggtggaggcggcagttcttacgtgctgactcaaccaccatcagtgtctgtagcaccaggccagaccgcacgtattacctgtggcggtaacctgatcggctctaagctggttcactggtatcagcaaaaaccaggccaggcaccagtactggttgtgtacgatgatagcgatcgtccaagccggattccagagcgtttcagcggctctaattccggcaacaccgctactctgactatttcccgtgttgaagccggcgatgaagccgactactattgccaggtctgggactccagctccgaccacgtagtctttggcgggggcaccaaactgaccgttttg

SEQ ID NO: 227 - DNA, 735 bp, Artificial Sequence
  Description of Artificial Sequence Synthetic polynucleotide
caaatgcagctggtcgagtctggcggtggggtagtgcaaccaggccgttctctgcgtcttagctgcgccgcatctggttttacctttcgtacctacggtatgcactgggtgcgtcaggcaccaggcaaaggtctggaatgggtcgcagtaatctggtatgatggtagcaataaacactatgctgactcagtcaaaggccgtttcaccatcacccgtgataacagcaagaacactcttaacttacagatgaactctctgcgtgccgaagacaccgccgtttactactgtgcccgtgcaccacagtgggaattagtagccgaggcattcgatatgtggggccagggcactatggtgaccgttagctctggcggtggtggtagcggaggcggaggatcaggtggaggcggcagttcttacgtgctgactcaaccaccatcagtgtctgtagcaccaggccagaccgcacgtattacctgtggcggtaacctgatcggctctaagctggttcactggtatcagcaaaaaccaggccaggcaccagtactggttgtgtacgatgatagcgatcgtccaagccggattccagagcgtttcagcggctctaattccggcaacaccgctactctgactatttcccgtgttgaagccggcgatgaagccgactactattgccaggtctgggactctagctccgaccatgtagtctttggcgggggcaccaaactgaccgttttg

SEQ ID NO: 228 - DNA, 735 bp, Artificial Sequence
  Description of Artificial Sequence Synthetic polynucleotide
caaatgcagctggtcgagtctggcggtggggtagtgcaaccaggccgttctctgcgtcttagctgcgccgcatctggttttacctttcgtacctacggtatgcactgggtgcgtcaggcaccaggcaaaggtctggaatgggtcgcagtaatctggtatgatggtagcaataaacactatgctgactcagtcaaaggccgtttcaccatcacccgtgataacagcaagaacactcttaacttacagatgaactctctgcgtgccgaagacaccgccgtttactactgtgcccgtgcaccacagtgggaattagtagccgaagcattcgatctgtggggccagggcactatggtgaccgttagctctggcggtggtggtagcggaggcggaggatcaggtggaggcggcagttcttacgtgctgactcaaccaccatcagtgtctgtagcaccaggccagaccgcacgtattacctgtggcggtaacctgatcggctctaagctggttcactggtatcagcaaaaaccaggccaggcaccagtactggttgtgtacgatgatagcgatcgtccaagcaaaattccagagcgtttcagcggctctaattccggcaacaccgctactctgactatttcccgtgttgaagccggcgatgaagccgactactattgccaggtctgggactccagctccgaccatgtagtctttggcgggggcaccaaactgaccgttttg

SEQ ID NO: 229 - DNA, 735 bp, Artificial Sequence
  Description of Artificial Sequence Synthetic polynucleotide
caaatgcagctggtcgagtctggcggtggggtagtgcaaccaggccgttctctgcgtcttagctgcgccgcatctggttttacctttcgtacctacggtatgcactgggtgcgtcaggcaccaggcaaaggtctggaatgggtcgcagtaatctggtatgatggtagcaataaacactatgctgactcagtcaaaggccgtttcaccatcacccgtgataacagcaagaacactcttaacttacagatgaactctctgcgtgccgaagacaccgccgtttactactgtgcccgtgcaccacagtgggaatacgtagcggaagcattcgatctgtggggccagggcactatggtgaccgttagctctggcggtggtggtagcggaggcggaggatcaggtggaggcggcagttcttacgtgctgactcaaccaccatcagtgtctgtagcaccaggccagaccgcacgtattacctgtggcggtaacctgatcggctctaagctggttcactggtatcagcaaaaaccaggccaggcaccagtactggttgtgtacgatgatagcgatcgtccaagcgatattccagagcgtttcagcggctctaattccggcaacaccgctactctgactatttcccgtgttgaagccggcgatgaagccgactactattgccaggtctgggacaccagctccgaccatgtagtctttggcgggggcaccaaactgaccgttttg

SEQ ID NO: 230 - DNA, 735 bp, Artificial Sequence
  Description of Artificial Sequence Synthetic polynucleotide
caaatgcagctggtcgagtctggcggtggggtagtgcaaccaggccgttctctgcgtcttagctgcgccgcatctggttttacctttcgtacctacggtatgcactgggtgcgtcaggcaccaggcaaaggtctggaatgggtcgcagtaatctggtatgatggtagcaataaacactatgctgactcagtcaaaggccgtttcaccatcacccgtgataacagcaagaacactcttaacttacagatgaactctctgcgtgccgaagacaccgccgtttactactgtgcccgtgcaccacagtgggaattaacggccgaagcattcgatctttggggccagggcactatggtgaccgttagctctggcggtggtggtagcggaggcggaggatcaggtggaggcggcagttcttacgtgctgactcaaccaccatcagtgtctgtagcaccaggccagaccgcacgtattacctgtggcggtaacatcatcggctctaagctggttcactggtatcagcaaaaaccaggccaggcaccagtactggttgtgtacgatgatggcgatcgtccaagcggtattccagagcgtttcagcggctctaattccggcaacaccgctactctgactatttcccgtgttgaagccggcgatgaagccgactactattgccaggtctgggacactagctccgaccacgtagtctttggcgggggcaccaaactgaccgttttg

SEQ ID NO: 231 - DNA, 735 bp, Artificial Sequence
  Description of Artificial Sequence Synthetic polynucleotide
caaatgcagctggtcgagtctggcggtggggtagtgcaaccaggccgttctctgcgtcttagctgcgccgcatctggttttacctttcgtacctacggtatgcactgggtgcgtcaggcaccaggcaaaggtctggaatgggtcgcagtaatctggtatgatggtagcaataaacactatgctgactcagtcaaaggccgtttcaccatcacccgtgataacagcaagaacactcttaacttacagatgaactctctgcgtgccgaagacaccgccgtttactactgtgcccgtgcaccacagtgggaattagtggcggaagcattcgatctttggggccagggcactatggtgaccgttagctctggcggtggtggtagcggaggcggaggatcaggtggaggcggcagttcttacgtgctgactcaaccaccatcagtgtctgtagcaccaggccagaccgcacgtattacctgtggcggtaacctgctgggctctaagctggttcactggtatcagcaaaaaccaggccaggcaccagtactggttgtgtacgatgatggcgatcgtccaagcctgattccagagcgtttcagcggctctaattccggcaacaccgctactctgactatttcccgtgttgaagccggcgatgaagccgactactattgccaggtctgggacactagctccgaccacgtagtctttggcgggggcaccaaactgaccgttttg

SEQ ID NO: 232 - DNA, 735 bp, Artificial Sequence
  Description of Artificial Sequence Synthetic polynucleotide
caaatgcagctggtcgagtctggcggtggggtagtgcaaccaggccgttctctgcgtcttagctgcgccgcatctggttttacctttcgtacctacggtatgcactgggtgcgtcaggcaccaggcaaaggtctggaatgggtcgcagtaatctggtatgatggtagcaataaacactatgctgactcagtcaaaggccgtttcaccatcacccgtgataacagcaagaacactcttaacttacagatgaactctctgcgtgccgaagacaccgccgtttactactgtgcccgtgcaccacagtgggaattaacggcggaagcattcgatatttggggccagggcactatggtgaccgttagctctggcggtggtggtagcggaggcggaggatcaggtggaggcggcagttcttacgtgctgactcaaccaccatcagtgtctgtagcaccaggccagaccgcacgtattacctgtggcggtaacctgctgggctctaagctggttcactggtatcagcaaaaaccaggccaggcaccagtactggttgtgtacgatgatagcgatcgtccaagcgaaattccagagcgtttcagcggctctaattccggcaacaccgctactctgactatttcccgtgttgaagccggcgatgaagccgactactattgccaggtctgggacaccagctccgacggtgtagtctttggcgggggcaccaaactgaccgttttg

SEQ ID NO: 233 - DNA, 735 bp, Artificial Sequence
  Description of Artificial Sequence Synthetic polynucleotide
caaatgcagctggtcgagtctggcggtggggtagtgcaaccaggccgttctctgcgtcttagctgcgccgcatctggttttacctttcgtacctacggtatgcactgggtgcgtcaggcaccaggcaaaggtctggaatgggtcgcagtaatctggtatgatggtagcaataaacactatgctgactcagtcaaaggccgtttcaccatcacccgtgataacagcaagaacactcttaacttacagatgaactctctgcgtgccgaagacaccgccgtttactactgtgcccgtgcaccacagtgggaattaacggccgaagcattcgatctttggggccagggcactatggtgaccgttagctctggcggtggtggtagcggaggcggaggatcaggtggaggcggcagttcttacgtgctgactcaaccaccatcagtgtctgtagcaccaggccagaccgcacgtattacctgtggcggtaacctgctgggctctaagctggttcactggtatcagcaaaaaccaggccaggcaccagtactggttgtgtacgatgatagcgatcgtccaagctggattccagagcgtttcagcggctctaattccggcaacaccgctactctgactatttcccgtgttgaagccggcgatgaagccgactactattgccaggtctgggacactagctccgaccatgtagtctttggcgggggcaccaaactgaccgttttg

SEQ ID NO: 234 - DNA, 735 bp, Artificial Sequence
  Description of Artificial Sequence Synthetic polynucleotide
caaatgcagctggtcgagtctggcggtggggtagtgcaaccaggccgttctctgcgtcttagctgcgccgcatctggttttacctttcgtacctacggtatgcactgggtgcgtcaggcaccaggcaaaggtctggaatgggtcgcagtaatctggtatgatggtagcaataaacactatgctgactcagtcaaaggccgtttcaccatcacccgtgataacagcaagaacactcttaacttacagatgaactctctgcgtgccgaagacaccgccgtttactactgtgcccgtgcaccacagtgggaattaacgtcggaagcattcgatctttggggccagggcactatggtgaccgttagctctggcggtggtggtagcggaggcggaggatcaggtggaggcggcagttcttacgtgctgactcaaccaccatcagtgtctgtagcaccaggccagaccgcacgtattacctgtggcggtaacctgatcggctctaagctggttcactggtatcagcaaaaaccaggccaggcaccagtactggttgtgtacgatgatagcgatcgtccaagcgcaattccagagcgtttcagcggctctaattccggcaacaccgctactctgactatttcccgtgttgaagccggcgatgaagccgactactattgccaggtctgggacaccagctccgaccacgtagtctttggcgggggcaccaaactgaccgttttg

SEQ ID NO: 235 - DNA, 735 bp, Artificial Sequence
  Description of Artificial Sequence Synthetic polynucleotide
caaatgcagctggtcgagtctggcggtggggtagtgcaaccaggccgttctctgcgtcttagctgcgccgcatctggttttacctttcgtacctacggtatgcactgggtgcgtcaggcaccaggcaaaggtctggaatgggtcgcagtaatctggtatgatggtagcaataaacactatgctgactcagtcaaaggccgtttcaccatcacccgtgataacagcaagaacactcttaacttacagatgaactctctgcgtgccgaagacaccgccgtttactactgtgcccgtgcaccacagtgggaattaacctccgaagcattcgatctttggggccagggcactatggtgaccgttagctctggcggtggtggtagcggaggcggaggatcaggtggaggcggcagttcttacgtgctgactcaaccaccatcagtgtctgtagcaccaggccagaccgcacgtattacctgtggcggtaacctgatcggctctaagctggttcactggtatcagcaaaaaccaggccaggcaccagtactggttgtgtacgatgatagcgatcgtccaagcgaaattccagagcgtttcagcggctctaattccggcaacaccgctactctgactatttcccgtgttgaagccggcgatgaagccgactactattgccaggtctgggactccagctccgacggtgtagtctttggcgggggcaccaaactgaccgttttg

SEQ ID NO: 236 - DNA, 735 bp, Artificial Sequence
  Description of Artificial Sequence Synthetic polynucleotide
caaatgcagctggtcgagtctggcggtggggtagtgcaaccaggccgttctctgcgtcttagctgcgccgcatctggttttacctttcgtacctacggtatgcactgggtgcgtcaggcaccaggcaaaggtctggaatgggtcgcagtaatctggtatgatggtagcaataaacactatgctgactcagtcaaaggccgtttcaccatcacccgtgataacagcaagaacactcttaacttacagatgaactctctgcgtgccgaagacaccgccgtttactactgtgcccgtgcaccacagtggctgttagtagcggaagcattcgatctctggggccagggcactatggtgaccgttagctctggcggtggtggtagcggaggcggaggatcaggtggaggcggcagttcttacgtgctgactcaaccaccatcagtgtctgtagcaccaggccagaccgcacgtattacctgtggcggtaacatcatcggctctaagctggttcactggtatcagcaaaaaccaggccaggcaccagtactggttgtgtacgatgatagcgatcgtccaagcggtattccagagcgtttcagcggctctaattccggcaacaccgctactctgactatttcccgtgttgaagccggcgatgaagccgactactattgccaggtctgggactctggctccgaccacgtagtctttggcgggggcaccaaactgaccgttttg

SEQ ID NO: 237 - DNA, 735 bp, Artificial Sequence
  Description of Artificial Sequence Synthetic polynucleotide
caaatgcagctggtcgagtctggcggtggggtagtgcaaccaggccgttctctgcgtcttagctgcgccgcatctggttttacctttcgtacctacggtatgcactgggtgcgtcaggcaccaggcaaaggtctggaatgggtcgcagtaatctggtatgatggtagcaataaacactatgctgactcagtcaaaggccgtttcaccatcacccgtgataacagcaagaacactcttaacttacagatgaactctctgcgtgccgaagacaccgccgtttactactgtgcccgtgcaccacagtgggaattagtagcggaagcattcgatctttggggccagggcactatggtgaccgttagctctggcggtggtggtagcggaggcggaggatcaggtggaggcggcagttcttacgtgctgactcaaccaccatcagtgtctgtagcaccaggccagaccgcacgtattacctgtggcggtaacatcatcggctctaagctggttcactggtatcagcaaaaaccaggccaggcaccagtactggttgtgtacgatgatagcgatcgtccaagcgatattccagagcgtttcagcggctctaattccggcaacaccgctactctgactatttcccgtgttgaagccggcgatgaagccgactactattgccaggtctgggactccggctccgacggtgtagtctttggcgggggcaccaaactgaccgttttg

SEQ ID NO: 238 - DNA, 735 bp, Artificial Sequence
  Description of Artificial Sequence Synthetic polynucleotide
caaatgcagctggtcgagtctggcggtggggtagtgcaaccaggccgttctctgcgtcttagctgcgccgcatctggttttacctttcgtacctacggtatgcactgggtgcgtcaggcaccaggcaaaggtctggaatgggtcgcagtaatctggtatgatggtagcaataaacactatgctgactcagtcaaaggccgtttcaccatcacccgtgataacagcaagaacactcttaacttacagatgaactctctgcgtgccgaagacaccgccgtttactactgtgcccgtgcaccacagtgggaattagtagcggaagcattcgatctgtggggccagggcactatggtgaccgttagctctggcggtggtggtagcggaggcggaggatcaggtggaggcggcagttcttacgtgctgactcaaccaccatcagtgtctgtagcaccaggccagaccgcacgtattacctgtggcggtaacctgatcggctctaagctggttcactggtatcagcaaaaaccaggccaggcaccagtactggttgtgtacgatgatggcgatcgtccaagctggattccagagcgtttcagcggctctaattccggcaacaccgctactctgactatttcccgtgttgaagccggcgatgaagccgactactattgccaggtctgggactctggctccgacggtgtagtctttggcgggggcaccaaactgaccgttttg

SEQ ID NO: 239 - DNA, 735 bp, Artificial Sequence
  Description of Artificial Sequence Synthetic polynucleotide
caaatgcagctggtcgagtctggcggtggggtagtgcaaccaggccgttctctgcgtcttagctgcgccgcatctggttttacctttcgtacctacggtatgcactgggtgcgtcaggcaccaggcaaaggtctggaatgggtcgcagtaatctggtatgatggtagcaataaacactatgctgactcagtcaaaggccgtttcaccatcacccgtgataacagcaagaacactcttaacttacagatgaactctctgcgtgccgaagacaccgccgtttactactgtgcccgtgcaccacagtgggtcttagtatccgaagcattcgatctttggggccagggcactatggtgaccgttagctctggcggtggtggtagcggaggcggaggatcaggtggaggcggcagttcttacgtgctgactcaaccaccatcagtgtctgtagcaccaggccagaccgcacgtattacctgtggcggtaacctgatcggctctaagctggttcactggtatcagcaaaaaccaggccaggcaccagtactggttgtgtacgatgatagcgatcgtccaagccgtattccagagcgtttcagcggctctaattccggcaacaccgctactctgactatttcccgtgttgaagccggcgatgaagccgactactattgccaggtctgggactccggctccgacggtgtagtctttggcgggggcaccaaactgaccgttttg

SEQ ID NO: 240 - DNA, 735 bp, Artificial Sequence
  Description of Artificial Sequence Synthetic polynucleotide
caaatgcagctggtcgagtctggcggtggggtagtgcaaccaggccgttctctgcgtcttagctgcgccgcatctggttttacctttcgtacctacggtatgcactgggtgcgtcaggcaccaggcaaaggtctggaatgggtcgcagtaatctggtatgatggtagcaataaacactatgctgactcagtcaaaggccgtttcaccatcacccgtgataacagcaagaacactcttaacttacagatgaactctctgcgtgccgaagacaccgccgtttactactgtgcccgtgcaccacagtgggaattagtagcggaggcattcgatctgtggggccagggcactatggtgaccgttagctctggcggtggtggtagcggaggcggaggatcaggtggaggcggcagttcttacgtgctgactcaaccaccatcagtgtctgtagcaccaggccagaccgcacgtattacctgtggcggtaacatcatcggctctaagctggttcactggtatcagcaaaaaccaggccaggcaccagtactggttgtgtacgatgatagcgatcgtccaagcgcaattccagagcgtttcagcggctctaattccggcaacaccgctactctgactatttcccgtgttgaagccggcgatgaagccgactactattgccaggtctgggactccagctccgacggtgtagtctttggcgggggcaccaaactgaccgttttg

SEQ ID NO: 241 - DNA, 735 bp, Artificial Sequence
  Description of Artificial Sequence Synthetic polynucleotide
caaatgcagctggtcgagtctggcggtggggtagtgcaaccaggccgttctctgcgtcttagctgcgccgcatctggttttacctttcgtacctacggtatgcactgggtgcgtcaggcaccaggcaaaggtctggaatgggtcgcagtaatctcttatgatggtagcaataaacactatgctgactcagtcaaaggccgtttcaccatcacccgtgataacagcaagaacactcttaacttacagatgaactctctgcgtgccgaagacaccgccgtttactactgtgcccgtagcccacagtgggaatgggtacacgaagcattcgatatgtggggccagggcactatggtgaccgttagctctggcggtggtggtagcggaggcggaggatcaggtggaggcggcagttcttacgtgctgactcaaccaccatcagtgtctgtagcaccaggccagaccgcacgtattacctgtggcggtaacaacatcggctctaagctggttcactggtatcagcaaaaaccaggccaggcaccagtactggttgtgtacgatgatagcgatcgtccaagcggtattccagagcgtttcagcggctctaattccggcaacaccgctactctgactatttcccgtgttgaagccggcgatgaagccgactactattgccaggtctgggactctagctccgacggtgtagtctttggcgggggcaccaaactgaccgttttg

SEQ ID NO: 242 - DNA, 735 bp, Artificial Sequence
  Description of Artificial Sequence Synthetic polynucleotide
caaatgcagctggtcgagtctggcggtggggtagtgcaaccaggccgttctctgcgtcttagctgcgccgcatctggttttacctttcgtacctacggtatgcactgggtgcgtcaggcaccaggcaaaggtctggaatgggtcgcagtaatctcttatgatggtagcaataaacactatgctgactcagtcaaaggccgtttcaccatcacccgtgataacagcaagaacactcttaacttacagatgaactctctgcgtgccgaagacaccgccgtttactactgtgcccgttcgccacagtgggaatgggtacacgaagcattcgatctctggggccagggcactatggtgaccgttagctctggcggtggtggtagcggaggcggaggatcaggtggaggcggcagttcttacgtgctgactcaaccaccatcagtgtctgtagcaccaggccagaccgcacgtattacctgtggcggtaacatcctgggctctaagctggttcactggtatcagcaaaaaccaggccaggcaccagtactggttgtgtacgatgatagcgatcgtccaagcgaaattccagagcgtttcagcggctctaattccggcaacaccgctactctgactatttcccgtgttgaagccggcgatgaagccgactactattgccaggtctgggacaccagctccgacggtgtagtctttggcgggggcaccaaactgaccgttttg

SEQ ID NO: 243 - DNA, 735 bp, Artificial Sequence
  Description of Artificial Sequence Synthetic polynucleotide
caaatgcagctggtcgagtctggcggtggggtagtgcaaccaggccgttctctgcgtcttagctgcgccgcatctggttttacctttcgtacctacggtatgcactgggtgcgtcaggcaccaggcaaaggtctggaatgggtcgcagtaatctggtatgatggtagcaataaacactatgctgactcagtcaaaggccgtttcaccatcacccgtgataacagcaagaacactcttaacttacagatgaactctctgcgtgccgaagacaccgccgtttactactgtgcccgtagcccacagtgggaatgggtacacgaagcattcgatctatggggccagggcactatggtgaccgttagctctggcggtggtggtagcggaggcggaggatcaggtggaggcggcagttcttacgtgctgactcaaccaccatcagtgtctgtagcaccaggccagaccgcacgtattacctgtggcggtaacaacatcggctctaagctggttcactggtatcagcaaaaaccaggccaggcaccagtactggttgtgtacgatgatagcgatcgtccaagccggattccagagcgtttcagcggctctaattccggcaacaccgctactctgactatttcccgtgttgaagccggcgatgaagccgactactattgccaggtctgggacactagctccgacggtgtagtctttggcgggggcaccaaactgaccgttttg

SEQ ID NO: 244 - DNA, 735 bp, Artificial Sequence
  Description of Artificial Sequence Synthetic polynucleotide
caaatgcagctggtcgagtctggcggtggggtagtgcaaccaggccgttctctgcgtcttagctgcgccgcatctggttttacctttcgtacctacggtatgcactgggtgcgtcaggcaccaggcaaaggtctggaatgggtcgcagtaatctggtatgatggtagcaataaacactatgctgactcagtcaaaggccgtttcaccatcacccgtgataacagcaagaacactcttaacttacagatgaactctctgcgtgccgaagacaccgccgtttactactgtgcccgttcgccacagtgggaatgggtacacgaagcattcgatctctggggccagggcactatggtgaccgttagctctggcggtggtggtagcggaggcggaggatcaggtggaggcggcagttcttacgtgctgactcaaccaccatcagtgtctgtagcaccaggccagaccgcacgtattacctgtggcggtaacctgatcggctctaagctggttcactggtatcagcaaaaaccaggccaggcaccagtactggttgtgtacgatgatggcgatcgtccaagctggattccagagcgtttcagcggctctaattccggcaacaccgctactctgactatttcccgtgttgaagccggcgatgaagccgactactattgccaggtctgggactccagctccgacggtgtagtctttggcgggggcaccaaactgaccgttttg

SEQ ID NO: 245 - protein, 8 aa, Artificial Sequence
  Description of Artificial Sequence Synthetic peptide
GFTFRTYG

SEQ ID NO: 246 - protein, 8 aa, Artificial Sequence
  Description of Artificial Sequence Synthetic peptide
  MOD_RES (2)..(2): Trp or Ser
IXYDGSNK

SEQ ID NO: 247 - protein, 15 aa, Artificial Sequence
  Description of Artificial Sequence Synthetic peptide
  MOD_RES (3)..(3): Ala or Ser
  MOD_RES (7)..(7): Glu, Gln, Met, Leu or Val
  MOD_RES (8)..(8): Leu, Trp or Tyr
  MOD_RES (9)..(9): Val or Thr
  MOD_RES (10)..(10): His, Ala or Ser
  MOD_RES (15)..(15): Ile, Leu or Met
ARXPQWXXXXEAFDX

SEQ ID NO: 248 - protein, 7 aa, Artificial Sequence
  Description of Artificial Sequence Synthetic peptide
  MOD_RES (1)..(1): Asn, Leu or Ile
  MOD_RES (2)..(2): Leu or Ile
  MOD_RES (6)..(6): Ser or Leu
XXGSKXV

SEQ ID NO: 249 - protein, 3 aa, Artificial Sequence
  Description of Artificial Sequence Synthetic peptide
  MOD_RES (3)..(3): Ser or Gly
DDX

SEQ ID NO: 250 - protein, 11 aa, Artificial Sequence
  Description of Artificial Sequence Synthetic peptide
  MOD_RES (5)..(5): Ser or Thr
  MOD_RES (6)..(6): Ser or Gly
  MOD_RES (9)..(9): His or Gly
QVWDXXSDXVV

SEQ ID NO: 251 - protein, 8 aa, Artificial Sequence
  Description of Artificial Sequence Synthetic peptide
  MOD_RES (2)..(2): Any amino acid
IXYDGSNK

SEQ ID NO: 252 - protein, 15 aa, Artificial Sequence
  Description of Artificial Sequence Synthetic peptide
  MOD_RES (3)..(12): Any amino acid
  MOD_RES (15)..(15): Any amino acid
ARXXXXXXXXXXFDX

SEQ ID NO: 253 - protein, 7 aa, Artificial Sequence
  Description of Artificial Sequence Synthetic peptide
  MOD_RES (1)..(2): Any amino acid
  MOD_RES (6)..(6): Any amino acid
XXGSKXV

SEQ ID NO: 254 - protein, 3 aa, Artificial Sequence
  Description of Artificial Sequence Synthetic peptide
  MOD_RES (3)..(3): Any amino acid
DDX

SEQ ID NO: 255 - protein, 11 aa, Artificial Sequence
  Description of Artificial Sequence Synthetic peptide
  MOD_RES (5)..(6): Any amino acid
  MOD_RES (9)..(9): Any amino acid
QVWDXXSDXVV

SEQ ID NO: 256 - protein, 15 aa, Artificial Sequence
  Description of Artificial Sequence Synthetic peptide
GGGGSGGGGSGGGGS

SEQ ID NO: 257 - DNA, 45 bp, Artificial Sequence
  Description of Artificial Sequence Synthetic oligonucleotide
ggcggtggtggtagcggaggcggaggatcaggtggaggcggcagt

SEQ ID NO: 258 - protein, 8 aa, Artificial Sequence
  Description of Artificial Sequence Synthetic peptide
GFAFRTYG

SEQ ID NO: 259 - protein, 8 aa, Artificial Sequence
  Description of Artificial Sequence Synthetic peptide
IWYDGSNT

SEQ ID NO: 260 - protein, 15 aa, Artificial Sequence
  Description of Artificial Sequence Synthetic peptide
ARAPQWYLSAEAFDL

SEQ ID NO: 261 - protein, 6 aa, Artificial Sequence
  Description of Artificial Sequence Synthetic peptide
LIGSKL

SEQ ID NO: 262 - protein, 3 aa, Artificial Sequence
  Description of Artificial Sequence Synthetic peptide
DDS

SEQ ID NO: 263 - protein, 11 aa, Artificial Sequence
  Description of Artificial Sequence Synthetic peptide
QVWDHSSDHVV

SEQ ID NO: 264 - protein, 122 aa, Artificial Sequence
  Description of Artificial Sequence Synthetic polypeptide
QMQLVESGGGVVQPGRSLRLSCAASGFAFRTYGMHWVRQAPGKGLEWVAVIWYDGSNTHYADSVKGRFTITRDNSKNTLNLQMNSLRAEDTAVYYCARAPQWYLSAEAFDLWGQGTMVTVSS

SEQ ID NO: 265 - protein, 108 aa, Artificial Sequence
  Description of Artificial Sequence Synthetic polypeptide
SYVLTQPPSVSVAPGQTARITCGGNLIGSKLVHWYQQKPGQAPVLVVYDDSDRPSRIPERFSGSNSGNTATLTISRVEAGDEADYYCQVWDHSSDHVVFGGGTKLTVL

SEQ ID NO: 266 - protein, 122 aa, Artificial Sequence
  Description of Artificial Sequence Synthetic polypeptide
QVQLVQSGAEVKKPGASVKVSCKASGFAFRTYGLSWVRQAPGQGLEWMGWIWYDGSNTNYGQEFQGRVTMTTDTSTSTAYMELRSLRSDDTAVYYCARAPQWYLSAEAFDLWGRGTLVTVSS

SEQ ID NO: 267 - protein, 108 aa, Artificial Sequence
  Description of Artificial Sequence Synthetic polypeptide
SYVLTQPPSVSVAPGKTARITCGGNLIGSKLVHWYQQKPGQAPVLVIYDDSDRPSRIPERFSGSNSGNTATLTISRVEAGDEADYYCQVWDHSSDHVVFGGGTKLTVL

SEQ ID NO: 268 - protein, 122 aa, Artificial Sequence
  Description of Artificial Sequence Synthetic polypeptide
QMQLVESGGGVVQPGRSLRLSCAASGFTFRTYGMHWVRQAPGKGLEWVADIWYDGSNKHYADSVKGRFTITRDNSKNTLNLQMNSLRAEDTAVYYCARAPQWYLVAEPFDLWGQGTMVTVSS

SEQ ID NO: 269 - protein, 108 aa, Artificial Sequence; identical to SEQ ID NO: 112
  Description of Artificial Sequence Synthetic polypeptide

SEQ ID NO: 270 - DNA, 366 bp, Artificial Sequence
  Description of Artificial Sequence Synthetic polynucleotide
caaatgcagctggtcgagtctggcggtggggtagtgcaaccaggccgttctctgcgtcttagctgcgccgcatctggttttgcgtttcgtacctacggtatgcactgggtgcgtcaggcaccaggcaaaggtctggaatgggtcgcagtaatctggtatgatggtagcaatacccactatgctgactcagtcaaaggccgtttcaccatcacccgtgataacagcaagaacactcttaacttacagatgaactctctgcgtgccgaagacaccgccgtttactactgtgcccgtgcaccacagtggtacttaagcgcggaagcattcgatctatggggccagggcactatggtgaccgttagctct

SEQ ID NO: 271 - DNA, 324 bp, Artificial Sequence
  Description of Artificial Sequence Synthetic polynucleotide
tcttacgtgctgactcaaccaccatcagtgtctgtagcaccaggccagaccgcacgtattacctgtggcggtaacctgatcggctctaagctggttcactggtatcagcaaaaaccaggccaggcaccagtactggttgtgtacgatgatagcgatcgtccaagccgtattccagagcgtttcagcggctctaattccggcaacaccgctactctgactatttcccgtgttgaagccggcgatgaagccgactactattgccaggtctgggaccacagctccgaccatgtagtctttggcgggggcaccaaactgaccgttttg

SEQ ID NO: 272 - DNA, 366 bp, Artificial Sequence
  Description of Artificial Sequence Synthetic polynucleotide
caaatgcagctggtcgagtctggcggtggggtagtgcaaccaggccgttctctgcgtcttagctgcgccgcatctggttttacctttcgtacctacggtatgcactgggtgcgtcaggcaccaggcaaaggtctggaatgggtcgcagacatctggtatgatggtagcaataaacactatgctgactcagtcaaaggccgtttcaccatcacccgtgataacagcaagaacactcttaacttacagatgaactctctgcgtgccgaagacaccgccgtttactactgtgcccgtgcaccacagtggtacttagtagcggaaccgttcgatctatggggccagggcactatggtgaccgttagctct

SEQ ID NO: 273 - DNA, 324 bp, Artificial Sequence; identical to SEQ ID NO: 167
  Description of Artificial Sequence Synthetic polynucleotide

SEQ ID NO: 274 - protein, 8 aa, Artificial Sequence
  Description of Artificial Sequence Synthetic peptide
GFTFRTYG

SEQ ID NO: 275 - protein, 8 aa, Artificial Sequence
  Description of Artificial Sequence Synthetic peptide
IWYDGSNK

SEQ ID NO: 276 - protein, 15 aa, Artificial Sequence
  Description of Artificial Sequence Synthetic peptide
ARAPQWYLVAEPFDL

SEQ ID NO: 277 - protein, 11 aa, Artificial Sequence
  Description of Artificial Sequence Synthetic peptide
QVWDSSSDGVV

SEQ ID NO: 278 - protein, 122 aa, Artificial Sequence
  Description of Artificial Sequence Synthetic polypeptide
QMQLVESGGGVVQPGRSLRLSCAASGFAFRTYGMHWVRQAPGKGLEWVAVIWYDGSNTHYADSVKGRFTITRDNSKNTLNLQMNSLRAEDTAVYYCVRAPQWYLSAEAFDLWGQGTMVTVSS

SEQ ID NO: 279 - protein, 108 aa, Artificial Sequence
  Description of Artificial Sequence Synthetic polypeptide
SYVLTQPPSVSVAPGQTARITCGGNLIGAKLVHWYQQKPGQAPVLVVYDDSDRPSRIPERFSGSNSGNTATLTISRVEAGDEADYYCQVWDHSSDHVVFGGGTKLTVL

SEQ ID NO: 280 - protein, 122 aa, Artificial Sequence
  Description of Artificial Sequence Synthetic polypeptide
QMQLVESGGGVVQPGRSLRLSCAASGFAFRTYGMHWVRQAPGKGLEWVAVIWDDGSNTHYADSVKGRFTITRDNSKNTLNLQMNSLRAEDTAVYYCARAPQWYLSAEAFDLWGQGTMVTVSS

SEQ ID NO: 281 - protein, 108 aa, Artificial Sequence; identical to SEQ ID NO: 265
  Description of Artificial Sequence Synthetic polypeptide

SEQ ID NO: 282 - protein, 122 aa, Artificial Sequence; identical to SEQ ID NO: 278
  Description of Artificial Sequence Synthetic polypeptide

SEQ ID NO: 283 - protein, 108 aa, Artificial Sequence
  Description of Artificial Sequence Synthetic polypeptide
SYVLTQPPSVSVAPGQTARITCGGNLIGSKLVHWYQQKPGQAPVLVVYDDSDRPSRIPERFSGSNSGNTATLTISRVEAGDEADYYCQVWDYSSNHVVFGGGTKLTVL

SEQ ID NO: 284 - protein, 122 aa, Artificial Sequence
  Description of Artificial Sequence Synthetic polypeptide
QMQLVESGGGVVQPGRSLRLSCAASGFAFDTYGMHWVRQAPGKGLEWVAVIWYDGSNTVYADSVKGRFTITRDNSKNTLNLQMNSLRAEDTAVYYCARAPQWYLSAEAFDLWGQGTMVTVSS

SEQ ID NO: 285 - protein, 108 aa, Artificial Sequence
  Description of Artificial Sequence Synthetic polypeptide
SYVLTQPPSVSVAPGQTARITCGGNLIGSKLVHWYQQKPGQAPVLVVYDDSDRPSRIPERFSGSNSGNTATLTISRVEAGDEADYYCQVWDHSSDHYVFGGGTKLTVL

SEQ ID NO: 286 - protein, 122 aa, Artificial Sequence; identical to SEQ ID NO: 278
  Description of Artificial Sequence Synthetic polypeptide

SEQ ID NO: 287 - protein, 108 aa, Artificial Sequence
  Description of Artificial Sequence Synthetic polypeptide
SYVLTQPPSVSVAPGETATITCGGNLIGSKLVHWYQQKPGQAPVLVVYDDSDRPSRIPERFSGSNSGNTATLTISRVEAGDEADYYCQVWDHSSDHVVFGGGTKLTVL

SEQ ID NO: 288 - protein, 122 aa, Artificial Sequence
  Description of Artificial Sequence Synthetic polypeptide
QMQLVESGGGVVQPGRSLRLSCAASGFAFRTYGMHWVRQAPGKGLEWVAVIWYDGSATHYADSVKGRFTITRDNSKNTLNLQMNSLRAEDTAVYYCVRAPQWYLSAEAFDLWGQGTMVTVSS

SEQ ID NO: 289 - protein, 108 aa, Artificial Sequence
  Description of Artificial Sequence Synthetic polypeptide
SYVLTQPPSVSVAPGQTATITCGGNLIGSKLVHWYQQKPGQAPVLVVYDDSDRPSRIPERFSGSNIGNTATLTISRVEAGDEADYYCQVWDHSSDHVVFGGGTKLTVL

SEQ ID NO: 290 - protein, 122 aa, Artificial Sequence
  Description of Artificial Sequence Synthetic polypeptide
QMQLVESGGGVVQPGRSLRLSCAASGFDFRTYGMHWVRQAPGKGLEWVAVIWYDGSITHYADSVKGRFTITRDNSKNTLNLQMNSLRAEDTAVYYCVRAPQWYLTAEAFDLWGQGTMVTVSS

SEQ ID NO: 291 - protein, 108 aa, Artificial Sequence
  Description of Artificial Sequence Synthetic polypeptide
SYVLTQPPSVSVAPGQTARITCGGNLIGTKLVHWYQQKPGQAPVLVVYDDSDRPSRIPERFSGSNSGNTATLTISRVEAGDEADYYCQVWDHNEDEVVFGGGTKLTVL

SEQ ID NO: 292 - DNA, 366 bp, Artificial Sequence
  Description of Artificial Sequence Synthetic polynucleotide
cagatgcagttggtggagtccggaggtggagtggtgcaaccagggcgttccttgcgtttgtcttgtgctgcttccggattcgcctttcgtacatatggcatgcattgggtgcgtcaagcacctggtaagggcctggagtgggttgccgttatttggtacgacggctccaacacccactacgcagatagcgtgaaaggacgtttcactattacccgtgataactccaagaatacccttaacctgcagatgaatagcttgcgtgctgaggacacagcagtatattactgcgtccgtgcaccacaatggtacctgagcgccgaggcctttgatctgtgggggcagggcacaatggtgaccgtttcctca

SEQ ID NO: 293 - DNA, 324 bp, Artificial Sequence
  Description of Artificial Sequence Synthetic polynucleotide
agttacgtgctgacacaacctccaagtgttagtgtcgcaccaggacaaacagcacgtattacatgtggaggaaatcttatcggtgccaagctggtgcactggtaccagcagaaacctggtcaggccccagtactggttgtgtatgatgacagcgaccgtccaagccgtatcccagaacgtttttctgggagcaactcaggtaatacagccactctgaccatttcacgtgttgaggcaggagatgaggccgattattattgccaagtatgggaccacagctctgaccatgttgtttttggcggagggactaagctgaccgtgctt

SEQ ID NO: 294 - DNA, 366 bp, Artificial Sequence
  Description of Artificial Sequence Synthetic polynucleotide
cagatgcaactggtggagtcaggaggcggcgtggtgcagccaggacgttctctgcgtctgtcttgcgcagcttccgggttcgcctttcgtacctatgggatgcattgggtgcgtcaggctccaggtaagggactggagtgggtcgctgttatttgggacgacggaagtaacactcattacgccgacagcgtgaagggccgtttcacaattacccgtgacaattccaagaataccttgaacctgcagatgaactctcttcgtgctgaagataccgccgtgtactattgcgcccgtgctccacagtggtatctgtcagcagaggccttcgacctgtggggacagggaacaatggtgaccgtatcttca

SEQ ID NO: 295 - DNA, 324 bp, Artificial Sequence
  Description of Artificial Sequence Synthetic polynucleotide
tcctatgtgctgacacagccacctagcgtgagcgtcgccccaggtcagaccgctcgtatcacttgtggcgggaaccttatcggcagcaagctggtgcactggtaccagcagaagcctggccaagcacctgtgctggtcgtttatgacgactctgaccgtccatcccgtatcccagaacgtttctctggctctaactctgggaataccgctaccctgacaatctcacgtgttgaagctggcgacgaggcagattattattgccaagtctgggatcactccagcgatcacgtcgtgttcggaggcggaacaaaattgactgtcctg

SEQ ID NO: 296 - DNA, 366 bp, Artificial Sequence
  Description of Artificial Sequence Synthetic polynucleotide
caaatgcagctggtggaatccgggggtggggtcgtccagcctggccgtagtctgcgtctttcctgtgccgcatcaggctttgctttccgtacctacgggatgcactgggtgcgtcaggccccaggaaagggacttgaatgggtggctgtcatctggtacgatggttccaacacacactatgccgattcagtgaaagggcgtttcaccattactcgtgacaatagtaagaatactctgaatctgcaaatgaattcactgcgtgctgaggacaccgctgtttattactgtgtgcgtgctcctcagtggtacctgagtgccgaagctttcgatttgtggggacagggcacaatggtgacagtcagttct

SEQ ID NO: 297 - DNA, 324 bp, Artificial Sequence
  Description of Artificial Sequence Synthetic polynucleotide
tcttacgtgttgacacaaccaccaagtgttagtgtcgcacctggccaaaccgctcgtatcacctgtggtgggaatcttattggctctaagctggtgcactggtatcagcagaaaccaggccaggctccagtactggtggtgtacgacgactctgaccgtccaagccgtatcccagagcgtttcagtggctctaactccgggaacacagcaactcttacaatttcacgtgtggaggccggtgatgaagccgactactattgccaggtttgggactacagtagtaatcacgtggttttcggtggtggtaccaagctgactgtgttg

SEQ ID NO: 298 - DNA, 366 bp, Artificial Sequence
  Description of Artificial Sequence Synthetic polynucleotide
cagatgcagctggttgaatctggcggcggtgtggtccagcctggtcgtagcctgcgtctgtcctgtgctgcaagcggatttgcctttgacacctatgggatgcactgggtacgtcaggccccaggaaagggcctggaatgggtggcagttatctggtatgatggttctaataccgtgtatgccgactccgttaaaggccgtttcactatcacccgtgataatagtaaaaacacactgaacctgcagatgaatagcttgcgtgctgaggacaccgcagtgtactactgtgcccgtgctcctcagtggtatctgtcagcagaggccttcgatctgtggggccaagggacaatggtgaccgtgtcttcc

SEQ ID NO: 299 - DNA, 324 bp, Artificial Sequence
  Description of Artificial Sequence Synthetic polynucleotide
agctatgttctgactcaaccacctagtgtgagtgtggcccctggtcagactgcacgtattacctgtggcggaaaccttatcggcagtaagctggttcattggtatcagcagaagccaggacaggcaccagtgctggtcgtttacgacgatagtgaccgtccatcacgtatcccagagcgttttagcgggtccaattccggaaatacagcaaccttgaccattagccgtgtggaagccggcgatgaagctgattattactgccaggtatgggaccattcctccgaccactacgtttttggtggcggaactaagctgacagtcttg

SEQ ID NO: 300 - DNA, 366 bp, Artificial Sequence
  Description of Artificial Sequence Synthetic polynucleotide
cagatgcagcttgttgagagcggcggaggcgtggtgcaaccaggccgttcattgcgtctgtcctgcgccgccagcggctttgcttttcgtacatacggcatgcactgggtgcgtcaggcccctggcaaggggctggaatgggtcgccgtgatttggtatgacggtagtaacacccattatgctgattccgtcaagggacgtttcactatcacccgtgacaatagcaaaaatacactgaatctgcaaatgaattcattgcgtgccgaagacaccgccgtatattactgtgtccgtgccccacagtggtacctgagcgctgaggccttcgatctgtggggtcaggggactatggtgaccgtatcatcc

SEQ ID NO: 301 - DNA, 324 bp, Artificial Sequence
  Description of Artificial Sequence Synthetic polynucleotide
tcttacgtgcttactcagcctcctagcgtctcagtggccccaggcgagacagcaaccattacatgcgggggtaatttgatcggtagcaagctggtgcattggtatcagcagaagcctggccaggccccagtgctggttgtatatgacgatagtgatcgtccaagtcgtatccctgagcgttttagcggatctaactccggcaacacagccacattgacaatcagccgtgtggaggcaggcgatgaggccgactactactgccaagtttgggaccactcctctgaccacgtggtatttggcggaggaacaaagcttacagttttg

SEQ ID NO: 302 - DNA, 366 bp, Artificial Sequence
  Description of Artificial Sequence Synthetic polynucleotide
cagatgcagctggtggagagtggaggtggtgtggtgcaacctgggcgtagcctgcgtttgagctgcgctgcctctggatttgccttccgtacctatggcatgcactgggtgcgtcaggctccaggaaaggggttggaatgggtggctgtgatttggtacgacgggagcgccacacattacgcagacagcgttaagggccgtttcacaattacccgtgacaatagcaaaaatacattgaacctgcagatgaattccctgcgtgcagaggatactgcagtgtactattgcgtccgtgccccacagtggtatctgtcagccgaagccttcgatctgtgggggcagggtactatggtcaccgtaagttcc

SEQ ID NO: 303 - DNA, 324 bp, Artificial Sequence
  Description of Artificial Sequence Synthetic polynucleotide
tcttatgttttgacccaacctccatccgttagcgtggctccaggtcaaacagctaccatcacatgtggcggtaaccttattggctcaaagctggttcattggtatcaacagaaaccaggccaagccccagtgctggtggtgtatgacgacagtgaccgtccttctcgtattcctgagcgtttttccggctctaatattggcaacactgccaccctgaccatttctcgtgtggaagcaggagatgaggcagactattattgtcaggtttgggatcactccagcgatcatgtggtattcggaggtgggacaaaacttactgttctt

SEQ ID NO: 304 - DNA, 366 bp, Artificial Sequence
  Description of Artificial Sequence Synthetic polynucleotide
cagatgcagctggtggaaagtggtgggggagtcgtgcaaccaggacgttccttgcgtctgtcatgcgctgcttcaggtttcgactttcgtacctacggcatgcattgggtgcgtcaggctccaggtaaaggacttgagtgggtcgcagtgatctggtacgacggatcaattactcactacgccgatagcgtgaaaggccgtttcaccatcacccgtgacaactccaagaacaccctgaacttgcagatgaacagtctgcgtgcagaagacactgcagtatattattgtgtccgtgccccacagtggtacttgaccgccgaggcttttgatctgtggggacagggcacaatggtgaccgtatctagc

SEQ ID NO: 305 - DNA, 324 bp, Artificial Sequence
  Description of Artificial Sequence Synthetic polynucleotide
agctacgtgcttacccagccaccatcagtcagtgtggctccaggccaaactgcccgtatcacctgcggcggcaatttgattggcaccaagcttgtgcactggtaccaacagaagccagggcaggcccctgtgctggttgtctacgacgatagtgatcgtccttcccgtattcctgaacgtttctctggaagcaattccggaaacacagccacacttaccatttctcgtgttgaggctggggatgaagccgactactattgccaggtttgggaccacaatgaagacgaagttgtttttggaggaggaactaagctgacagttctg

SEQ ID NO: 306 - protein, 8 aa, Artificial Sequence
  Description of Artificial Sequence Synthetic peptide
GFAFDTYG

SEQ ID NO: 307 - protein, 8 aa, Artificial Sequence
  Description of Artificial Sequence Synthetic peptide
GFDFRTYG

SEQ ID NO: 308 - protein, 8 aa, Artificial Sequence
  Description of Artificial Sequence Synthetic peptide
IWYDGSAT

SEQ ID NO: 309 - protein, 8 aa, Artificial Sequence
  Description of Artificial Sequence Synthetic peptide
IWYDGSIT

SEQ ID NO: 310 - protein, 15 aa, Artificial Sequence
  Description of Artificial Sequence Synthetic peptide
VRAPQWYLSAEAFDL

SEQ ID NO: 311 - protein, 15 aa, Artificial Sequence
  Description of Artificial Sequence Synthetic peptide
VRAPQWYLTAEAFDL

SEQ ID NO: 312 - protein, 6 aa, Artificial Sequence
  Description of Artificial Sequence Synthetic peptide
LIGAKL

SEQ ID NO: 313 - protein, 6 aa, Artificial Sequence
  Description of Artificial Sequence Synthetic peptide
LIGTKL

SEQ ID NO: 314 - protein, 11 aa, Artificial Sequence
  Description of Artificial Sequence Synthetic peptide
QVWDYSSNHVV

SEQ ID NO: 315 - protein, 11 aa, Artificial Sequence
  Description of Artificial Sequence Synthetic peptide
QVWDHSSDHYV

SEQ ID NO: 316 - protein, 11 aa, Artificial Sequence
  Description of Artificial Sequence Synthetic peptide
QVWDHNEDEVV

SEQ ID NO: 317 - protein, 8 aa, Artificial Sequence
  Description of Artificial Sequence Synthetic peptide
IWDDGSNT